(19) United States
(12) Patent Application Publication
(10) Pub. No.: US 2024/0150396 A1
(43) Pub. Date: May 9, 2024

(54) INHIBITORS OF CYSTEINE PROTEASES

(71) Applicants: The Texas A&M University System, College Station, TX (US); The Board of Regents of the University of Texas System, Austin, TX (US)

(72) Inventors: Thomas D. Meek, College Station, TX (US); Linfeng Li, College Station, TX (US); Chien-Te Kent Tseng, Galveston, TX (US); Aleksandra Drelich, Galveston, TX (US)

(21) Appl. No.: 18/280,400

(22) PCT Filed: Mar. 3, 2022
(86) PCT No.: PCT/US2022/018714

§ 371 Date: Sep. 5, 2023

Related U.S. Application Data
(60) Provisional application No. 63/156,198, filed on Mar. 3, 2021.
(60) Provisional application No. 63/156,211, filed on Mar. 3, 2021.

(51) Int. Cl. C07K 5/078; A61P 31/14; A61P 33/02

(52) U.S. Cl. CPC C07K 5/06139; A61P 31/14; A61P 33/02; A61K 38/00

(57) ABSTRACT

Provided herein are pharmaceutical compositions including compounds having Formula (I) in an effective amount to inhibit cysteine protease as well as methods of using thereof.

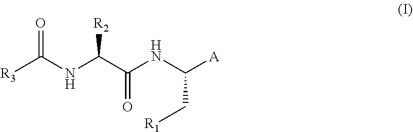

### CROSS-REFERENCE TO RELATED APPLICATIONS

The application claims the benefit of U.S. Provisional Application No. 63/156,198, filed Mar. 3, 2021, and U.S. Provisional Application No. 63/156,211, filed Mar. 3, 2021, which are hereby incorporated herein by reference in their entirety.

### STATEMENT REGARDING FEDERALLY SPONSORED RESEARCH OR DEVELOPMENT

This disclosure was made with Government Support under Grant No. 5R21AI127634 awarded by National Institutes of Health. The Government has certain rights to this disclosure.

### TECHNICAL FIELD

This disclosure relates to inhibitors of cysteine proteases.

### BACKGROUND

Cysteine proteases comprise a large family of proteases that activate other proteases, modify, activate, or inactivate other proteins, and have been associated with a large panel of diseases. The papain sub-family of cysteine proteases have similar active-site structures, containing a conserved cysteine residue and a closely-associated, conserved histidine residue, suggesting that cysteine proteases of the papain sub-family have similar chemical mechanisms. Aberrant activities of the papain-like cysteine proteases (names in parentheses below) result in many diseases, many of which are of unmet or under-met medical needs, including malaria (falcipains-2 and -3), Chagas disease (cruzain/cruzipain), African sleeping sickness (TbCatB, TbCatL, rhodesain), some cancers (caspases and cathepsin L), osteoporosis (cathepsin K), and respiratory ailments including COPD and asthma (cathepsins C and S). In addition, coronaviruses such as SARS CoV have two essential cysteine proteases, the 3-chymotrypsin-like protease (3CL-PR) and the papain-like protease (PL-PR) for which existing inhibitors of other cysteine proteases may be adapted to inhibit or inactivate these enzymes. Furthermore, human cathepsin L is also involved in cellular uptake of SARS CoV-2, and inhibitors of this cysteine protease exert anti-CoV-2 activity. It has been difficult to discover inhibitors for these enzymes using high-throughput screening methodology, which has encouraged the discovery of inhibitors of these enzymes by rational design.

It would be advantageous to have an inhibitor of cysteine proteases that has low nanomolar potency for its enzyme target and is a reversible inactivator of the enzyme.

### SUMMARY

A method for treating, inhibiting, decreasing, reducing, ameliorating and/or preventing a Chagas disease, African trypanosomiasis, malaria, and coronavirus infections and/or method for treating, inhibiting, decreasing, reducing, ameliorating and/or preventing the disease and/or symptoms associated with said infections in a subject in need thereof is provided including administering a therapeutically effective amount of a protease inhibitor described herein, or a pharmaceutically acceptable salt or derivatives thereof. The compounds described herein can inhibit the cruzain, cruzipain, human cathepsin B, human cathepsin L, TbCatB, TbCatL, rhodesain, brucipain, falcipain-2, falcipain-3, and/or coronaviral cysteine proteases 3CL-PR and PL-PR.

In some embodiments, the coronavirus causing the infection may be an alphacoronavirus, a betacoronavirus, a gammacoronavirus, or a deltacoronavirus. The coronavirus disease may be selected from a common cold, pneumonia, pneumonitis, bronchitis, severe acute respiratory syndrome (SARS), coronavirus disease 2019 (COVID19), Middle East respiratory syndrome (MERS), sinusitis, porcine diarrhea, porcine epidemic diarrhea, avian infections bronchitis, otitis and pharyngitis. In particular embodiments, the coronavirus disease comprises COVID-19. In some embodiments, the coronavirus disease is caused by an infection with avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV.

The protease inhibitor as used in the methods described herein may be administered as a pharmaceutical composition. The protease inhibitor as used in the methods described herein may also be administered with one or more additional active agents, for example an antimicrobial agent, an anti-inflammatory agent, or an antiseptic agent.

In some embodiments, the compound is present in an effective amount to inhibit a viral or non-viral cysteine protease.

Another embodiment is a method of killing or inhibiting the growth of a protozoan, the method comprising: contacting the protozoan with a compound above, in an amount effective to kill or inhibit the growth of the protozoan.

In another embodiment, the protozoan is Trypanosoma cruzi, Trypanosoma brucei brucei, Trypanosoma brucei rhodiense, Trypanosomo brucei gambiense, Plasmodium falciparum, or Plasmodium vivax.

An embodiment of the disclosure is a method of identifying an inhibitor of cruzain, cruzipain, cathepsin B, cathepsin L, TbCatB, TbCatL, rhodesain, brucipain, falcipain-2, falcipain-3, and/or coronaviral cysteine proteases 3CL-PR and PL-PR comprising adding a peptide side chain to a PVH or masked aldehyde (MA); and assaying whether the peptide side chain-PVH or peptide side chain-MA inhibits at least one selected from the group consisting of cruzain, cruzipain, cathepsin B, cathepsin L, TbCatB, TbCatL, rhodesain, brucipain, falcipain-2, falcipain-, and/or coronaviral cysteine proteases 3CL-PR and PL-PR.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts the X-ray structure of cruzain and the K11777 analogue Cbz-Phe-Phe-2VS (Compound 1; Table 1) demonstrating the presence of a C—S bond between the vinyl sulfone and Cys-25. P₁, P₂, and P₃ indicate the peptide sidechains and the P₂:S₂ interaction with Glu-208 is depicted. For clarity, a peptide or peptide analogues binding to a protease with a cleavage site of —CO*NH₂—, amino acid sidechains are designated as: P₃-P₂-P₃-P₂-P₁—CO*NH₂—P_{1′}-P_{2′}-P_{3′}, according to nomenclature of Schechter and Berger (Schechter I, Berger A. On the size of the active site in proteases. I. Papain Biochem Biophys Res Commun. 1967 Apr. 20; 27(2):157-62. doi: 10.1016/s0006-291x(67)80055-x. PMID: 6035483.)

FIG. 2A-2B FIG. 2A depicts a graph of time-dependent inhibition of cruzain by Compound 12 (Table 1). The reaction was initiated by adding cruzain (100 pM) to 20 μM Cbz-Phe-Arg-AMC and 0-20 μM Compound 12 (pH 7.5) (2A). Data were fitted to [P]=vₛt+[(vᵢ−vₛ)/k_{obs}][1−e^{-(kobs}*^{t)}] in which product AMC (P) is measured as relative fluorescence units (RFUs), vᵢ and vₛ are, respectively, initial and steady-state enzymatic rates, t is sec, and k_{obs} is the observed rate constant of conversion of vᵢ to vₛ. Replots of k_{obs} vₛ. [Compound 12] allow calculation of values of Kᵢ and Kᵢ* as reported (Table 1). FIG. 2B depicts a graph of time-dependent inhibition of cruzain by Compound 12. Following 1 hr pre-incubation of 100 μM cruzain with 0-6.0 μM Compound 12, the reaction was initiated by addition of 20 μM Cbz-Phe-Arg-AMC. Data were fitted to [P]=vₛt−[(vᵢ−)/k_{obs}][1−e^{-(kobs}*^{t)}] in which product AMC (P) is measured as fluorescence (RFUs), vᵢ and vₛ are, respectively, initial and steady-state enzymatic rates, t is sec, and k_{obs} is the observed rate constant of conversion of vᵢ to vₛ. Replots of k_{obs} vₛ. [Compound 12] allow calculation of values of Kᵢ and Kᵢ* as reported (Table 1).

FIG. 3A-3D FIG. 3A depicts a graph of the effects of cruzain inhibitor (K11777) on growth of axenic cultures of T. brucei brucei epimastigotes. Cells were counted, and re-fed with medium containing inhibitors at indicated times, and plotted data are average values (n=2) with error bars. FIG. 3B depicts a graph of the effects of cruzain inhibitor (Compound 12) on growth of axenic cultures of T. brucei brucei trypomastigotes. Cells were counted, and re-fed with medium containing inhibitors at indicated times, and plotted data are average values (n=2) with error bars. FIG. 3C depicts a graph of the effects of cruzain inhibitor (K11777) on growth of axenic cultures of T. cruzi epimastigotes. Cells were counted, and re-fed with medium containing inhibitors at indicated times, and plotted data are average values (n=2) with error bars. FIG. 3D depicts a graph of the effects of cruzain inhibitor (Compound 10) on growth of axenic cultures of T. cruzi epimastigotes. Cells were counted, and re-fed with medium containing inhibitors at indicated times, and plotted data are average values (n=2) with error bars.

FIG. 4. illustrates the structures of K11777, GSK2793660, and Cbz-Phe-Phe-vinyl-2Pyrmd 7 with thia-Michael addition of Cys25 to the vinyl groups in these compounds, including the putative adduct 7a, which reverts to the fully conjugated 7 upon the reverse of adduct formation. The common bioisosteric atoms of K11777, GSK2793660, and 7 are highlighted in red. X is an electron-donating or electron-withdrawing substituent.

FIG. 5 illustrates a new series of reversible but time-dependent inhibitors of cruzain, composed of a dipeptide scaffold appended to vinyl heterocycles meant to provide replacements for the irreversible reactive “warheads” of vinyl sulfone inactivators of cruzain.

FIG. 6A-B illustrates molecular models of compound 9 bound to cruzain. (A) K11777 is superimposed with a binding pose in which a covalent bond is formed between the β-carbon of the vinyl group of 9. (B) Binding pose of 9 in which no covalent bond is formed with Cys25. Dashed lines represent hydrogen bonds with surrounding residues for 9 and K11777. Red dashed lines are measurements between the catalytic dyad and the vinyl moiety of 9.

FIG. 7 is a scheme illustrating the general synthetic route to PVHIs.³⁵

FIG. 8 is a graph showing time courses of depletion of 0.5 mM K11777 (circle), compound 12 (circle), and compound 15 (circle) upon formation of adducts (squares) with 1 mM glutathione. Lines drawn through the curves for substrate depletion and adduct formation were respectively [substrate]=(0.5 mM−A)(1−exp(−k×t))+C and [adduct]=A×(1−exp(−k×t))+C, with resulting kinetic parameters found in Table 3.

FIG. 9. Is a scheme illustrating the kinetic depiction of inhibition and inactivation.

FIG. 10A-10C are graphs showing the time-dependent inhibition of cruzain by 15. (FIG. 10A) Reaction initiated by addition of cruzain (100 μM) with Cbz-Phe-Arg-AMC (10 μM) and 0-20 μM 15 (pH 7.5). Lines drawn through the experimental data points were from fitting of each inhibitor concentration to eq 3, from which the replot of k_{obs} vₛ [15] is shown in the inset (fitting to eq 4: Kᵢ=2.0±0.9 μM, k₃=0.004±0.001 s⁻¹, and k₄≈0). (FIG. 16B) Following 1 h of preincubation of cruzain (100 μM) with 0-6 μM 15, the reaction was initiated by addition of Cbz-Phe-Arg-AMC (10 μM). (FIG. 16C) Fitting of cruzain inhibition by compound 15 for vᵢ/v₀ and vₛ/v₀ using eq 5 with results of this found in Table 1.

FIG. 11A-11F are graphs showing the effects of cruzain inhibitors on growth of T. cruzi-infected murine cardiomyoblasts in which growth of inhibition of T. cruzi (circle) is superimposed with the viability of the cardiomyoblasts (square). (11A) K11777. (11B) Compound 7. (11C) Compound 11. (11D) Compound 12. (11E) Compound 13. (11F) Compound 15.

FIG. 12A-12D are graphs showing the cell growth inhibition of T. b. brucei. (FIG. 12A) Inhibition of bloodstream forms by 15. (FIG. 12B) Inhibition of bloodstream forms by 7. (FIG. 12C) Inhibition of procyclic forms by 15. (FIG. 12D) Correlation plot of values of EC50 for trypanocidal activity versus T. b. brucei BSFs (closed circles, r²=0.979, slope=0.80) and/in murine cardiomyoblasts (open circles).

FIG. 13 is a scheme illustrating compounds having Formula I and II and their substituents found in masked aldehydes.

FIG. 14 is a scheme illustrating generic double-displacement mechanism of cysteine proteases exhibiting acylation and de-acylation half-reaction steps. AMC is 7-amino-4-methyl-coumarin, which becomes fluorescent upon cleavage of its carboxamide bond (P).

FIG. 15 is a scheme illustrating the mechanism by which di-peptide aldehyde 28 (Table 2) adds to the active-site cysteine of cruzain, and the mechanism by which the lactol of masked aldehyde 30 (Table 2) is opened by the catalytic mechanism of cruzain, elaborating an aldehyde than then forms a reversible hemithioacetal with cruzain.

FIG. 16 is a scheme illustrating electrophilic substituents that form irreversible and reversible covalent adducts with active-site cysteines in cysteine proteases.

FIG. 17 is a scheme illustrating how Cbz-Phe-Phe-vinyl-2-pyrimidine (Compound 7 (Table 1); BC-239; Chenna et al (2020)35) acts as a reversible, putatively-covalent inhibitor of cruzain (Kᵢ*=310 nM).

FIG. 18 shows a scheme illustrating the synthesis of pro-drugs of compound 36. (a) DBU, DCM. (b) Pd/C, H₂, MeOH. (c) TFA, DCM. (d) Cbz-Phe-OH or NMePip-Phe-OH (S1j), DIPEA, T3P, DCM. (e) TBSCl, imidazole, DCM. (f) K₂CO₃, CH₃I, DMF. (g) NaBH₄, MeOH. (h) Dess-Martin periodinane, NaHCO₃, DCM, 0° C. (i) TBAF, THF, 0° C. (j) Et₃N, THF, 0° C. (k) mCPBA, chloroform. (1) NaN₃, MeOH/H₂O, 60° C.

FIG. 19 shows a scheme illustrating the synthesis of 37-41. (i) acetic/propionic/isobutyric anhydride, Et₃N, DMAP, DCM. (ii) BF₃OEt₂, EtOH/iPrOH.

FIG. 20 shows a scheme illustrating the synthesis of 2-pyridone-based MAIs (Compounds 42-48). (a) DBU, DCM. (b) Pd/C, H2, MeOH. (c) LiOH, MeOH/H2O. (d) N,O-dimethylhydroxylamine, DIPEA, T3P, DCM. (e) TFa, DCM; NMePip-Phe-OH, DIPEA, T3P, DCM. (f) LAH, THF, 0° C.

FIG. 21A-21C shows inactivator and inhibitors of cysteine proteases. (21A) Structure of K777 and its mechanism of covalent inactivation of cysteine proteases. (21B) 1,2,4-trioxolane inhibitor PG4b undergoes Fe(II)-catalyzed fragmentation to elaborate aldehyde PG3b, a potent inhibitor of falcipain, and a radical byproduct. (21C) γ-lactol inhibitor MN3 is a masked aldehyde of SJA0617. It exhibits higher transcorneal permeability and releases the free aldehyde to inhibit μ-calpain.

FIG. 22A-22C shows rationale for the design of SMAIs. (22A) In compound 30, the aldehyde group is expected to be masked by the 2′-phenol group via spontaneous formation of a lactol. It is anticipated that the SMAI will undergo enzyme-catalyzed opening of the lactol ring, and subsequently form the hemithioacetal adduct with Cys₂₅. The scheme describes a two-step inhibition mechanism in which rapid formation of an EI complex precedes isomerization to EI*, which slowly converts back to EL. (22B) Lactol form of 30 (green) non-covalently docked to cruzain. (22C) Opened form of 30 covalently docked to form a hemithioacetal with Cys₂₅. Both structures are superimposed with a covalently-bound K777 (white) from crystal structure (PDB ID: 2OZ2).²⁰ Colored dashed lines represent corresponding cruzain-inhibitor interactions.

FIG. 23A-23D shows kinetic analysis of inhibition of cruzain by 28, 30, and 36. (23A-23C) Time-courses of cruzain inhibition by 28, 30, and 36. Insets for (23A) and (23C): the k_{obs} values were obtained by fitting progress curves to eq. 1. Replot of k_{obs} vₛ. [I] with the line drawn through data points from fitting to eq. 3. 28: k₄=(7.6±0.6)×10⁻⁴ s⁻¹; 36: k₄=(1.8±0.7)×10⁻⁴ s⁻¹. Inset for (23B): the steady-state rates with (vₛ) and without inhibitor (v₀) were obtained at t≥20 mins. Plot of vₛ/v₀ vₛ. [30] with the line drawn through data points from fitting to eq. 4. (23D) A proposed mechanism of phenoxy-assisted conversion of EI* back to EI.

FIG. 24A-24F shows rapid dilution assay for SMAIs. (24A) Dilution scheme for testing the reversibility of a SMAI. Upon 100-fold dilution, the inhibitor concentration decreases from 10-fold>Kᵢ*^{app} (91% inhibition) to 10-fold<Kᵢ*app (9% inhibition). (24B, 24C) Cruzain activity rapidly recovered from inhibition by 30 and 31-35. (24D-24F) Cruzain activity recovery showed a significant lag for 28, 52, and 36. All curves were fitted to eq. 1 to provide k_{obs} values.

FIG. 25A-25B shows studies on chemical mechanism of SMAI binding to cruzain. (25A) Expansion of the superposed ¹H-¹³C HSQC NMR spectra of ¹³C-labeled compound 36 with (blue) and without (red) an approximately equimolar concentration of cruzain, which were obtained at 800 MHz (H) at 25° C. (25B) Time-course of phenylhydrazone formation by treating 0.2 mM 52 (aldehyde) or 36 (lactol) with 1 mM phenylhydrazine, with a control sample containing DMSO. Data for the curve with compound 52 were fitted to Abs_{282 nm}=a[1−exp(−k_{obs}t)]+C, from which a=0.279±0.002, k_{obs}=(2.61±0.05)×10⁻⁴ s⁻¹, and C=2.632±0.001 s⁻¹, while fitting the other data to this expression led to negligible values of k_{obs}.

FIG. 26A-26E shows development and assessment of SMAI prodrugs. (26A) Pralnacasan is a SMAI prodrug for VRT-18858, an inhibitor for caspase-1. (26B) Proposed metabolic routes of O-derivatized SMAIs to 36. (26C) Time-course of remaining 37-39 in reactions with or without addition of porcine esterase. (26D) HPLC traces of compound 39 in buffer (control). (26E) HPLC traces of 39 treated with esterase.

FIG. 27A-27D shows compound 43 is a potential SMAI for SARS-CoV-2 3CL^{pro}. (A) The 2-pyridone is a surrogate for the P₁ γ-lactam. (B) Structure and activity of compound 43. (C) Surface representation of the 3CL^{pro}-43 complex (PDB ID: 7M2P). The 2Fₒ−F_{c} electron density map contoured at 1.06 indicated C—S bond formation. (D) Interaction between 3CL^{pro} and 43. Dashed lines depict hydrogen bonds; the oxyanion hole is circled.

Like reference symbols in the various drawings indicate like elements.

### DETAILED DESCRIPTION

The following description of the disclosure is provided as an enable teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

The most successful inhibitors of the papain sub-family of cysteine proteases are peptide analogues that form covalent bonds, both reversible and irreversible, with the active-site cysteines of these enzyme targets. The types of substituents that form these covalent bonds include but are not limited to epoxides, nitriles, activated ketones, aldehydes, propenamides and vinyl sulfones. These electrophilic “warheads” have been installed in peptidomim“etic” or “organic” scaffolds, and have led to clinical candidates for the treatment of malaria, Chagas disease, cystic fibrosis, and osteoporosis. However, these covalent inactivators generally suffer from low selectivity due to presumed covalent attachment to other enzymes leading to untoward toxicities.

New inhibitors (or covalent inactivators) of cysteine proteases have been developed, which contain electrophilic “warheads” that may be “tuned” for selectivity and reactivity with the target cysteine protease, or, covalent inhibitors which are unveiled in the active site of the target enzyme. Peptide analogues that contain two types of inhibitory warheads have been designed, synthesized, and characterized enzymatically: (a) dipeptide analogues bearing a vinyl group which replaces the scissile amide group, and is conjugated with one of several heterocyclic groups, and (b) dipeptide analogues containing a C-terminal aldehyde group which forms a delta-lactol with a 2-hydroxyl group on the phenylalanyl sidechain proximal to the aldehyde. The inhibitors include two classes, peptidomimetic vinyl heterocycles (PVHs) and masked aldehydes (MAs, also called self-masked aldehydes (SMAs)), respectively.

Few inhibitors of cysteine proteases with reversible modes of action have been characterized to date that have low nanomolar potency for their enzyme targets. Covalent inhibitors of therapeutically-important cysteine proteases, including cruzain and the falcipains, have been previously identified but none of the compounds contain vinyl 2-heterocycles or masked aldehydes as described herein. Using a heterocycle as an isosteric surrogate of an amide or sulfone, allowing conjugation between the reactive vinyl group that would allow substitution in order to modify the electrophilicity of the vinyl group is described herein. The concept of a masked aldehyde in the forms of (oxo) 2-hydroxyphenyl or 2-carboxyphenyl-lactols is unprecedented. Patents exist for covalent inactivators of cysteine proteases, some of which include a vinyl substituent located in the exemplified peptidomimetic compounds in the same position as compounds 2-27. One such patent is WO95/02322 where sulfones, esters, amides and other electron-withdrawing groups are attached to the vinyl group, but heterocycles are not found within the claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs.

### General Definitions

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula S(O)₂NH.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, -nRaRb, —NRaC(═O)Rb, -nRaC(═O)NraNRb, -nRaC(═O)ORb, —NRaSO₂Rb, —C(═O)Ra, —C(═O)ORa, —C(═O)NRaRb, —OC(═O)nRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

Compounds

Described herein is an unprecedented class of tight-binding, reversible inhibitors of the cysteine proteases (e.g., cruzain and cathepsin L) known as masked aldehyde inhibitors (MAIs) (or self-masked aldehyde inhibitors).⁶¹ Examples of this class of inhibitors are effective in a cellular model of Chagas disease and in a viral infection assay of COVID-19. These inhibitors have novel, nascent electrophilic “warheads” which form reversible covalent adducts with the active cysteine of cruzain, human cathepsin L and SARS CoV-2 3CL protease.

The compounds described herein are cysteine protease inhibitors. In some embodiments, the compounds have a structure according to the following Formula I.

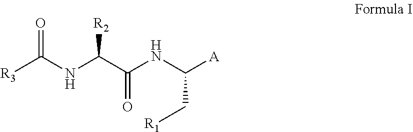

wherein
R₁ is a substituted or unsubstituted aryl, heteroaryl, or alkyl;
R₂ and R₃ are independently hydrogen, substituted or unsubstituted alkyl, aryl, cycloalkyl, or heteroaryl; or R₂ and the adjacent N atom, together with the atoms to which they are attached, combine to form a 3 to 6 membered heterocycle;

A is

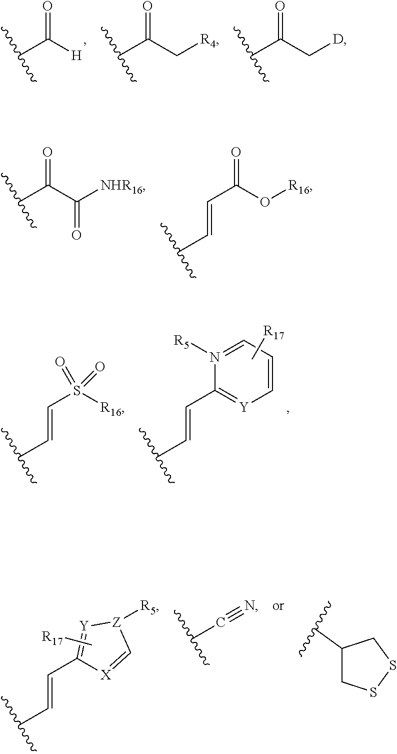

D is a halogen; X and Y are independently NR₁₉ or CR₁₉; Z is NR₁₉, S, O, or CR₁₉; R₄ is substituted or unsubstituted alkyl, substituted or unsubstituted aryl, hydroxyl, thiol, or amino; R₅ is absent, or substituted or unsubstituted alkyl; R₁₆ is substituted or unsubstituted alkyl or aryl; R₁₇ is a hydrogen or an electron-donating such as an alkyl, alkoxy, amino, or electron-withdrawing group such as a F, Cl, Br, trifluoromethyl, cyano, or nitro; and R₁₉ is absent or hydrogen.

In some embodiments, R₃ is (D-) or (L-) amino acid, acyl-amino acid, 4N-alkyl-piperazinyl, alkyloxycarbonyl, or aryloxycarbonyl. In some embodiments, R₃ is benzyloxy 4-N-methyl-piperazinyl, or benzyloxycarbonyl. In some embodiments, R₃ can be:

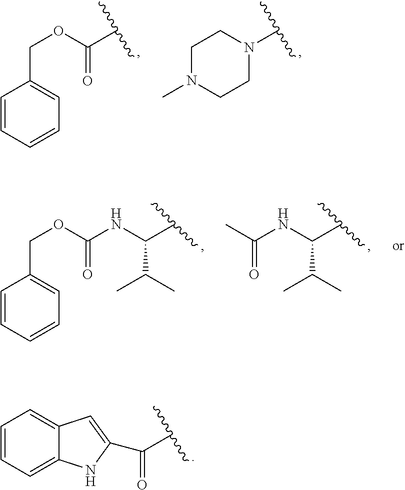

In some embodiments, R₂ is benzyl, substituted benzyl, 4-pyridine, 3-amino-propyl, cyclohexyl, isopropyl, or 4-nitrobenzyl.

In some embodiments, R₁ is

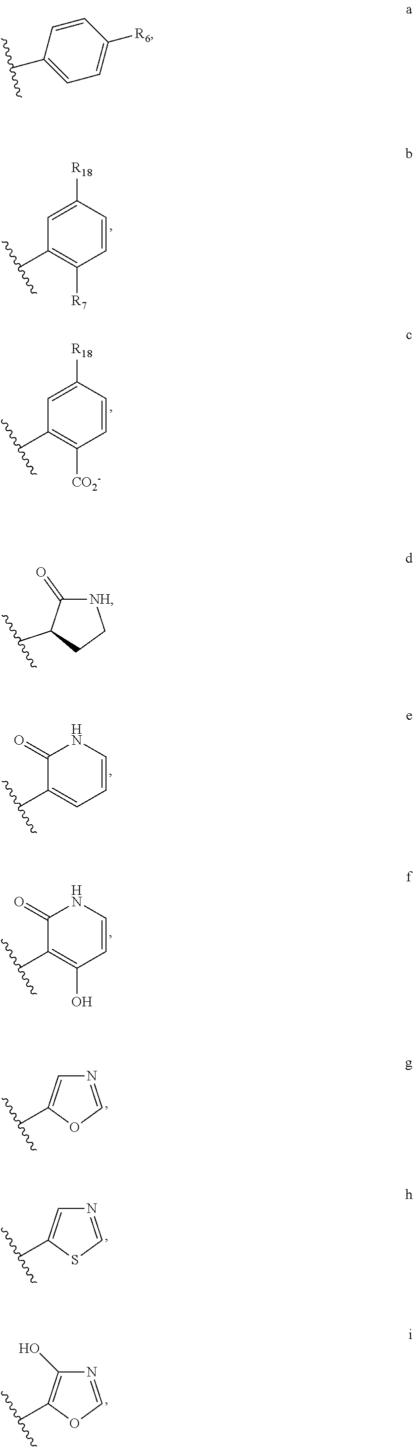

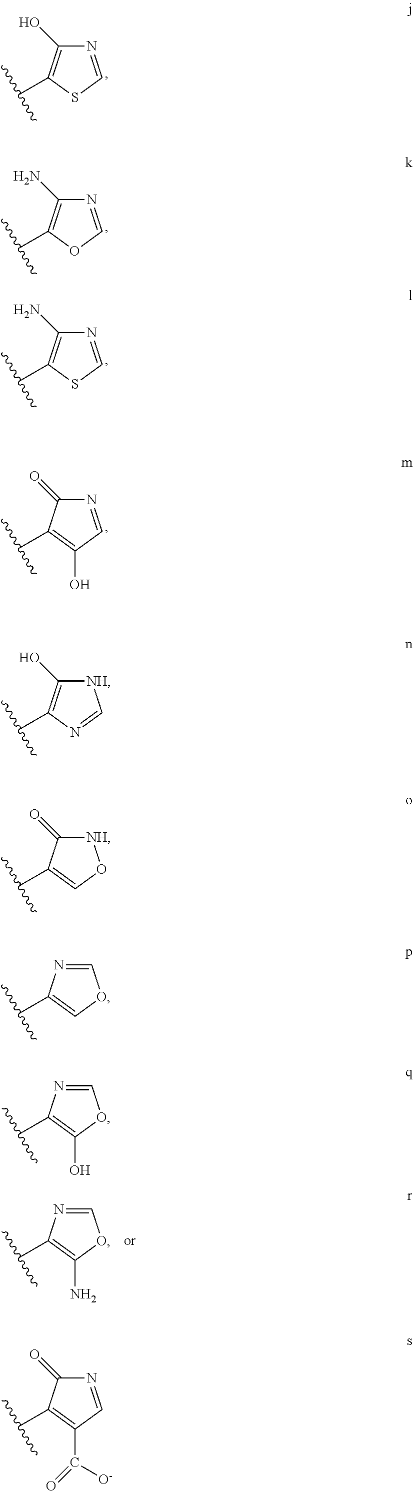

In some embodiments, R₁ is

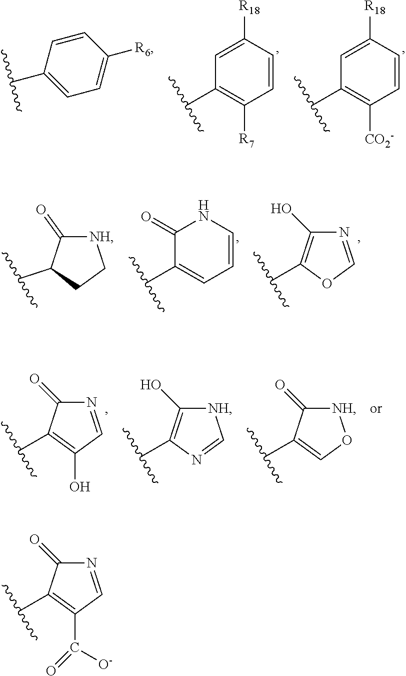

wherein,
R₆, R₇, and R₁₈ are independently hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl.

In some embodiments, R₁₈ is hydrogen, substituted or unsubstituted alkyl, substituted or unsubstituted alkoxy, amino, halogen, nitro, cyano, —CF. In some embodiments, R₇ is hydrogen, amino, hydroxy, or thiol.

In some embodiments, R₁ is benzyl, 2-benzyl, 2-carboxybenzyl, (3R)-pyrrolidin-2-on-3-yl, pyridin-2(1H)-on-3-yl, 4-hydroxy-pyridin-2(1H)-on-3-yl, 1,3-oxazo-5-yl, 1,3-thiazo-5-yl, 4-hydroxy-1,3-oxazo-5-yl, 4-hydroxy-1,3-thiazo-5-yl, 4-amino-1,3-oxazo-5-yl, 4-amino-1,3-thiazo-5-yl, 4-hydroxy-2H-pyrrol-2-on-3-yl, 5-hydroxy-1H-imidazo-4-yl, isoxazole-3(2H)-on-4-yl, 1,3-oxazo-4-yl, 5-hydroxy-1,3-oxazo-4-yl, 5-hydroxy-1,3-oxazo-4-yl, or carboxy-2H-pyrrol-2-on-3-yl.

In some embodiments, R₁ is benzyl, 2-benzyl, 2-carboxybenzyl, (3R)-pyrrolidin-2-one, 3-pyridin-2-one, 4-hydroxy-3-pyridin-2-one, 5-(4-hydroxy)-oxazoyl, 3-(4-hydroxy)-2H-pyrrol-2-one, 3-(5-hydroxy)-imidazoyl, 4-isoxazol-3-one, or 3-(4-carboxy-2-oxo-2H-pyrrole.

In some embodiments, the compounds have a structure according to the following Formula Ia:

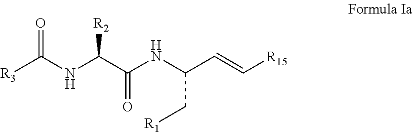

wherein
R₁ is a substituted or unsubstituted aryl, heteroaryl, or alkyl;
R₂ and R₃ are independently hydrogen, substituted or unsubstituted alkyl, aryl, cycloalkyl, or heteroaryl; or R₂ and the adjacent N atom, together with the atoms to which they are attached, combine to form a 3 to 6 membered heterocycle;
R₁₅ is selected from the group consisting of:

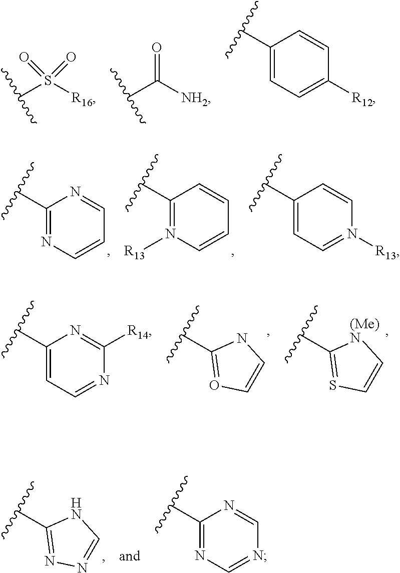

R₁₂ and R₁₄ are independently hydrogen, substituted or unsubstituted alkyl, amino, or nitro; and
R₁₆ is a substituted or unsubstituted alkyl or aryl;
R₁₃ is a substituted or unsubstituted alkyl.

In some embodiments, R₁₂ is hydrogen or nitro. In some embodiments, R₁₃ is methyl. In some embodiments, R₁₄ is hydrogen or —NH₂.

In some embodiments, R₃ is (D-) or (L-) amino acid, acyl-amino acid, 4N-alkyl-piperazinyl, alkyloxycarbonyl, or aryloxycarbonyl. In some embodiments, R₃ is benzyloxy 4-N-methyl-piperazinyl, or benzyloxycarbonyl.

In some embodiments, R₃ can be:

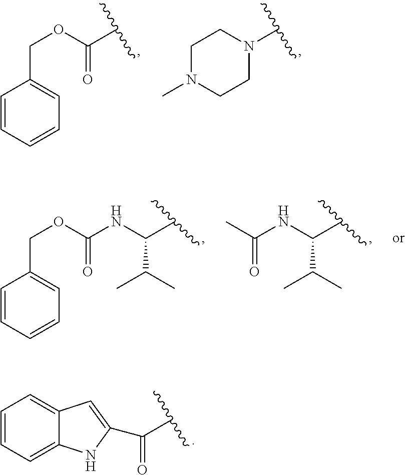

In some embodiments, R₂ is benzyl, substituted benzyl, 4-pyridine, 3-amino-propyl, cyclohexyl, isopropyl, or 4-nitrobenzyl.

In some embodiments, the compound of Formula Ia, wherein R₁₅ is selected from the group consisting of:

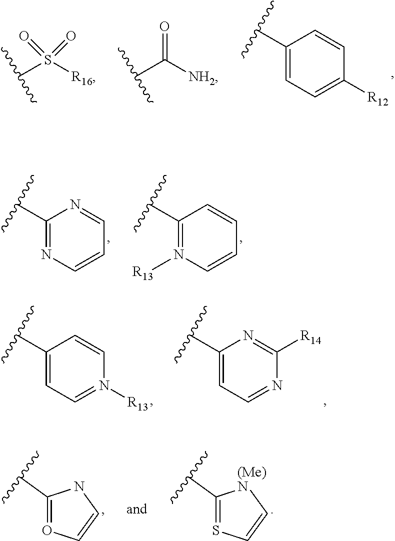

In some embodiments, R₁ is

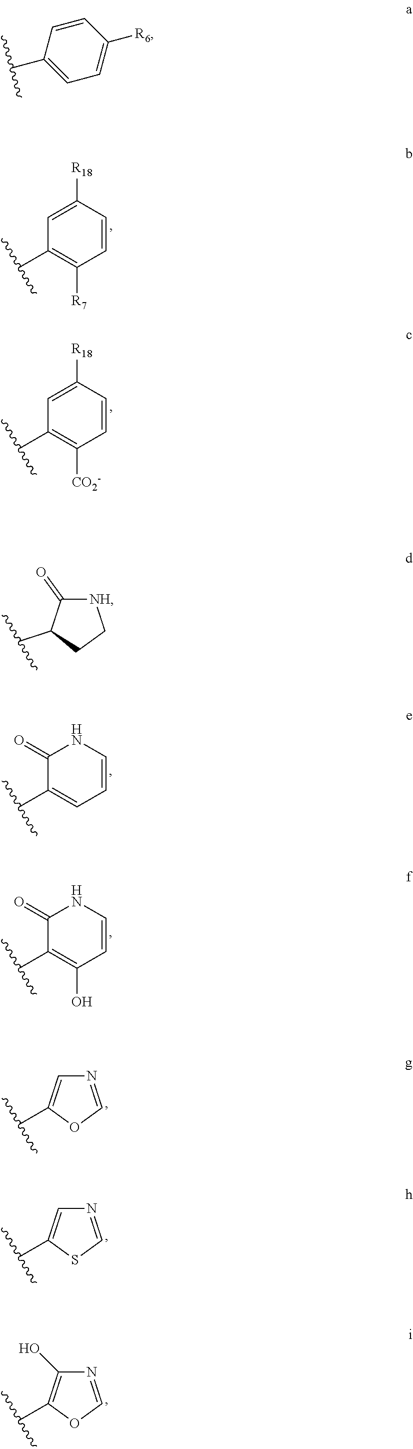

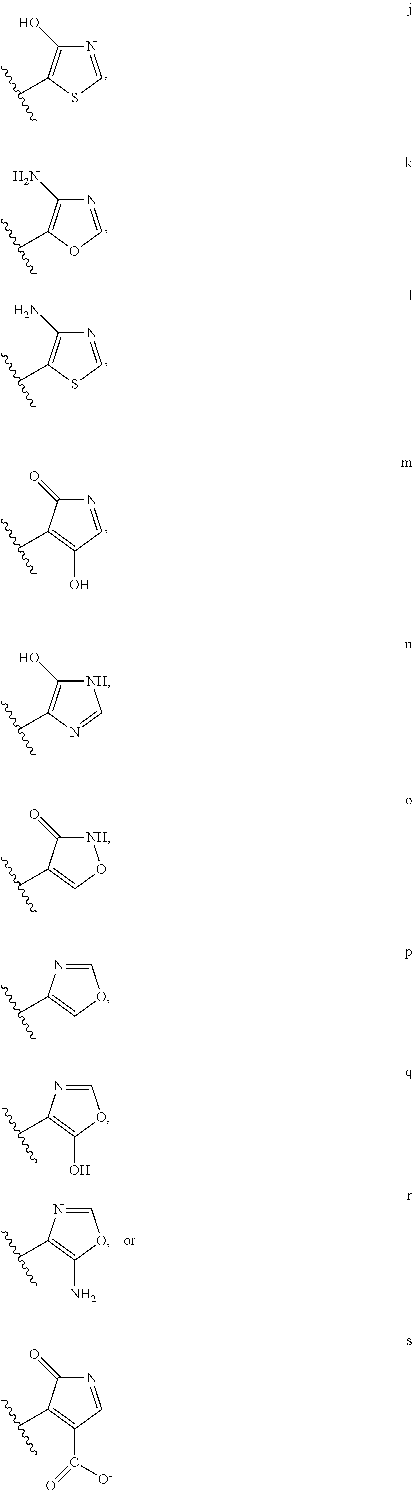

In some embodiments, R₁ is

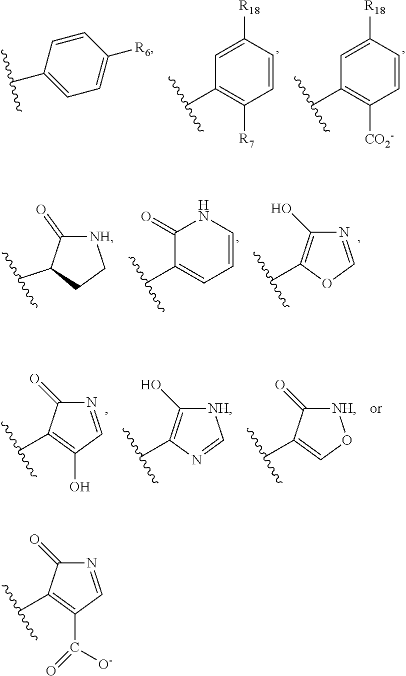

wherein,
R₆, R₇, and R₁₈ are independently hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl.

In some embodiments, R₁₈ is hydrogen, substituted or unsubstituted alkyl, substituted or unsubstituted alkoxy, amino, halogen, nitro, cyano, —CF. In some embodiments, R₇ is hydrogen, amino, hydroxy, or thiol.

In some embodiments, R₁ is benzyl, 2-benzyl, 2-carboxybenzyl, (3R)-pyrrolidin-2-on-3-yl, pyridin-2(1H)-on-3-yl, 4-hydroxy-pyridin-2(1H)-on-3-yl, 1,3-oxazo-5-yl, 1,3-thiazo-5-yl, 4-hydroxy-1,3-oxazo-5-yl, 4-hydroxy-1,3-thiazo-5-yl, 4-amino-1,3-oxazo-5-yl, 4-amino-1,3-thiazo-5-yl, 4-hydroxy-2H-pyrrol-2-on-3-yl, 5-hydroxy-1H-imidazo-4-yl, isoxazole-3(2H)-on-4-yl, 1,3-oxazo-4-yl, 5-hydroxy-1,3-oxazo-4-yl, 5-hydroxy-1,3-oxazo-4-yl, or carboxy-2H-pyrrol-2-on-3-yl.

In some embodiments, R₁ is benzyl, 2-benzyl, 2-carboxybenzyl, (3R)-pyrrolidin-2-one, 3-pyridin-2-one, 4-hydroxy-3-pyridin-2-one, 5-(4-hydroxy)-oxazoyl, 3-(4-hydroxy)-2H-pyrrol-2-one, 3-(5-hydroxy)-imidazoyl, 4-isoxazol-3-one, or 3-(4-carboxy-2-oxo-2H-pyrrole.

Examples of compounds of Formula Ia are in Tables 1 and 2.

In some other embodiments, the compounds have a structure according to the following Formula Ib:

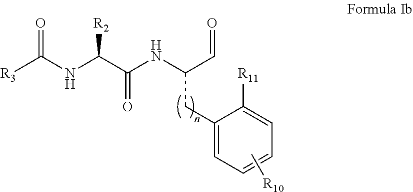

wherein
R₂ and R₃ are independently hydrogen, substituted or unsubstituted alkyl, aryl, cycloalkyl, or heteroaryl; or R₂ and the adjacent N atom, together with the atoms to which they are attached, combine to form a 3 to 6 membered heterocycle;
R₁₁ is hydroxy, thiol, or —COO;
R₁₀ is hydrogen, hydroxy, thiol, substituted or unsubstituted alkyl, substituted or unsubstituted alkoxy, amino, halogen, nitro, cyano, —CF₃, —CO₂Ra, —COOH, or —CONH₂, Ra is a substituted or unsubstituted alkyl or aryl;
n is an integer from 1-3.

In some other embodiments, R₁₀ is hydrogen, methyl, or other alkyl groups, —CF₃, —NO₂, —CN, —F, —Cl, —Br, —OMe, NH₂, —COOH, —CO₂Ra, or —CONH₂; Ra is substituted or unsubstituted alkyl or aryl; and n is an integer from 1-3.

In some embodiments, R₃ is (D-) or (L-) amino acid, acyl-amino acid, 4N-alkyl-piperazinyl, alkyloxycarbonyl, or aryloxycarbonyl. In some embodiments, R₃ is benzyloxy 4-N-methyl-piperazinyl, or benzyloxycarbonyl.

In some embodiments, R₃ can be:

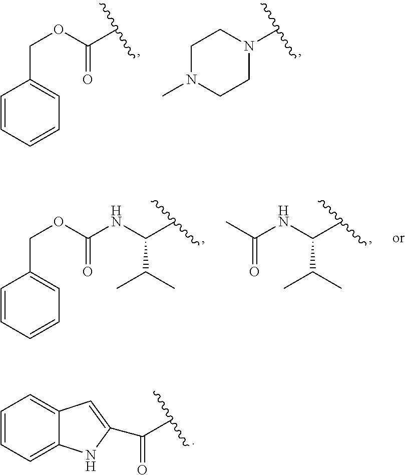

In some embodiments, R₂ is benzyl, substituted benzyl, 4-pyridine, 3-amino-propyl, cyclohexyl, isopropyl, or 4-nitrobenzyl.

Examples of these compounds are shown below:

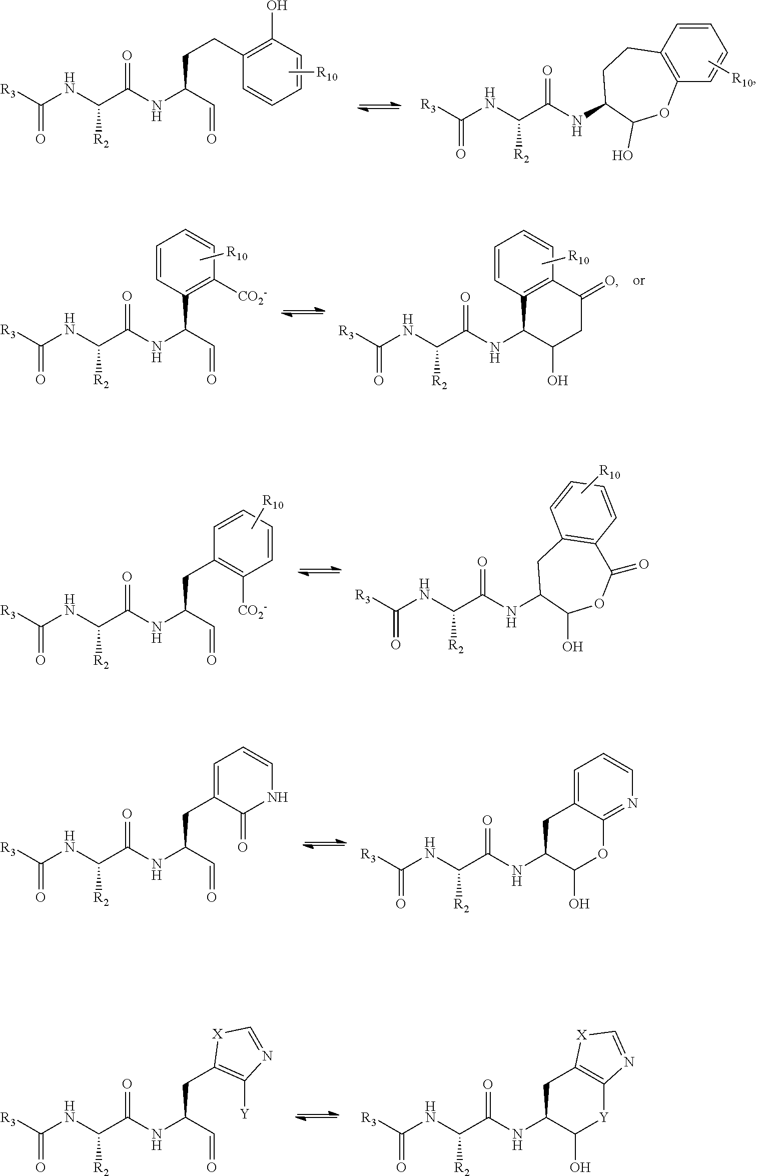

wherein
X is O, NH, or S;
Y is OH or NH₂;
R₂ and R₃ are independently hydrogen, substituted or unsubstituted alkyl, aryl, cycloalkyl, or heteroaryl; or R₂ and the adjacent N atom, together with the atoms to which they are attached, combine to form a 3 to 6 membered heterocycle;
R₁₁ is hydroxy, thiol, or —COO;
R₁₀ is hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl;
n is an integer from 1-3.

In some other embodiments, R₁₀ is hydrogen, methyl, or other alkyl groups, —CF₃, NO₂, CN, F, Cl, Br, —OMe, NH₂, —COOH, —CO₂Rₐ, or —CONH₂; Rₐ is substituted or unsubstituted alkyl or aryl; and n is an integer from 1-3.

In some embodiments, R₃ is (D-) or (L-) amino acid, acyl-amino acid, 4N-alkyl-piperazinyl, alkyloxycarbonyl, or aryloxycarbonyl. In some embodiments, R₃ is benzyloxy 4-N-methyl-piperazinyl, or benzyloxycarbonyl.

In some embodiments, R₃ can be:

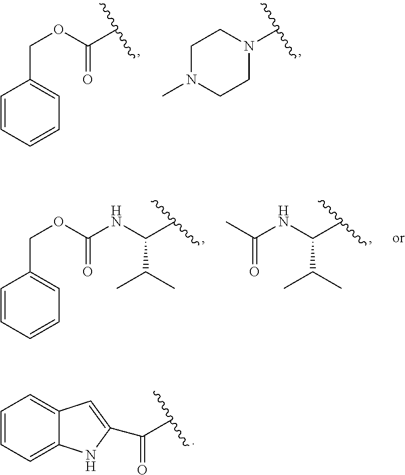

In some embodiments, R₂ is benzyl, substituted benzyl, 4-pyridine, 3-amino-propyl, cyclohexyl, isopropyl, or 4-nitrobenzyl.

In some other embodiments, the compounds have a structure according to the following Formula II:

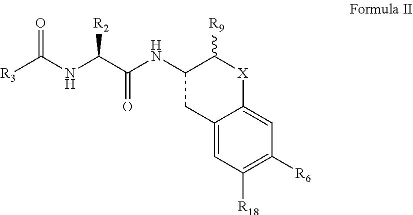

### Wherein

X is O, NH, and S,
R₂ and R₃ are independently hydrogen, substituted or unsubstituted alkyl, aryl, cycloalkyl, or heteroaryl, or R₂ and the adjacent N atom, together with the atoms to which they are attached, combine to form a 3 to 6 membered heterocycle;
R₁₈ and R₆ are independently hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl; and R₉ is hydroxy, or alkoxy.

In some embodiments, R₁₈ and R₆ are independently hydrogen, —OMe, -Me, —CF₃, —Cl, —F, —CO₂Et, —CO₂Me, —COOH, —CONH₂, or —NO₂.

In some embodiments, R₃ is (D-) or (L-) amino acid, acyl-amino acid, 4N-alkyl-piperazinyl, alkyloxycarbonyl, or aryloxycarbonyl. In some embodiments, R₃ is benzyloxy 4-N-methyl-piperazinyl, or benzyloxycarbonyl.

In some embodiments, R₃ can be:

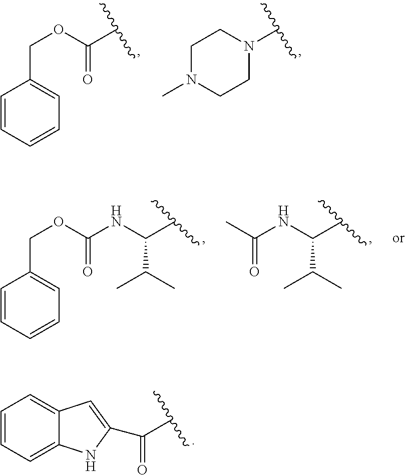

In some embodiments, R₂ is benzyl, substituted benzyl, 4-pyridine, 3-amino-propyl, cyclohexyl, isopropyl, or 4-nitrobenzyl.

Examples of compounds of Formula I and II are shown below and in Table 2:

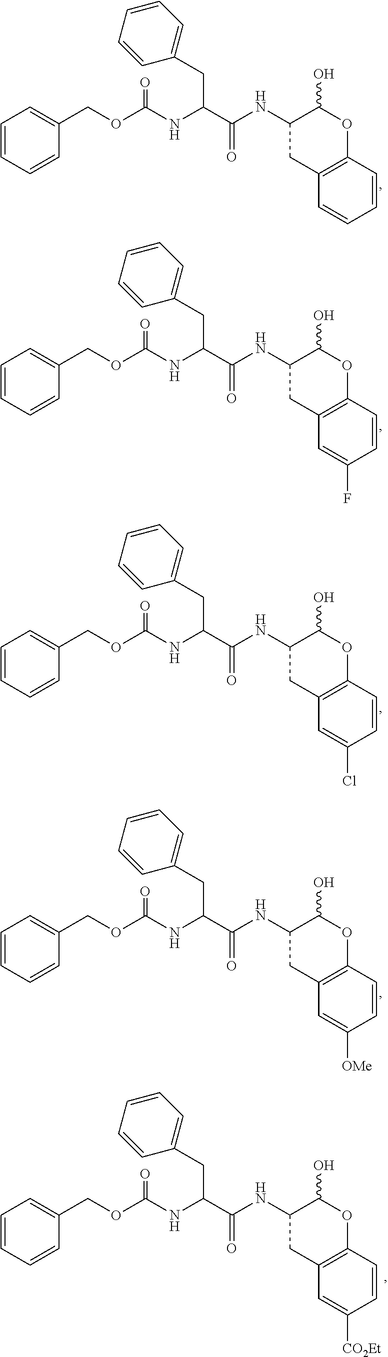

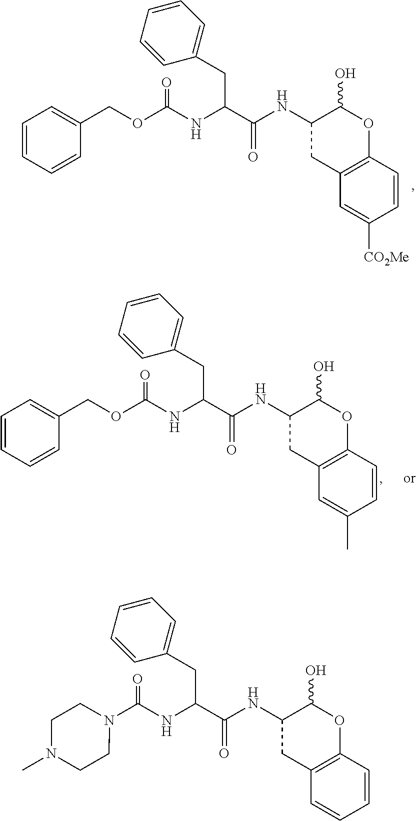

or pharmaceutically acceptable salts thereof.

In some other embodiments, the compounds have a structure according to the following Formula IIa:

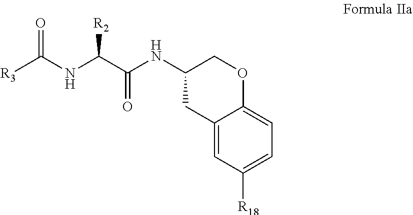

wherein
R₂ and R₃ are independently hydrogen, substituted or unsubstituted alkyl, aryl, cycloalkyl, or heteroaryl; or R₂ and the adjacent N atom, together with the atoms to which they are attached, combine to form a 3 to 6 membered heterocycle; and
R₁₈ is hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl.

In some embodiments, R₁₈ is independently hydrogen, —OMe, -Me, —CF₃, Cl, F, —CO₂Et, —CO₂Me, —COOH, —CONH₂, or —NO₂.

In some embodiments, R₃ is (D-) or (L-) amino acid, acyl-amino acid, 4N-alkyl-piperazinyl, alkyloxycarbonyl, or aryloxycarbonyl. In some embodiments, R₃ is benzyloxy 4-N-methyl-piperazinyl, or benzyloxycarbonyl.

In some embodiments, R₃ can be:

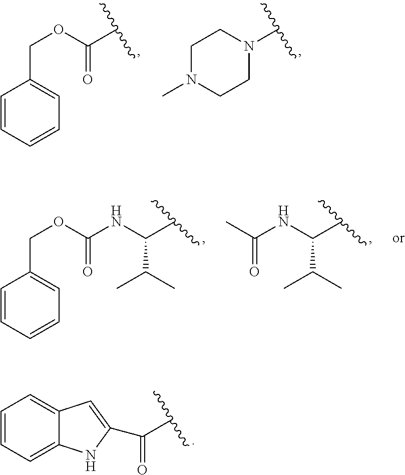

In some embodiments, R₂ is benzyl, substituted benzyl, 4-pyridine, 3-amino-propyl, cyclohexyl, isopropyl, or 4-nitrobenzyl.

The metabolic hydroxylation of the C-terminal carbon of Formula II or IIa will produce a δ-lactol (masked aldehyde) identical to Formula I, Ia, or Ib.

In some embodiments, the cysteine protease can be selected from cruzain, cruzipain, human cathepsin B, human cathepsin L, TbCatB, TbCatL, rhodesain, brucipain, falcipain-2, falcipain-3, and/or coronaviral cysteine proteases 3CL-PR and PL-PR. In some embodiments, the compound can be a human cathepsin L inhibitor.

The compounds described herein can be made through well know methods in the art.

Pharmaceutical Compositions

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

Methods of Treating

The present disclosure provides methods for treating, inhibiting, decreasing, reducing, ameliorating and/or preventing Chagas disease, African trypanosomiasis, malaria, or a coronavirus infections in a subject in need thereof, the method comprising administering a therapeutically effective amount of the protease inhibitor, or a pharmaceutically acceptable salt or derivative thereof. In another aspect, the present disclosure provides methods for treating, inhibiting, decreasing, reducing, ameliorating and/or preventing the disease and/or symptoms associated with Chagas disease, African trypanosomiasis, malaria, or a coronavirus infection in a subject in need thereof, comprising administering a therapeutically effective amount of the protease inhibitor, or a pharmaceutically acceptable salt or derivative thereof.

A “coronavirus infection” as used herein refers to an infection caused by or otherwise associated with growth of coronavirus in a subject, in the family Coronaviridae (subfamily Coronavirinae).

Coronaviruses are species of virus belonging to the subfamily Coronavirinae in the family Coronaviridae, in the order Nidovirales. Coronaviruses are enveloped viruses with a positive-sense single-stranded RNA genome and with a nucleocapsid of helical symmetry.

In one embodiment, the coronavirus infection is an infection of the upper and/or lower respiratory tract. The “upper respiratory tract” includes the mouth, nose, sinus, middle ear, throat, larynx, and trachea. The “lower respiratory tract” includes the bronchial tubes (bronchi) and the lungs (bronchi, bronchioles and alveoli), as well as the interstitial tissue of the lungs.

In another embodiment, the coronavirus infection is an infection of the gastrointestinal tract. The “gastrointestinal tract” may include any area of the canal from the mouth to the anus, including the mouth, esophagus, stomach, and intestines.

In yet another embodiment, the coronavirus infection is a renal infection.

It is understood and herein contemplated that the coronavirus infections disclosed herein can cause a pathological state associated with the coronavirus infection referred to herein as a “coronavirus disease.” In some embodiments, the coronavirus disease is selected from a common cold, pneumonia, pneumonitis, bronchitis, severe acute respiratory syndrome (SARS), coronavirus disease 2019 (COVID-2019), Middle East respiratory syndrome (MERS), sinusitis, porcine diarrhea, porcine epidemic diarrhea, avian infections bronchitis, otitis and pharyngitis. In particular embodiments, the coronavirus infection is a common cold. In particular embodiments, the coronavirus infection is selected from SARS, COVID-19, and MERS. In a particular embodiment, the coronavirus infection is COVID-19. In another particular embodiment, the coronavirus infection is IBV, PorCoV HKU15, or PEDV.

Other indications associated with coronavirus infections are described in Gralinski & Baric, 2015, J. Pathol. 235:185-195 and Cavanagh, 2005, “Coronaviridae: a review of coronavirus and toroviruses”, Coronaviruses with Special Emphasis on First Insights Concerning SARS 1, ed. By A. Schmidt, M. H. Wolff and O. Weber, Birkhauser Verlag Baser, Switzerland, each of which is incorporated herein by reference in their entirety.

The coronavirus causing the infection may be selected from an alphacoronavirus, a betacoronavirus, a gammacoronavirus, or a deltacoronavirus.

Representative examples of alphacoronaviruses include, but are not limited to, a colacovirus (e.g., Bat coronavirus CDPHE15), a decacovirus (e.g., Bat coronavirus HKU10, Rhinolophus ferrumequinum alphacoronavirus Hub-2013), a duvinacovirus (e.g., Human coronavirus 229E), a luchacovirus (e.g., Lucheng Rn rat coronavirus), a minacovirus (e.g., Ferret coronavirus, Mink coronavirus 1), a minunacovirus (e.g., Miniopterus bat coronavirus 1, Miniopterus bat coronavirus HKU8), a myotacovirus (e.g., Myotis rickettii alphacoronavirus Sax-2011), a nyctacovirus (e.g., Nyctalus velutinus alphacoronavirus SC-2013), a pedacovirus (e.g., Porcine epidemic diarrhea virus (PEDV), Scotophilus bat coronavirus 512), a rhinacovirus (e.g., Rhinolophus bat coronavirus HKU2), a setracovirus (e.g., Human coronavirus NL63, NL63-related bat coronavirus strain BtKYNL63-9b), and a tegacovirus (e.g. Alphacoronavirus 1).

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang 2013), a merbecovirus (e.g., Hedgehog coronavirus 1, Middle East respiratory syndrome-related coronavirus (MERS-CoV), Pipistrellus bat coronavirus HKU5, Tylonycteris bat coronavirus HKU4), a nobecovirus (e.g., Rousettus bat coronavirus GCCDC1, Rousettus bat coronavirus HKU9), a sarbecovirus (e.g., severe acute respiratory syndrome coronavirus (SARS-CoV), severe acute respiratory syndrome coronavirus 2 (SARS-CoV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

In one embodiment, a method is provided for treating coronavirus disease 2019 (COVID-2019) including administering a therapeutically effective amount of the protease inhibitor, or a pharmaceutically acceptable salt or analog thereof.

In one embodiment, a method is provided for preventing coronavirus disease 2019 (COVID-2019) including administering a therapeutically effective amount of the protease inhibitor, or a pharmaceutically acceptable salt or analog thereof.

In another embodiment, a method is provided for inhibiting, decreasing, reducing, ameliorating and/or preventing one or more symptoms associated with coronavirus disease 2019 (COVID-2019) including administering a therapeutically effective amount of the protease inhibitor, or a pharmaceutically acceptable salt or analog thereof.

Another embodiment is a method of inhibiting cruzain, cruzipain, human cathepsin B, human cathepsin L, TbCatB, TbCatL, rhodesain, brucipain, falcipain-2, falcipain-3, 3CL-PR, or PL-PR comprising administering a therapeutically effective amount of the protease inhibitor, or a pharmaceutically acceptable salt or derivative thereof.

In another embodiment, a method is also provided for inhibiting cathepsin L responsible for the cleavage of the S1 subunit of the coronavirus surface spike glycoprotein necessary for coronavirus entry into human host cells.

In another embodiment, a method is provided for reducing viral load of a coronavirus in a coronavirus-infected cell, the method comprising contacting the cell with a therapeutically effective amount of the protease inhibitor, or a pharmaceutically acceptable salt or analog thereof. In some embodiments, the coronavirus is SARS-CoV-2. In some embodiments, the cell is a human cell.

In another embodiment, a method is provided for reducing inflammation in a tissue of a subject infected with a coronavirus, the method comprising administering a therapeutically effective amount of the protease inhibitor, or a pharmaceutically acceptable salt or analog thereof. In some embodiments, the coronavirus is SARS-CoV-2. In some embodiments, the tissue is lung tissue.

Another embodiment is a method of treating the disease and/or symptoms associated with Chagas disease, African trypanosomiasis, malaria, or a coronavirus infection in a subject in need thereof, comprising administering a therapeutically effective amount of the protease inhibitor, or a pharmaceutically acceptable salt or derivative thereof.

Another embodiment is a method of killing or inhibiting the growth of a protozoan, the method comprising: contacting the protozoan with a protease inhibitor compound described herein, in an amount effective to kill or inhibit the growth of the protozoan. In some embodiments, the protozoan can be Trypanosoma cruzi, Trypanosoma brucei brucei, Trypanosoma brucei rhodiense, Trypanosoma brucei gambiense, Plasmodium falciparum, or Plasmodium vivax.

Another embodiment is a method of killing or inhibiting the growth of SARS CoV-2, the method comprising: contacting the coronavirus with the compound above, in an amount effective to kill or inhibit the growth of the virus.

Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of an infection with a Mycobacterium.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the protease inhibitor as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, antiplatelet agents, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of additional active agents include chloroquine, hydrochloroquine, Vitamin D, and Vitamin C

In some embodiments, the protease inhibitor as used in the methods described herein may be administered in combination or alternation with one or more anticytokine or immunomodulatory agents, representative examples of which include, but are not limited to, tocilizumab, sarilumab, bevacizumab, fingolimod, imiquimod, and eculizumab.

In some embodiments, the protease inhibitor as used in the methods described herein may be administered in combination or alternation with an immunoglobulin therapy.

An embodiment of the disclosure is a method of identifying an inhibitor of cruzain, cruzipain, cathepsin B, cathepsin L, TbCatB, TbCatL, rhodesain, brucipain, falcipain-2, falcipain-3, and/or coronaviral cysteine proteases 3CL-PR and PL-PR comprising adding a peptide side chain to a PVH or masked aldehyde (MA); and assaying whether the peptide side chain-PVH or peptide side chain-MA inhibits at least one selected from the group consisting of cruzain, cruzipain, cathepsin B, cathepsin L, TbCatB, TbCatL, rhodesain, brucipain, falcipain-2, falcipain-3, and/or coronaviral cysteine proteases 3CL-PR and PL-PR.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

### Example 1: Peptidomimetic Vinyl Heterocyclic and Masked Aldehydes Inhibitors

The medicinal chemistry for cruzain, the falcipains, TbCatB, brucipain, and rhodesain was examined to develop lead compounds against these enzyme targets. Testing in cell cultures of Trypanosoma ssp. and Plasmodium spp, analysis of lead compounds in terms of their efficacy against animal models of malaria, Chagas disease, and African sleeping sickness will be performed. Molecular modelling and computer-aided drug design will also be utilized. X-ray crystallographic descriptions of cruzain, the falcipains, and the cysteine proteases of Trypanosoma brucei with the lead compounds bound to them will be obtained, to assist in the design of more specific and potent lead compounds. Physical organic chemistry will be used to probe the electrophilicity of vinyl groups conjugated with heterocyclic substituents, not involving enzymes, in order to quickly probe the chemistry for producing suitable electrophiles for our target enzymes.

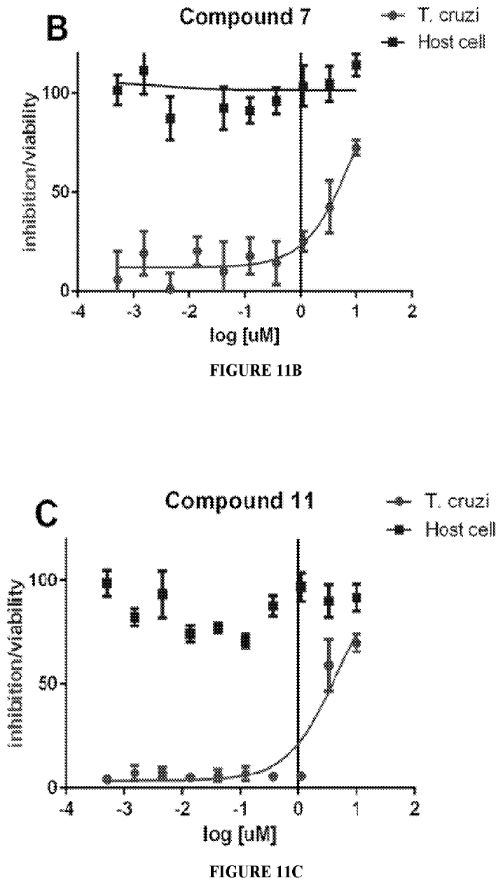

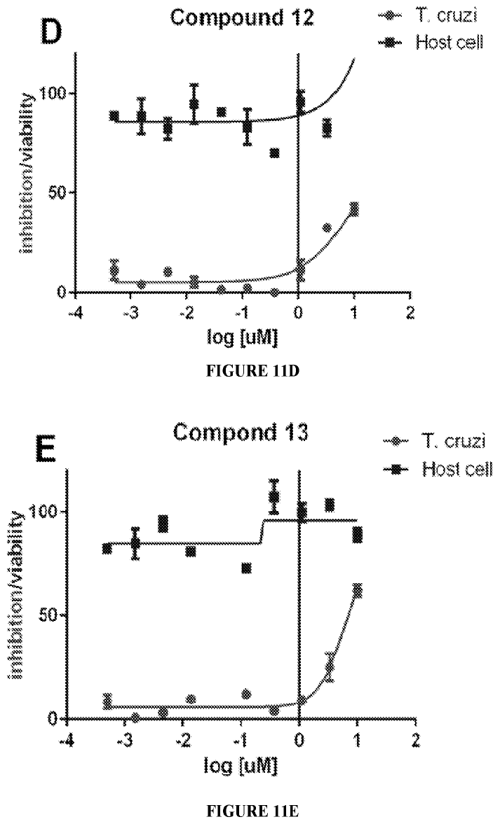

Details of the catalytic mechanism of cruzain in terms of the rate-limiting steps of the enzymatic reaction and the protonation states of the active-site Cys and His residues were recently published.⁴⁰ The results from this study demonstrated that for peptide substrates with optimal values of k_{cat}/Kₘ and k_{cat}, (for peptides S1, S6, and S12; FIG. 11; Zhai, et al.) the rate of de-acylation of the enzyme-thioester is rate-limiting, and that substrates bind to a free enzyme form in which both the active-site cysteine (Cys-25) and histidine (His-162) are neutral. This latter result is unusual for cysteine proteases in that the Cys-His catalytic dyad is generally in its more reactive thiolate-imidazolium form, and the findings should inform strategies for the design of new inhibitors. Importantly, values of k_{cat}/Kₘ report on the rates of substrate binding and catalysis up to and including the acylation of active-site Cys-25, and so this kinetic parameter comprises a useful guide to select optimal di-peptide scaffolds for inhibitors which are meant to form reversible covalent complexes with Cys-25 upon binding, which chemically mimics acylation.

Enzyme kinetics of cruzain, falcipain-2, other trypanosomal cysteine proteases, and in the future, the Trypanosoma brucei cysteine proteases, are used to optimize the enzyme's recognition of peptide substrates. This will provide the peptide sequence that is the most selective for the target enzymes. In parallel, a number of vinyl-heterocycles on a single peptide scaffold, such as Cbz-Phe-Phe-vinyl-2-heterocycle and the Cbz-Phe-ortho-Tyr-CHO (Compound 30) will be produced in order to assess the inhibitory properties of these compounds. Research continues to identify additional inhibitors of these cysteine proteases that are covalent, but reversible, owing to the re-establishment of aromatic conjugation of the vinyl-heterocycle upon cleavage of the C—S bond of the enzyme and inactivator, or by the reversal of the hemithioacetal formed between the peptide aldehydes and Cys-25. Specificity for the target enzymes will be afforded by selecting the optimal peptide recognition sequence as obtained from kinetic studies. The overall size of the cysteine protease inhibitors will be reduced in accord with Lipinski rules for drug development, and the compounds evaluated against cellular cultures of Trypanosoma cruzi, Trypanosoma brucei, and Plasmodium falciparum. Compounds that kill parasites will be further advanced to testing in animal models of malaria, African sleeping sickness, and Chagas disease.

An aim is to identify new peptidomimetic 2-vinyl heterocycle inhibitors (P2VHIs) and optimize their activity against cruzain and TbCatL. The data has established the feasibility of the synthesis of novel P2VHIs, and as shown, they are potent and reversible inhibitors of cruzain. New P2VHIs of cruzain and TbCatL will be synthesized and characterized, kinetically and structurally, by: (a) identification of selective peptide scaffolds by analysis of 20-30 tripeptide substrates; (b) synthesis of additional 2-vinyl heterocycles, and test as new P2VHIs in a single peptide scaffold; (c) substitution of the 2-vinyl heterocycles to improve their electrophilicity; (d) assessment of the reversibility of covalent bond formation between cruzain, TbCatL, and selected P2VHIs by X-ray crystallography, and ¹³C-NMR analysis; and (e) integration of the results of (a-d) to develop a panel of 10-20 first-generation P2VHIs which are selective and potent inhibitors of cruzain and TbCatL. A further aim is to evaluate the trypanocidal activity of new P2VHIs. P2VHIs of cruzain and TbCatL will be assessed for parasitidal activity in cell cultures of T. cruzi and T. brucei spp., and assessed for toxicity in human cells.

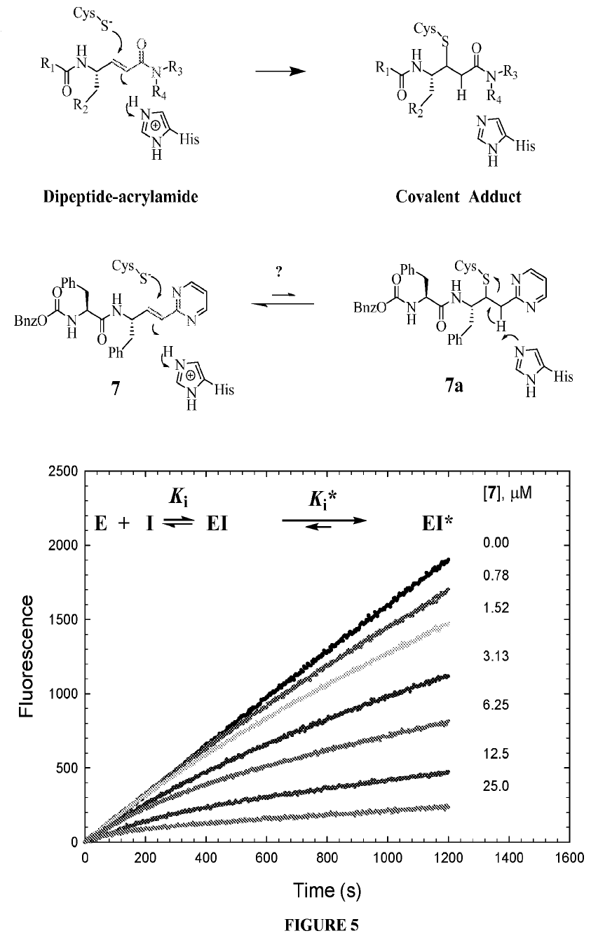

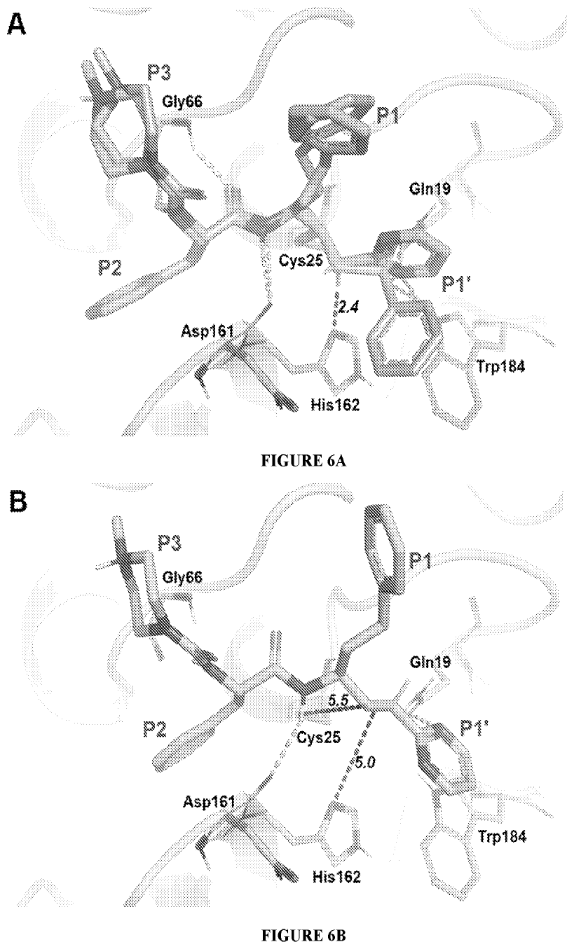

Nearly 60 new cysteine protease inhibitors of two classes: peptidomimetic vinyl heterocycles (PVHs) and masked aldehydes (MA) have been synthesized and characterized. Examples have been proven to kill trypanosomes in cell cultures of T. brucei and T. cruzi, and in a cell model of human infection for T. cruzi. Examples of potent inhibitors of cruzain show limited or significant selectivity over homologous human cysteine proteases (Cathepsins B, L, and S), and no toxicity when tested in human dermal fibroblasts at concentrations of 100 micromolar or less, and with apparent cellular activity against a cysteine protease of Trypanosoma brucei. Compounds 7, 11, 12, 13, 15, 30, 35, and 36 (FIGS. 5 and 6) nearly match or exceed the potency of the clinical candidate K11777 in cell cultures of trypanosomes and could be considered compounds suitable for pre-clinical development and testing in animal models of Chagas disease.

The most potent cruzain and cathepsin L inhibitors, respectively, of the MA class are: compounds 30, 32-36; (Kᵢ=18-100 nM and Kᵢ=11-58 nM). For inhibition of cruzain by the PVH class are compounds 7, 11, 12, 13, and 15 (Kᵢ=120-400 nM). The vinyl heterocycle inhibitors may be substituted with electron-withdrawing groups (compare compounds 11 and 13 with compounds 12 and 15) which facilitate reaction with thiols. Many of the most potent cruzain inhibitors of the PVH class of inhibitors were, respectively, ≥2 selective vs. human cathepsin L, S, and ≥20 selective vs. human cathepsin B. None of these cruzain inhibitors demonstrated toxicity in human fibroblasts. Several PVH inhibitors and two MA inhibitors inhibited in a dose-dependent manner growth of axenic cultures of both Trypanosoma cruzi (EC₅₀=2-20 μM) and Trypanosoma brucei brucei (EC₅₀=4-15 μM), which was either comparable or exceeded the activity of the clinical candidate inhibitor, K11777 (EC₅₀=>20 (T. cruzi) and 2 μM (T b. brucei)).

Peptidomimetic Vinyl Heterocycles (PVHs)

The heterocyclic groups of the PVHs include, as shown in FIG. 5: a benzyl group (R₃=III), a 2-pyrimidine group (R₃=IV), a 2-pyridine group (R₃=V), an N-methyl-2-pyridine group (R₃=V, R₄=methyl), a 4-pyrimidine group (R₃=VII), a 4-pyridine group (R₃=VI), a vinyl-2-oxazole (R₃=VIII), a 2-thiazole group (R₃=IX), an N-methyl-2-thiazole group (R₃=IX, R₄=methyl) and other vinyl-2-heterocycles in progress (2-triazole, and 2-triazine, for which it is expected that the heterocyclic moiety would provide an isosteric and isoelectronic replacement for an electrophilic propenamide (acrylamide) or a vinyl-sulfone. The ability to modify the electrophilicity of the vinyl group is by the addition of either electron-releasing or electron-withdrawing groups on the heterocyclic ring, which is expected to be in conjugation with the vinyl electrophile. Substituents are included on the heterocycles. Included are naturally-occurring phenylalanine (Phe) and non-natural-occurring (homo-Phe, 4-pyridyl-alanine) amino acids at the two sidechain positions of the PVHs and MA inhibitors. Current inhibitors are compiled in Table 1 below.

TABLE 1
Peptidomimetic Vinyl-Heterocycle Inhibitors of Cruzain and anti-trypanosomal activities.

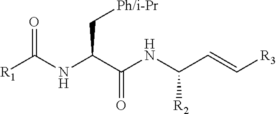

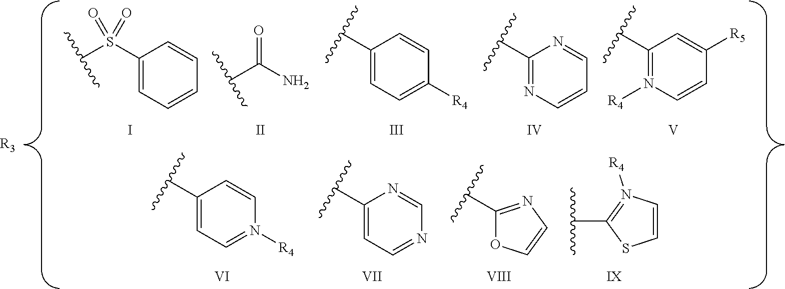

| | | | | | Cruzain Inhibition or Inactivation | | |
|---|---|---|---|---|---|---|---|
| | | | | | | | k_{inact}/K_{inact} |
| Compound | Structure | R₁ | R₂ | R₃ | Kᵢ (μM) | Kᵢ* (μM) | (M⁻¹s⁻¹) |
| K11777 | NMePip-Phe-hPhe-VSPh | NMePip | CH₂Bn | I | NA | 0.002^{b} | 234,000^{b,c} |
| 1 | Cbz-Phe-Phe-VSPh | BnO | Bn | I | ND | 0.0036 ± 0.0001 | ND |
| 2 | Cbz-Phe-Phe-vinyl-CONH₂ | BnO | Bn | II | 37 ± 2 | NA | 21.7 ± 0.8 |
| 3 | Cbz-Phe-hPhe-vinyl-CONH₂ | BnO | CH₂Bn | II | 3 ± 1 | NA | 1700 ± 500 |
| 4 | NMePip-Phe-hPhe-vinyl-CONH₂ | NMePip | CH₂Bn | II | 3.4 ± 0.4 | NA | 1900 ± 200 |
| 5 | Cbz-Phe-Phe-vinyl-Ph | BnO | Bn | III | 1.8 ± 0.1 | 0.87 ± 0.05 | NA |
| 6 | Cbz-Phe-Phe-vinyl-(4-NO₂)Ph | BnO | Bn | III, R₄ = NO₂ | ND | 0.37 ± 0.02 | NA |
| 7 | Cbz-Phe-Phe-vinyl-2Pyrmd | BnO | Bn | IV | 28 ± 1 | 0.364 + 0.004 | NA |
| 8 | Cbz-Phe-hPhe-vinyl-2Pyrmd | BnO | CH₂Bn | IV | >35 | NA | NA |
| 9 | NMePip-Phe-hPhe-vinyl-2Pyrmd | NMePip | CH₂Bn | IV | >10 | 2.2 ± 0.1 | NA |
| 10 | Cbz-Phe-Ala-vinyl-2Pyrmd | BnO | Me | IV | 58 6 | 25 ± 1 | NA |
| 11 | Cbz-Phe-Phe-vinyl-2Pyr | BnO | Bn | V | 5.5 ± 0.4 | 0.31 ± 0.01 | NA |
| 12 | Cbz-Phe-Phe-vinyl-2PyrNMe | BnO | Bn | V, R₄ = Me | 3.8 ± 0.4 | 0.28 ± 0.08 | NA |
| 13 | Cbz-Phe-hPhe-vinyl-2Pyr | BnO | CH₂Bn | V | 1.06 ± 0.07 | 0.171 ± 0.004 | NA |
| 14 | NMePip-Phe-hPhe-vinyl-2Pyr | NMePip | CH₂Bn | V | ND | 3.4 ± 0.1 | NA |
| 15 | Cbz-Phe-hPhe-vinyl-2PyrNMe | BnO | CH₂Bn | V, R₄ = Me | 0.76 ± 0.04 | 0.126 ± 0.004 | NA |
| 16 | Cbz-Phe-hPhe-vinyl-2-(4-OMe)-Pyr | BnO | CH₂Bn | V, R₅ = OMe | >5 | NA | NA |
| 17 | Cbz-Phe-hPhe-vinyl-2-(4-CF₃)-Pyr | BnO | CH₂Bn | V, R₅ = CF₃ | NA | 0.57 ± 0.05 | NA |
| 18 | Cbz-Leu-hPhe-vinyl-2Pyr | BnO | CH₂Bn | V | 7.8 ± 0.6 | 1.42 ± 0.09 | NA |
| 19 | Cbz-Phe-Ala-vinyl-2Pyr | BnO | Me | V | ND | 4.8 ± 0.2 | NA |
| 20 | Cbz-Phe-Lys-vinyl-2Pyr | BnO | (CH₂)₄NH₂ | V | 17.3 ± 0.3 | 0.87 ± 0.02 | NA |
| 21 | Cbz-Phe-Phe-vinyl-4Pyr | BnO | Bn | VI | ND | 5.5 ± 0.2 | NA |
| 22 | Cbz-Phe-Phe-vinyl-4PyrNMe | BnO | Bn | VI, R₄ = Me | 92 ± 5 | 4.0 ± 0.1 | NA |
| 23 | Cbz-Phe-Phe-vinyl-4Pyrmd | BnO | Bn | VII | 10.8 ± 1.4 | 1.14 ± 0.07 | NA |
| 24 | Cbz-Phe-Phe-vinyl-20xz | BnO | Bn | VIII | 10 ± 1 | 0.71 ± 0.01 | NA |
| 25 | Cbz-Phe-Phe-vinyl-2Thz | BnO | Bn | IX | ND | 1.71 ± 0.09 | NA |
| 26 | Cbz-Phe-Phe-vinyl-2ThzNMe | BnO | Bn | IX, R₄ = Me | ND | 0.94 ± 0.06 | NA |

ªData obtained at 25° C., pH 7.5; ^{b}Reported as apparent IC₅₀ in Ref. 32; ^{c}Reported as 32,500 M⁻¹s⁻¹ (pH 8.0) in Ref. 53; NA, not applicable; ND, not determined; app Kᵢ
and Kᵢ* are respectively, the apparent initial and tight-binding inhibition constants.

The PVHs demonstrated time-dependent inhibition of cruzain (and many inhibit falcipain-2) with inhibition constants ranging in values of 0.12-5.5 micromolar. Many of these inhibitors have also been profiled in terms of inhibition of human cathepsins B, L, and S. The heterocycles that provided the most potent inhibition were the vinyl 2-pyridine, the vinyl 2-pyrimidine, and the vinyl 2-(N-methylpyridine) within dipeptide scaffolds of Cbz-Phe-Phe, Cbz-Phe-hPhe, and NMe-piperazinyl-Phe-hPhe, the latter of which is the same scaffold found in the clinical candidate K11777, an irreversible cruzain inactivator containing a vinyl-(phenyl)sulfone “warhead” (apparent Kᵢ=4 nM). K11777 is approaching Phase I trials for the treatment of Chagas disease. PVH compounds 7, 11, 12-13, and 24 have been tested in cell cultures of Trypanosoma brucei brucei, associated with African sleeping sickness, and Trypanosoma cruzi, the causative agent of Chagas Disease, and exerted anti-trypanosomal activity in both species at concentrations ranging from 2-27 micromolar, in accord with the rank order of cruzain inhibition.

Masked Aldehydes (MAs)

For the masked aldehydes, all peptide analogues contain an aldehyde group on the C-terminus (Compounds 30-48, Table 2). A compound of the structure Cbz-Phe-Phe-CHO (Compound 28) is an (unmasked) aldehyde that inhibits cruzain and cathepsin L at respective inhibition constants of 0.44 and 0.31 nM. Masked aldehydes are similar dipeptide analogues which contain a 2-hydroxy group on the C-terminal sidechain (an ortho-tyrosine) and could include electron-donating and electron-withdrawing groups (R1) at carbon-4. MAs 30-36 display inhibition constants vs. cruzain of 18-350 nM, and compounds 30, 32-34-36 display anti-trypanocidal activities vs. T. brucei brucei (EC50=0.5-6.0 micromolar) and T. cruzi (EC50=3-30 micromolar) in T. cruzi-infected cell cultures. MAs 30-36 inhibit cathepsin L with inhibition constants 10.8-58 nM. Additional MA inhibitors will include the substitution of 2-carboxyl groups on the C-terminal phenylalanyl sidechain which may form a seven-membered-ring oxo-lactol with the terminal aldehyde, and which may be more amenable to ring opening (Aldehydes b and c). Additional MA inhibitors will also include a 2-carboxy-substituted L-phenylglycyl C-terminal residue and a 2-carboxy-homo-phenylalanyl C-terminal residue, which will allow the respective formation of a delta-lactol (six-membered-ring) and an epsilon-lactol (a seven-membered ring), the latter of which may also allow more facile opening of the lactol ring to afford the reactive aldehyde group. Substitution of the C-terminal phenyl ring with hydrogen or electron-donating or electron-withdrawing groups (R₁) at carbon-4 and other positions will allow exploration of the substitutions on the facility of ring opening of these lactols.

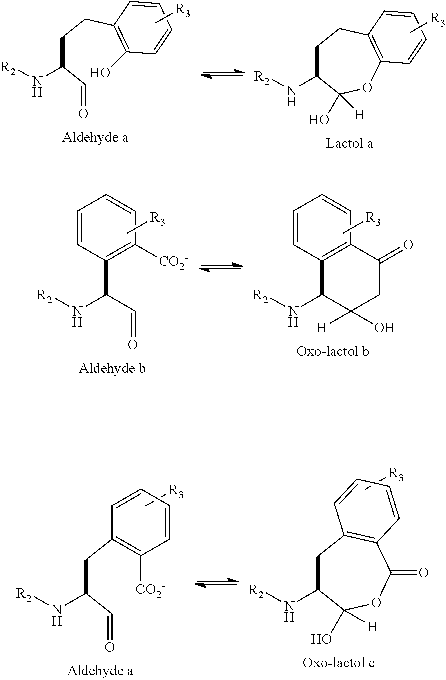

where
R₂ is NMe-Pip-Phe or Cbz-Phe; and
R₃ is hydrogen, methyl and other alkyl groups, —CF₃, —NO₂, —CN, —F, —Cl, —Br, —OMe, —NH₂, —COOH, —CO₂R (R=alkyl or aryl), —CONH₂.

TABLE 2
“Masked” Aldehyde Inhibitors of Cruzain, Cathepsins L and B , and SARS-
CoV-2 3CL protease, and anti-trypanosomal and anti-CoV-2 activities.
Aldehyde and Masked Inhibitors of Cruzain, Human Cathepsins L and B, 3CL protease, and Anti-trypanosomidal and anti-CoV-2 A

| Com- pound Number and | 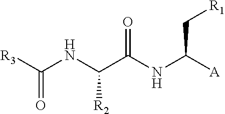 Formula I | | | | Kᵢ* (nM) | | | | EC₅₀ T. cruzi- infected cardiomyo- | EC₅₀ T. b. brucei PCF | EC₅₀ T. b. brucei BSF | Anti- CoV- 2 EC₅₀ (μM) Vero E6 | Anti- CoV- 2 EC50 (μM) A549/ ACE 2 |
|---|---|---|---|---|---|---|---|---|---|---|---|---|---|
| Name | R₆ | R₃ | R₂ | R₁-A | Cruzain | hCatL | hCatB | 3CLpro | blasts (μM) | (μM) | (μM) | Cells | Cells |
| 28 LL205 | H | 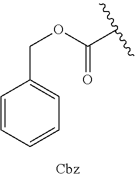 [SMILES: *C(=O)OCc1ccccc1] | 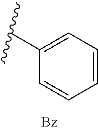 [SMILES: *c1ccccc1] | 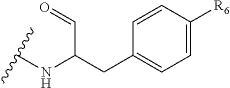 | 0.44 ± 0.02 | 0.31 ± 0.01 | ND | >10,000 | 0.5 ± 0.4 | 8 | 3 | 0.5 | ND |
| 29 LL127 | OMe | Cbz | Bz | 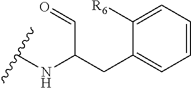 | 22 ± 2 | ND | ND | >10,000 | ND | ND | ND | ND | ND |
| 30 LL166 | H | Cbz | Bz | 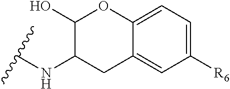 | 49 ± 2 | 28 ± 0.9 | 4500 ± 100 | >10,000 | 3.0 ± 0.9 | 17 | ND | 5 | ND |
| 31 LL232 | OMe | Cbz | Bz | 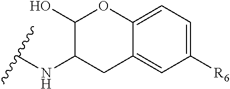 | 350 ± 32 | 38 ± 1.6 | 5500 ± 400 | >10,000 | ND | 11 | 3 | 2.5 | ND |
| 32 LL254 | Me | Cbz | Bz | 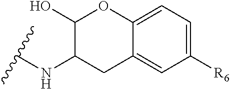 | 103 ± 5 | 58 ± 2.2 | 6100 ± 900 | >10,000 | 5.7 ± 0.6 | 7 | 0.5 | ND | ND |
| 33 LL291 | Cl | Cbz | Bz | 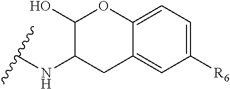 | 74 ± 10 | 27 ± 0.7 | 1400 ± 100 | >10,000 | ND | 10 | 6 | ND | ND |
| 34 LL294 | F | Cbz | Bz | 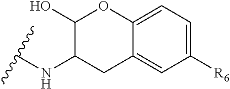 | 48 ± 2 | 23 ± 1.3 | 2300 ± 200 | >10,000 | 3.5 ± 0.6 | 11.1 | 2.7 | 4 | ND |
| 35 LL322 | CO,Me | Cbz | Bz | 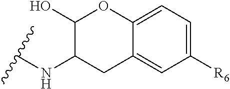 | 18 ± 0.5 | 10.8 ± 0.8 | 670 ± 30 | >10,000 | 3.5 ± 0.3 | 6.7 | ND | 2.5 | ND |
| 36 BC552A | H | 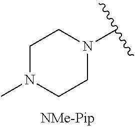 [SMILES: *N1CCN(C)CC1] | Bz | 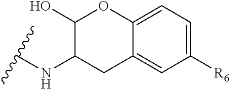 | 47 ± 2 | 20 ± 0.9 | 1270 ± 70 | >10,000 | 29 ± 0.3 | 4 | 0.6 | 7.5 | ND |
| 37 LL497 | H | NMe-Pip | Bz | 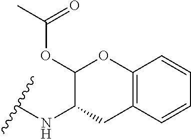 | ND | ND | ND | ND | 13 ± 2 | ND | 3.7 | 2.5 | 0.31 |
| 38 LL494 | H | NMe-Pip | Bz | 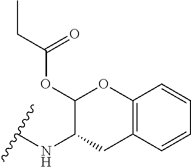 | ND | ND | ND | ND | ND | ND | ND | 2.5 | 0.62 |
| 39 LL490 | H | NMe-Pip | Bz | 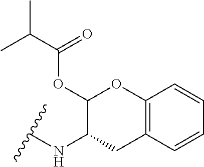 | ND | ND | ND | ND | 14 ± 6 | ND | ND | 4 | 0.31 |
| 40 LL490 | H | NMe-Pip | Bz | 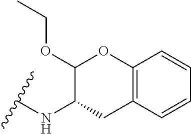 | ND | ND | ND | ND | ND | ND | ND | ND | ND |
| 41 LL491 | H | NMe-Pip | Bz | 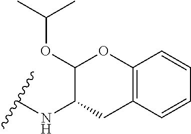 | ND | ND | ND | ND | ND | ND | ND | >20 | 5 |
| 42 BC666 | H | NMe-Pip | Bz | 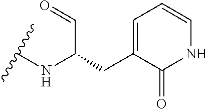 [SMILES: *N[C@H](C=O)Cc1ccc[nH]c1=O] | 2.1 ± 0.1 | 2.3 ± 0.1 | ND | 860 ± 90 | ND | ND | ND | >20 | >20 |
| 43 LL- 478 | NA | 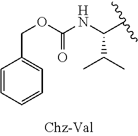 [SMILES: *[C@@H](NC(=O)OCc1ccccc1)C(C)C] | 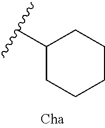 [SMILES: *C1CCCCC1] | 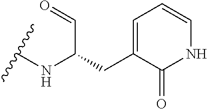 [SMILES: *N[C@H](C=O)Cc1ccc[nH]c1=O] | ND | 50 ± 10 | ND | 9 ± 2 | ND | ND | ND | ND | 5 |
| 44 LL- 482 | NA | 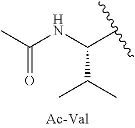 [SMILES: *[C@@H](NC(C)=O)C(C)C] | 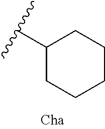 [SMILES: *C1CCCCC1] | 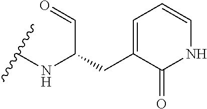 [SMILES: *N[C@H](C=O)Cc1ccc[nH]c1=O] | ND | 140 ± 30 | ND | 60 ± 20 | ND | ND | ND | >20 | ND |
| 45 BC- 671 | NA | 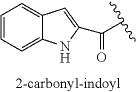 [SMILES: *C(=O)c1cc2ccccc2[nH]1] | 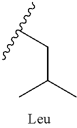 [SMILES: *CC(C)C] | 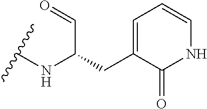 [SMILES: *N[C@H](C=O)Cc1ccc[nH]c1=O] | ND | 39 ± 3 | ND | 190 ± 30 | ND | ND | ND | >20 | >20 |
| 46 BC- 674 | NA | 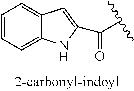 [SMILES: *C(=O)c1cc2ccccc2[nH]1] | 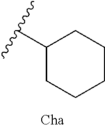 [SMILES: *C1CCCCC1] | 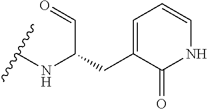 [SMILES: *N[C@H](C=O)Cc1ccc[nH]c1=O] | ND | ND | ND | 60 ± 10 | ND | ND | ND | >20 | >20 |
| 47 LL- 482 | NA | 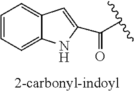 [SMILES: *C(=O)c1cc2ccccc2[nH]1] | 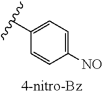 | 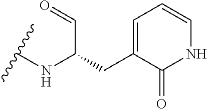 [SMILES: *N[C@H](C=O)Cc1ccc[nH]c1=O] | ND | ND | ND | 260 ± 40 | ND | ND | ND | >20 | >20 |
| 48 LL- 753A | NA | 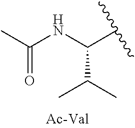 [SMILES: *[C@@H](NC(C)=O)C(C)C] | 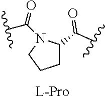 | 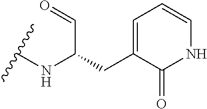 [SMILES: *N[C@H](C=O)Cc1ccc[nH]c1=O] | ND | ND | ND | 1160 ± 80 | ND | ND | ND | >20 | >20 |
| 49 BC- 742B | NA | 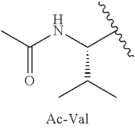 [SMILES: *[C@@H](NC(C)=O)C(C)C] | 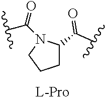 | 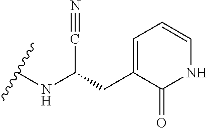 [SMILES: *N[C@H](C#N)Cc1ccc[nH]c1=O] | ND | ND | ND | 143 ± 3 | ND | ND | ND | >20 | >20 |

Kᵢ* is the inhibition constant obtained >10 minutes following initiation of reaction by addition of enzyme; ND, no data

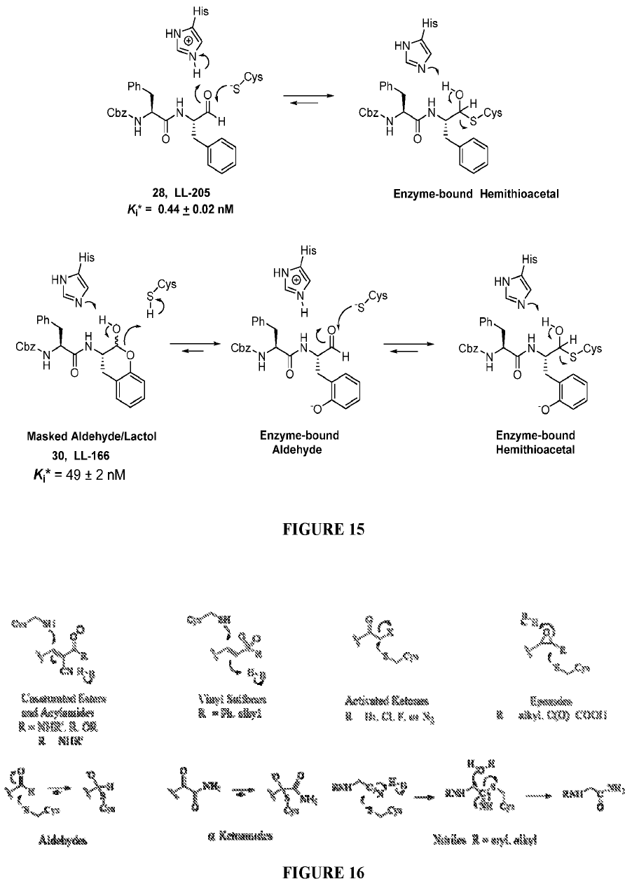

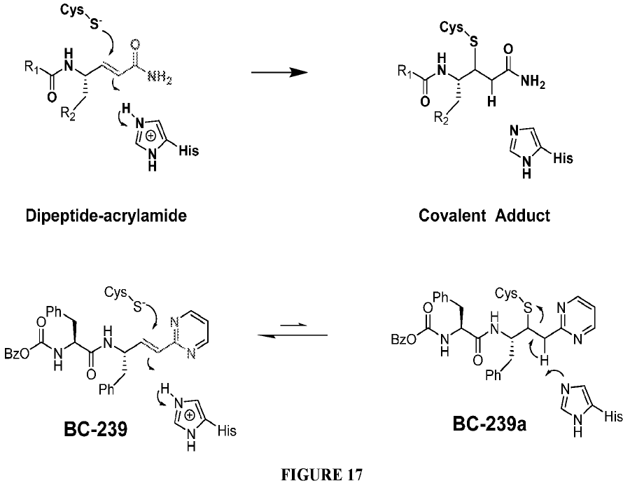

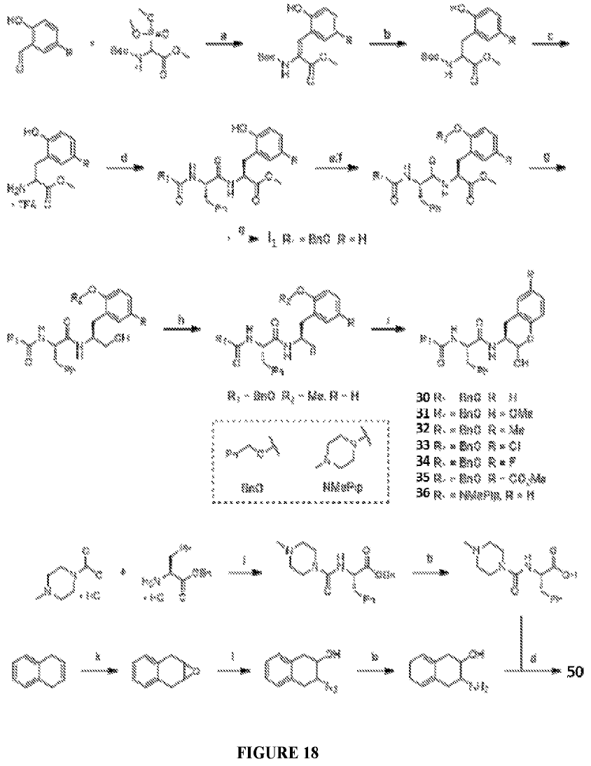

Dipeptide Lactols/Masked Aldehydes. The extraordinary potency of the dipeptide aldehyde (compound 28, Kᵢ*=0.44 nM) vs. cruzain was utilized to discover inhibitors. While free aldehydes are unsuitable as drug candidates, a compound that elaborates an aldehyde only in an enzyme active site provides a novel approach to the inhibition of cruzain and other cysteine proteases. Compounds 30-36, which contain terminal aldehyde groups and an ortho-substituted Tyr group in the P₁ position were synthesized and characterized. It was expected that these compounds would form a δ-lactol between the free ortho-hydroxy group and the aldehyde (FIG. 15), masking the latter substituent and possibly allowing its unmasking to an aldehyde in the active site of cruzain. Indeed, analysis of a non-polar solution of Compounds 30 and 36 showed no presence of free aldehyde, and both exhibited time-dependent inhibition of cruzain (Kᵢ*=49 and 47 nM, respectively). For Compound 36, no free aldehyde could be detected in aqueous buffer (pH=7) upon treatment with phenylhydrazine, Furthermore, carbon-13 nuclear magnetic resonance analysis of Compound 36 in which the aldehydic carbon was 100% ¹³C revealed only the presence of a lactols in aqueous buffer (pH=7.5). Addition of equimolar amounts of cruzain to this sample resulted in changes in the chemical shifts to those consistent with the formation of an enzyme-bound hemithioacetal. Substitution of carbon-5 of the o-Tyr group with a —CO₂Me group provided Compound 25 (Kᵢ*=19 nM), which is the most potent reversible cruzain inhibitor to date. Analysis of a crystal structure of cruzain-Compound 36 is ongoing.

Pro-Drugs of Masked Aldehydes

Compounds 37-41 are pro-drugs of the self-masked aldehyde, compound 36. For compounds 37-39, the lactol hydroxyl group has been O-acetylated (37), O-propanoylated (38), and O-isobutanoylated (39).⁶¹ We have demonstrated that these acyl groups are removed by cellular esterases to afford Compound 36 both in vitro, and apparently, in cellular culture.⁶¹ As shown in Table 2, compounds 37-39 were significantly more effective at blocking cellular infection by SARS-CoV-2 than its parent masked aldehyde 36. Compounds 40 and 41 are pro-drug forms of compound 36 in which mixed acetals have been prepared.

Optimization of Peptide Scaffolds

The purpose of this analysis was to ascertain the most favorable peptide sidechains for cruzain substrates, and then incorporate the optimal sidechains into peptidomimetic vinyl-heterocycles to improve their potency and selectivity. If the dipeptidic vinyl-heterocycle inhibitors do indeed inhibit cruzain by addition of the thiolate of Cys25 to the double bond, then the values of k_{cat}/Kₘ (the specificity constant) for the fluorogenic substrates should provide an approximate linear correlation with values of k_{inact}/K_{I} (which is the “specificity constant” of inactivation).

Dipeptide fluorogenic substrates of cruzain (Cbz-P₂-P₁-AMC; P₂=Phe, Leu, Arg, Val; P₁=Arg, Ala; AMC=7-amino-4-methyl coumarin) (see Zhai, et al.,⁴⁰ Table 1; descending order of relative k_{cat}/Kₘ) have been kinetically characterized. From Zhai, et al.,⁴⁰ Table 1, the most optimal of these substrates was Cbz-Phe-Arg-AMC (rel. k_{cat}/Kₘ=1.0) and Cbz-Leu-Phe-AMC (rel. k_{cat}/Kₘ=0.27). An additional panel of fluorogenic substrates was prepared and characterized to explore the effect of substitutions of un-natural amino acid sidechains, including sidechains found in the current drug-candidate cruzain inactivator K11777, for which P₃=N-methyl-piperazinyl (NMePip), P₂=Phe, and P₁=homophenylalanyl (hPhe). Cbz-Phe/Leu-hPhe-AMC were both good substrates (rel. k_{cat}/Kₘ=0.56/0.29); comparable in activity to Cbz-Phe/Leu-Arg-AMC. Cbz-Phe-Phe-AMC, while a poor substrate (rel. k_{cat}/Kₘ=0.054) has a Kₘ value of high affinity (Kₘ=0.34 μM). Accordingly, both the Cbz-Phe-Phe and Cbz-Phe-hPhe dipeptide scaffolds for new peptidomimetic vinyl heterocycle inhibitors (Table 1) have been utilized. The viability of a 4-pyridyl-Ala residue (a potential mimic of both Phe and Arg (when the pyridine is protonated)) was explored at both the P₂ and P₁ positions, and Cbz-Phe-4PyrAla-AMC was found to be a good substrate (relative k_{cat}/Kₘ=0.14). Interestingly, N-Me-piperazinyl-Phe-hPhe-AMC, which contains an identical di-peptide scaffold to K11777, was a poor substrate (rel. k_{cat}/Kₘ=0.093). These results indicate that a dipeptide scaffold of P₂=Cbz-Phe or NMe-Pip-Phe and P₁=Arg, hPhe, or 4PyrAla may be among the best to incorporate into the vinyl heterocycle framework, as is manifest in the most potent cruzain inhibitors displayed in Table 1.

The potency of dipeptide aldehyde 28 was utilized to establish a new strategy for cruzain inhibition. A new class of cruzain inhibitor was prepared and evaluated, in which the P1 sidechain Phe bears a 2-hydroxyl group (ortho-tyrosine) capable of forming a lactol substituent with a terminal aldehyde—a “masked” aldehyde—that may be able to reestablish the inhibitory aldehyde group in the active site of cruzain (Compounds 30-26 in Table 2).

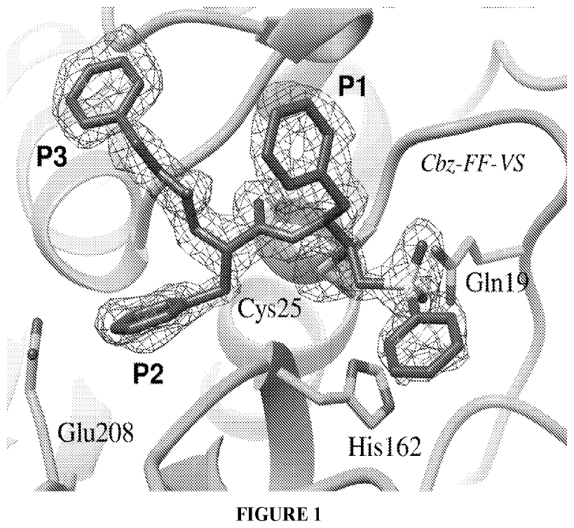

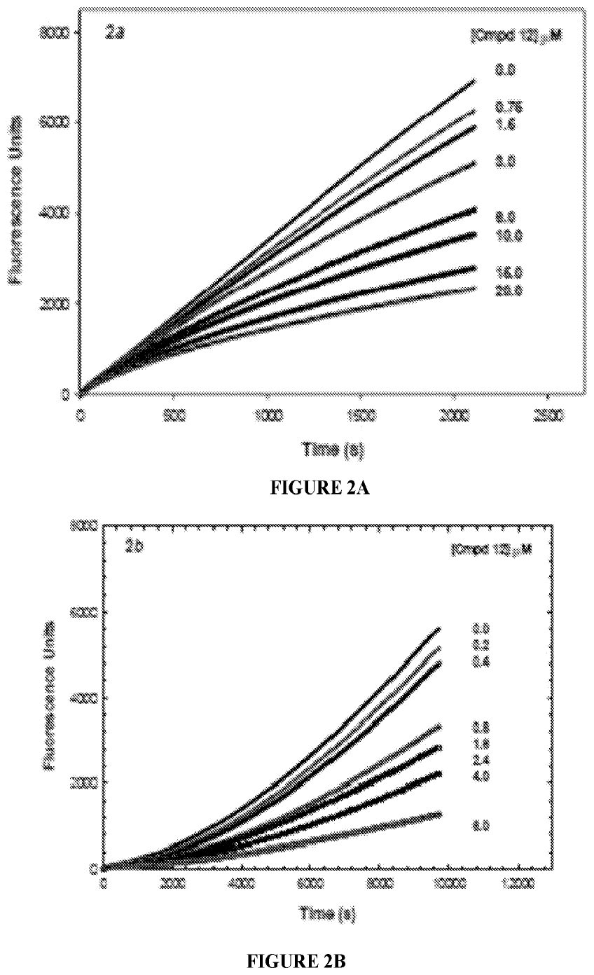

Scheme 1 is a kinetic depiction of the initial, rapid collision between enzyme (E) and inhibitor (I) to form complex EI, characterized by the inhibition constant Kᵢ. For time-dependent inhibitors or covalent inactivators, EI progresses to a second, tighter complex EI* over the course of minutes, for which the tighter inhibition is expressed by Kᵢ*. The situation of Kᵢ*<Kᵢ exists when k₄<k₃, as for reversible covalent inhibitors, and k₄˜0 and Kᵢ*˜0 for irreversible covalent inactivators. Values of k₃ and k₄ may be obtained, allowing calculation of the residence time of the inhibitor (0.693/[k₂k₄/(k₂+k₃+k₄)]. For time-dependent inhibitors, initiation of reaction by adding enzyme to substrate and inhibitors leads to curvilinear time courses as exemplified FIG. 2A, in which reaction rates demonstrably decrease as the EI* complex forms. Alternatively, extended pre-incubation of enzyme and inhibitor, followed by dilution of the inhibitor and initiation of reaction with high concentrations of substrate leads to curvilinear plots as exemplified in FIG. 2B, in which E reforms from EI*. Results of this analysis for cruzain inhibitors are collected in Table 1. Only those inhibitors that effected reversible, time-dependent inhibition as depicted in FIG. 2 provided values of Kᵢ*, others provided Kᵢ, while covalent, irreversible inactivators, are characterized by values of k_{inact}/K_{I}, for which K₁ is similar to Kᵢ*. Nearly 60 cruzain inhibitors of the peptidomimetic vinyl heterocycle and “masked” aldehyde inhibitor classes have been synthesized and characterized to date. Compounds that exerted irreversible inactivation are characterized by the second order rate-constant k_{inact}/K_{I}, while reversible inhibitors either did (Kᵢ and Kᵢ*; FIG. 2) or did not (Kᵢ only) elicit time-dependent inhibition. The replacement of the P1 homo-Phe (hPhe) group of K11777 with a Phe sidechain resulted in an inhibitor of low-nanomolar potency (Compound 1), but which exhibited kinetically reversible inhibition of cruzain. However, the crystal structure obtained for Compound 1 bound to cruzain indicated the formation of a C—S bound between Cys-25 and Compound 1 (FIG. 1). This may indicate that a Phe group at the P₁ position may partly impede the ability of an adjacent vinyl electrophile to access Cys-25. Three terminal acrylamides (R₄=2) within the NMePip-Phe-hPhe, Cbz-Phe-Phe-hPhe, and Cbz-Phe-Phe scaffolds (Compounds 2-4), were evaluated and demonstrated that, like Compound 1, acrylamides bearing the Cbz-Phe-hPhe and NMe-Pip-Phe-hPhe scaffolds afforded apparently irreversible covalent inactivators, while the Cbz-Phe-Phe-acrylamide (2) is a poor inhibitor with time-dependent properties that are only established after long incubation periods. As with Compound 1, a Phe, rather than a hPhe group at the P₁ position may retard covalent formation.

Substitution of these terminal acrylamides with isoelectronic, conjugated vinyl-heterocycles would afford inhibitors which form covalent, but reversible, adducts of the cysteine proteases, owing to the re-establishment of aromatic conjugation upon reversal of adduct formation. Such inhibitors would exhibit the kind of time-dependent behavior as seen in FIG. 2 dependent on the stability of the covalent complex. Of the PVH class, 7 different vinyl heterocyclic or vinyl phenyl groups have been evaluated. For those heterocycles leading to the most potent PVH inhibitors (R₄=1, 4, and 5), the inhibitory activity within three peptide scaffolds (Cbz-Phe-Phe, Cbz-Phe-hPhe, and NMe-Pip-Phe-hPhe) has been ascertained, this last scaffold being that found in K11777, and also with other amino acids in the P₁ and P₂ positions. The Phe-Phe-vinyl-phenyl inhibitor Compound 5 is a poor inhibitor but substitution of the para position with a nitro group elicits time-dependent inhibition (Compound 6) with an improvement in potency by 50-fold.

This suggests that the substitution of the vinyl-phenyl group with an electronegative substituent may increase the electrophilicity of the vinyl group to thiolation by Cys-25. The vinyl-phenyl substituent (Compounds 5 and 6) led to potent inhibitors, but with poor aqueous solubility. Cruzain inhibitors 7-27 explored the inhibitory properties of six heterocyclic groups (substituents R₃=IV-IX in Table 1) appended to the presumed electrophilic vinyl group within several dipeptide scaffolds. The vinyl-2-pyrimidine (R₃—IV), vinyl-2-pyridine (R₃=V), vinyl-2-oxazole (R₃=VIII) and vinyl-2-thiazole (R₃=IX) groups, unlike the heterocycles R₃=VI and R₃=VII, maintain isoelectronic similarity to the reactive acrylamides, and provided time-dependent inhibitors, many of nanomolar potency. The potent, time-dependent inhibition of cruzain displayed by these PVH inhibitors may be due to the reversible formation of an adduct with active-site Cys-25, but importantly, to date there is no chemical data demonstrating the temporal formation of a C—S bond between cruzain and the PVHIs. Most of these compounds induce time-dependent inhibition on cruzain, but all are kinetically reversible with dissociation half-lives of 6-20 minutes. In FIG. 2B, a significant lag phase for recovery of cruzain activity was observed, indicative of the slow desorption of the inhibitor, with or without the formation of a covalent bond with Cys-25. Of these PVHIs, the 2-pyridyl, the charged 2-N-methyl-pyridyl, and the 2-pyrimidinyl present the most interesting heterocycles for further exploration.

To date, there is no evidence of the formation of a thiolate adduct of Cys25-SH with the vinyl bond of our compounds, either through analysis by ¹³C-NMR or x-ray crystallography, despite unsuccessful attempts to produce a structure. The chemical reactivity of the vinyl group was evaluated in several PVHIs with the cysteine group of glutathione by LCMS, and kinetic and equilibrium constants were ascertained for addition of the thiol to this double bond in K11777, Compounds 1, 7, 11, and 12, which respectively contain a vinyl sulfone (R₃=I), a vinyl-2-pyrimidine (R₃=IV), a vinyl-2-pyridine (R₃=V) and a vinyl-2-N-methylpyridine (R₃=V, R₄=methyl). Formation of glutathione (GSH) adducts was slow with K11777 (Table 3), and at an apparent equilibrium constant of 160 M⁻¹; the fraction of the adduct of GSH-K11777 was 17% that of the inputted compounds.

TABLE 3
Second-order rate constants of thiolation
of PVH inhibitors with glutathione.
Table. Kinetic Constants of
Thiolation of Cruzain Inhibitors^{a}

| Compound | Rate of Thiolation, k (mM⁻¹min⁻¹) | K_{eq} (M⁻¹) |
|---|---|---|
| K11777 | 0.00028 ± 0.0004 | NA |
| 7 | Negligible | NA |
| 11 | Negligible | NA |
| 12 | 0.037 ± 0.002 | 7400 |
| 15 | 0.054 ± 0.004 | 2400 |
| 17^{b} | Negligible | NA |
| 25 | Negligible | NA |
| 26 | 0.015 ± 0.005 | 930 |

^{a}1 mM glutathione was mixed with 0.5 mM K11777 and compounds in Tris (pH 8.0), 10% DMSO (v/v) at room temperature. Aliquots were analyzed by LCMS as described; ^{b}5 mM glutathione was used for 17; NA, not applicable.

This is interesting given that K11777 forms an irreversible adduct with cruzain, but not with GSH. Reaction between GSH was negligible with both the vinyl-2-pyrimidine of Compound 7 and vinyl-pyridine of Compound 11. However, N-methylation of the vinyl-2-pyridine resulted in a much more electrophilic vinyl group: addition of GSH to the vinyl group of Compound 12 (and Compound 15) occurred at an apparent rate which was 8-fold greater than that of K11777, and at equilibrium (K_{eq}=1300 M⁻¹); the GSH-Compound 12 adduct comprises 62% of the total added reactants. These results demonstrate that the reactivity of vinyl-heterocycles with GSH and presumably Cys-25 varies with the nature of the heterocycle, and that N-methylation of the pyridine of Compound 12 converts an unreactive vinyl-pyridine to one which reacts with GSH to form a thiolated adduct. However, Compound 12 and its un-methylated analogue 11 are equipotent as cruzain inhibitors, suggesting that the enhanced electrophilicity of 12 provides no improvement in binding to cruzain.

Selectivity Studies

Cruzain inhibitors vs. human cysteine proteases cathepsins L, B, and S (Table 4) were evaluated. Cruzain has, respectively, 25%, 15%, and 23% amino acid identity with human cathepsins L, B, and S, wherein TbCatB, an essential cysteine protease in T. brucei, has 40% identity with human cathepsin B. Cruzain inactivator K11777 containing the vinyl-sulfone warhead showed limited selectivity for cruzain vs. the human cathepsins. However, as recently reported the lack of toxicity of K11777 in dogs, rats, and primates in extensive studies underscores the potential irrelevance of its potent inactivation of cathepsins B, L, and S (McKerrow J H (2018) Update on drug development targeting parasite cysteine proteases. PLoS Negl Trop Dis 12(8): e0005850). The PVH inhibitors 7 and 11-13, and 15 are 3-14 selective vs. human cathepsin L and S (equipotent for 7). While the masked aldehyde 30 is more than 10-fold selective for cruzain vs. hCat L and S, its analogue Compound 35 inhibits cathepsin L 6-fold more potently than cruzain. All inhibitors in Table 4 are 40-130-fold selective vs. human cathepsin B.

TABLE 4
Enzymatic selectivity of cruzain inhibitors.
Table. Enzymatic Selectivity of Cruzain Inhibitors^{a}

| | | Kᵢ* (μM) | | | |
|---|---|---|---|---|---|
| | | | Human | Human | Human |
| Compound | Structure | Cruzain | Cathepsin L | Cathepsin B | Cathepsin S |
| K11777 | NMePip-Phe-hPhe-VSPh | IC₅₀ = 0.2 nM^{b} | IC₅₀ = 0.2 nM^{b} | IC₅₀ = 5.7 nM^{b} | IC₅₀ = 0.6 nM^{b} |
| 7 | Cbz-Phe-Phe-vinyl-2Pyrmd | 0.28 ± 0.01 | 1.1 ± 0.1 | 32 ± 3 | 0.37 ± 0.02 |
| 11 | Cbz-Phe-Phe-vinyl-2Pyr | 0.28 ± 0.02 | 4.3 ± 0.5 | 28 ± 4 | 1.8 ± 0.3 |
| 12 | Cbz-Phe-Phe-vinyl-2PyrNMe | 0.30 ± 0.02 | 0.70 ± 0.04 | 19 ± 4 | 0.87 ± 0.07 |
| 13 | Cbz-Phe-hPhe-vinyl-2Pyr | 0.123 ± 0.004 | 1.9 ± 0.2 | 6.5 ± 0.9 | 1.41 ± 0.06 |
| 15 | Cbz-Phe-hPhe-vinyl-2PyrNMe | 0.088 ± 0.002 | 0.88 ± 0.06 | 37 ± 6 | 0.32 ± 0.04 |

^{a}Inhibition data obtained at pH 5.5, 25° C. in 10% DMSO (v/v); ^{b}Reported as apparent IC₅₀ in Ref. 32.

Importantly, none of the new cruzain inhibitors, where tested, exhibited toxicity in human dermal fibroblasts at 0.1 mM or less (Table 5). In contrast, the K11777 demonstrated toxicity when evaluated in human dermal fibroblasts with a value of EC₅₀=0.07 mM.

TABLE 5
Effects of cruzain inhibitors on trypanosome and human cell growth.
Table. Effects of Cruzain Inhibitors on Trypanosome and Human Cell Growth^{a}

| | | | T. cruzi - | | | | | |
|---|---|---|---|---|---|---|---|---|
| | | T. cruzi | infected | T. brucei | T. brucei | Human | | |
| | | axenic | cardiomyoblasts | brucei | brucei | Cell | C2C12 | Selectivity |
| | Cruzain | culture | (C2C12) | PCFs | BSFs | Cytotoxicity | Cytotoxicity | Index |
| Compound | Kᵢ* (μM) | EC₅₀ (μM) | EC₅₀ (μM) | EC₅₀ (μM) | EC₅₀ (μM) | CC₅₀ (μM) | CC₅₀ (μM) | CC₅₀/EC₅₀ |
| K11777 | IC₅₀ = 2 nM^{b} | >20 | 0.7 ± 0.2 | 1.7 ± 0.5 | 0.09 ± 0.06 | 60-100 | >10 | 140 |
| 7 | 0.364 ± 0.004 | 20 | 9.0 ± 0.5 | 7.1 ± 0.9 | 10.4 ± 0.2 | >100 | >10 | >10 |
| 9 | 2.2 ± 0.1 | 20 | ND | 15 ± 2 | >20 | ND | ND | ND |
| 11 | 0.31 ± 0.01 | >20 | 4.9 ± 0.2 | 5 ± 1 | 5 ± 4 | >100 | >10 | >20 |
| 12 | 0.28 ± 0.08 | 8.7 ± 0.1 | 9.9 ± 0.5 | 13 ± 3 | 6.6 ± 0.6 | >100 | >10 | >10 |
| 13 | 0.171 ± 0.004 | >20 | 5.9 ± 0.3 | >10 | 4 ± 2 | >100 | >10 | >20 |
| 15 | 0.126 ± 0.004 | 2.1 ± 0.1 | 5.4 ± 0.9 | 5.9 ± 0.2 | 2.8 ± 0.1 | >100 | >10 | >20 |
| 24 | 0.71 ± 0.01 | >20 | ND | 27 ± 5 | ND | >100 | ND | ND |

^{a}Effects of inhibitors were evaluated as EC₅₀ for axenic T. cruzi, PCFs/BSFs of T. b. brucei and T. cruzi -infected murine cardiomyoblasts. The Selectivity Index is the ratio of inhibitor cytotoxicity in human dermal fibroblasts (CC₅₀)/trypanosomacidal activity (EC₅₀) in infected cardiomyoblasts.
^{b}Ref. 32.

Evaluation of the trypanocidal activity of new P2VHIs. Selected cruzain inhibitors in axenic cell cultures of Trypanosoma brucei brucei (procyclic trypomastigotes; ATCC PRA-381) and Trypanosoma cruzi (epimastigote forms; strain Y, ATCC 50832GFP) were tested. T. b. brucei is grown in SDM-79 medium, and T. cruzi is grown in ATCC medium (1029 LIT medium). Both media included fetal calf serum (10%) and penicillin/streptomycin (50 U/mL). Test compounds, including the clinical candidate K11777, were dissolved in 100% DMSO, and added at final concentrations of 0.5-20 μM (maximum DMSO=1% (v/v)). T. brucei and T. cruzi (5 mL in flask cultures) at 26° C. were seeded at ˜3×10⁶ cells, and diluted daily maintaining a mid-log growth phase for up to 100 hrs. Cell counts were scored using a Z2 Coulter Counter. After each cell dilution, fresh compound or an equal volume of DMSO (control samples), was supplemented into the cultures. Effects of the most potent cruzain inhibitors on cell growth of cultures of T. brucei brucei, T cruzi, and human dermal fibroblasts are compiled in FIG. 9. Cruzain inhibitors where the highest concentration reflects the upper limit of solubilities of the inhibitors in 1% (v/v) DMSO solutions were evaluated. These inhibitors are likely to bind to proteins in fetal calf serum, and for these reasons, fresh inhibitors were added each time cells were re-fed. K11777 exhibited potent growth inhibition of procyclic trypomastigotes of T. brucei brucei (EC₅₀=2.3 μM), but surprisingly, was a poor inhibitor of cell growth of epimastigotes of T. cruzi (EC₅₀˜60 μM). Ndao et al. evaluated K11777 and nitrile-containing cruzain inhibitors in T. cruzi epimastigotes, uimportantly, nonesing culture conditions similar to those here, and found K11777 to have trypanocidal activity at concentrations consistent with the data here (EC₅₀10-100 μM; Ndao et al. (2014) Antimicrobial Agents & Chemother 58, 1167-1178; PMCID: PMC3910870).

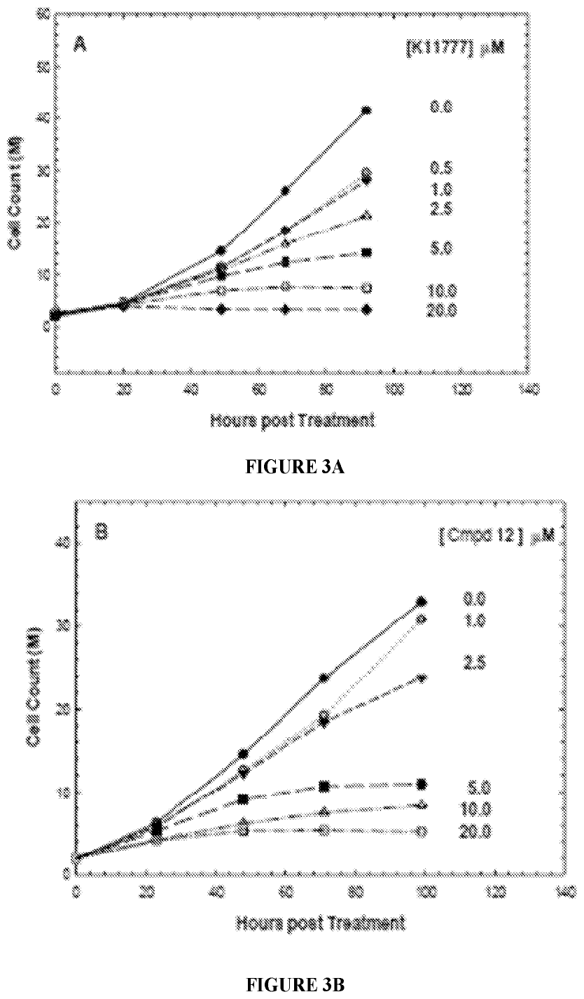

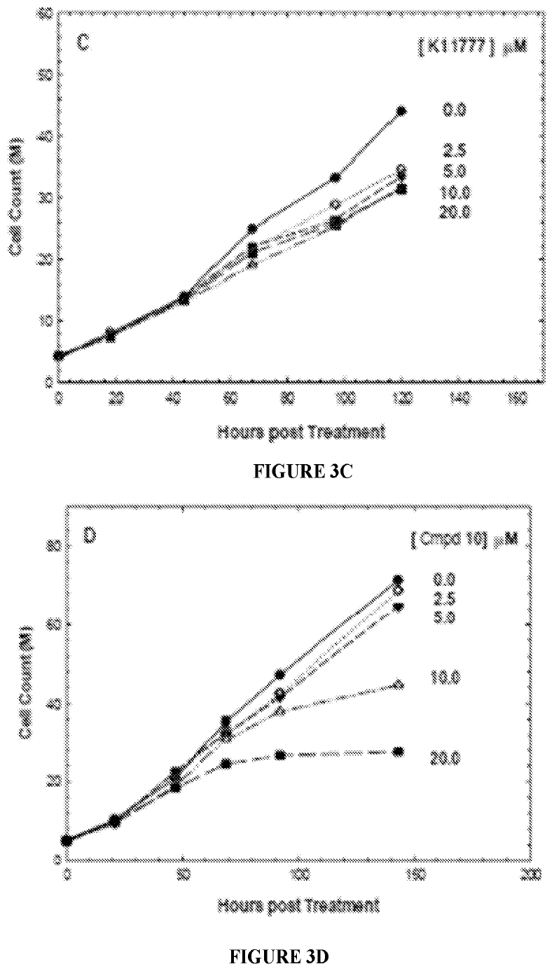

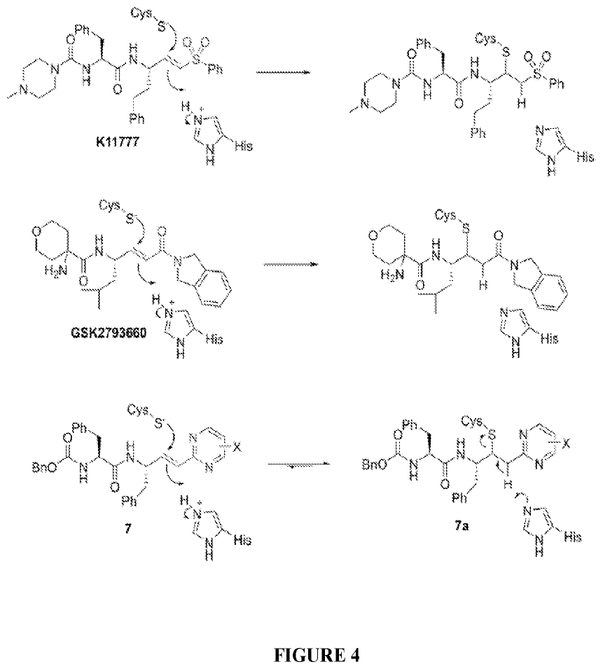

In T. b. brucei, Compounds 11 and 17 were poorly effective, wherein cell growth was inhibited by ≤50% at 20 μM. However, Compounds 7, 9, 11-13, 15, 30, and 35 inhibited T. brucei growth with values of EC₅₀=4-15 μM. Values of EC₅₀ for these PVHIs roughly correlate with their values of Kᵢ*. Importantly, Compounds 13 and 35 (EC₅₀=5.5 and 3.7 μM, respectively) the most potent PVH and MA inhibitors of cruzain, are nearly equipotent with K11777 in terms of growth inhibition of T. b. brucei (FIG. 9; FIG. 3). This is a surprising result given that these PVHIs are poor inhibitors of human cathepsin B, and presumably the putatively essential cysteine protease of T. brucei, TbCatB. This result suggests that a cathepsin L-like cysteine protease in T. b. brucei, such as brucipain (or TbCatL), (Nkemgu et al (2003) Intl. Jour. Antimicrob. Agents 22, 155-159, PMID: 12927956) is essential for growth of trypomastigotes, and investigation of this protease(s) will be the focus of future studies. Compounds 7, 10, 12, 30, and 35 likewise inhibited growth of epimastigotes of T. cruzi (EC₅₀=2-20 μM) while K11777, compounds 9, 11, and 17 were poorly effective (Table 5; FIG. 3). In particular, compounds 10 and 12, both of which contain N-methyl-2-pyridine vinyl heterocycles, were comparably potent (EC₅₀=8.6 and 2.1 μM, respectively), and were, at minimum, 10-fold more active than K11777. That both compounds 10 (Kᵢ*=0.28 μM) and 12 (Kᵢ*=0.12 μM) possess a common vinyl heterocycle (which readily forms an adduct with a thiol), and are cytotoxic to both T. b. brucei and T. cruzi, encourages further optimization of this class of PVH.

Selected cruzain inhibitors were further evaluated in a cell model of T. cruzi infection (Table 5). PVH inhibitors 7, 9, 10, 11, and 12, and MA 30 exhibited efficacy against 72-hr T. cruzi infection of murine cardiomyoblasts at values of EC₅₀=5-8 μM, while displaying no cytotoxicity of the host cardiomyoblasts (CC₅₀>10 μM). Importantly, the MA 36 (EC₅₀=0.54 μM) was more trypanocidal than K11777 in cultures of T. cruzi epimastigotes.

Of the inhibitors studied, EC₅₀ values in the infection T. cruzi are comparable to those obtained in axenic cultures of T. b. brucei.

Where tested, none of the new cruzain inhibitors displayed cytotoxicity when added to cultures of human dermal fibroblasts (no evidence of toxicity at ≤100 μM, whereas K11777 exhibited toxicity at concentrations of 70 μM. Notably, inhibitors from both structural classes (Compounds 7, 9, 10, and 20) inhibited cell death of T. cruzi-infected murine cardiomyoblasts at EC50=5-8 μM with no apparent cytotoxicity of the host cell.

Future directions include continuing to develop new peptide scaffolds for vinyl 2-pyrimidine (Compound 7), vinyl 2-pyridine (Compounds 11), and especially the vinyl 2-N-methyl pyridine (Compounds 13 and 15) that will improve initial binding to cruzain (lower Ki), particularly the P₁ sidechain. This will include a vinyl 2-triazole, a vinyl-(N-methyl)thiazole, and electron-donating and electron-withdrawing substitutions of selected heterocycles. The solubilities of PVH inhibitors will be improved by substitution of the Cbz groups with morpholino and N-methyl piperazinyl urea substituents.

Efforts to obtain crystal structures of the more potent PVH inhibitors will continue in order to investigate the presence of covalent bond formation and to guide future inhibitor design including molecular modeling. Mass spectrometric analysis will be performed on cruzain-inhibitor complexes to identify covalent bond formation where it occurs.

The masked aldehyde inhibitor class will continue to be studied, including a new 7-membered-ring MAs that may more easily open to form a reactive aldehyde in the cruzain active site. Also, we will explore the replacement of the hydroxy group of existing MAs with amino and thio groups to determine if the resulting aminals or lactols provide other classes of masked aldehydes. Crystal structures of the masked aldehydes in cruzain and cathepsin L will be obtained to guide future inhibitor design and to ascertain their chemical nature in the active site.

Selective potent, non-toxic P2VH and MA inhibitors will be evaluated in cellular models of mammalian infection, and in murine models of Chagas disease and African sleeping sickness as proof-of-concept for P2VHIs. Efficacious cruzain inhibitors will be progressed through safety and toxicity profiling to characterize drug-quality candidates.

Evaluation of Dipeptide Substrates

We have kinetically characterized 12 dipeptide fluorogenic substrates of cruzain, which are of the form Cbz/NMePip-P₂-P₁-AMC, for which Cbz is benzyloxycarbonyl; NMePip is N-Methyl-piperazinyl; P₂=Phe, Leu, Arg, or 4-pyridyl-alanine ((4-Pyr)Ala); P1=Phe, hPhe (homoPhe), Arg, Ala, and (4-Pyr)Ala; and AMC is 7-amino-4-methylcoumarin (Table 6).

TABLE 6
Kinetic parameters of peptide substrantes for cruzain.
Table. Kinetic Parameters of Peptide Substrates for Cruzain^{a}

| | | | | k_{cat}/Kₘ | k_{cat}/ | |
|---|---|---|---|---|---|---|
| Substrate | Structure | k_{cat} (s⁻¹) | Kₘ (μM) | (μM⁻¹ s⁻¹) | K_{m rel} | k_{cat rel} |
| S1 | Cbz-Phe-Arg-AMC | 9.6 ± 0.2 | 0.89 ± 0.09 | 11 ± 1 | 1.00 | 1.00 |
| S2 | Cbz-Phe-hPhe-AMC | 2.0 ± 0.2 | 0.26 ± 0.06 | 8 ± 2 | 0.73 | 0.21 |
| S3 | Cbz-Leu-hPhe-AMC | 3.0 ± 0.2 | 0.8 ± 0.2 | 4 ± 1 | 0.36 | 0.21 |
| S4 | Cbz-Leu-Arg-AMC | 8.4 ± 0.4 | 2.2 ± 0.3 | 3.8 ± 0.7 | 0.35 | 0.88 |
| S5 | Cbz-Phe-(4-Pyr)Ala-AMC | 7.5 ± 0.5 | 3.7 ± 0.5 | 2.0 ± 0.4 | 0.18 | 0.76 |
| S6 | Cbz-Arg-Arg-AMC^{b} | 7.2 ± 0.1 | 3.7 ± 0.4 | 1.9 ± 0.2 | 0.17 | 0.75 |
| S7 | NMePip-Phe-hPhe-AMC | 4.0 ± 0.1 | 3.1 ± 0.3 | 1.3 ± 0.1 | 0.12 | 0.42 |
| S8 | Cbz-(4-Pyr)Ala-hPhe-AMC | 5.3 ± 0.2 | 4.6 ± 0.5 | 1.1 ± 0.1 | 0.10 | 0.55 |
| S9 | Cbz-Phe-Phe-AMC | 0.26 ± 0.01 | 0.34 ± 0.06 | 0.8 ± 0.2 | 0.07 | 0.03 |
| S10 | Cbz-Arg-hPhe-AMC | 4.9 ± 0.1 | 6.8 ± 0.4 | 0.7 ± 0.04 | 0.06 | 0.51 |
| S11 | NMePip-Phe-Phe-AMC | 2.8 ± 0.06 | 14 ± 4 | 0.2 ± 0.04 | 0.02 | 0.29 |
| S12 | Cbz-Phe-Ala-AMC^{b} | 0.89 ± 0.06 | 38 ± 2 | 0.023 ± 0.003 | 0.00 | 0.09 |

^{a}Data obtained at pH 7.5, 25° C., 10% DMSO (v/v);
^{b}Data obtained at 2% DMSO (v/v) from Ref. 40.

The largest specificity constant (k_{cat}/Kₘ) measured was that of Cbz-Phe-Arg-AMC (11 μM⁻¹ s⁻¹, relative k_{cat}/Kₘ=1.0). Substrates S2-S4 have values of k_{cat}/Kₘ, which are 73-35% of that of Cbz-Phe-Arg-AMC, indicating that Cbz-Phe-hPhe-AMC, Cbz-Leu-hPhe-AMC, and Cbz-Leu-Arg-AMC all comprise highly competent substrates likely effecting rapid acylation of cruzain. The poorest substrate, Cbz-Phe-Ala-AMC (S12), demonstrated that a small side chain in the P1 position is less preferable than more bulky hydrophobic moieties. Conversely, the dipeptide Cbz-Phe-hPhe-AMC (S2) exhibited a value of k_{cat}/Kₘ=8 μM⁻¹ s⁻¹, indicating that a peptide substrate with the same P₂ and P₁ amino acids as K11777 is an excellent substrate of cruzain and likely involves rapid acylation. Interestingly, when the Cbz group of substrate S2 is replaced with NMePip (S7), a substrate mimic of K11777, k_{cat}/Kₘ is 8-fold lower (1.3 μM⁻¹ s⁻¹). However, its value of k_{cat} exceeded that of Cbz-Phe-hPhe-AMC (S2), suggesting that the NMePip N-terminus of S7 acts to retard cruzain acylation in comparison to the Cbz group of S2. While Cbz-Phe-Phe-AMC (S9) had a value of k_{cat}/Kₘ that was only 7% of Cbz-Phe-Arg-AMC, its Kₘ value of 0.34 μM indicated potent binding, albeit with slow turnover. This may indicate that a P₁ Phe substitution leads to favorable binding, but this side chain impedes either the acylation or deacylation step in catalysis. This was observed for dipeptide-AMC substrates for the papain-like protease, human cathepsin C.⁴³

We explored the viability of the (4-Pyr)Ala residue as a potential mimic of both Phe and Arg, for the latter residue when the pyridine is protonated. Cbz-Phe-(4-Pyr)Ala-AMC (S5) was found to be a good substrate (relative k_{cat}/Kₘ=0.10) but with a higher value of Kₘ (3.7 μM) compared to substrates containing Phe and hPhe in the P₁ residue. To leverage the ability of cruzain to tolerate both charged basic and hydrophobic residues in the P2 position, we prepared Cbz-(4-Pyr)Ala-hPhe-AMC and Cbz-Arg-hPhe-AMC (S8 and S10). These substrates displayed efficient turnover numbers (k_{cat}=5.3 and 4.9 s⁻¹, respectively) but poor values of Kₘ (4.6 and 6.8 μM) in comparison to Cbz-Phe-Phe/hPhe-AMC (0.26-0.34 μM). This demonstrates that the enzyme has a strong preference for substrates that contain a hydrophobic P₂ residue. Together, these results indicated that dipeptide scaffolds in which P₂=Cbz-Phe or NMePip-Phe and P1=Arg, hPhe, or (4-Pyr)Ala may be among the best to incorporate into the vinyl heterocyclic framework. Accordingly, for our new peptidomimetic vinyl heterocyclic inhibitors, we have primarily utilized the Cbz-Phe-Phe and NMePip/Cbz-Phe-hPhe dipeptide scaffolds.

Computer-Assisted Inhibitor Design

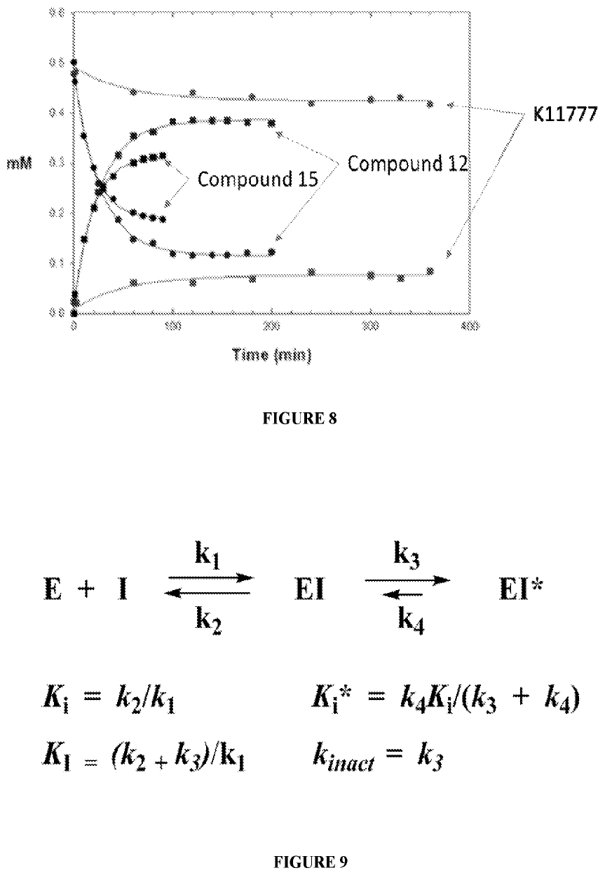

To aid in the rational design of our vinyl heterocyclic inhibitors, we employed molecular docking of these compounds to a model constructed from the crystal structure of K11777-cruzain (PDB accession code: 20Z2), which contains a covalent bond between the inactivator and Cys25.³¹ Owing to our hypothesis that the vinyl heterocyclic inhibitors have the ability to undergo a reversible thia-Michael addition with the active site Cys25 of cruzain, it is necessary to consider scenarios of both noncovalent and covalent binding. To this end, we first predicted the binding patterns for NMePip-Phe-hPhe-vinyl-2Pyrmd (9), which has the same scaffold as K11777 using Glide⁴⁴⁻⁴⁶ and CovDock⁴⁷ modules embedded in the Schrodinger software package. In the covalent model, the binding of 9 with cruzain was highly conserved when compared to that of K11777 (FIG. 6A). Pyrimidine N₁ of 9 was within hydrogen bonding distance of Gln19 and Trp184, allowing the stabilization of the vinyl heterocycle in a nearly analogous fashion to the sulfone moiety in K11777. In addition, the α-carbon of the inhibitor is positioned within 2.4 Å of His162, an interatomic distance that would easily allow facile proton transfer between this carbon and the imidazole nitrogen, supporting our hypothesis that a reversible adduct could be formed with cruzain. The noncovalent model (FIG. 6B) shared similar shape complementarity with the covalent binding pose, except that it was slightly shifted away from the binding site as militated by the docking algorithm to avoid clashing with Cys25. This suggested that covalent bond formation would only slightly perturb the noncovalent binding conformation. Overall, these data suggested that the binding of our newly designed compounds containing a vinyl heterocyclic warhead have the ability to interact with cruzain in a very similar fashion to the characterized, irreversible inactivators of the enzyme. Similarly, we carried out docking for five other PVH compounds (7, 11, 12, 13, and 15). The corresponding inhibition constants (predicted Kᵢ) converted from these affinity values ranged from 0.79 to 6.1 μM, with the exception of compound 7, were similar with the experimental values found in FIG. 8.

Synthesis of PVHIs

The general synthetic routes employing either Wittig⁴⁸ or Horner-Wadsworth-Emmons⁴⁹ reactions shown in Scheme 1 were used to synthesize peptidomimetic vinyl heterocyclic compounds from aldehydes and halomethyl heterocycles.

Commercially available Boc-protected L-amino acids phenylalanine, homophenylalanine, and alanine (a) were converted to Weinreb amides⁵⁰ by T3P-catalyzed coupling to N,O-dimethylhydroxylamine hydrochloride to afford b (GP1, General Procedure 1 in Experimental Section). Reduction of the Weinreb amide using LAH at −10° C. in anhydrous THE provided the Boc-amino acid aldehyde (c, GP2), generally in overall yields of ˜80% (a-c).

Phosphonium salts of methyl heterocycles were, in general, prepared by derivatization of either the 2-methylcarboxy or 2-hydroxymethyl heterocycle (d-f, Scheme 1). Methyl 2-carboxy-pyrimidine (or pyridine, oxazole, and thiazole) was reduced using sodium borohydride to the primary alcohol, followed by conversion of the alcohol to the 2-chloromethylpyrimidine (e) using SOCl₂ or POCl₃ in DCM or CHCl₃ (GP3). The reaction of e with triphenylphosphine provided the Wittig reagent phosphonium salt (f) at overall yields of 28-80% (GP4). Wittig coupling of f with a peptide aldehyde (c) using LHMDS in anhydrous THE or sodium methoxide in benzene as the base provided the peptide vinyl heterocyclic product h (GP6), with general overall yields of 13-54%. Typically, the ratio of E/Z was 4:1, and the separation of these regioisomers was readily achieved using silica gel column chromatography.

Alternatively, the 2-chloromethyl-heterocyclic group e was converted to its phosphonate g by use of the Arbuzov reaction with triethylphosphite (˜80% yields, GP5). The resulting phosphonate was deprotonated with LHDMS in THE and then coupled with aldehyde c to provide the peptide vinyl heterocycle h at 20-80% yield (GP7). The Boc group was removed quantitatively by treatment with TFA in DCM, and then the free amine was coupled with the P₃-P₂ fragment (R1-Xaa-OH) using T3P to give the inhibitor i (GP8).

In addition, some of the PVHIs underwent N-methylation of the heterocycle (j, GP9). Further, we also prepared several acrylamides (k) through hydrolysis of the corresponding acrylate ester and subsequent treatment with ethyl chloroformate and NH4Cl (GP10). Final products were confirmed structurally by NMR and LCMS, as described in the Experimental Section and Supporting Information. It is important to note that proton NMR analysis of the products (i-k) indicated negligible epimerization at the α-carbon in these products, as evidenced by the absence of diastereomers.

Kinetic Analysis of Cruzain Inhibitors and Inactivators

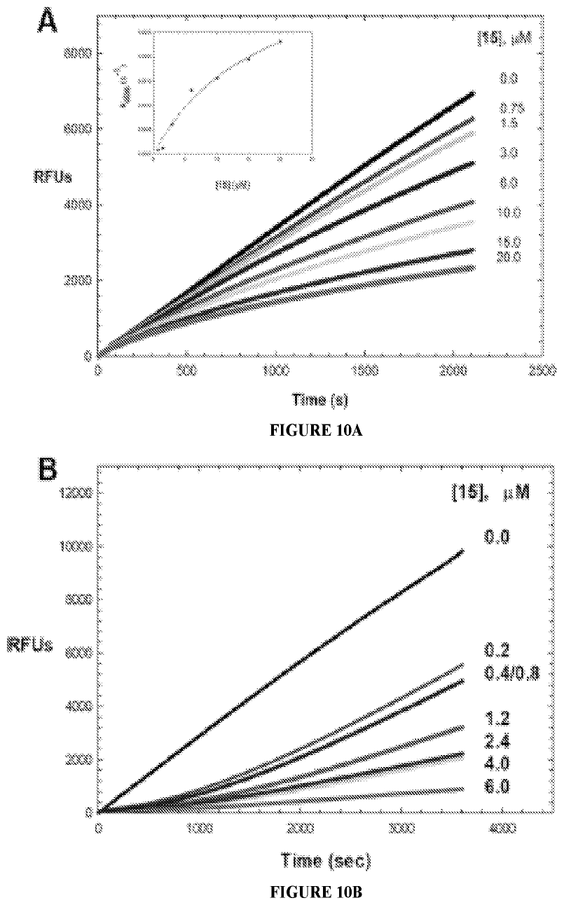

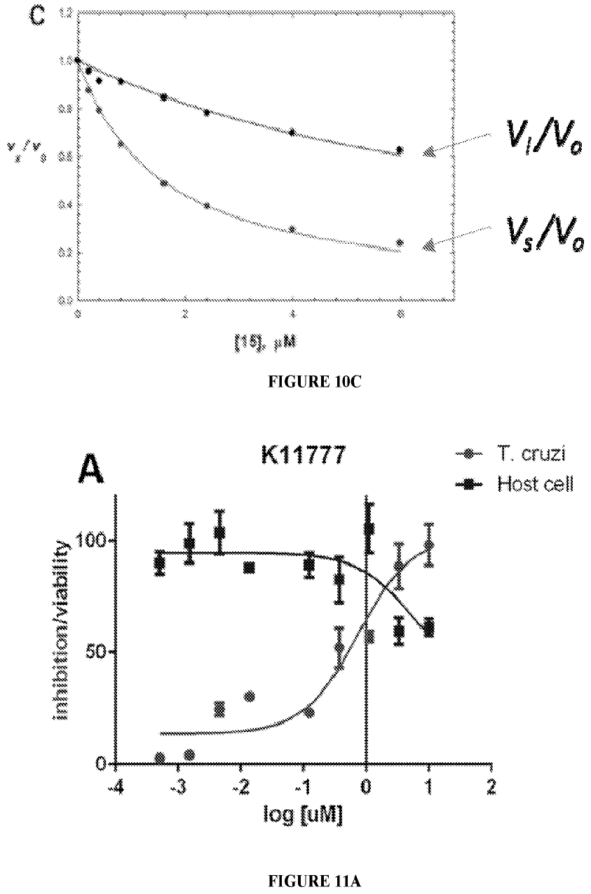

FIG. 9 is a kinetic depiction of inhibition and inactivation of cruzain and the relevant kinetic parameters.⁵² The initial, and usually rapid, formation of EI is characterized by the inhibition constant Kᵢ. For time-dependent inhibitors, EI progresses to a second, tighter complex EI*, generally over the course of minutes, characterized by Kᵢ*, for which Kᵢ*<Kᵢ when k₄<k₃. For irreversible covalent inactivators, k₄ and Kᵢ*≈0, and the kinetic parameter k_{inact}/K_{I} is generally reported. For reversible time-dependent inhibitors, initiation of the reaction by adding enzyme to the substrate and inhibitor leads to concave-downward, curvilinear time courses of product formation in which reaction rates demonstrably decrease as the EI* complex forms. Typical data, as exemplified for compound 15, are shown in FIG. 10. Alternatively, extended preincubation of the enzyme and inhibitor, followed by dilution of the inhibitor and initiation of the reaction with high concentrations of the substrate, leads to concave-upward curvilinear plots of product formation as E reforms from EI* (FIG. 10B). Results of this analysis for cruzain inhibitors and inactivators are collected in Tables 1 and 2.

K11777 comprises a useful benchmark compound despite the fact that it is an irreversible inactivator of cruzain (reported kinetic data: apparent IC₅₀ of 2 nM, k_{inact}/Kᵢ=234,000 M⁻¹ s⁻¹).^{32,53} We replaced the P₁ hPhe group of K11777 with a Phe side chain to provide vinyl sulfone 1, which had apparently equivalent potency (Kᵢ*=3.6 nM) to that of K11777, but which, interestingly, exhibited kinetically reversible inhibition of cruzain. However, a crystal structure we obtained for 1 bound to cruzain indicated the formation of a C—S bound between Cys25 and 1 (FIG. 1). This may indicate that a phenylalanyl group at the P₁ position partly impedes the ability of an adjacent vinyl electrophile to access Cys25, as was observed with the solution phase GSH addition studies to our PVH compounds.

We next evaluated three C-terminal acrylamides (R₃=II) within the Cbz-Phe-Phe, Cbz-Phe-hPhe, and NMePip-Phe-hPhe scaffolds (2-4). The acrylamides within the Cbz-PhehPhe and NMePip-Phe-hPhe scaffolds afforded apparently irreversible covalent inactivation (k_{inact}/K_{I}=1700-1900 M⁻¹ s⁻¹), while Cbz-Phe-Phe-acrylamide (2) was less effective (k_{inact}/K_{I}=22 M⁻¹ s⁻¹). Comparing the values of k_{inact}/K_{I} for K11777 and 4 indicated that the vinyl sulfone is overwhelmingly more effective as a covalent inactivator than its acrylamide counterpart, possibly owing to hydrogen bond contacts of the sulfone oxygen with Gln19, which position the vinyl group proximal to Cys25 of cruzain. As with 1, a Phe rather than an hPhe group at the P₁ position may retard covalent formation over the time course of kinetic analysis when one compares the rates of apparent inactivation of 2 versus 3 and 4, as was also seen with peptide substrates.

We therefore sought to explore the effects of replacement of both the vinyl-phenylsulfone and acrylamide groups with phenyl and heterocyclic groups conjugated to the vinyl group. The Cbz-Phe-Phe-vinyl-benzene compound 5 is a time dependent inhibitor of cruzain (Kᵢ*=0.87 μM), but substitution of the para position of the phenyl ring with an electron-withdrawing nitro group (compound 6) led to a nearly 3-fold improvement in potency (Kᵢ*=0.34 μM), suggesting that the vinyl group of 6 is more capable of thiolation by the cruzain. As seen with 1, these compounds also demonstrated reversible inhibition of cruzain, possibly due to the P₁ phenylalanine. Due to poor aqueous solubility (solubility of 5 and 6, ≤2 μM in 10% DMSO), the inhibitors containing vinyl-benzene were not explored further.

Subsequently, six heterocyclic groups (R₃=IV-IX) conjugated to the presumed electrophilic vinyl group were evaluated within several dipeptide scaffolds. The vinyl-2-pyrimidine (R3=IV), vinyl-2-pyridine (R₃=V), vinyl-2-oxazole (R₃=VIII), and vinyl-2-thiazole (R₃=IX) groups, unlike the vinyl-4-pyridine (R₃=VI) and vinyl-4-pyrimidine (R3=VII), maintain bioisosteric similarity to the reactive acrylamides and vinyl sulfones, which is reflected in their more potent inhibition of cruzain as detailed below. Most of these compounds induced time-dependent inhibition on cruzain and were found to be kinetically reversible with residence times (τ) of 6-20 min.

The vinyl-2-pyrimidine moiety (R₃=IV) in the Cbz-Phe-Phe scaffold afforded compound 7, which exerted time-dependent inhibition of cruzain with an initial value of Kᵢ=5 μM and subsequent tight-binding inhibition of Kᵢ*=0.38 μM. Substitution of the phenyl group of 5 by a pyrimidine group greatly improved the solubility of 7 (≥100 μM in 10% DMSO). Extended preincubation with 7, followed by dilution, and addition of an excess of substrate, resulted in slow recovery of cruzain activity, indicating that any covalent reaction between cruzain and 7 was kinetically reversible (k₄=0.0018±0.0003 s⁻¹; τ=9 min). Interestingly, when the 2-pyrimidinyl moiety is appended to Cbz-Phe-hPhe (8), the resulting compound is a poor inhibitor of cruzain (Kᵢ>35 μM); however, when the 2-pyrimidinyl group is attached to afford the same scaffold as K11777, we obtained an inhibitor of low micromolar potency (9, Kᵢ*=2.2 μM). Substitution of the P₁ Phe with Ala (10, Kᵢ=25 μM) produced a poor inhibitor of cruzain, indicating the essentiality of a larger side chain in the P₁ position, as was observed with dipeptide substrates. To probe the importance of the vinyl group for the inhibition of cruzain, we prepared an analogue in which the vinyl group of 7 was reduced (compound 27). This inhibitor lacked time dependent behavior (Kᵢ=22 μM) and was 100-fold less potent than its vinyl analogue 7, which demonstrated the importance of the vinyl group for the inhibition of cruzain. We prepared inhibitor 23, which contains a vinyl-4-pyrimidinyl (R₃=VII) group that does not maintain bioisosteric similarity to the acrylamides. 23 exhibited 3-fold less potency than the bioisosteric vinyl-2-pyrimidine (7). Similarly, inhibitors containing the vinyl-4-pyridyl (R₃=VI) (21 and 22) lack bioisosteric equivalence to the acrylamides and were found to be only modest inhibitors of cruzain.

Inhibitors containing a vinyl-2-pyridinyl group (R₃=V) were explored more widely. Cbz-Phe-Phe-vinyl-2-pyridine 11 exhibited time-dependent inhibition of cruzain with an initial value of Kᵢ=5.5 μM and subsequent tight-binding inhibition of Kᵢ*=0.31 μM (k₄=0.0012±0.0002 s⁻¹; τ=13 min), and solubility of 11 was ≤30 μM in 10% DMSO. Unlike the vinylpyrimidinyl group of 7, placement of the vinyl-2-pyridinyl group in the Cbz-Phe-hPhe scaffold improved inhibition by 3-fold (13, Kᵢ*=0.17 μM), while the vinyl-2-pyridinyl group was much less effective in the NMePip-Phe-Phe scaffold (14, Kᵢ*=3.4 μM). Substitution of an electron-donating methoxy group on the pyridine ring (16) of the Cbz-Phe-hPhe scaffold diminished the inhibitory activity of the vinyl-2-pyridinyl heterocycle compared to its unsubstituted counterpart 13 by >50-fold, suggesting that the methoxy group is large enough to create a steric barrier to inhibitor binding. In contrast, the substitution at C-4 of the pyridine with the electron withdrawing trifluoromethyl group resulted in better inhibition (17, Kᵢ*=0.57 μM), but nonetheless was less potent than the unsubstituted pyridine 13. Apparently, this result arises from steric crowding as OMe>CF3>H, implicating that substitution at the C-4 position of the pyridine heterocycles are not well tolerated.

We next investigated how the P₁ and P₂ side chains of these vinyl-2-pyridinyl inhibitors affect inhibition. The replacement of the P₂ Phe with Leu resulted in diminished potency (18, Kᵢ*=1.42 μM) compared to the Cbz-Phe-Phe and Cbz-Phe-hPhe scaffolds, overall demonstrating that inhibitors with bulky hydrophobic substituents in P₁ and P₂ enhanced binding to cruzain. To analyze how short alkyl and charged groups effected inhibition, we prepared Cbz-Phe-Ala-vinyl-2-pyridine (19) and Cbz-Phe-Lys-vinyl-2-pyridine (20). We found that the Cbz-Phe-Lys scaffold, which mimics our most optimal substrate, Cbz-Phe-Arg-AMC, exhibited good inhibition (Kᵢ*=0.87 μM), whereas 19 was a poor inhibitor (Kᵢ*=4.8 μM), in concert with the poor substrate activity of Cbz-Phe-Ala-AMC.

Seeking to improve the electrophilicity of the vinyl-2-pyridinyl group, we prepared N-methylated analogues 12 and 15. This modification resulted in improved aqueous solubility (≥50 μM in 10% DMSO) and provided potent time dependent inhibition of cruzain (12, Kᵢ*=0.28 μM; 15, Kᵢ*=0.126 μM) comparable to, or exceeding, the inhibition exerted by their unmethylated counterparts (11 and 13).

Inhibition data for compound 15 were fitted by all methods outlined in Experimental Section, as shown in FIG. 10. We fitted each curve in FIG. 10A to eq 3, and the resulting values of k_{obs} were replotted versus [15] (FIG. 10A, inset), which demonstrated a hyperbolic dependence of the inhibitor (fitting to eq 4: Kᵢ=2.0±0.9 μM, k₃=0.004±0.001 s⁻¹, and k4≈0). Alternatively, global fitting of these curves to eq 6 provided values of Kᵢ=4.3±0.1 μM, k₃=0.0012±0.0004 s⁻¹, and k₄=0.00019±0.00005 s⁻¹, from which was calculated a value of Kᵢ*=0.6±0.2 μM. Finally, analysis of inhibition of cruzain by 15 at early and late phases of the time courses in FIG. 10A by fitting to eq 5 provided values of Kᵢ=0.76±0.04 μM and Kᵢ*=0.126±0.004 μM. Of note, in preincubation studies, all PVHIs, which contain the vinylpyridinyl substituent, displayed kinetic reversibility.

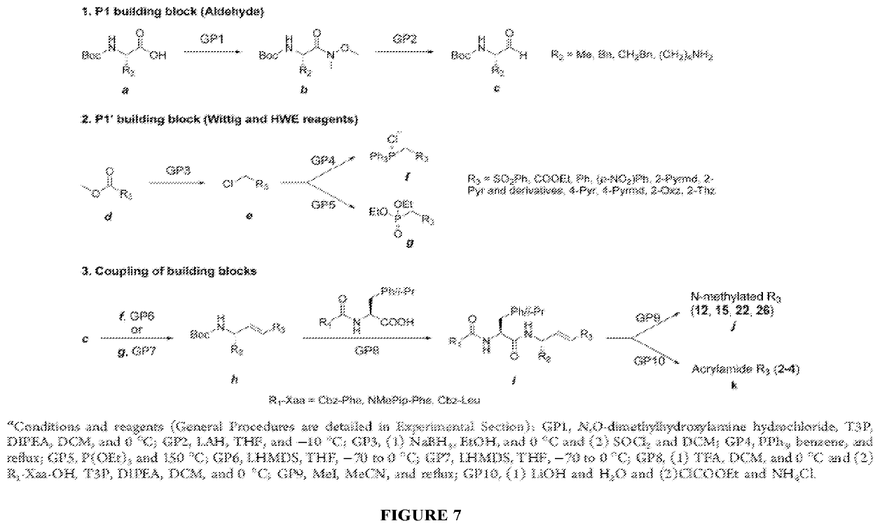

We investigated five-membered ring heterocycles that are bioisosteric with acrylamide inactivators. The syntheses of vinyl-2-oxazole (24), vinyl-2-thiazole (25), and its N-methylated counterpart (26) into the Cbz-Phe-Phe scaffold proved facile and provided useful inhibitors. Vinyl-2-oxazole 24 was a submicromolar inhibitor of cruzain (Kᵢ*=0.71 μM). Vinyl-2-thiazole inhibitors 25 and 26 were inhibitors of similar potency (Kᵢ*=1.71 and 0.94 μM, respectively), for which N-methylation of the thiazole improved potency by nearly 2-fold. A scheme for the syntheses of the compounds in Table 1 is found in FIG. 7, and detailed below.

Cruzain inhibitors 5-26 allowed the evaluation of six heterocyclic groups (R₃=IV-IX) appended to the presumed electrophilic vinyl group within several dipeptide scaffolds. The vinyl-2-pyrimidine, vinyl-2-pyridine, vinyl-2-N-methylpyridinium, vinyl-2-oxazole, and vinyl-2-thiazole substituents, unlike the vinyl-4-pyrimidine and vinyl-4-pyridine heterocycles, maintained bioisosteric similarity to the reactive acrylamides and provided potent, time-dependent inhibitors in accord with our hypothesis. Of these PVHIs, 2-pyridine, charged 2-Nmethylpyridine, and vinyl-2-pyrimidine presented the most interesting heterocycles for further exploration. The inhibition of cruzain displayed by these PVHIs may be due to the reversible formation of an adduct with active site Cys25, as is supported by the loss of time-dependent inhibition when the vinyl group is saturated. Importantly, we have no evidence that such a reversible covalent bond is formed, and ongoing studies are underway to address this point.

Selectivity of PVHIs for Cruzain Over Homologous Human Cathepsins

Cruzain has 25, 15, and 23% amino acid identity with human cathepsins L, B, and S respectively.^{31,55} It is preferable to proceed with cruzain inhibitors that do not readily inhibit these human lysosomal cathepsins, which might engender cellular toxicity. We evaluated selected cruzain inhibitors versus the human cysteine proteases cathepsins L, B, and S (FIG. 8). For this selectivity comparison, all inhibition data were obtained at pH 5.5 for which Kᵢ* values were invariant for all inhibitors except compound 15 (Kᵢ*=88 nM). The cruzain inhibitors demonstrated moderate selectivity versus cathepsins L and S (generally, 3-fold or greater), while all of these inhibitors displayed 40-fold or higher selectivity versus cathepsin B. The vinyl-2-pyridine inhibitors 13 and 15 are particularly selective as their Kᵢ values are over 10-fold lower than the corresponding values with the three human cathepsins. In contrast, K11777 showed potent inactivation at nanomolar concentrations for all three human cathepsins; this apparent lack of selectivity possibly arising from its irreversible mode of inactivation. These results suggest that suitable selectivity for reversible cruzain inhibitors may be more easily attained than for irreversible ones.

Effects of PVHIs in Axenic Cultures of T. cruzi and in a Cell Model of T. cruzi Infection. Initially, we tested selected compounds against epimastigotes of T. cruzi (strain Y, ATCC 50832GFP) in axenic cultures. As is observed here (Table 5) and has been shown previously, K11777 weakly inhibited the growth of T. cruzi epimastigotes (EC50≈60 μM).⁵⁴ PVHIs 7, 12, and 15 inhibited the growth of epimastigotes of T. cruzi (EC50=2-20 μM), while 11, 13, and 24 were poorly effective. Compounds 12 and 15 were comparably potent against cultures of T. cruzi (EC₅₀=8.6 and 2.1 μM, respectively), and were, at a minimum, 10-fold more active than K11777.

Selected cruzain inhibitors were further evaluated in a more relevant cellular model of Chagas disease: T. cruzi-infected murine cardiomyoblasts (C2C12 cells) (FIG. 11). Inhibitors 7, 11, 12, 13, and 15 exhibited antiparasitic efficacy at values of EC₅₀=5-10 μM while displaying no cytotoxicity against the host cardiomyoblasts (CC₅₀>10 μM). These EC50 values demonstrated that the antitrypanosomal activities of the reversible PVHIs are within an order of magnitude of potency of the irreversible inactivator, K11777 (EC₅₀=0.7 μM), despite the large difference in activity versus cruzain. Accordingly, the PVHIs, while reversible in action and with no apparent mammalian or human cytotoxicity, are nearly as effective as the potent, irreversible inactivator K11777. Further, the best of the PVHIs is less than 3-fold less potent than the currently used antichagasic drug benznidazole (LD₅₀=1.5 μM),⁵⁵ suggesting that a second generation of PVHIs may provide clinical candidates.

Effects of PVHIs in Axenic Cultures of T. b. brucei

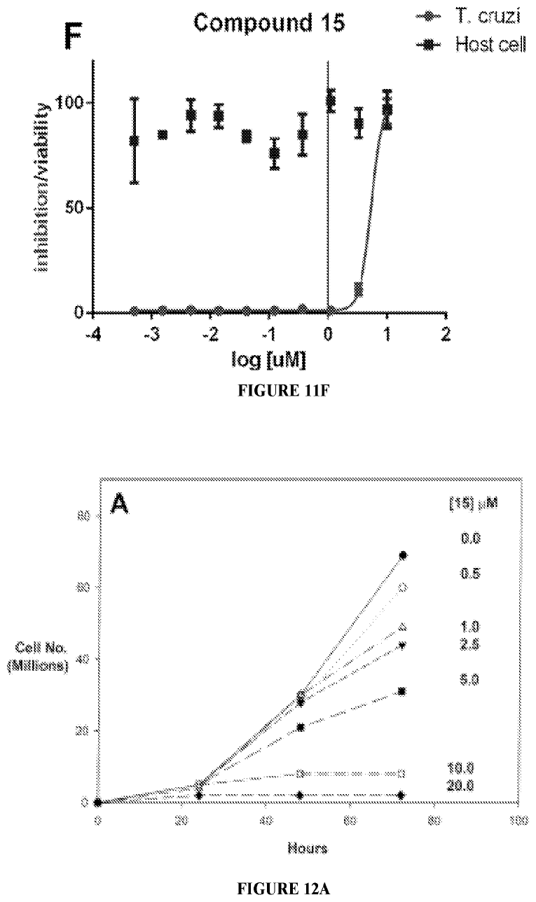

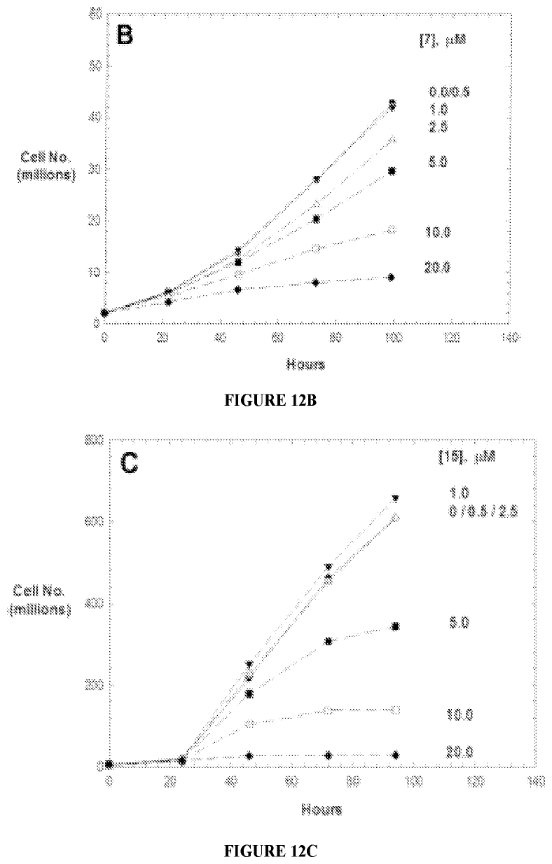

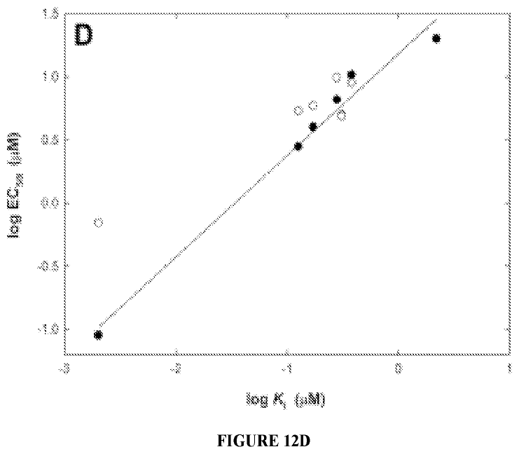

We additionally tested our cruzain inhibitors in axenic cultures of the related protozoan T. b. brucei owing to the high structural similarity and reported essentiality of the cysteine protease brucipain (TbCatL) in T. b. brucei.^{24,34,57} It has been demonstrated that the cruzain inhibitor K11777 is active in cellular cultures of both T. b. brucei and T. cruzi, supporting the notion that our PVHIs could be effective in growth inhibition of both species of parasite. For insect procyclic forms (PCFs) of T. b. brucei (ATCC PRA-381), compounds 7, 9, 11, 12, and 15 demonstrated growth inhibition at EC₅₀ values of 5-15 μM (FIGS. 3 and 12). When compared to K11777 (EC₅₀=1.7 μM), these PVHIs exhibited potent cell growth inhibition. For example, compound 15 (EC₅₀=5.9 μM) was only 3-fold less potent versus T. b. brucei than K11777. Values of EC₅₀ for these PVHIs roughly correlated with their values of Kᵢ*, with the exception of compound 13.

We next evaluated these inhibitors in axenic cultures of human bloodstream forms (BSFs) of T. b. brucei (ATCC PRA-383). All PVHIs that were active versus procyclic forms of T. b. brucei were also trypanocidal versus the bloodstream forms but with equal or lower EC50 values compared to the procyclic forms (FIG. 12). Compared to PVHIs that had similar potencies in both PCFs and BSFs, K11777 was nearly 20-fold more potent in T. b. brucei BSFs than in PCFs. These results suggested that a cathepsin L-like cysteine protease in T. b. brucei, such as brucipain (or TbCatL),²⁴ is essential for growth of procyclic and bloodstream T. b. brucei, but perhaps an additional cysteine protease, such as TbCatB, is also essential in BSFs of T. b. brucei as this enzyme is sensitive to K11777 but not to the PVHIs. This is similar to the findings of Yang et al.⁵⁸ who showed using an activity-based protein probe of K11777 that TbCatB and brucipain (TbCatL) are both labeled in BSFs of T. b. brucei while only brucipain is labeled in PCFs. This could explain the exceptional trypanocidal activity of K11777 in BSFs. This will be the focus of our future studies. Nonetheless, the activity of the PVHIs versus T. b. brucei BSFs may hold promise for progression to their evaluation in models of African trypanosomiasis.

Interestingly, the values of EC₅₀ obtained for PCFs of T. b. brucei and amastigotes of T. cruzi were nearly identical for most PVHIs despite their more modest inhibition of cruzain. Shown in FIG. 12D is a correlation plot of log EC50 for antitrypanosomal activity for bloodstream forms of T. b. brucei and the amastigote forms of T. cruzi from the murine cardiomyoblast infection model. For the former, the correlation is excellent (r²=0.979, slope=0.80), and the activity against parasites is nearly a 1:1 correlation with log Kᵢ with these inhibitors. This result provided support that our PVHIs are targeting a cruzain-like protease in T. b. brucei. For T. cruzi, this correlation is not as strong, in part, due to the absence of a sufficient range of data. We have also compared the cytotoxicity of selected inhibitors in human dermal fibroblasts versus T. cruzi-infected cardiomyoblasts (selectivity index in FIG. 11), which demonstrates that the PVHIs are more than 10-fold selective for trypanosomes versus human cells.

### Conclusions

We have developed a novel class of reversible inhibitors for the essential cysteine protease of T. cruzi, cruzain. These compounds, peptidomimetic vinyl heterocycles, contain bioisosteric replacements for the acrylamide and vinyl sulfone warheads present in irreversible, covalent inactivators such as K11777. We also demonstrated that PVHIs containing vinyl-2-N-methylpyridine or vinyl-2-N-methylthiazole groups, unlike other inhibitors, readily form Michael adducts with glutathione. Our survey demonstrated that the most optimal cruzain inhibitors contained vinyl-2-pyrimidine, vinyl-2-pyridine, and vinyl-2-N-methylpyridinium groups. These PVHIs proved to be potent, time-dependent inhibitors of cruzain, albeit fully reversible in terms of the mode of action. These PVHIs are significantly active in both axenic cultures of T. b. brucei and in a cell infection model of T. cruzi, and further optimization may produce more potent antitrypanosomal agents. Importantly, the concept of reversible covalent inactivation by vinyl heterocycles is potentially expandable to other enzymes, which contain active site cysteines, such as EGFR, G12C KRas, and other protein kinases for which irreversible acrylamide inactivators comprise effective drugs.⁵⁶

Experimental Section

General Synthetic Chemistry Methods and Compound Characterization. All reagents and starting materials were obtained from commercial suppliers and used without further purification unless otherwise stated. Reactions were run under an atmosphere of nitrogen or argon and at ambient temperature unless otherwise noted. Reaction progress was monitored using thin-layer chromatography and by analysis employing an HPLC-MS (UltiMate 3000 equipped with a diode array coupled to an MSQ Plus single quadrupole mass spectrometer, Thermo Fisher Scientific) using electrospray positive and negative ionization detectors. Reported liquid chromatography retention times (tR) were established using the following conditions: column: Phenomenex Luna 5 μm C18(2) 100 Å, 4.6 mm, 50 mm; mobile phase A: water with 0.1% formic acid (v/v); mobile phase B: MeCN with 0.1% formic acid (v/v); temperature: 25° C.; gradient: 0-100% B over 6 min, then a 2 min hold at 100% B; flow: 1 mL min-1; and detection: MS and UV at 254, 280, 214, and 350 nm.

Semi-preparative HPLC purification of compounds was performed on a Thermo Fisher Scientific UltiMate 3000 with a single wavelength detector coupled to a fraction collector. Purifications were conducted using the following conditions: column: Phenomenex Luna 5 μm C18(2) 100 Å, 21.2 mm, 250 mm; mobile phase A: water with 0.1% formic acid (v/v); mobile phase B: MeCN with 0.1% formic acid (v/v); temperature: room temperature; gradient: 0-100% B over 30 min, then a 5 min hold at 100% B; flow: 20 mL min-1; and detection: UV (254 nm).

¹H/¹³C NMR magnetic resonance spectra were obtained in CDCl₃, CD₃OD, or DMSO-d₆ at 400 MHz/100 MHz at 298 K on a Bruker AVANCE III Nanobay console with an Ascend magnet unless otherwise noted. The following abbreviations were utilized to describe peak patterns when appropriate: br=broad, s=singlet, d=doublet, q=quartet, t=triplet, and m=multiplet. All final compounds used for testing in assays and biological studies had purities that were determined to be >95% as evaluated by their proton NMR spectra and their HPLC/MS based on ultraviolet detection at 254 nm. Similar RP-HPLC conditions were used for the experiments of GSH addition to vinyl heterocycles. Masses detected were in the range of 100-1000 Da and were detected in the positive or negative mode, depending on the ionization of the molecule.

Synthetic Procedures for the Preparation of PVH Inhibitors

General procedures (GP1-GP10 in FIG. 7) of synthesizing PVHIs are detailed below. Each GP described the synthesis of one representative compound. In addition, substrate synthesis and characterization are provided in the Supporting Information.

GP1: Synthesis of Weinreb Amides (a to b). A solution of Boc-L-homophenylalanine (12.02 g, 43.03 mmol) in anhydrous DCM (200 mL) was cooled to 0° C. under a N2 atmosphere. Et₃N (18.1 mL, 129.09 mmol, 3 equiv) was added slowly, followed by the addition of N,O-dimethylhydroxylamine hydrochloride (6.3 g, 64.5 mmol, 1.5 equiv) and dropwise addition of T3P (50% (w/v) in MeCN, 41.1 mL, 64.55 mmol, 1.5 equiv). The resulting mixture was stirred at 0° C. for 30 min to 1 h until TLC analysis (EtOAc/hexane=1:1, v/v) showed the disappearance of the starting material. The reaction mixture was diluted with DCM and washed with H₂O. The organic layer was dried over anhydrous Na₂SO₄ and filtered. The filtrate was concentrated in vacuo to afford the crude product. Purification of the crude product by silica gel column chromatography using a gradient of 5-50% of EtOAc in hexane as the eluent yielded the pure Weinreb amide tertbutyl (S)-(1-(methoxy(methyl)amino)-1-oxo-4-phenylbutan-2-yl)-carbamate (b, 13.3 g, 41.31 mmol, 96% yield) as a colorless gum.

GP2: LAH Reduction of Weinreb Amides (b to c). To a solution of tert-butyl (S)-(1-(methoxy(methyl)amino)-1-oxo-4-phenylbutan-2-yl)carbamate (b, 6.7 g, 20.78 mmol) in anhydrous THE (120 mL) at −10° C. under a N₂ atmosphere was added dropwise LAH (2.0 M in THF, 12.5 mL, 24.93 mmol, 1.2 equiv). The resulting mixture was stirred at −10° C. for 30 min. Upon completion of the reaction as shown by TLC analysis (EtOAc/hexane=1:1, v/v), the reaction was quenched at the same temperature by adding dropwise 1 N HCl, followed by removal of THE by rotary evaporation. Diethyl ether (500 mL) was added to the solid residue, and the solution was washed with aqueous NaHCO₃ (1×50 mL) and brine (1×50 mL). The organic layer was dried over anhydrous Na₂SO₄ and filtered. The filtrate was concentrated in vacuo to afford the crude product. Purification of the crude material by silica gel column chromatography using a gradient of 10-60% of EtOAc in hexane as the eluent yielded the pure aldehyde tert-butyl (S)-(1-oxo-4-phenylbutan-2-yl)carbamate (c, 4.89 g, 18.57 mmol, 89% yield) as a white solid.

GP3: Preparation of Chloromethyl Heterocycles (d to e). To a suspension of methyl pyrimidine-2-carboxylate (d, 1.156 g, 8.37 mmol) in anhydrous EtOH (20 mL) at 0° C. under a N₂ atmosphere was added portionwise NaBH₄ (0.443 g, 11.72 mmol, 5 equiv). The reaction mixture was stirred at 25° C. for 2 h. Upon completion of the reaction as shown by TLC analysis (EtOAc/hexane=1:1, v/v), the reaction solvents were removed by rotary evaporation. To the resultant colorless gummy residue was added ice cold H₂O (20 mL) followed by extraction with DCM (5×50 mL). The organic layer was dried over anhydrous Na2SO4 and filtered. The filtrate was concentrated in vacuo to afford the crude product pyrimidin-2-ylmethanol (0.900 g, 8.17 mmol). To this pyrimidin-2-yl-methanol in CHCl₃ (20 mL) at 0° C. under a N₂ atmosphere was added dropwise POCl₃ (1.95 mL, 3.21 g, 2.5 equiv). The reaction mixture was stirred at 25° C. for 1 h, followed by refluxing for an additional 3 h under gentle heating until TLC analysis (EtOAc/hexane=3:1, v/v) showed the completion of the reaction. The reaction was quenched by a careful addition of aqueous NaHCO₃ and further addition of solid NaHCO₃ to afford a basic pH. The aqueous layer was extracted with CHCl₃ (3×50 mL), and the organic layer was dried over anhydrous Na₂SO₄ and filtered. The filtrate was concentrated in vacuo to afford the pure product 2-(chloromethyl)pyrimidine (e, 0.948 g, 7.43 mmol, 63% yield) as a light yellow semi-solid, which was used further without any purification.

GP4: Preparation of Heterocyclic Phosphonium Ylides (e to f, Wittig Reagents). A mixture of 2-(chloromethyl)pyrimidine (e, 0.92 g, 7.22 mmol) and triphenylphosphine (2.1 g, 7.94 mmol, 1.1 equiv) in anhydrous benzene (25 mL) was refluxed under a N2 atmosphere for 24 h until TLC analysis (MeOH/DCM=1:19, v/v) showed the completion of the reaction. The reaction mixture was concentrated via rotary evaporation, and the gummy residue was triturated with diethyl ether (3×10 mL). The solid obtained was purified by silica gel column chromatography using a gradient of 1-10% of MeOH in DCM as the eluent to afford the pure product triphenyl(pyrimidin-2-ylmethyl)phosphonium chloride (g, 0.797 g, 2.039 mmol, 28% yield).

GP5: Preparation of Heterocyclic Phosphonates (e to g, HWE Reagents). 2-(Chloromethyl)pyridine hydrochloride (e, 16.5 g, 100.6 mmol) in DCM (100 mL) was treated with aqueous NaHCO₃ (20 mL), and the DCM layer was dried over anhydrous Na₂SO₄. The filtrate was concentrated by rotary evaporation. The alkyl halide thus obtained along with triethyl phosphite (35 mL, 201.2 mmol, 2.0 equiv) was heated at 150° C. under a N₂ atmosphere for 5 h until TLC analysis (MeOH/DCM=1:19, v/v) showed the completion of the reaction. The reaction mixture was purified by silica gel column chromatography using a gradient of 10-100% of EtOAc in hexane and later 1-10% of MeOH in DCM as the eluent to yield the pure product 2-pyridyl methyl phosphonate (g, 18.26 g, 79.66 mmol, 79% yield).

GP6: Wittig Reaction (c+f to h). To a suspension of the Wittig reagent triphenyl(pyrimidin-2-ylmethyl)phosphonium chloride (f, 0.719 g, 1.839 mmol) in anhydrous THE (40 mL) at −70° C. under a N₂ atmosphere was added dropwise LHMDS (1.0 M in THF, 2.03 mL, 2.024 mmol, 1.1 equiv), which was stirred at the same temperature for 15 min. To this mixture a solution of Boc-Phe-H (c, 0.321 g, 1.287 mmol, 0.7 equiv) in THE (10 mL) was added and stirred over 2 h until the temperature reached −40° C. Upon completion of the reaction as revealed by TLC analysis (EtOAc/hexane=1:1, v/v), the reaction was quenched by the addition of 0.1 mL of glacial acetic acid, followed by aqueous NaHCO₃. Most of the THF was removed carefully using a rotary evaporator, and the residue was extracted with EtOAc (2×). The organic layer was dried over anhydrous Na₂SO₄ and filtered. The filtrate was concentrated in vacuo to afford the crude material, which was purified by silica gel column chromatography using a gradient of 5-30% of EtOAc in hexane as the eluent, yielding the pure olefin tert-butyl (S,E)-(1-phenyl-4-(pyrimidin-2-yl)but-3-en-2-yl)carbamate (h, E isomer, 0.060 g, 14% yield). The other Z isomer (0.014 g) was isolated as a side product, and the ratio of E to Z isomers was typically 4:1.

GP7: Horner-Wadsworth-Emmons Reaction (c+g to h). To a solution of the 2-pyridyl methyl phosphonate ester (g, 1.30 g, 5.65 mmol) in anhydrous THE (25 mL) at −70° C. under a N₂ atmosphere was added dropwise LHMDS (1.0 M in THF, 6.22 mL, 6.22 mmol, 1.1 equiv). The reaction was stirred at the same temperature for 15 min, followed by the dropwise addition of a solution of Boc-hPhe-H (c, 1.34 g in 10 mL THF, 5.09 mmol, 0.9 equiv). The reaction was stirred until it reached a temperature of −20° C. over 2 h. Upon completion of the reaction as revealed by TLC analysis (EtOAc/hexane=1:1, v/v), to the reaction mixture at 0° C. was added glacial acetic acid (0.5 mL), followed by the addition of 20 mL of saturated NaHCO₃. The aqueous layer was extracted with EtOAc (3×100 mL). Extracts were washed with brine (1×50 mL), and the organic layer was dried over anhydrous Na₂SO₄ and filtered. The filtrate was concentrated in vacuo to afford the crude product, which was purified by silica gel column chromatography using a gradient of 10-50% of EtOAc in hexane as the eluent to yield the pure product tert-butyl (S,E)-(5-phenyl-1-(pyridin-2-yl)pent-1-en-3-yl)carbamate (h, 0.344 g, 1.016 mmol, 20% yield).

GP8: Amide Coupling with P3-P2 Fragment (h to j). To a solution of tert-butyl (S,E)-(5-phenyl-1-(pyridin-2-yl)pent-1-en-3-yl)carbamate (h, 0.143 g, 0.423 mmol) in anhydrous DCM (5 mL) at 0° C. was added dropwise TFA (1.5 mL in 1 mL DCM) with stirring at the same temperature for 1 h. Upon completion of the reaction as revealed by TLC analysis (EtOAc/hexane=1:1, v/v), the reaction solvent was removed by a rotary evaporator. The resulting oil was coevaporated on a rotary evaporator with CHCl₃ (3×) and ether (3×). The solid product was dried on high vacuum to yield the TFA salt (S,E)-5-phenyl-1-(pyridin-2-yl)pent-1-en-3-aminium trifluoroacetate (0.149 g, 0.423 mmol), which was used in subsequent synthetic steps without further purification. To a solution of the above TFA salt in anhydrous DCM (5 mL) at −10° C. under a N₂ atmosphere was added dropwise DIPEA (0.6 mL, 0.3.44 mmol, 8 equiv), followed by the addition of Cbz-Phe-OH (0.13 g, 0.43 mmol, 1 equiv) and T3P (50% in EtOAc, 0.41 mL, 1.5 equiv). The reaction was stirred at 0° C. for an additional 1 h. Upon completion of the reaction as revealed by TLC analysis (EtOAc/hexane=1:1, v/v), the reaction mixture was diluted with DCM (50 mL) and then washed with H₂O (3×) and brine (3×). The organic layer was dried over anhydrous Na₂SO₄ and filtered. The filtrate was concentrated in vacuo to afford the crude product, which was purified by silica gel column chromatography using a gradient of 10-50% of EtOAc in hexane as the eluent to yield the pure product benzyl ((S)-1-oxo-3-phenyl-1-(((S,E)-5-phenyl-1-(pyridin-2-yl)pent-1-en-3-yl)amino)propan-2-yl)carbamate (i, 0.113 g, 0.217 mmol, 51% yield).

GP9: N-Methylation Using Methyl Iodide (i to j). To a suspension of benzyl ((S)-1-oxo-3-phenyl-1-(((S,E)-1-phenyl-4-(pyridin-2-yl)-but-3-en-2-yl)amino)propan-2-yl)carbamate (i, 0.049 g, 0.098 mmol) in anhydrous MeCN (5 mL) under a N₂ atmosphere was added Mel (0.03 mL, 0.490 mmol, 5 equiv), and the reaction mixture was heated under reflux for 9 h. Upon completion of the reaction as revealed by TLC analysis (EtOAc/hexane=1:1, v/v), the solvents were removed by rotary evaporation. The resulting gummy residue was dissolved in CHCl₃ (1 mL) and precipitated with ether (5 mL). The solvents were decanted, and this procedure was repeated twice. The solid obtained was dried under high vacuum to give pure product 2-((S,E)-3-((S)-2-(((benzyloxy)carbonyl)amino)-3-phenylpropanamido)-4-phenylbut-1-en-1-yl)-1-methylpyridin-1-ium iodide as a yellow solid (j, 0.039 g, 61% yield).

GP10: Preparation of Peptide Acrylamide (i to k). A solution of ethyl (S,E)-4-((S)-2-(((benzyloxy)carbonyl)amino)-3-phenylpropanamido)-5-phenylpent-2-enoate (i, 0.346 g, 0.69 mmol) in THE (6 mL) at 0° C. was treated with LiOH (1 N in H₂O, 0.83 mL, 0.83 mmol, 1.2 equiv) and stirred overnight. The reaction was concentrated by rotary evaporation, and the aqueous layer was added to water, acidified to pH 1-2, and extracted with EtOAc (3×). The combined organic layers were dried and concentrated to yield the crude acrylic acid. To a solution of this acrylic acid (0.124 g, 0.262 mmol) in THE (6 mL) at −15° C. were added Et₃N (0.11 mL, 0.787 mmol, 3 equiv) and the dropwise addition of ClCO₂Et (0.035 mL, 0.367 mmol), which resulted in a white precipitate. The reaction mixture was stirred at the same temperature for an additional 30 min, and then aqueous 1 M NH₄Cl (0.4 mL) was added dropwise with continuous stirring over 3 h until a temperature of 25° C. was attained. Upon completion of the reaction as revealed by TLC analysis (EtOAc/hexane=1:1, v/v), most of the reaction solvent was removed using a rotary evaporator, and the solid residue was extracted with EtOAc. The organic layer was washed with aqueous NaHCO₃ (2×), H2O (1×), and brine (1×), dried over anhydrous Na₂SO₄, and filtered. The filtrate was concentrated in vacuo to afford the crude product. Purification of the crude product by silica gel chromatography using a gradient of 20-100% of EtOAc in hexane as the eluent yielded the pure product benzyl ((S)-1-(((S,E)-5-amino-5-oxo-1-phenylpent-3-en-2-yl)amino)-1-oxo-3-phenylpropan-2-yl)carbamate (0.027 g, 0.057 mmol, 22% yield).

4-Methyl-N—((S)-1-oxo-3-phenyl-1-(((S,E)-1-phenyl-4-(phenylsulfonyl)but-3-en-2-yl)amino)propan-2-yl)piperazine-1-carboxamide (1, Cbz-Phe-Phe-VSPh). White solid, 0.115 g, 0.202 mmol, 56% yield. 1H NMR (400 MHz, CDCl₃): δ 2.79 (d, J=6.8 Hz, 2H), 2.85-3.12 (m, 2H), 4.26 (q, J=7.3 Hz, 1H), 4.79-4.95 (m, 1H), 5.04 (s, 2H), 5.13 (s, 1H), 5.75 (s, 1H), 5.96 (dd, J1=1.8 Hz, J2=15.1 Hz, 1H), 6.78 (dd, J1=4.8 Hz, J2=15.1 Hz, 1H), 6.95-7.03 (m, 2H), 7.05-7.11 (m, 2H), 7.12-7.23 (m, 6H), 7.27-7.39 (m, 5H), 7.47-7.56 (m, 2H), 7.57-7.67 (m, 1H), 7.72-7.84 (m, 2H). ¹³C NMR (100 MHz, CDCl₃): δ 38.4, 40.3, 50.4, 56.7, 67.4, 127.3, 127.4, 127.8, 128.2, 128.5, 128.7 (2C), 128.8, 129.0, 129.3, 129.4, 131.1, 133.6, 135.5, 136.1, 136.2, 140.2, 144.6, 156.0, 170.5. LC-MS m/z: 569.31 [M+H]+(calcd for C₃₃H₃₂N₂O₅S, 569.21); tR=7.27 min.

Benzyl ((S)-1-(((S,E)-5-Amino-5-oxo-1-phenylpent-3-en-2-yl)-amino)-1-oxo-3-phenylpropan-2-yl)carbamate (2, Cbz-Phe-Phevinyl-CONH₂). White solid, 0.027 g, 0.057 mmol, 22% yield. 1H-NMR (400 MHz, DMSO-d₆): δ 2.64-2.77 (m, 1H), 2.84 (d, J=7.2 Hz, 2H), 2.96 (dd, J1=3.9 Hz, J2=13.7 Hz, 1H), 4.19-4.31 (m, 1H), 4.62 (pentet, J=6.8 Hz, 1H), 4.95 (s, 1H), 5.85 (d, J=15.5 Hz, 1H), 6.56 (dd, J1=5.9 Hz, J2=15.5 Hz, 1H), 6.93 (s, 1H), 7.12-7.46 (m, 17H), 8.25 (d, J=8.2 Hz, 1H). ¹³C NMR (100 MHz, DMSOd₆): δ 28.7, 37.6, 50.9, 56.07, 65.1, 124.1, 126.2, 126.4, 127.4, 127.6, 127.9, 128.1, 128.2, 129.2, 129.5, 137.0, 137.8, 138.0, 142.0, 155.6, 166.2, 170.7. LC-MS m/z: 472.46 [M+H]⁺ (calcd for C₂₈H₂₉N₃O₄⁺, 472.22); tR=4.71 min.

Benzyl ((S)-1-(((S,E)-6-Amino-6-oxo-1-phenylhex-4-en-3-yl)-amino)-1-oxo-3-phenylpropan-2-yl)carbamate (3, Cbz-Phe-hPhe-vinyl-CONH₂). White solid, 0.013 g, 0.027 mmol, 15% yield. ¹H NMR (400 MHz, DMSO-d₆): δ 1.64-1.92 (m, 2H), 2.54-2.72 (m, 2H), 2.81 (dd, J1=10.6 Hz, J2=13.6 Hz, 1H), 3.03 (dd, J1=4.0 Hz, J2=13.6 Hz, 1H), 4.25-4.33 (m, 1H), 4.35-4.43 (m, 1H), 4.85-5.04 (m, 2H), 5.90 (d, J=15.5 Hz, 1H), 6.54 (dd, J1=5.7 Hz, J2=15.5 Hz, 1H), 6.94 (s, 1H), 7.15-7.35 (m, 15H), 7.41 (s, 1H), 7.49 (d, J=8.5 Hz, 1H), 8.22 (d, J=8.2 Hz, 1H). LC-MS m/z: 486.24 [M+H]⁺ (calcd for C₂₉H₃₁N₃O₄⁺, 486.24); tR=4.89 min.

N—((S)-1-(((S,E)-6-Amino-6-oxo-1-phenylhex-4-en-3-yl)amino)-1-oxo-3-phenylpropan-2-yl)-4-methylpiperazine-1-carboxamide (4,NMePip-Phe-hPhe-vinyl-CONH₂). White solid, 0.022 g, 0.048 mmol, 18% yield. ¹H NMR (400 MHz, CDCl₃): δ 1.66-1.91 (m, 2H), 2.23 (s, 3H), 2.25-2.34 (m, 4H), 2.50-2.64 (m, 2H), 2.99-3.15 (m, 2H), 3.25-3.41 (m, 4H), 4.44-4.57 (m, 1H), 4.62 (q, J=7.4 Hz, 1H), 5.37 (d, J=7.6 Hz, 1H), 5.60 (dd, J1=1.3 Hz, J2=15.3 Hz, 1H), 5.75 (s, 1H), 6.01 (s, 1H), 6.62 (dd, J1=5.5 Hz, J2=15.3 Hz, 1H), 6.93 (d, J=8.2 Hz, 1H), 7.04-7.31 (m, 11H). ¹³C NMR (100 MHz, DMSO-d6): δ 32.0, 36.1, 38.5, 43.9, 46.1, 49.9, 54.6, 56.2, 122.9, 126.2, 126.9, 128.5, 128.6, 128.8, 129.7, 137.3, 141.1, 144.4, 157.3, 167.5, 172.0. LC-MS m/z: 478.36 [M+H]⁺ (calcd for C₂₇H₃₅N₅O₃⁺, 478.28); tR=2.54 min.

Benzyl ((S)-1-(((S,E)-1,4-Diphenylbut-3-en-2-yl)amino)-1-oxo-3-phenylpropan-2-yl)carbamate (5, Cbz-Phe-Phe-vinyl-Ph). Off-white solid, 0.054 g, 0.107 mmol, 35% yield. ¹H NMR (400 MHz, CDCl₃): δ 2.87 (dt, J=2.8, 6.3 Hz, 2H), 2.96-3.16 (m, 2H), 4.35 (q, J=7.6 Hz, 1H), 4.80-4.96 (m, 1H), 5.29 (d, J=9.6 Hz, 1H), 5.69 (d, J=9.9 Hz, 1H), 5.94 (ddt, J=3.0, 6.3, 15.9 Hz, 1H), 6.27 (d, J=15.9 Hz, 1H), 7.03-7.14 (m, 2H), 7.14-7.28 (m, 10H), 7.28-7.40 (m, 9H). ¹³C NMR (100 MHz, CDCl₃): δ 29.7, 38.6, 41.3, 51.8, 67.1, 126.4, 126.6, 127.1, 127.6, 128.0, 128.2, 128.3, 128.4, 128.5, 128.7, 129.3, 129.4, 130.9, 136.5, 136.8, 169.8. LC-MS m/z: 505.31 [M+H]⁺ (calcd for C₃₃H₃₂N₂O₃⁺, 505.25); tR=6.20 min.

Benzyl ((S)-1-(((S,E)-4-(4-Nitrophenyl)-1-phenylbut-3-en-2-yl)-amino)-1-oxo-3-phenylpropan-2-yl)carbamate (6, Cbz-Phe-Phe-vinyl-(4-NO₂)Ph). White fluffy solid, 0.530 g, 0.964 mmol, 55% yield. ¹H NMR (400 MHz, DMSO-d₆): δ 2.76 (dd, J1=9.7 Hz, J2=13.4 Hz, 1H), 2.85-2.97 (m, 3H), 4.18-4.35 (m, 1H), 4.68 (pentet, J=6.7 Hz, 1H), 4.99 (s, 2H), 6.41 (d, J=16.1 Hz, 1H), 6.50 (dd, J1=5.4 Hz, J2=16.1 Hz, 1H), 7.13-7.37 (m, 15H), 7.42 (d, J=8.5 Hz, 1H), 7.58 (d, J=8.5 Hz, 2H), 8.14-8.25 (m, 3H). ¹³C NMR (100 MHz, DMSO-d₆): δ 37.7, 39.0, 51.9, 56.2, 65.2, 123.9, 126.1, 126.2, 127.0, 127.4, 127.6, 127.9, 128.0, 128.1, 128.2, 129.2, 129.3, 135.6, 137.0, 137.8, 138.0, 143.3, 146.2, 155.6, 170.6. LC-MS m/z: 550.28 [M+H]⁺ (calcd for C₃₃H₃₁N₃O₅+, 550.23); tR=6.12 min.

Benzyl ((S)-1-Oxo-3-phenyl-1-(((S,E)-1-phenyl-4-(pyrimidin-2-yl)-but-3-en-2-yl)amino)propan-2-yl)carbamate (7, Cbz-Phe-Phevinyl-2Pyrmd). White solid, 0.026 g, 0.0513 mmol, 33% yield. 1H NMR (400 MHz, CDCl₃): δ 2.73-2.96 (m, 2H), 3.01 (d, J=4.8 Hz, 2H), 4.23-4.50 (m, 1H), 4.91-5.02 (m, 1H), 5.05 (s, 2H), 5.30 (s, 1H), 6.05 (s, 1H), 6.48 (d, J=15.7 Hz, 1H), 7.00 (dd, J1=5.6 Hz, J2=15.7 Hz, 1H), 7.05-7.37 (m, 16H), 8.63 (d, J=4.0 Hz, 2H). 13C NMR (100 MHz, CDCl₃): δ 38.6, 41.0, 51.6, 56.5, 67.3, 119.0, 126.9, 127.2, 128.2, 128.3, 128.6, 128.7, 128.9, 129.4, 129.5, 130.3, 136.3. 136.5, 136.7, 139.2, 156.1, 157.1, 164.2, 170.3. LC-MS m/z: 507.26 [M+H]⁺ (calcd for C₃₁H₃₀N₄O₃⁺, 507.24); tR=5.14 min.

Benzyl ((S)-1-Oxo-3-phenyl-1-(((S,E)-5-phenyl-1-(pyrimidin-2-yl)-pent-1-en-3-yl)amino)propan-2-yl)carbamate (8, Cbz-Phe-hPhevinyl-2Pyrmd). Off-white solid, 0.280 g, 0.538 mmol, 29% yield. ¹H-NMR (400 MHz, CDCl₃): δ 1.76-2.03 (m, 2H), 2.63 (t, J=7.9 Hz, 2H), 3.10 (t, J=8.4 Hz, 2H), 4.47 (s, 1H), 4.74 (h, J=7.3 Hz, 1H), 5.10 (d, J=7.9 Hz, 2H), 5.53 (s, 1H), 6.27 (s, 1H), 6.56-6.72 (m, 1H), 6.96-7.05 (m, 1H), 7.06-7.14 (m, 3H), 7.19 (d, J=7.3 Hz, 3H), 7.21-7.34 (m, 10H), 8.66 (dd, J=4.9, 15.8 Hz, 2H). 13C NMR (100 MHz, CDCl₃): δ 32.0, 36.2, 38.6, 50.5, 56.6, 67.1, 118.9, 126.0, 127.0, 128.0, 128.1, 128.3, 128.4, 128.5, 128.7, 128.8, 129.3, 129.4, 130.1, 136.4, 139.6, 141.2, 156.9, 164.1, 170.3. LC-MS m/z: 521.24 [M+H]⁺ (calcd for C₃₂H₃₂N₄O₃⁺, 521.26); tR=5.56 min.

4-Methyl-N—((S)-1-oxo-3-phenyl-1-(((S,E)-5-phenyl-1-(pyrimidin-2-yl)pent-1-en-3-yl)amino)propan-2-yl)piperazine-1-carboxamide (9, NMePip-Phe-hPhe-vinyl-2Pyrmd). Off-white gum, 0.054 g, 0.105 mmol, 54% yield. ¹H NMR (400 MHz, CDCl₃): δ 1.82-1.99 (m, 2H), 2.29 (s, 3H), 2.34-2.46 (m, 4H), 2.61 (t, J=7.5 Hz, 1H), 3.09 (d, J=7.5 Hz, 2H), 3.39 (s, 4H), 4.58-4.74 (m, 2H), 5.47 (d, J=6.3 Hz, 1H), 6.61 (d, J=15.7 Hz, 1H), 6.87 (d, J=8.2 Hz, 1H), 6.98 (dd, J1=6.3 Hz, J2=15.7 Hz, 1H), 7.06-7.31 (m, 11H), 8.66 (d, J=4.8 Hz, 2H). ¹³C NMR (100 MHz, CDCl₃): δ 32.1, 36.5, 38.8, 43.5, 45.6, 50.6, 54.3, 56.1, 118.9, 126.0, 126.9, 128.4, 128.5 (2C), 128.6, 129.6, 130.1, 137.2, 140.1, 141.3, 157.0, 164.3, 171.8. LC-MS m/z: 513.17 [M+H]⁺ (calcd for C₃₀H₃₆N₆O₂⁺, 513.30); tR=2.96 min.

Benzyl ((S)-1-Oxo-3-phenyl-1-(((S,E)-4-(pyrimidin-2-yl)but-3-en-2-yl)amino)propan-2-yl)carbamate (10, Cbz-Phe-Ala-vinyl-2Pyrmd). White fluffy solid, 0.017 g, 0.039 mmol, 14% yield. ¹H NMR (400 MHz, CDCl₃+MeOD): δ 1.32 (d, J=6.6 Hz, 3H), 2.96 (dd, J1=8.0 Hz, J2=13.6 Hz, 1H), 3.10 (dd, J1=6.4 Hz, J2=13.6 Hz, 1H), 4.36-4.44 (m, 4H), 4.71 (pentet, J=6.4 Hz, 1H), 4.97-5.12 (m, 2H), 6.52 (d, J=15.7 Hz, 1H), 6.98 (dd, J1=5.8 Hz, J2=15.7 Hz, 1H), 7.13-7.36 (m, 11H), 8.70 (d, J=4.9 Hz, 2H). 13C NMR (100 MHz, CDCl₃+MeOD): δ 19.6, 38.7, 46.1, 56.1, 66.7, 119.0, 126.7, 127.6, 127.9, 128.1, 128.3 (2C), 129.2, 136.3, 141.6, 156.9 (2C), 157.0, 163.9, 171.0. LC-MS m/z: 430.91 [M+H]⁺ (calcd for C₂₅H₂₆N₄O₃⁺, 431.21); tR=4.68 min.

Benzyl ((S)-1-Oxo-3-phenyl-1-(((S,E)-1-phenyl-4-(pyridin-2-yl)-but-3-en-2-yl)amino)propan-2-yl)carbamate (11, Cbz-Phe-Phevinyl-2Pyr). White solid, 0.554 g, 1.096 mmol, 81% yield. ¹H NMR (400 MHz, CDCl₃): δ 2.87 (dq, J1=6.8 Hz, J2=13.7 Hz, 1H), 3.01 (d, J=7.1 Hz, 2H), 4.35 (q, J=7.0 Hz, 1H), 4.91 (pentet, J=6.8 Hz, 1H), 5.06 (s, 2H), 5.24 (s, 1H), 5.88 (d, J=8.4 Hz, 1H), 6.32 (d, J=15.7 Hz, 1H), 6.58 (dd, J1=6.1 Hz, J2=15.7 Hz, 1H), 7.08-7.36 (m, 17H), 7.59 (dt, J1=1.6 Hz, J2=7.7 Hz, 1H), 8.53 (d, J=4.3 Hz, 1H). 13C NMR (100 MHz, CDCl₃): δ 38.4, 41.1, 51.7, 56.4, 67.1, 122.1, 122.2, 126.7, 127.0, 128.0, 128.2, 128.4, 128.5 (2C), 128.8, 129.4 (2C), 130.5, 133.0, 136.1, 136.4, 136.8, 149.5, 154.8, 155.9, 170.0. LC-MS m/z: 506.24 [M+H]⁺ (calcd for C₃₂H₃₁N₃O₃⁺, 506.24); tR=4.39 min.

2-((S,E)-3-((S)-2-(((Benzyloxy)carbonyl)amino)-3-phenylpropanamido)-4-phenylbut-1-en-1-yl)-1-methylpyridin-1-ium Iodide (12, Cbz-Phe-Phe-Vinyl-2PyrNMe). Yellow solid, 0.039 g, 0.060 mmol, 61% yield. ¹H NMR (400 MHz, CDCl₃): δ 3.01-3.15 (m, 3H), 3.19 (dd, J1=7.7 Hz, J2=13.6 Hz, 1H), 4.19 (s, 3H), 4.51 (q, J=7.0 Hz, 1H), 4.90-5.02 (m, 2H), 5.06 (s, 1H), 5.81 (s, 1H), 6.69 (d, J=15.7 Hz, 1H), 6.86 (dd, J1=4.6 Hz, J2=15.7 Hz, 1H), 7.08-7.28 (m, 14H), 7.65-7.79 (m, 2H), 7.89-8.01 (m, 1H), 8.21-8.31 (m, 1H), 8.97-9.09 (m, 1H). 13C NMR (100 MHz, CDCl₃): δ 38.1, 39.8, 47.5, 52.6, 57.1, 66.8, 119.9, 126.1, 126.3, 126.9, 127.1, 127.5, 127.6, 127.9, 128.5, 128.6, 128.8, 129.6, 129.7, 136.7, 136.9, 144.9, 146.0, 147.6, 152.8, 156.2, 171.5. LC-MS m/z: 520.32 [M+H]⁺ (calcd for C₃₃H₃₄N₃O₃⁺, 520.26); tR=3.25 min.

Benzyl ((S)-1-Oxo-3-phenyl-1-(((S,E)-5-phenyl-1-(pyridin-2-yl)-pent-1-en-3-yl)amino)propan-2-yl)carbamate (13, Cbz-Phe-hPhevinyl-2Pyr). White solid, 0.113 g, 0.217 mmol, 51% yield. ¹H NMR (400 MHz, CDCl3): δ 1.73-1.95 (m, 2H), 2.59 (t, J=7.9 Hz, 2H), 3.04 (d, J=7.0 Hz, 2H), 4.38-4.52 (m, 1H), 4.65 (pentet, J=7.1 Hz, 1H), 5.03 (s, 2H), 5.59 (d, J=6.4 Hz, 1H), 6.34 (d, J=5.1 Hz, 1H), 6.43 (d, J=15.7 Hz, 1H), 6.53 (dd, J1=6.2 Hz, J2=15.7 Hz, 1H), 7.02-7.32 (m, 17H), 7.57 (dt, J1=1.4 Hz, J2=7.7 Hz, 1H), 8.51 (d, J=4.4 Hz, 1H). 13C NMR (100 MHz, CDCl₃): δ 32.1, 36.4, 38.7, 50.8, 56.6, 67.1, 122.1, 122.3, 126.0, 127.0, 128.0, 128.2, 128.4, 128.5 (2C), 128.7, 129.5, 130.5, 133.8, 136.2, 136.5 (2C), 141.4, 149.5, 155.0, 156.1, 170.4. LC-MS m/z: 518.74, 520.41 [M+H]⁺ (calcd for C₃₃H₃₃N₃O₃⁺, 520.26); tR=4.68 min.

4-Methyl-N—((S)-1-oxo-3-phenyl-1-(((S,E)-5-phenyl-1-(pyridin-2-yl)pent-1-en-3-yl)amino)propan-2-yl)piperazine-1-carboxamide (14, NMePip-Phe-hPhe-vinyl-2Pyr). Off-white solid, 0.090 g, 0.176 mmol, 44% yield. ¹H NMR (400 MHz, CDCl₃): δ 1.77-1.94 (m, 2H), 2.21 (s, 3H), 2.22-2.27 (m, 4H), 2.59 (t, J=8.0 Hz, 2H), 3.08 (d, J=7.0 Hz, 2H), 3.28 (m, 4H), 4.61 (pentet, J=7.3 Hz, 1H), 4.7 (q, J=7.3 Hz, 1H), 5.39 (d, J=7.7 Hz, 1H), 6.46 (d, J=15.8 Hz, 1H), 6.56 (dd, J1=6.2 Hz, J2=15.8 Hz, 1H), 6.98 (d, J=8.4 Hz, 1H), 7.06-7.24 (m, 12H), 7.59 (dt, J1=1.8 Hz, J2=7.7 Hz, 1H), 8.53 (d, J=4.3 Hz, 1H). ¹³C NMR (100 MHz, CDCl₃): δ 32.1, 36.6, 39.0, 43.8, 46.1, 50.7, 54.6, 55.9, 122.0, 122.2, 125.9, 126.8, 128.4 (2C), 128.5, 129.6, 130.3, 134.2, 136.4, 137.2, 141.4, 149.5, 155.1, 157.0, 171.6. LC-MS m/z: 512.28 [M+H]⁺ (calcd for C₃₁H₃₇N₅O₂⁺, 512.30); tR=2.60 min.

2-((S,E)-3-((S)-2-(((Benzyloxy)carbonyl)amino)-3-phenylpropanamido)-5-phenylpent-1-en-1-yl)-1-methylpyridin-1-ium (15, Cbz-Phe-hPhe-vinyl-2PyrNMe). Yellow solid, 0.029 g, 0.044 mmol, 89% yield. 1H NMR (400 MHz, CDCl₃): δ 1.98-2.10 (m, 1H), 2.11-2.25 (m, 1H), 2.56-2.85 (m, 2H), 3.04-3.35 (m, 2H), 4.22 (s, 3H), 4.62 (d, J=5.5 Hz, 1H), 4.79 (s, 1H), 4.92-5.09 (m, 2H), 5.88 (s, 1H), 6.70 (d, J=15.8 Hz, 1H), 6.77 (dd, J1=3.6 Hz, J2=15.8 Hz, 1H), 7.09-7.34 (m, 14H), 7.66-7.79 (m, 2H), 7.90 (d, J=6.0 Hz, 1H), 8.17-8.30 (m, 1H), 8.98 (d, J=4.5 Hz, 1H). LC-MS m/z: 534.25 [M+H]⁺ (calcd for C₃₄H₃₆N₃O₃⁺, 534.28); tR=3.48 min.

Benzyl ((S)-1-(((S,E)-1-(4-Methoxypyridin-2-yl)-5-phenylpent-1-en-3-yl)amino)-1-oxo-3-phenylpropan-2-yl)carbamate (16, Cbz-Phe-hPhe-vinyl-2-(4-OMe)-Pyr). Pale yellow solid, 0.072 g, 0.131 mmol, 33% yield. ¹H NMR (400 MHz, CDCl₃): δ 1.73-2.01 (m, 2H), 2.59 (dt, J=7.9, 37.9 Hz, 2H), 3.11 (dd, J=7.0, 11.7 Hz, 2H), 3.87 (d, J=9.2 Hz, 3H), 4.50 (dd, J=7.6, 23.1 Hz, 1H), 4.68 (t, J=7.1 Hz, 1H), 5.08 (d, J=4.6 Hz, 2H), 5.74 (dd, J=8.1, 65.8 Hz, 1H), 6.39-6.58 (m, 1H), 6.67-6.81 (m, 2H), 7.11-7.15 (m, 2H), 7.16-7.26 (m, 8H), 7.27-7.34 (m, 7H), 8.37 (dd, J=5.8, 23.3 Hz, 1H). ¹³C NMR (100 MHz, CDCl₃): δ 32.0, 36.3, 38.7, 50.6, 55.2, 56.5, 67.1, 108.3, 108.5, 126.0, 126.6, 127.0, 127.9, 128.0, 128.1, 128.1, 128.3, 128.4, 128.4, 128.4, 128.5, 128.5, 128.7, 129.4, 129.4, 134.7, 135.1, 136.5, 141.3, 150.0, 156.1, 166.6, 170.3. LC-MS m/z: 550.16 [M+H]⁺ (calcd for C₃₄H₃₅N₃O₄⁺, 549.26); tR=3.57 min.

Benzyl ((S)-1-Oxo-3-phenyl-1-(((S,E)-5-phenyl-1-(4-(trifluoromethyl)pyridin-2-yl)pent-1-en-3-yl)amino)propan-2-yl)-carbamate (17, Cbz-Phe-hPhe-vinyl-2-(4-CF₃)-Pyr). White solid, 0.260 g, 0.442 mmol, 79% yield. ¹H NMR (400 MHz, CDCl₃): δ 1.76-1.95 (m, 2H), 2.59 (t, J=7.8 Hz, 2H), 3.06 (d, J=7.1 Hz, 2H), 4.36-4.51 (m, 1H), 4.65 (pentet, J=7.1 Hz, 1H), 5.05 (s, 2H), 5.49 (s, 1H), 6.13 (s, 1H), 6.35 (d, J=15.7 Hz, 1H), 6.59 (dd, J1=6.0 Hz, J2=15.7 Hz, 1H), 7.06-7.11 (m, 2H), 7.14-7.33 (m, 15H), 8.67 (d, J=4.9 Hz, 1H). 13C NMR (100 MHz, CDCl₃): δ 32.1, 36.4, 38.7, 50.7, 56.8, 67.3, 117.5, 121.6, 124.3, 126.2, 127.0 (C—F), 127.2, 128.1, 128.3, 128.5, 128.6 (3C), 128.8, 129.2, 129.5, 136.2, 136.6, 138.7, 139.1, 141.2, 150.5, 156.2 (C—F), 156.4, 170.4. LC-MS m/z: 587.95 [M+H]⁺ (calcd for C₃₄H₃₂F₃N₃O₃⁺, 588.25); tR=5.16 min.

Benzyl ((S)-4-Methyl-1-oxo-1-(((S,E)-5-phenyl-1-(pyridin-2-yl)-pent-1-en-3-yl)amino)pentan-2-yl)carbamate (18, Cbz-Leu-hPhevinyl-2Pyr). White solid, 0.038 g, 0.078 mmol, 26% yield. ¹H NMR (400 MHz, CDCl3): δ 0.92 (t, J=6.4 Hz, 6H), 1.45-1.57 (m, 1H), 1.59-1.73 (m, 2H), 1.79 (s, 1H), 1.86-2.05 (m, 2H), 2.68 (t, J=7.9 Hz, 2H), 4.03-4.27 (m, 1H), 4.71 (pentet, J=6.9 Hz, 1H), 5.10 (s, 2H), 5.14 (s, 1H), 6.26 (d, J=6.2 Hz, 1H), 6.58 (d, J=15.8 Hz, 1H), 6.69 (dd, J1=6.0 Hz, J2=15.8 Hz, 1H), 7.10-7.34 (m, 12H), 7.60 (dt, J1=1.7 Hz, J2=7.7 Hz, 1H), 8.54 (d, J=4.6 Hz, 1H). 13C NMR (100 MHz, CDCl₃): δ 23.1, 24.9, 32.3, 36.7, 41.2, 50.9, 54.0, 67.3, 122.3, 122.4, 126.1, 128.2, 128.4, 128.6 (2C), 128.7, 130.6, 134.0, 136.3, 136.7, 141.6, 149.7, 155.1, 156.5, 171.6. LC-MS m/z: 484.46, 485.49, 486.38 [M+H]⁺ (calcd for C₃₀H₃₅N₃O₃⁺, 486.28); tR=4.58 min.

Benzyl ((S)-1-Oxo-3-phenyl-1-(((S,E)-4-(pyridin-2-yl)but-3-en-2-yl)amino)propan-2-yl)carbamate (19, Cbz-Phe-Ala-vinyl-2Pyr). White solid, 0.590 g, 1.374 mmol, 75% yield. ¹H NMR (400 MHz, CDCl3): δ 1.58 (s, 3H), 2.96-3.23 (m, 2H), 4.36 (q, J=7.4 Hz, 1H), 4.63-4.80 (m, 1H), 5.10 (d, J=1.4 Hz, 2H), 5.33 (s, 1H), 5.67 (d, J=8.3 Hz, 1H), 6.40 (dd, J=1.3, 15.8 Hz, 1H), 6.51 (dd, J=5.5, 15.8 Hz, 1H), 7.13 (ddd, J=1.1, 4.8, 7.4 Hz, 1H), 7.16-7.21 (m, 3H), 7.21-7.37 (m, 8H), 7.62 (td, J=1.8, 7.7 Hz, 1H), 8.45-8.61 (m, 1H). 13C NMR (100 MHz, CDCl₃): δ 20.0, 38.8, 46.4, 56.2, 66.8, 121.8, 122.3, 126.7, 127.7, 128.0, 128.3, 128.7, 129.3, 135.6, 136.4, 137.0, 148.8, 155.0, 170.8. LC-MS m/z: 430.35 [M+H]+ (calcd for C₂₆H₂₇F₃N₃O₃⁺, 430.21); tR=3.49 min.

(S,E)-5-((S)-2-(((Benzyloxy)carbonyl)amino)-3-phenylpropanamido)-7-(pyridin-2-yl)hept-6-en-1-aminium Chloride (20, Cbz-Phe-Lys-vinyl-2Pyr). White solid, 0.027 g, 0.048 mmol, 51% yield. ¹H NMR (400 MHz, CDCl₃): δ 1.44-1.59 (m, 2H), 1.65-1.84 (m, 4H), 2.89-3.02 (m, 3H), 3.09-3.19 (m, 1H), 4.43 (t, J=7.5 Hz, 1H), 4.64-4.70 (m, 1H), 5.01-5.11 (m, 2H), 6.54 (d, J=16.1 Hz, 1H), 6.92 (dd, J1=5.7 Hz, J2=16.1 Hz, 1H), 7.11 (t, J=7.5 Hz, 1H), 7.21 (t, J=7.5 Hz, 2H), 7.25-7.40 (m, 7H), 7.91 (t, J=6.8 Hz, 1H), 8.10 (d, J=8.0 Hz, 1H), 8.54 (t, J=8.0 Hz, 1H), 8.69 (d, J=5.2 Hz, 1H). ¹³C NMR (100 MHz, MeOD): δ 23.8, 27.9, 34.0, 39.1, 40.6, 51.9, 58.2, 67.6, 121.6, 125.9, 126.5, 127.7, 128.5, 128.9, 129.5, 129.7, 130.5, 138.1, 138.4, 142.0, 146.3, 147.8, 151.2, 158.3, 174.1. LC-MS m/z: 486.98 [M+H]+, 508.94 [M+Na]+ (calcd for C₂₉H₃₅N₄O₃⁺, 487.27 and calcd for C₂₉H₃₅N₄O₃Na⁺, 509.25, respectively); tR=3.16 min.

Benzyl ((S)-1-Oxo-3-phenyl-1-(((S,E)-1-phenyl-4-(pyridin-4-yl)-but-3-en-2-yl)amino)propan-2-yl)carbamate (21, Cbz-Phe-Phevinyl-4Pyr). Off-white solid, 0.051 g, 0.109 mmol, 50% yield. ¹H NMR (400 MHz, CDCl₃): δ 2.83 (d, J=6.8 Hz, 2H), 3.02 (d, J=7.8 Hz, 2H), 4.36 (s, 1H), 4.86 (s, 1H), 5.08 (s, 2H), 5.90 (s, 1H), 6.13 (s, 1H), 7.10 (dd, J=6.5, 31.0 Hz, 6H), 7.18-7.27 (m, 6H), 7.31 (dd, J=3.6, 6.4 Hz, 5H), 8.50 (d, J=6.0 Hz, 2H). 13C NMR (100 MHz, CDCl₃): δ 38.4, 41.0, 51.5, 52.1, 67.1, 105.0, 120.9, 126.9, 127.1, 127.9, 128.3, 128.4, 128.5, 128.6, 128.8, 129.3, 129.4, 133.4, 136.4, 150.0, 170.1. LC-MS m/z: 506.29 [M+H]+ (calcd for C₃₂H₃₁N₃O₃⁺, 506.24); tR=3.49 min.

4-((S,E)-3-((S)-2-(((Benzyloxy)carbonyl)amino)-3-phenylpropanamido)-4-phenylbut-1-en-1-yl)-1-methylpyridin-1-ium Iodide (22, Cbz-Phe-Phe-vinyl-4PyrNMe). Yellow solid, 0.020 g, 0.030 mmol, 87% yield. ¹H NMR (400 MHz, MeOD+CDCl₃): δ 2.75-3.09 (m, 4H), 4.28-4.43 (m, 51H), 4.87 (q, J=6.3 Hz, ^{1H}), 5.08 (s, 2H), 6.28 (d, J=15.9 Hz, 1H), 6.83 (dd, J1=5.0 Hz, J2=15.9 Hz, 1H), 7.16-7.35 (m, 15H), 7.82 (d, J=6.5 Hz, 2H), 8.71 (d, J=6.5 Hz, 2H). ¹³C NMR (100 MHz, CDCl₃+MeOD): δ 38.2, 40.1, 47.8, 52.2, 56.3, 66.8, 124.5, 126.8 (2C), 127.6, 128.0, 128.3, 128.4 (3C), 128.5, 129.1, 129.2, 136.1, 136.4, 143.9, 144.7, 153.1, 156.2, 171.3. LC-MS m/z: 521.33 [M+H]+(calcd for C₃₃H₃₄N₃O₃⁺, 521.27); tR=3.36 min.

Benzyl ((S)-1-Oxo-3-phenyl-1-(((S,E)-1-phenyl-4-(pyrimidin-4-yl)-but-3-en-2-yl)amino)propan-2-yl)carbamate (23, Cbz-Phe-Phevinyl-4Pyrmd). Off-white solid, 0.800 g, 1.579 mmol, 64% yield. ¹H NMR (400 MHz, CDCl₃): δ 2.79-2.93 (m, 2H), 3.01 (d, J=6.9 Hz, 2H), 4.35-4.48 (m, 1H), 4.93 (pentet, J=6.7 Hz, 1H), 5.04 (s, 2H), 5.49 (s, 1H), 6.16 (d, J=15.6 Hz, 1H), 6.29 (s, 1H), 6.88 (dd, J1=5.8 Hz, J2=15.6 Hz, 1H), 6.97 (d, J=4.6 Hz, 1H), 7.05-7.33 (m, 15H), 8.56 (d, J=5.2 Hz, 1H), 9.06 (s, 1H). ¹³C NMR (100 MHz, CDCl₃): δ 38.5, 40.9, 51.6, 56.6, 67.2, 118.8, 126.9, 127.1, 128.0, 128.2, 128.3, 128.6 (3C), 128.8, 129.4 (2C), 136.2, 136.5, 138.6, 156.1, 157.4, 158.8, 161.5, 170.4. LC-MS m/z: 507.35 [M+H]+(calcd for C₃₁H₃₀N₄O₃⁺, 507.24); tR=5.27 min.

Benzyl ((S)-1-(((S,E)-4-(Oxazol-2-yl)-1-phenylbut-3-en-2-yl)-amino)-1-oxo-3-phenylpropan-2-yl)carbamate (24, Cbz-Phe-Phevinyl-2Oxz). White solid, 0.020 g, 0.040 mmol, 29% yield. ¹H NMR (400 MHz, CDCl3): δ 2.72-2.88 (m, 2H), 2.93-3.11 (m, 2H), 4.35 (q, J=7.0 Hz, 1H), 4.88 (pentet, J=6.7 Hz, 1H), 5.05 (s, 2H), 5.30 (s, 1H), 5.99 (d, J=5.8 Hz, 1H), 6.14 (d, J=16.1 Hz, 1H), 6.48 (dd, J1=5.9 Hz, J2=16.1 Hz, 1H), 7.04-7.35 (m, 16H), 7.54 (s, 1H). ¹³C NMR (100 MHz, CDCl3): δ 38.6, 40.9, 51.5, 56.7, 67.3, 117.3, 127.1, 127.4, 128.2, 128.4, 128.7 (3C), 129.0, 129.4 (2C), 136.2, 136.4 (2C), 137.1, 138.3, 156.1, 160.8, 170.3. LC-MS m/z: 496.25 [M+H]⁺ (calcd for C₃₀H₂₉N₃O₄⁺, 496.22); tR=5.41 min.

Benzyl ((S)-1-Oxo-3-phenyl-1-(((S,E)-1-phenyl-4-(thiazol-2-yl)-but-3-en-2-yl)amino)propan-2-yl)carbamate (25, Cbz-Phe-Phevinyl-2Thz). Light yellow solid, 0.206 g, 0.403 mmol, 54% yield. ¹H NMR (400 MHz, CDCl₃): δ 2.75-2.89 (m, 2H), 2.99 (d, J=7.1 Hz, 2H), 4.28-4.46 (m, 1H), 4.87 (d, J=6.4 Hz, 1H), 4.96-5.11 (m, 2H), 5.44 (s, 1H), 6.19 (s, 1H), 6.39 (dd, J1=5.1 Hz, J2=16.0 Hz, 1H), 6.45 (d, J=16.0 Hz, 1H), 7.01-7.39 (m, 16H), 7.71 (d, J=3.2 Hz, 1H). ¹³C NMR (100 MHz, CDCl₃): δ 38.7, 41.0, 51.5, 56.6, 67.2, 118.5, 123.9, 126.9, 127.2, 128.1, 128.3, 128.6 (2C), 128.8, 129.4 (2C), 135.0, 136.2, 136.5 (2C), 143.4, 156.0, 166.0, 170.4. LC-MS m/z: 512.18 [M+H]+ (calcd for C₃₀H₂₉N₃O₃⁺, 512.20); tR=5.64 min.

2-((S,E)-3-((S)-2-(((Benzyloxy)carbonyl)amino)-3-phenylpropanamido)-4-phenylbut-1-en-1-yl)-3-methylthiazol-3-ium Iodide (26, Cbz-Phe-Phe-vinyl-2ThzNMe). Yellow solid, 0.003 g, 0.005 mmol, 15% yield. ¹H NMR (400 MHz, CDCl₃): δ 2.51 (s, 1H), 2.81-3.96 (m, 1H), 2.98-3.18 (m, 3H), 3.71 (s, 3H), 3.93 (s, 2H), 4.36-4.49 (m, 1H), 4.99 (s, 2H), 5.85-6.11 (m, 1H), 6.81 (d, J=15.6 Hz, 1H), 6.91 (dd, J1=3.4 Hz, J2=15.6 Hz, 1H), 7.06-7.33 (m, 15H), 7.76 (s, 1H), 7.96 (m, 1H). ¹³C NMR (100 MHz, CDCl₃): δ 38.1, 39.8, 52.5, 54.7, 57.3, 66.7, 115.1, 121.6, 126.9, 127.1, 127.6, 128.0 (2C), 128.5, 128.7, 128.8, 129.5 (2C), 136.9, 137.0, 138.8, 150.6, 156.4, 168.7, 171.9. LC-MS m/z: 526.18 [M+H]+(calcd for C₃₁H₃₂N₃O₃S⁺, 526.22); tR=3.31 min.

Benzyl ((S)-1-Oxo-3-phenyl-1-(((S)-1-phenyl-4-(pyrimidin-2-yl)-butan-2-yl)amino)propan-2-yl)carbamate (27, Cbz-Phe-Phe-(CH2)2-2Pyrmd). To a solution of Boc-Phe-vinyl-2Pyrmd (h, prepared following GP1-GP7, 0.05 g, 0.15 mmol) in anhydrous EtOAc (8 mL) was added Pd/C (10% wt, 0.016 mg) under a H2 atmosphere and stirred overnight. The reaction was filtered, and the filtrate was concentrated. The product was coupled with the P3-P2 fragment following GP8 to give compound 27. White solid, 0.031 g, 0.060 mmol, 48% yield. ¹H NMR (400 MHz, CDCl₃): δ 1.63-1.82 (m, 1H), 1.85-2.01 (m, 1H), 2.63-2.77 (m, 2H), 2.78-2.94 (m, 2H), 2.99 (d, J=6.8 Hz, 2H), 4.05-4.23 (m, 1H), 4.31 (pentet, J=7.1 Hz, 1H), 5.07 (s, 2H), 5.34 (s, 1H), 6.43 (d, J=6.4 Hz, 1H), 7.04-7.38 (m, 16H), 8.59 (d, J=4.9 Hz, 2H). ¹³C NMR (100 MHz, CDCl₃): δ 31.5, 35.8, 38.8, 41.1, 51.1, 56.8, 67.2, 118.6, 126.6, 127.0, 128.2, 128.3, 128.5, 128.7, 128.8, 129.4, 129.6, 136.4, 136.7, 137.9, 155.9, 157.0, 170.5, 170.9. LC-MS m/z: 509.15, 509.28 [M+H]+(calcd for C₃₁H₃₂N₄O₃⁺, 509.26); tR=5.10 min.

Synthetic Procedures for the Preparation of Masked Aldehyde Inhibitors

Methyl 2-((tert-butoxycarbonyl)amino)-3-(2-hydroxy-5-methylphenyl)acrylate (Sla, R Me). To a solution of (±)-Boc-α-phosphonoglycine trimethyl ester (4.22 g, 14.2 mmol, 1.2 eq) in DCM (20 mL) was added DBU (2.12 mL, 14.2 mmol, 1.2 eq) dropwise at −10° C., and the resulting mixture was stirred for 20 min. Then 4-methylsalicylaldehyde (1.61 g, 11.8 mmol, 1.0 eq) in DCM (10 mL) was slowly added to the mixture over 10 min. The reaction was stirred at room temperature overnight. The resulting mixture was concentrated under reduced pressure, diluted with EtOAc (100 mL), and washed successively with saturated aqueous NH₄Cl (20 mL), saturated aqueous NaHCO₃ (20 mL) and brine (20 mL). The organic layer was dried over Na₂SO₄, filtered, and concentrated under reduced pressure. The obtained crude product was purified by FCC (30-50% EtOAc in hexane, v/v) to give S1a (R=Me, 2.91 g, 80%). ¹H NMR (400 MHz, CD₃OD) δ 1.45 (s, 9H, Boc), 2.25 (s, 3H, PhCH₃), 3.83 (s, 3H, COOCH₃), 6.77 (d, J=8.3 Hz, 1H, olefin), 7.02 (dd, J=2.2, 8.4 Hz, 1H, Ph), 7.41 (d, J=2.2 Hz, 1H, Ph), 7.53 (s, 1H, Ph). ¹³C NMR (100 MHz, CD₃OD) δ 19.22, 27.18, 51.36, 79.97, 115.23, 120.46, 124.58, 128.22, 129.63, 131.02, 153.42, 154.76, 166.73. LC-MS: t_{R}=4.93 min; C₁₁H₁₄NO₃⁺[M+H−Boc]⁺, m/z calcd 208.10, found 208.08.

Methyl 2-((tert-butoxycarbonyl)amino)-3-(2-hydroxy-5-methylphenyl)propanoate (S1b, R Me). S1a (R=Me, 1.15 g, 3.74 mmol, 1.0 eq) was placed in a two-necked round bottom and charged with N₂ gas. 10% palladium on carbon powder (Pd/C, 110 mg, cat.) was quickly added to the flask, followed by addition of MeOH (20 mL). The flask was degassed and backfilled with H₂ for three cycles. The reaction mixture was stirred at room temperature overnight with a balloon of H₂ for replenishment. The balloon was removed, and the mixture was filtered under reduced pressure. Notice that the operation should be rapid, and the Pd/C powder must be kept wet to avoid catching fire, and was appropriately disposed of in a water-filled, cap-closed container. The filtrate was concentrated under reduced pressure and purified by FCC (30% EtOAc in hexane, then 6% MeOH in DCM) to give S1b (R=Me, 1.1 g, 95%). ¹H NMR (400 MHz, CDCl₃) δ 1.39 (s, 9H, Boc), 2.36 (s, 3H, PhCH₃), 2.79-3.02 (m, 1H, βCH₂), 3.07 (m, 1H, βCH₂), 3.75 (s, 3H, COOCH₃), 4.43 (d, J=6.2 Hz, 1H, αCH), 5.54 (d, J=7.4 Hz, 1H, NH), 6.65-6.73 (m, 2H, Ph), 6.73-6.79 (m, 1H, Ph). ¹³C NMR (100 MHz, CDCl₃) δ 20.57, 28.24, 32.84, 51.98, 54.97, 79.50, 113.30, 116.22, 119.28, 127.84, 147.51, 153.95, 155.33, 172.70. LC-MS: t_{R}=4.98 min; C₁₆H₂₄NO₅⁺ [M+H]⁺, m/z calcd 310.16, found 310.14.

Methyl 2-amino-3-(2-hydroxy-5-methylphenyl)propanoate TFA salt (S1c, R=Me). To a suspension of S1b (R=Me, 1.1 g, 3.56 mmol, 1.0 eq) in DCM (6 mL) was added TFA (3 mL) at 0° C., and the resulting mixture was stirred for 30 min. The mixture was then co-concentrated with toluene three times to remove most of the residual TFA. The obtained crude product S1c (R=Me) was used without further purification.

Methyl (S)-2-((S)-2-(((benzyloxy)carbonyl)amino)-3-phenylpropanamido)-3-(2-hydroxy-5-methylphenyl)propanoate (S1d, R=Me, R₁=BnO). A mixed suspension of Sic (R=Me, ˜1.15 g, 3.56 mmol, 1.0 eq), Cbz-Phe-OH (1.07 g, 3.56 mmol, 1.0 eq) and DIPEA (2.17 mL, 12.46 mmol, 3.5 eq) in DCM (20 mL) was cooled to 0° C. followed by dropwise addition of T3P (>50% in MeCN, 3.53 mL, 5.34 mmol, 1.5 eq). The resulting mixture was stirred at room temperature for 2 h. The reaction was concentrated under reduced pressure, diluted with EtOAc (75 mL), and washed successively with 5% citric acid (15 mL), saturated aqueous NaHCO₃ (15 mL) and brine (15 mL). The organic layer was dried over Na₂SO₄, filtered, and concentrated under reduced pressure. The obtained crude product was purified by FCC (40% EtOAc in hexane) to give S1d (R=Me, R₁=BnO, 1.11 g, 63% for two steps). ¹H NMR (400 MHz, CDCl₃) δ 2.30 (s, 3H, PhCH₃), 2.83-3.16 (m, 4H, 2×βCH₂), 3.69 (s, 3H, COOCH₃), 4.50 (s, 1H, αCH), 4.75 (s, 1H, αCH), 4.98-5.14 (m, 2H, Cbz CH₂), 5.70 (s, 1H, OH), 6.68 (t, J=8.8 Hz, 1H, NH), 6.91-7.10 (m, 3H, Ph), 7.22 (dd, J=20.7, 26.3 Hz, 7H, Ph), 7.33 (s, 3H, Ph), 8.08 (s, 1H, NH). ¹³C NMR (100 MHz, CDCl₃) δ 20.66, 33.10, 38.37, 52.47, 53.19, 56.10, 67.24, 117.11, 124.49, 127.01, 127.88, 128.22, 128.32, 128.42, 128.53, 128.61, 129.25, 130.87, 135.98, 136.14, 153.57, 156.27, 171.72, 172.08. LC-MS: t_{R}=5.39 min; C₂₈H₃₁N₂O₆⁺ [M+H]⁺, m/z calcd 491.22, found 491.19.

Methyl (S)-2-((S)-2-(((benzyloxy)carbonyl)amino)-3-phenylpropanamido)-3-(2-((tert-butyldimethylsilyl)oxy)-5-methylphenyl)propanoate (S1e, R=Me, R₁=BnO, R₂=TBS). A mixed solution of S1d (R=Me, R₁=BnO, 990 mg, 2.02 mmol, 1.0 eq), TBSCl (609 mg, 4.04 mmol, 2.0 eq), and imidazole (412 mg, 6.06 mmol, 3.0 eq) was stirred at room temperature overnight. The reaction was quenched by addition of 0.5M HCl (10 mL), and was stirred for another 15 min. The mixture was concentrated under reduced pressure, and was partitioned between EtOAc (50 mL) and water (10 mL). The organic layer was washed with saturated aqueous NaHCO₃ (10 mL) and brine (10 mL), and was dried over Na₂SO₄, filtered, and concentrated under reduced pressure. The obtained crude product was purified by FCC (20% EtOAc in hexane) to give S1e (R=Me, R₁=BnO, R₂=TBS, 897 mg, 73%). ¹H NMR (400 MHz, CDCl₃) δ 0.16 (s, 6H, TBS Si(CH₃)₂), 0.93 (s, 9H, TBS C(CH₃)₃), 2.37 (s, 3H, PhCH₃), 2.59-2.87 (m, 2H, βCH₂), 2.89-3.05 (m, 2H, βCH₂), 3.54 (d, J=4.3 Hz, 3H, COOCH₃), 4.36 (d, J=22.6 Hz, 1H, αCH), 4.64 (q, J=8.3 Hz, 1H, αCH), 4.95 (p, J=12.1 Hz, 2H, Cbz CH₂), 5.32-5.55 (m, 1H, NH), 6.56-6.65 (m, 1H, Ph), 6.83-7.02 (m, 3H, Ph), 7.05-7.25 (m, 9H, Ph). ¹³C NMR (100 MHz, CDCl₃) δ −3.50, 18.27, 20.53, 25.81, 32.94, 38.57, 52.20, 52.57, 56.08, 66.96, 119.79, 125.91, 126.90, 127.89, 127.95, 128.02, 128.04, 128.07, 128.14, 128.44, 128.47, 128.57, 128.71, 129.28, 129.35, 130.82, 136.25, 136.47, 152.62, 155.90, 170.76, 171.62, 172.00. LC-MS: t_{R}=7.23 min; C₃₄H₄₅N₂O₆Si⁺[M+H]⁺, m/z calcd 605.30, found 605.24.

Methyl (S)-2-((S)-2-(((benzyloxy)carbonyl)amino)-3-phenylpropanamido)-3-(2-methoxyphenyl)propanoate (S1e, R=H, R₁=BnO, R₂=Me). To a solution of S1d (R=H, R₁=BnO, 172 mg, 0.36 mmol, 1.0 eq) in DMF (2 mL) was successively added K₂CO₃ (100 mg, 0.72 mmol, 2.0 eq) and iodomethane (67 μL, 1.08 mmol, 3.0 eq) at 0° C. The resulting mixture was stirred at room temperature for 20 h during which the system should be kept securely sealed to avoid evaporation of iodomethane. The mixture was diluted with EtOAc (50 mL), and washed with water extensively (5×10 mL). The organic layer was dried over Na₂SO₄, filtered, and concentrated under reduced pressure. The obtained crude product was purified by FCC (30% EtOAc in hexane) to give S1e (R=H, R₁=BnO, R₂=Me, 167 mg, 95%). ¹H NMR (400 MHz, CDCl₃) δ 3.02 (qd, J=5.7, 13.6, 16.9 Hz, 4H, 2×βCH₂), 3.61 (s, 3H, COOCH₃), 3.93 (s, 3H, PhOCH₃), 4.48 (s, 1H, αCH), 4.72 (s, 1H, αCH), 4.88-5.12 (m, 2H, Cbz CH₂), 5.69 (d, J=8.2 Hz, 1H, NH), 6.64-6.88 (m, 2H, Ph), 6.92-7.06 (m, 3H, Ph), 7.08-7.18 (m, 4H, Ph), 7.24 (d, J=18.6 Hz, 5H, Ph), 7.77 (d, J=43.8 Hz, 1H, NH). ¹³C NMR (100 MHz, CDCl₃) δ 32.65, 32.96, 38.40, 52.33, 52.40, 53.70, 56.04, 67.11, 115.84, 120.35, 122.73, 126.88, 126.90, 127.88, 128.16, 128.52, 128.72, 129.30, 131.30, 136.14, 154.71, 156.21, 156.23, 171.98, 172.34. LC-MS: t_{R}=5.71 min; C₂₈H₃₁N₂O₆⁺[M+H]⁺, m/z calcd 491.22, found 491.33.

Benzyl ((S)-1-(((S)-1-(2-((tert-butyldimethylsilyl)oxy)-5-methylphenyl)-3-hydroxypropan-2-yl)amino)-1-oxo-3-phenylpropan-2-yl)carbamate (S1f, R=Me, R₁=BnO, R₂=TBS). To a solution of S1e (R=Me, R₁=BnO, R₂=TBS, 897 mg, 1.48 mmol, 1.0 eq) in MeOH (10 mL) was added NaBH₄ (1.2 g, 31.7 mmol, >20 eq) in multiple portions every 30 min. The reaction was stirred at room temperature for another 2 h, and then quenched by addition of saturated aqueous NH₄Cl (10 mL). The mixture was concentrated under reduced pressure, and diluted with EtOAc (50 mL). The organic layer was washed with saturated aqueous NaHCO₃ (10 mL) and brine (10 mL), and was dried over Na₂SO₄, filtered, and concentrated under reduced pressure. The obtained crude product was purified by FCC (35% EtOAc in hexane) to give S1f (R=Me, R₁=BnO, R₂=TBS, 450 mg, 53%). ¹H NMR (400 MHz, CDCl₃) δ 0.26 (s, 6H, TBS Si(CH₃)₂), 1.03 (s, 9H, TBS C(CH₃)₃), 2.40 (s, 3H, PhCH₃), 2.74 (dt, J=7.0, 13.1 Hz, 2H, βCH₂), 2.97 (dd, J=6.8, 40.5 Hz, 2H, βCH₂), 3.25-3.60 (m, 2H, CH₂OH), 4.02-4.15 (m, 1H, αCH), 4.39 (s, 1H, αCH), 4.97-5.15 (m, 2H, Cbz CH₂), 5.69 (dd, J=7.8, 66.8 Hz, 1H, NH), 6.54 (dd, J=6.8, 121.3 Hz, 1H, Ph), 6.72-6.81 (m, 1H, Ph), 7.04-7.14 (m, 2H, Ph), 7.15-7.30 (m, 6H, Ph & NH), 7.34 (q, J=6.2, 7.1 Hz, 4H, Ph). ¹³C NMR (100 MHz, CDCl₃) δ −4.05, 18.27, 21.02, 25.87, 31.17, 39.00, 52.34, 56.69, 63.66, 67.03, 119.96, 126.28, 127.00, 127.56, 127.93, 128.03, 128.16, 128.47, 128.65, 129.29, 130.27, 131.00, 136.24, 136.53, 152.38, 155.94, 170.98, 171.35. LC-MS: t_{R}=6.90 min; C₃₃H₄₅N₂O₅Si⁺ [M+H]⁺, m/z calcd 577.31, found 577.37.

Benzyl ((S)-1-(((S)-1-(2-((tert-butyldimethylsilyl)oxy)-5-methylphenyl)-3-oxopropan-2-yl)amino)-1-oxo-3-phenylpropan-2-yl)carbamate (S1g, R=Me, R₁=BnO, R₂=TBS). To a solution of S1f (R=Me, R₁=BnO, R₂=TBS, 151 mg, 0.26 mmol, 1.0 eq) in DCM (8 mL) at 0° C. was added Dess-Martin periodinane (133 mg, 0.31 mmol, 1.2 eq) and NaHCO₃ powder (55 mg, 0.65 mmol, 2.5 eq). The resulting mixture was stirred at 0° C. for 1 h, and then quenched by addition of saturated aqueous Na₂S₂O₃ (2 mL). The reaction was concentrated under reduced pressure, diluted with EtOAc (50 mL), and washed with brine (3×10 mL). The organic layer was dried over Na₂SO₄, filtered, and concentrated under reduced pressure. The obtained crude product was purified by FCC (25% EtOAc in hexane) to give S1g (R=Me, R₁=BnO, R₂=TBS, 68 mg, 45%). ¹H NMR (400 MHz, CDCl₃) δ 0.24 (d, J=4.6 Hz, 6H, TBS Si(CH₃)₂), 1.01 (s, 9H, TBS C(CH₃)₃), 2.24 (s, 3H, PhCH₃), 2.77-3.20 (m, 4H, 2×βCH₂), 4.51 (q, J=6.7 Hz, 1H, αCH), 5.02-5.15 (m, 2H, Cbz CH₂), 5.35 (s, 1H, αCH), 6.52 (d, J=5.5 Hz, 1H, NH), 6.71 (d, J=8.2 Hz, 1H, Ph), 6.85 (d, J=2.2 Hz, 1H, Ph), 6.94 (dd, J=2.3, 8.2 Hz, 1H, Ph), 7.16 (d, J=7.1 Hz, 2H, Ph), 7.21-7.28 (m, 3H, Ph), 7.35 (dt, J=4.7, 6.9 Hz, 5H, Ph), 9.41 (s, 1H, CHO). ¹³C NMR (100 MHz, CDCl₃) δ −4.14, 18.28, 20.43, 25.90, 29.92, 38.80, 56.11, 59.57, 67.01, 118.77, 125.68, 127.09, 128.01, 128.16, 128.50, 128.67, 128.91, 129.26, 130.94, 131.91, 136.13, 151.28, 170.96, 198.75. LC-MS: t_{R}=6.81 min; C₃₃H₄₃N₂O₅Si⁺ [M+H]⁺, m/z calcd 575.29, found 575.36.

Benzyl ((2S)-1-(((3S)-2-hydroxy-6-methylchroman-3-yl)amino)-1-oxo-3-phenylpropan-2-yl)carbamate (S1h, R=Me, R₁=BnO, R₂=TBS). To a solution of S1g (R=Me, R₁=BnO, R₂=TBS, 68 mg, 0.12 mmol, 1.0 eq) in THE (3 mL) was slowly added 1.0M TBAF in THF (131 μL, 0.13 mmol, 1.1 eq) at 0° C. The resulting mixture was stirred at 0° C. for 1 h, and concentrated under reduced pressure. The residue was diluted with EtOAc (50 mL) and washed with saturated aqueous NH₄Cl (10 mL) and brine (10 mL). The organic layer was dried over Na₂SO₄, filtered, and concentrated under reduced pressure. The obtained crude product was purified by FCC (35% EtOAc in hexane) to give S1h (R=Me, R₁=BnO, R₂=TBS, i.e., compound 31, 20 mg, 36%). ¹H NMR (400 MHz, CDCl₃) δ 2.10-2.18 (m, 3H, PhCH₃), 2.37-3.10 (m, 4H, 2×βCH₂), 4.10-4.45 (m, 2H, αCH & NH), 4.71-5.10 (m, 3H, Cbz CH₂ & NH), 5.53 (dd, J=7.2, 51.9 Hz, 1H, αCH), 5.97-6.41 (m, 1H, lactol C(O)OH), 6.59 (td, J=10.3, 31.2, 32.4 Hz, 2H, Ph), 6.78 (dd, J=8.2, 18.7 Hz, 1H, Ph), 6.98-7.28 (m, 10H, Ph). ¹³C NMR (100 MHz, CDCl₃) δ 20.46, 25.96, 39.10, 46.02, 56.24, 67.20, 91.04, 91.93, 116.68, 118.34, 119.01, 119.23, 128.04, 128.12, 128.20, 128.52, 128.67, 128.77, 129.19, 129.32, 129.64, 130.50, 130.63, 136.02, 136.26, 148.52, 156.15, 171.23. LC-MS: t_{R}=5.32 min; C₂₇H₂₉N₂O₅⁺ [M+H]⁺, m/z calcd 461.21, found 461.27.

(3S)-3-((S)-2-(4-methylpiperazine-1-carboxamido)-3-phenylpropanamido)chroman-2-yl acetate (S2a, 37, R₁=Me). To a solution of compound 36 (50 mg, 0.114 mmol, 1.0 eq) in DCM (3 mL) was added acetic anhydride (32 μL, 0.342 mmol, 3.0 eq), Et₃N (48 μL, 0.342 mmol, 3.0 eq) and DMAP (2.8 mg, 0.023 mmol, 0.2 eq). The resulting mixture was stirred at room temperature overnight, and concentrated under reduced pressure. The residue was diluted with EtOAc (50 mL), and washed successively with saturated aqueous NH₄Cl (10 mL), saturated aqueous NaHCO₃ (10 mL) and brine (10 mL). The organic layer was dried over Na₂SO₄, filtered, and concentrated under reduced pressure. The obtained crude product was purified by FCC (10% MeOH in DCM) to give S2a (R₁=Me, i.e., compound 37, 31 mg, 57%). ¹H NMR (400 MHz, CDCl₃) δ 2.01 (d, J=23.4 Hz, 3H, CH₃CO), 2.29 (d, J=3.5 Hz, 3H, NCH₃), 2.30-2.38 (m, 4H, 2×piperazinyl CH₂), 2.69-2.97 (m, 2H, lactol βCH₂), 2.98-3.15 (m, 2H, Phe βCH₂), 3.25-3.41 (m, 4H, 2×piperazinyl CH₂), 4.49 (dt, J=6.9, 13.8 Hz, 1H, αCH), 5.18 (dd, J=7.4, 40.0 Hz, 1H, Phe αCH), 6.12 (dd, J=2.5, 17.4 Hz, 1H, lactol CH), 6.31-6.54 (m, 1H, NH), 6.82-6.91 (m, 1H, Ph), 6.91-7.07 (m, 2H, Ph), 7.07-7.19 (m, 2H, Ph), 7.19 (s, 3H, Ph & NH), 7.26-7.34 (m, 2H, Ph). ¹³C NMR (100 MHz, CDCl₃) δ 20.99, 26.61, 29.67, 38.69, 43.71, 45.99, 54.48, 56.23, 88.88, 89.33, 117.07, 119.40, 121.89, 127.09, 128.06, 128.70, 128.74, 129.15, 129.20, 136.94, 150.22, 157.02, 169.38, 171.96. LC-MS: t_{R}=3.29 min; C₂₆H₃₃N₄O₅⁺ [M+H]⁺, m/z calcd 481.24, found 481.3.

N-((2S)-1-(((3S)-2-ethoxychroman-3-yl)amino)-1-oxo-3-phenylpropan-2-yl)-4-methylpiperazine-1-carboxamide (S2b, 40, R₂=Et). To a solution of compound 36 (40 mg, 0.091 mmol, 1.0 eq) in EtOH (2 mL) was added BF₃OEt₂ (300 μL, 2.43 mmol, >20 eq) dropwise at 0° C. The resulting mixture was stirred at room temperature overnight, and quenched by addition of saturated aqueous NH₄Cl (2 mL). The reaction was concentrated under reduced pressure and then partitioned between DCM (20 mL) and water (20 mL). The water layer was further washed with DCM (2×20 mL). The combined organic layers were dried over Na₂SO₄, filtered, and concentrated under reduced pressure. The obtained crude product was purified by FCC (8% MeOH in DCM) to give S2b (R₂=Et, i.e., compound 16, 18.3 mg, 43%). ¹H NMR (400 MHz, CDCl₃) δ 1.00 (t, 3H, OCH₂CH₃), 2.16-2.36 (m, 7H, NCH₃ & 2×piperazinyl CH₂), 2.69 (dd, J=9.2, 14.2 Hz, 1H, βCH₂), 2.83-2.95 (m, 1H, βCH₂), 3.10 (dt, J=7.2, 13.4 Hz, 1H, Phe (3CH₂), 3.21 (dd, J=4.0, 6.1 Hz, 1H, Phe βCH₂), 3.31 (dt, J=6.0, 11.1 Hz, 3H, OCH₂CH₃ & piperazinyl CH₂), 3.58-3.76 (m, 1H, piperazinyl CH₂), 4.13-4.27 (m, 1H, piperazinyl CH₂), 4.41 (q, J=7.3 Hz, 1H, piperazinyl CH₂), 4.68 (dd, J=2.3, 49.7 Hz, 1H, αCH), 4.90-5.15 (m, 1H, Phe αCH), 5.78 (dd, J=8.9, 24.8 Hz, 1H, lactol CH), 6.73 (d, J=8.1 Hz, 1H, Ph), 6.80 (q, J=6.8, 7.4 Hz, 1H, Ph), 6.90 (d, J=7.2 Hz, 1H, Ph), 7.03 (t, J=7.2 Hz, 1H, Ph), 7.07-7.30 (m, 5H, Ph). ¹³C NMR (100 MHz, CDCl₃) δ 14.94, 14.98, 27.08, 39.59, 43.72, 45.10, 46.06, 54.58, 56.30, 64.01, 96.02, 96.34, 116.79, 120.48, 121.16, 126.99, 127.67, 128.65, 128.75, 129.20, 129.33, 136.84, 137.17, 150.58, 150.70, 156.56, 156.68, 171.26, 171.66. LC-MS: t_{R}=3.53 min; C₂₆H₃₅N₄O₄⁺ [M+H]⁺, m/z calcd 467.27, found 467.3.

As shown in FIG. 20, the preparation of S4a and S4b was similar to the procedures for Sla and Sib, respectively; the preparation of S4d, S4e, and S4f was similar to the procedures for S3f, S3g and S3h, respectively. Therefore, only the preparation of S4c is described in detail as below.

2-((tert-butoxycarbonyl)amino)-3-(2-oxo-1,2-dihydropyridin-3-yl)propanoic acid (S4c). A mixed solution of S4b (1.18 g, 4.0 mmol, 1.0 eq) and LiOH (192 mg, 8.0 mmol, 2.0 eq) in MeOH (10 mL) and H₂O (5 mL) was stirred at room temperature for 2 h. The reaction was acidified by addition of 0.1M HCl to pH 2-3, and then concentrated under reduced pressure. The residue was extracted with DCM (3×50 mL). The organic layers were combined, dried over Na₂SO₄, filtered, and concentrated under reduced pressure. The obtained crude product S4c was used without further purification. ¹H NMR (400 MHz, CDCl₃) δ 1.39 (s, 9H, Boc), 2.86 (dd, J=8.9, 14.0 Hz, 1H, βCH₂), 3.11 (dd, J=4.3, 13.9 Hz, 1H, βCH₂), 4.42 (dd, J=4.3, 8.8 Hz, 1H, αCH), 6.32 (t, J=6.7 Hz, 1H, pyridine), 7.30 (dd, J=2.0, 6.6 Hz, 1H, pyridine), 7.39-7.54 (m, 1H, pyridine).

Solubility of Cruzain Inhibitors

Synthetic PVHIs such as 5-16 and 22-24 at final concentrations of 1-200 μM were added to 0.25 mL solutions of 10% DMSO (v/v) in clear 96-well Greiner plates, and absorbance at 620 nm was measured using a BioTek M2 Synergy plate reader at t=0 and 120 min to evaluate increased light scattering and precipitated inhibitor.

Evaluation of Covalent Adducts of Glutathione and Cruzain Inhibitors

The compounds 7, 11, 12, 15, 25, 26, and K1777 (0.5 mM) were added to 100 mM Tris (pH 8.0), 10% (v/v) DMSO, and 1 mM or 5 mM reduced glutathione to a final volume of 0.2 mL at room temperature. Samples were analyzed by HPLC-MS (as described above) by injecting 0.01 mL aliquots onto a Luna 5 mm C18(2) 100 Å, 4.6 mm, 50 mm column (Phenomenex) using the HPLC method described in the chemistry section at 0-6 h time points. The chromatographic peaks for each cruzain inhibitor and its covalent adduct with glutathione were characterized by their values of m/z using electrospray positive ionization detection and UV absorbance at 254 nm: K11777, retention time: 4.75 min, m/z: 575.05; K11777-GSH, retention time: 4.64 min, m/z: 882.30; 7, retention time: 6.71 min, m/z: 507.11; 11, retention time: 5.48 min, m/z: 506.06; 12, retention time: 4.77 min, m/z: 520.10; 15, retention time: 4.81 min, m/z: 535.23; 15-GSH, retention time: 4.72 min, m/z: 842.42; 17, retention time: 5.03 min, m/z: 587.95; 25, retention time: 4.43 min, m/z: 511.87; 26, retention time: 4.42 min, m/z: 525.90; 26-GSH, retention time: 3.61 min, m/z: 833.97. Integration of the chromatographic peaks of the inhibitors and their GSH adducts at each time point was used to determine the rate of GSH adduct formation.

Enzyme Expression and Purification

Recombinant human cathepsins B and L were purchased from Millipore Sigma and used without further treatment. The solid proteins were dissolved into a solution of 50 mM sodium acetate (pH 5.5), 1 mM EDTA, 1 mM CHAPS, 10% (v/v) DMSO, and 5 mM DTT to final concentrations of 1-10 mM in 20-mL aliquots, and stored at −80° C. until needed. These samples were then diluted into the same buffer to concentrations of −100 nM, and these dilutions were stored at 4° C. and used daily until depletion.

General procedures for cruzain expression, purification, and activation were performed according to published protocols⁵⁹ with modifications as described.⁴⁰ Activated cruzain at>95% purity was stored either as MMTS-conjugated samples, as described, at −80° C. in a buffer containing 50 mM Tris (pH 8.0) and 20% glycerol, at protein concentrations of 5 mg mL-¹. Prior to use, MMTS was removed by successive dialysis in the presence of 5 mM DTT. Activated samples of cruzain were used immediately or stored after use at 4° C. for a period of 1-2 months.

Recombinant SARS CoV-2 3CL protease (also known as Main protease) was expressed and purified as described in Mellott et al.⁶⁰ In brief, an expression construct of CoV-2 3CL-PR contained a GST domain at the N-terminus of the 3CL-PR coding sequence, followed by the 3CL-PR cleavage sequence (SAVLQ*SGF) preceding the sequence encoding the remaining 303 amino acids of the 3CL-PR monomer, followed at its C-terminus by a modified PreScission protease sequence (SGVTFQ*GP), that preceded a His6 sequence. Upon expression, auto-proteolysis from 3CL-PR removed the N-terminal GST tag, yielding the authentic N-terminus (Ser-Gly-Phe). After binding of this processed protein to a nickel-NTA column, eluted fractions were pooled and dialyzed to remove imidazole. Proteolysis of the C-terminal H6 tag was conducted by incubation with HRV 3C Protease (Thermo Fisher Scientific) per mg of 3CL-PR at 4° C. overnight. Subsequently, the protein mixture was subjected to chromatography on a 5-mL GSTrap HP column, and then a 5-mL HisTrap HP column (GE Healthcare), to remove, respectively, the GST-fused HRV 3C protease and undigested H6-tagged protein. The tag-free 3CL-PR was pooled and concentrated (10 kDa molecular weight cutoff filter, GE Healthcare). The protein was deemed to be ≥95% pure by SDS-PAGE, and was stored at −80° C. in 12 mM Tris-HCl, 120 mM NaCl, 0.1 mM EDTA, 2 mM DTT, (pH 7.5) with 50% glycerol (v/v). Analytical gel filtration indicated that native 3CLpro was the expected homodimer.

Enzyme Assays and Evaluation of Inhibitors

All enzyme assays were performed at 25° C. Initial rates of the peptidolytic reaction catalyzed by cruzain were measured by monitoring the fluorescence generated by cleavage of the dipeptide-AMC bond. Assays were conducted in 96-well plates (Greiner; flat-bottom, clear black plates) in a total volume of 250 μL, containing either 50 mM MES (pH 7.5), 50 mM TAPSO, 100 mM DEA, 1 mM CHAPS, 1 mM Na2EDTA, 5 mM DTT, and 10% DMSO (v/v) or 50 mM sodium acetate (pH 5.5), 50 mM MES, 100 mM TEA, 1 mM CHAPS, 1 mM Na2EDTA, 5 mM DTT, and 10% DMSO (v/v). Substrates were dissolved in 100% DMSO and were then diluted 10-fold such that when added to reaction mixtures, final DMSO concentration was 10% (v/v). Reactions were initiated with the addition of 1-10 μL of cruzain (final concentrations: 0.1-3.0 nM (preincubation studies)). Fluorescence was measured on either a SpectraMax M5 (Molecular Devices) or a Synergy HTX (BioTek, Winooski, VT) microplate reader (λₑₓ=360 nm, λₑₘ=460 nm). Initial rates were determined from continuous kinetic time courses and calculated from the earliest time points, typically at less than 10 min.

Compounds were evaluated as inhibitors or inactivators of cruzain in two ways: (1) enzyme was added to reaction mixtures containing the substrate (typically, 10 μM Cbz-Phe-Arg-AMC) and inhibitor, and reaction time courses were measured for 0-40 min. In addition to other methods, the effects of all inhibitors on reaction rates were determined at t=0-200 s (vi) and at longer incubation times (t>1000 s; vs), to ascertain the respective inhibition constants Kᵢ and Kᵢ*. (2) Enzyme and compound were preincubated over extended periods of time, and then aliquots were removed and diluted 50-fold to 100-fold into reaction mixtures containing 10 μM Cbz-Phe-Arg-AMC, followed by the assessment of the resulting time courses.

Cruzain inhibitors were added at seven concentrations and one fixed concentration of Cbz-Phe-Arg-AMC, and time courses of AMC formation were analyzed as with cruzain.

For assays of cathepsin L and B, inhibitors were evaluated in 0.25-mL reaction mixtures containing a buffer of sodium acetate (pH 5.5), 1 mM CHAPS, 1 mM Na₂EDTA, and 5 mM DTT at 25° C. The fluorogenic substrate Cbz-Phe-Arg-7-amino-4-methyl-coumarin (Z-FR-AMC) was dissolved in 100% DMSO, as were all inhibitors, and aliquots of both substrates and inhibitors were added to reaction mixtures to final concentrations of DMSO of 10% (v/v). Reactions were initiated by addition of the proteases to final concentrations of 1-2 nM of either cathepsin L or cathepsin B. Michaelis constants for Z-FR-AMC were determined for cathepsin L (Kₘ=2.9 μM), and cathepsin B (Kₘ=150 μM), and fixed concentrations of Z-FR-AMC of 1× or 2×Kₘ were used to evaluate inhibitors. Formation of the fluorescent product AMC was monitored over 30-60-minute time courses for reaction mixtures in 96-well black microplates (Corning Costar®). Rates of peptidolysis of the dipeptide-AMC substrate(s) were measured on either a SpectraMax M5 (Molecular Devices) or a Synergy HTX (Biotek, Wisnooki, VT) microplate reader with λₑₓ=360 nm, λₑₘ=460 nm in ≥8-sec intervals. Control samples excluded substrate. The measured relative fluorescence units (RFUs) of generated AMC were converted to reaction rates of μM/s by use of a standard curve of known AMC concentrations obtained for both plate readers.

Kinetic analysis of SARS-CoV-2 3CL-PR and characterization of its inhibitors. In 50-μL reaction mixtures containing 20 mM Tris-HCl (pH 7.5), 150 mM NaCl, 0.1 mM EDTA, 2 mM DTT, 10% (v/v) DMSO (a final concentration arising from addition of substrates and inhibitors added from 100% (v/v) DMSO solutions), and variable concentrations of the FRET-based substrate Abz-SAVLQ*SGFRK(DNP)-NH₂ (Mellott et al. (2021), reaction was initiated by the addition of 25-50 nM 3CL-PR in 96-well plates (Greiner, flat-bottom half volume, clear black plates). Rates of peptidolysis of the Abz-SAVLQ*SGFRK(DNP)-NH₂ substrate was measured on either a SpectraMax M5 (Molecular Devices) or a Synergy HTX (Biotek, Wisnooki, VT) microplate reader with λₑₓ=320 nm, λₑₘ=420 nm in 8-60 sec intervals, and time courses of inhibition were obtained for either 30 or 60 min intervals. Control samples excluded substrate. The measured relative fluorescence units (RFUs) of generated AMC were converted to reaction rates of μM/s by use of a standard curve of known AMC concentrations obtained for both plate readers.

Evaluation of Cruzain Inhibitors in Axenic Cell Cultures of T. b. brucei and T. cruzi.

Selected cruzain inhibitors were evaluated in axenic cell cultures of T. b. brucei and T. cruzi. Procyclic trypomastigotes of T. b. brucei (ATCC PRA-381) were grown in the SDM-79 medium at 26° C., and bloodstream forms (ATCC PRA-383) were grown at 37° C. in the HMI-9 medium at 5% C02. T. cruzi (epimastigote forms, strain Y, ATCC 50832GFP) was grown in the ATCC medium (1029 LIT medium). Both forms of T. b. brucei and T. cruzi were grown in media containing 10% fetal bovine serum (FBS) and penicillin/streptomycin (50 U mL⁻¹). Test compounds, including K11777, were dissolved in 100% DMSO and added to cell cultures at final concentrations of 0.5-20 μM (maximum DMSO=1% (v/v)). Control samples contained equal amounts of DMSO. T. b. brucei and T. cruzi (5 mL in flask cultures) were seeded at ˜3×10⁶ cells and diluted daily, maintaining a mid-log growth phase for up to 120 h. Treated cells were typically grown for 4 days (T. b. brucei) or 5 days (T. cruzi). After each cell dilution, fresh compound or an equal volume of DMSO (control samples) was supplemented into the cultures, while maintaining a constant concentration of each inhibitor. Cell counts were scored using a Z2 Coulter Counter.

Evaluation of Cruzain Inhibitors in T. cruzi-Infected Murine Cardiomyoblasts.

For the evaluation of the antitrypanocidal activity of cruzain inhibitors, we infected a C2C12 mouse cardiomyoblast cell line (ATCC CRL-1772) with T. cruzi strain Ca-I/72 (a gift from James Dvorak, National Institutes of Health) in 1536-microwell plates. In each well were added 103 cells and 104 parasites in a total volume of 10 μL including the test compounds in 10-point dose-response dilutions starting at 10 μM (3-fold dilutions). The plates were incubated at 37° C. for 48 h, and the wells were fixed with 2% paraformaldehyde in PBS and stained with 5 μg mL-1 4,6-diamidino-2-phenylindole. After at least 30 min of incubation at room temperature in the dark, the plates were read in an automated microscope, ImageXpress Micro XL (Molecular Devices), and the images were analyzed by custom-built software to quantify and assess viability of the parasites, as well as the host cells independently. The compilation of data was used to calculate the antiparasitic activity (EC₅₀) and host cytotoxicity (CC₅₀).

Evaluation of Human Cell Toxicity.

Primary human dermal fibroblast (HDF) cells were used to evaluate human cell toxicity of cruzain inhibitors. HDF cells were plated in a 384-well plate at 2400 cells per well (62,000 cells mL⁻¹). Inhibitors in 100% DMSO were added in duplicate to final concentrations of 0.001-0.1 mM and 1% DMSO (v/v) with 1% DMSO as a control sample, and cells were cultured at 37° C. for 48 h, followed by the addition of resazurin. Cell viability was then assessed by reading of fluorescence (excitation/emission: 544 nm/590 nm) after an additional 24 h of incubation.

Kinetic Data Analysis.

Initial velocity data for cruzain-catalyzed reactions of fluorogenic peptide substrates were determined by fitting to eq 1 using GraphPad Prism 6.0 or SigmaPlot 12.0. For eq 1, k_{cat} is the turnover number, [E]ₜ is the concentration of active sites of cruzain, and Kₐ is the Michaelis constant for the substrate A. Cruzain concentrations were determined by spectrophotometric analysis of purified sample solutions as described.⁴⁴

$$\frac{v}{{[ E ]}_{t}} = \frac{{k}_{cat} [ A ]}{{K}_{a} + [ A ]} \qquad ( 1 )$$

Competitive inhibition was fitted to eq 2, in which A and I are concentrations of substrate and inhibitor, respectively, Vmax is the maximal velocity, and Kᵢₛ is the slope inhibition constant.

$$v = \frac{{V}_{max} [ A ]}{{K}_{a} ( 1 + \frac{[ I ]}{{K}_{is}} ) + [ A ]} \qquad ( 2 )$$

Data for time-dependent inhibition were fitted by several methods. All time-course data were fitted to eq 3 for studies in which the reaction was initiated by the addition of enzyme, wherein P is the fluorescence generated by AMC formation, C is a nonzero constant, vs and vi are respectively the steady-state and initial enzymatic rates, t is time, and kobs is the observed rate of conversion of the initial inhibited rate to the final inhibited rate.52 In cases for which the reaction was initiated with an excess of substrate, following preincubation of enzyme and inhibitor, for eq 3, vᵢ=0.

$$P = {v}_{s} t + [ \frac{{v}_{i} - {v}_{s}}{{k}_{obs}} ] [ 1 - exp ( - {k}_{obs} \times t ) ] + C \qquad ( 3 )$$

Values of k_{obs} versus [inhibitor] were then replotted and fitted to eq 4 for which k₃ and k₄ represent the respective rates of formation and dissolution of the EI* complex as depicted in FIG. 9.

$${k}_{obs} = {k}_{4} + \frac{{k}_{3} [ I ]}{{K}_{i} ( 1 + \frac{[ A ]}{{K}_{a}} ) + [ I ]} \qquad ( 4 )$$

Inhibition constants were also obtained by fitting vi and vs data to eq 5, in which vₓ is the rate in the presence of inhibitor I for either early (vᵢ) or late (vₛ) phases of each time course, v₀ is the rate in the absence of inhibitor, Kₐ is the Michaelis constant of the substrate A, and Kᵢₓ is the apparent inhibition constant, Kᵢ or Kᵢ*, obtained from fitting of vᵢ or vₛ, respectively.

$$\frac{{v}_{x}}{{v}_{0}} = \frac{1}{1 + [ I ] / [ {K}_{ix} ( 1 + \frac{[ A ]}{{K}_{a}} ) ]} \qquad ( 5 )$$

$$P = [ \frac{{V}_{max} [ A ] t}{{K}_{a} ( 1 + \frac{[ I ]}{{K}_{i} ( \frac{{k}_{4}}{{k}_{3} + {k}_{4}} )} + [ A ]} ] + [ \frac{\frac{{V}_{max} [ A ]}{{K}_{a} ( 1 + \frac{[ I ]}{{K}_{i}} ) + [ A ]}}{{k}_{4} + \frac{{k}_{3} [ I ]}{{K}_{i} ( 1 + \frac{[ A ]}{{K}_{a}} ) + [ I ]}} ] \\ [ 1 - exp ( - t ( {k}_{4} + \frac{{k}_{3} [ I ]}{{K}_{i} ( 1 + \frac{[ I ]}{{K}_{a}} ) + [ I ]} ) ) ] + C \qquad ( 6 )$$

In addition, time-course data were fitted globally by two methods. First, expressions for vₛ, vᵢ, and k_{obs} were substituted into eq 3 to generate eq 6, which was used to fit time-course data at all concentrations of inhibitors simultaneously. Second, time-course data were fitted using KinTek Explorer as either a single-binding step model with the two kinetic parameters k₃/Kᵢ and k₄ or a two-step binding mechanism as shown in FIG. 9 from which rate constants k₁-k₄ are determined (Supporting Information).

Abbreviations

AMC, 7-amino-4-methylcoumarin; BSF, bloodstream form; CHAPS, 3-[3-(cholamidopropyl)dimethylammonio]-1-propanesulfonate; DIPEA, N,N-diisopropylethylamine; hPhe, homophenylalanine; MES, 2-(N-morpholino)ethanesulfonic acid; MMTS, S-methyl methanethiosulfonate; Oxz, oxazolyl; NMePip, N-methylpiperazinyl; PCF, procyclic form; PVHI, peptidomimetic vinylheterocyclic inhibitor; Pyr, pyridinyl; Pyrmd, pyrimidinyl; Thz, thiazolyl; T3P, propylphosphonic anhydride; VS, vinyl sulfone

### REFERENCES

(1) Muñoz-Saravia, S. G.; Haberland, A.; Wallukat, G.; Schimke, I. Chronic Chagas' Heart Disease: a Disease on its Way to Becoming a Worldwide Health Problem: Epidemiology, Etiopathology, Treatment, Pathogenesis and Laboratory Medicine. Heart Fail. Rev. 2012, 17, 45-64.
(2) Centers for Disease Control and Prevention. https://www.cdc.gov/parasites/chagas (Accessed October 2019).
(3) Coura, J. R.; Vinas, P. A. Chagas Disease: a New Worldwide Challenge. Nature 2010, 465, S6-S7. USA: High-Risk Patient Populations for Screening. Curr. Trop. Med. Rep. 2019, 6, 8-12.
(5) Montgomery, S. P.; Parise, M. E.; Dotson, E. M.; Bialek, S. R. What Do We Know About Chagas Disease in the United States? Am. J. Trop. Med. Hyg. 2016, 95, 1225-1227.
(6) Rassi, A., Jr.; Rassi, A.; Marcondes de Rezende, J. American Trypanosomiasis (Chagas Disease). Infect. Dis. Clin. North Am. 2012, 26, 275-291.
(7) Lejon, V.; Bentivoglio, M.; Franco, J. R. Human African Trypanosomiasis. In Handbook of Clinical Neurology; Garcia, H. H.; Tanowitz, H. B.; Del Brutto, O. H., Eds; Elsevier: Cambridge, M A, 2013; pp 169-181.
(8) World Health Organization. Trypanosomiasis, human African (sleeping sickness) https://www.who.int/news-room/fact-sheets/detail/trypanosomiasis-human-african-(sleeping-sickness) (Accessed October 2019).
(9) Drugs for Neglected Diseases initiative. https://www.dndi.org/wpcontent/uploads/2019/09/Factsheet2019_SleepingSickness.pdf (Accessed October 2019).
(10) Simarro, P. P.; Jannin, J.; Cattand, P. Eliminating Human African Trypanosomiasis: Where Do We Stand and What Comes Next? PLOS Medicine 2008, 5, No. e55.
(11) Kennedy, P. G. E. Clinical Features, Diagnosis, and Treatment of Human African Trypanosomiasis (Sleeping Sickness). Lancet Neurol. 2013, 12, 186-194.
(12) Salomon, C. J. First Century of Chagas' Disease: An Overview on Novel Approaches to Nifurtimox and Benznidazole Delivery Systems. J. Pharm. Sci. 2012, 101, 888-894.
(13) Cianni, L.; Feldmann, C. W.; Gilberg, E.; Gutschow, M.; Juliano, L.; Leitão, A.; Bajorath, J.; Montanari, C. A. Can Cysteine Protease Cross-Class Inhibitors Achieve Selectivity? J. Med. Chem. 2019, 62, 10497-10525.
(14) Choe, Y.; Leonetti, F.; Greenbaum, D. C.; Lecaille, F.; Bogyo, M.; Bromme, D.; Ellman, J. A.; Craik, C. S. Substrate Profiling of Cysteine Proteases Using a Combinatorial Peptide Library Identifies Functionally Unique Specificities. J Biol. Chem. 2006,281, 12824-12832.
(15) Lecaille, F.; Authie, E.; Moreau, T.; Serveau, C.; Gauthier, F.; Lalmanach, G. Subsite Specificity of Trypanosomal Cathepsin L-Like Cysteine Proteases. Eur. J. Biochem. 2001, 268, 2733-2741.
(16) Sudhan, D. R.; Siemann, D. W. Cathepsin L Targeting in Cancer Treatment. Pharmacol. Ther. 2015, 155, 105-116.
(17) Miller, B. E.; Mayer, R. J.; Goyal, N.; Bal, J.; Dallow, N.; Boyce, M.; Carpenter, D.; Churchill, A.; Heslop, T.; Lazaar, A. L. Epithelial Desquamation Observed in a Phase I Study of an Oral Cathepsin C Inhibitor (GSK2793660). Br. J. Clin. Pharmacol. 2017, 83, 2813-2820.
(18) Wilkinson, R. D. A.; Williams, R.; Scott, C. J.; Burden, R. E. Cathepsin S: Therapeutic, Diagnostic, and Prognostic Potential. Biol. Chem. 2015, 396, 867-882.
(19) Drake, M. T.; Clarke, B. L.; Oursler, M. J.; Khosla, S. Cathepsin K Inhibitors for Osteoporosis: Biology, Potential Clinical Utility, and Lessons Learned. Endocr. Rev. 2017, 38, 325-350.
(20) Sajid, M.; Robertson, S. A.; Brinen, L. S.; McKerrow, J. H. Cruzain. In Cysteine Proteases of Pathogenic Organisms; Robinson, M. W.; Dalton, J. P., Eds; Springer: Boston, M A, 2011; pp 100-115.
(21) Eakin, A. E.; Mills, A. A.; Harth, G.; McKerrow, J. H.; Craik, C. S. The Sequence, Organization, and Expression of the Major Cysteine Protease (Cruzain) from Trypanosoma cruzi. J. Biol. Chem. 1992, 267, 7411-7420.
(22) McGrath, M. E.; Eakin, A. E.; Engel, J. C.; McKerrow, J. H.; Craik, C. S.; Fletterick, R. J. The Crystal Structure of Cruzain: A Therapeutic Target for Chagas' Disease. J. Mol. Biol. 1995, 247, 251-259.
(23) Caffrey, C. R.; Hansell, E.; Lucas, K. D.; Brinen, L. S.; Hernandez, A. A.; Cheng, J.; Gwaltney, S. L., II; Roush, W. R.; Stierhof, Y. D.; Bogyo, M.; Steverding, D.; McKerrow, J. H. Active Site Mapping, Biochemical Properties and Subcellular Localization of Rhodesain, the Major Cysteine Protease of Trypanosoma brucei rhodesiense. Mol. Biochem. Parasitol 2001, 118, 61-73.
(24) Steverding, D.; Sexton, D. W.; Wang, X.; Gehrke, S. S.; Wagner, G. K.; Caffrey, C. R. Trypanosoma brucei: Chemical Evidence that Cathepsin L is Essential for Survival and a Relevant Drug Target. Int. J. Parasitol. 2012, 42, 481-488.
(25) O'Brien, T. C.; Mackey, Z. B.; Fetter, R. D.; Choe, Y.; O'Donoghue, A. J.; Zhou, M.; Craik, C. S.; Caffrey, C. R. A Parasite Cysteine Protease is Key to Host Protein Degradation and Iron Acquisition. J. Biol. Chem. 2008, 283, 28934-28943.
(26) Doyle, P. S.; Zhou, Y. M.; Hsieh, I.; Greenbaum, D. C.; McKerrow, J. H.; Engel, J. C. The Trypanosoma cruzi Protease Cruzain Mediates Immune Evasion. PLoS Pathog. 2011, 7, No. e1002139.
(27) Harth, G.; Andrews, N.; Mills, A. A.; Engel, J. C.; Smith, R.; McKerrow, J. H. Peptide-fluoromethyl Ketones Arrest Intracellular Replication and Intercellular Transmission of Trypanosoma cruzi. Mol. Biochem. Parasitol. 1993, 58, 17-24.
(28) Doyle, P. S.; Zhou, Y. M.; Engel, J. C.; McKerrow, J. H. A Cysteine Protease Inhibitor Cures Chagas Disease in an Immunodeficient-Mouse Model of Infection. Antimicrob. Agents Chemother. 2007, 51, 3932-3939.
(29) Engel, J. C.; Doyle, P. S.; McKerrow, J. H. Trypanocidal Effect of Cysteine Protease Inhibitors in vitro and in vivo in Experimental Chagas Disease. Medicina 1999, 59, 171-175.
(30) Engel, J. C.; Doyle, P. S.; Hsieh, I.; McKerrow, J. H. Cysteine Protease Inhibitors Cure an Experimental Trypanosoma cruzi Infection. J. Exp. Med. 1998, 188, 725-734.
(31) Kerr, I. D.; Lee, J. H.; Farady, C. J.; Marion, R.; Rickert, M.; Sajid, M.; Pandey, K. C.; Caffrey, C. R.; Legac, J.; Hansell, E.; McKerrow, J. H.; Craik, C. S.; Rosenthal, P. J.; Brinen, L. S. Vinyl Sulfones as Antiparasitic Agents and a Structural Basis for Drug Design. J. Biol. Chem. 2009, 284, 25697-25703.
(32) Chen, Y. T.; Brinen, L. S.; Kerr, I. D.; Hansell, E.; Doyle, P. S.; McKerrow, J. H.; Roush, W. R. In vitro and in vivo Studies of the Trypanocidal Properties of WRR-483 against Trypanosoma cruzi. PLoS Negl. Trop. Dis. 2010, 4, No. e825.
(33) Mott, B. T.; Ferreira, R. S.; Simeonov, A.; Jadhav, A.; Ang, K. K. H.; Leister, W.; Shen, M.; Silveira, J. T.; Doyle, P. S.; Arkin, M. R.; McKerrow, J. H.; Inglese, J.; Austin, C. P.; Thomas, C. J.; Shoichet, B. K.; Maloney, D. J. Identification and Optimization of Inhibitors of Trypanosomal Cysteine Proteases: Cruzain, Rhodesain, and TbCatB. J. Med. Chem. 2010, 53, 52-60.
(34) McKerrow, J. H.; Doyle, P. S.; Engel, J. C.; Podust, L. M.; Robertson, S. A.; Ferreira, R.; Saxton, T.; Arkin, M.; Kerr, I. D.; Brinen, L. S.; Craik, C. S. Two Approaches to Discovering and Developing New Drugs for Chagas Disease. Mem. Inst. Oswaldo Cruz 2009, 104, 263-269.
(35) Chenna, B. C.; Li, L.; Mellott, D. M.; Zhai, X.; Siqueira-Neto, J. L.; Calvet Alvarez, C.; Bernatchez, J. A.; Desormeaux, E.; Alvarez Hernandez, E.; Gomez, J.; McKerrow, J. H.; Cruz-Reyes, J.; Meek, T. D. Peptidomimetic vinyl heterocyclic inhibitors of cruzain effect antitrypanosomal activity. J. Med. Chem. 2020, 63, 3298-3316
(36) Bradshaw, J. M.; McFarland, J. M.; Paavilainen, V. O.; Bisconte, A.; Tam, D.; Phan, V. T.; Romanov, S.; Finkle, D.; Shu, J.; Patel, V.; Ton, T.; Li, X.; Loughhead, D. G.; Nunn, P. A.; Karr, D. E.; Gerritsen, M. E.; Funk, J. O.; Owens, T. D.; Verner, E.; Brameld, K. A.; Hill, R. J.; Goldstein, D. M.; Taunton, J. Prolonged and Tunable Residence Time Using Reversible Covalent Kinase Inhibitors. Nat. Chem. Biol. 2015, 11, 525-531.
(37) Krishnan, S.; Miller, R. M.; Tian, B.; Mullins, R. D.; Jacobson, M. P.; Taunton, J. Design of Reversible, Cysteine-Targeted Michael Acceptors Guided by Kinetic and Computational Analysis. J. Am. Chem. Soc. 2014, 136, 12624-12630.
(38) Copeland, R. A.; Pompliano, D. L.; Meek, T. D. Drug-Target Residence Time and its Implications for Lead Optimization. Nat. Rev. Drug Discovery 2006, 5, 730-739.
(39) Barr, S. C.; Warner, K. L.; Kornreic, B. G.; Piscitelli, J.; Wolfe, A.; Benet, L.; McKerrow, J. H. A Cysteine Protease Inhibitor Protects Dogs from Cardiac Damage during Infection by Trypanosoma cruzi. Antimicrob. Agents Chemother. 2005, 49, 5160-5161.
(40) Zhai, X.; Meek, T. D. Catalytic Mechanism of Cruzain from Trypanosoma cruzi As Determined from Solvent Kinetic Isotope Effects of Steady-State and Pre-Steady-State Kinetics. Biochemistry 2018, 57, 3176-3190.
(41) Silverman, R. B. Mechanism-Based Enzyme Inactivators. In Methods in Enzymology; Johnson, M. L.; Brand, L., Eds; Academic Press: Cambridge, M A, 1995; pp 240-283.
(42) Brady, K.; Abeles, R. H. Inhibition of Chymotrypsin by Peptidyl Trifluoromethyl Ketones: Determinants of Slow-Binding Kinetics. Biochemistry 1990, 29, 7608-7617.
(43) Rubach, J. K.; Cui, G.; Schneck, J. L.; Taylor, A. N.; Zhao, B.; Smallwood, A.; Nevins, N.; Wisnoski, D.; Thrall, S. H.; Meek, T. D. The Amino-Acid Substituents of Dipeptide Substrates of Cathepsin C Can Determine the Rate-Limiting Steps of Catalysis. Biochemistry 2012, 51, 7551-7568.
(44) Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K.; Shaw, D. E.; Francis, P.; Shenkin, P. S. Glide: A New Approach for Rapid, Accurate Docking and Scoring. 1. Method and Assessment of Docking Accuracy. J. Med. Chem. 2004, 47, 1739-1749.
(45) Halgren, T. A.; Murphy, R. B.; Friesner, R. A.; Beard, H. S.; Frye, L. L.; Pollard, W. T.; Banks, J. L. Glide: A New Approach for Rapid, Accurate Docking and Scoring. 2. Enrichment Factors in Database Screening. J. Med. Chem. 2004, 47, 1750-1759.
(46) Friesner, R. A.; Murphy, R. B.; Repasky, M. P.; Frye, L. L.; Greenwood, J. R.; Halgren, T. A.; Sanschagrin, P. C.; Mainz, D. T. Extra Precision Glide: Docking and Scoring Incorporating a Model of Hydrophobic Enclosure for Protein-Ligand Complexes. J. Med. Chem. 2006, 49, 6177-6196.
(47) Zhu, K.; Borrelli, K. W.; Greenwood, J. R.; Day, T.; Abel, R.; Farid, R. S.; Harder, E. Docking Covalent Inhibitors: A Parameter Free Approach To Pose Prediction and Scoring. J. Chem. Inf. Model. 2014, 54, 1932-1940.
(48) Wittig, G.; Schollkopf, U. Ober Triphenyl-phosphin-methylene als Olefinbildende Reagenzien I. Mitteil. Chem. Ber. 1954, 87, 1318-1330.
(49) Wadsworth, W. S.; Emmons, W. D. The Utility of Phosphonate Carbanions in Olefin Synthesis. J. Am. Chem. Soc. 1961, 83, 1733-1738.
(50) Nahm, S.; Weinreb, S. M. N-methoxy-N-Methylamides as Effective Acylating Agents. Tetrahedron 1981, 22, 3815-3818. (51) Klaus, J. L.; Palmer, J. T.; Rasnick, D. Irreversible Cysteine Protease Inhibitors Containing Vinyl Groups Conjugated to Electron Withdrawing Groups. U.S. Pat. No. 6,287,840, 2001.
(52) Morrison, J. F.; Walsh, C. T. The Behavior and Significance of Slow-Binding Enzyme Inhibitors. Adv. Enzymol. Relat. Areas Mol. Biol. 1988, 61, 201-301.
(53) Jones, B. D.; Tochowicz, A.; Tang, Y.; Cameron, M. D.; McCall, L. I.; Hirata, K.; Siqueira-Neto, J. L.; Reed, S. L.; McKerrow, J. H.; Roush, W. R. Synthesis and Evaluation of Oxyguanidine Analogues of the Cysteine Protease Inhibitor WRR-483 against Cruzain. ACS Med. Chem. Lett. 2015, 7, 77-82.
(54) Ndao, M.; Beaulieu, C.; Black, W. C.; Isabel, E.; Vasquez-Camargo, F.; Nath-Chowdhury, M.; Masse, F.; Mellon, C.; Methot, N.; Nicoll-Griffith, D. A. Reversible Cysteine Protease Inhibitors Show Promise for a Chagas Disease Cure. Antimicrob. Agents Chemother. 2014, 58, 1167-1178.
(55) Boudreau, P. D.; Miller, B. W.; McCall, L. I.; Almaliti, J.; Reher, R.; Hirata, K.; Le, T.; Siqueira-Neto, J. L.; Hook, V.; Gerwick, W. H. Design of Gallinamide A Analogs as Potent Inhibitors of the Cysteine Proteases Human Cathepsin L and Trypanosoma cruzi Cruzain. J. Med. Chem. 2019, 62, 9026-9044.
(56) Zhao, Z.; Bourne, P. E. Progress with Covalent Small-Molecule Kinase Inhibitors. Drug Discov. Today 2018, 23, 727-735.
(57) Nkemgu, N. J.; Grande, R.; Hansell, E.; McKerrow, J. H.; Caffrey, C. R.; Steverding, D. Improved Trypanocidal Activities of Cathepsin L Inhibitors. Int. J. Antimicrob. Agents 2003, 22, 155-159.
(58) Yang, P. Y.; Wang, M.; He, C. Y.; Yao, S. Q. Proteomic Profiling and Potential Cellular Target Identification of K11777, a Clinical Cysteine Protease Inhibitor, in Trypanosoma brucei. Chem. Commun 2012, 48, 835-837.
(59) Lee, G. M.; Balouch, E.; Goetz, D. H.; Lazic, A.; McKerrow, J. H.; Craik, C. S. Mapping Inhibitor Binding Modes on an Active Cysteine Protease via Nuclear Magnetic Resonance Spectroscopy. Biochemistry 2012, 51, 10087-10098.
(60) Mellott D M, Tseng C T, Drelich A, Fajtovi P, Chenna B C, Kostomiris D H, Hsu J, Zhu J, Taylor Z W, Kocurek K I, Tat V, Katzfuss A, Li L, Giardini M A, Skinner D, Hirata K, Yoon M C, Beck S, Carlin A F, Clark A E, Beretta L, Maneval D, Hook V, Frueh F, Hurst B L, Wang H, Raushel F M, O'Donoghue A J, de Siqueira-Neto J L, Meek T D, McKerrow J H. A Clinical-Stage Cysteine Protease Inhibitor blocks SARS-CoV-2 Infection of Human and Monkey Cells. ACS Chem Biol. 2021, 16, 642-650.
(61) Li L, Chenna B C, Yang K S, Cole T R, Goodall Z T, Giardini M, Moghadamchargari Z, Hernandez E A, Gomez J, Calvet C M, Bernatchez J A, Mellott D M, Zhu J, Rademacher A, Thomas D, Blankenship L R, Drelich A, Laganowsky A, Tseng C K, Liu W R, Wand A J, Cruz-Reyes J, Siqueira-Neto J L, Meek T D. Self-Masked Aldehyde Inhibitors: A Novel Strategy for Inhibiting Cysteine Proteases. J Med Chem. 2021, 64, 11267-11287.

### Example 2: Aldehyde and Masked Aldehyde Inhibitors of Cruzain, Human Cathepsins L and B, SARS-CoV-2

Research continues to identify additional inhibitors of these cysteine proteases that are covalent irreversible and covalent reversible. Specificity for the target enzymes will be afforded by selecting the optimal peptide recognition sequence as obtained from kinetic studies. The overall size of the cysteine protease inhibitors will be reduced in accord with Lipinski rules for drug development, and the compounds evaluated against cellular cultures of Trypanosoma cruzi, Trypanosoma brucei, and SARS-CoV-2. Compounds that kill parasites and/or virus will be further advanced to testing in animal models of COVID-19, African sleeping sickness, and Chagas disease.

Few inhibitors of cysteine proteases with reversible modes of action have been characterized to date that have low nanomolar potency for their enzyme targets. The masked aldehyde inhibitors disclosed herein represent a new niche in the discovery of new inhibitors of this important class of enzymes. Covalent inhibitors of therapeutically-important cysteine proteases, including SARS-CoV-2 3CLpro, cruzain and cathepsin L, have been previously identified but none of the compounds contain masked aldehydes described herein. The concept of using a heterocycle as an isosteric surrogate of an amide or sulfone, allows conjugation between the reactive vinyl group that would allow substitution in order to modify the electrophilicity of the vinyl group. The concept of a masked aldehyde in the forms of (oxo) 2-hydroxyphenyl- or 2-carboxyphenyl-lactols is unprecedented. Patents exist for covalent inactivators of cysteine proteases, some of which include a vinyl substituent located in the exemplified peptidomimetic compounds in the same position as compounds 1-27. One such patent is WO95/02322 where sulfones, esters, amides and other electron-withdrawing groups are attached to the vinyl group, but heterocycles are not found within the claims.

Cysteine proteases catalyze the cleavage of peptide and protein amide bonds which convert their protein substrates to protein fragments that are further proteolyzed to polypeptides. One result of the action of cysteine proteases on protein substrate is to activate proteins as a result of their peptidolytic action.

Cysteine proteases of parasitic protozoa, such as cruzain, brucipain, the falcipains, and rhodesain, among others, catalyze the cleavage of both parasitic and host proteins for the purpose of (a) evasion of the immune response of the mammalian host, (b) degradation of iron- and other metal-binding proteins such as hemoglobin to scavenge metal ions, and (c) to effect changes in protozoan morphology to adapt to different host environments.

Cysteine proteases of viruses, specifically, coronaviruses, catalyze the cleavage of coronaviral polyproteins in the process of maturation of the coronaviral particles, which is essential to survival of the virus.

Mammalian and human cysteine proteases such as cathepsin B and cathepsin L have been associated with proteolytic processing of the Spike protein of the betacoronaviruses, and this action assists the penetrance of the virus (such as SARS CoV-2) into mammalian and human host cells, thereby facilitating coronaviral infection.

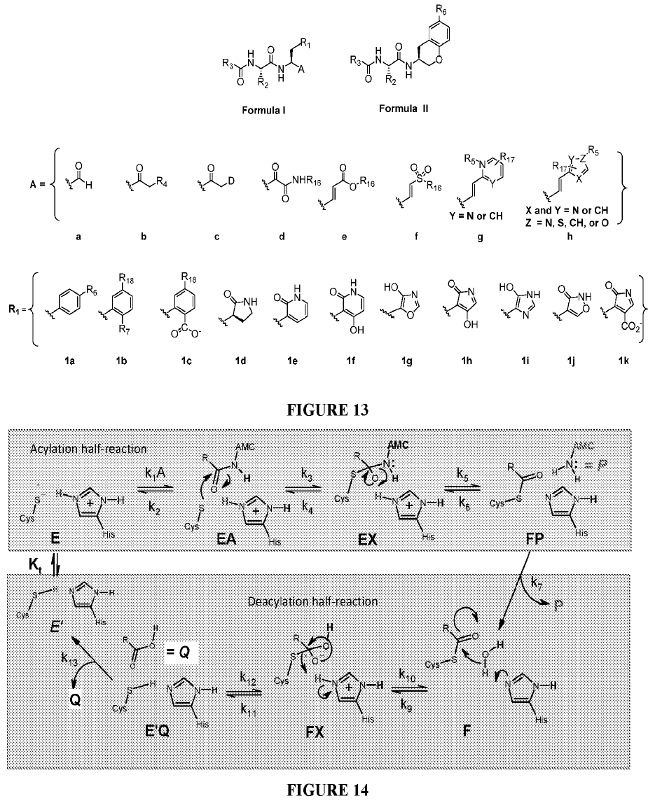

The chemical (also known as the catalytic) mechanisms of the cysteine proteases are highly similar (FIG. 14). In short, the active sites of these enzymes consist of a conserved pair of amino acids, the eponymous cysteine group, and a conserved histidine residue, which collaborate to effect a double-displacement mechanism in which an acylation half-reaction is followed by a de-acylation half-reaction.

In the mechanism, the thiol group of the cysteine residue undergoes de-protonation by the proximal histidine residue, elaborating a thiolate group that subsequently attacks the carbonyl of a peptide or protein carboxamide group (EA to EX). The resulting tetrahedral intermediate (EX) resulting from this thiolation reaction progresses to protonation of the amide nitrogen by the imidazolium species of the conserved histidine, which results in collapse of this intermediate to produce a product amine-peptide (P), and a semi-stable enzyme-substrate thioester (F).

The now-un-protonated conserved histidine residue de-protonates a bound water molecule from which the product hydroxide ion attacks the peptide-carbonyl group of the enzyme-substrate thioester to form a second tetrahedral intermediate (FX), which subsequently collapses to produce the peptide-carboxylic acid product (E′Q), and enzyme is restored to its pre-catalysis form.

Exemplification of the proposed chemical mechanism has been demonstrated with cruzain. Cruzain is an essential cysteine protease of Trypanosoma cruzi, the parasitic protozoan that causes Chagas disease, and comprises perhaps the most significant discrete drug target for the discovery of anti-chagaisic therpaies. Extensive details of the catalytic mechanism of the cysteine protease cruzain, a target for the treatment of Chagas disease, in terms of the rate-limiting steps of the enzymatic reaction and the protonation states of the active-site Cys and His residues were recently published (Zhai, X. and Meek, T. D. (2018)⁴⁰ and is shown in FIG. 14. The results from this study demonstrated that for peptide substrates with optimal values of k_{cat}/Kₘ and k_{cat}, the rate of de-acylation of the enzyme-thioester is rate-limiting, and that substrates bind to a free enzyme form in which both the active-site cysteine (Cys-25) and histidine (His-159) are neutral. This latter result is unusual for cysteine proteases in that the Cys-His catalytic is generally in its more reactive thiolate-imidazolium form, and the findings should inform strategies for the design of new inhibitors. Importantly, values of k_{ca}t/Kₘ report on the rates of substrate binding and catalysis up to and including the acylation of active-site Cys-25, and so this kinetic parameter comprises a useful guide to select optimal di-peptide scaffolds for inhibitors which are meant to form reversible covalent complexes with Cys-25 upon binding, which chemically mimics acylation.

The most successful inhibitors of cysteine proteases are peptide analogues that form covalent bonds, both reversible and irreversible, with the active-site cysteines of these enzyme targets. The types of substituents that form these covalent bonds include but are not limited to epoxides, α-ketoamides, α,β-unsaturated ketones, nitriles, ketones, aldehydes, propenamides and vinyl sulfones (FIG. 16).

These electrophilic “warheads” have been installed in peptidomimetic or “organic” scaffolds, and have led to clinical candidates for the treatment of malaria, Chagas disease, cystic fibrosis, COVID-19, and osteoporosis. However, these covalent inactivators generally suffer from low selectivity due to presumed covalent attachment to other enzymes leading to untoward toxicities.

New inhibitors (or covalent inactivators) of cysteine proteases have been developed, which contain electrophilic “warheads” that may be “tuned” for selectivity and reactivity with the target cysteine protease, or, covalent inhibitors which are unveiled in the active site of the target enzyme. Peptide analogue inhibitors exemplified in Formula 1 contain several types of inhibitory warheads, including aldehydes, masked aldehydes (lactols, a-keto-lactols), vinyl-heterocycles, α-keto-amides, α,β-unsaturated ketones, α-halo-ketones, α-oxycarbonyl-lactols, and vinyl-sulfones.

The chemical mechanism of cruzain was thereafter exploited to develop a novel class of cruzain inhibitors, known as peptidomimetic vinyl-heterocyclic inhibitors (Chenna et al (2020)³⁵, The concept of reversible covalent inhibition exemplified by the vinyl-heterocyclic inhibitors, some of which exert potent anti-chagaisic activity in a cellular model of Chagas disease, may now be adapted to inhibition of the SARS CoV-2 cysteine proteases, 3CL protease (3CLpro or Main pro) and the papain-like protease (PLpro) when installed in the proper peptide/peptidomimetic scaffold. Such new compounds show selectivity for the target SARS-CoV-2 proteases. These vinyl-heterocyclic moieties are shown in FIG. 13.

Included in the peptide vinyl-heterocyclic inhibitors characterized in Chenna et al (2020)³⁵ are compounds that have anti-trypanosomal activity vs. axenic cultures of T. brucei brucei and in T. cruzi-infected murine cardiomyoblasts at low micromolar concentrations. Another class of reversible covalent inhibitors of cruzain, cathepsin L, and now adapted to CoV-2 3CLpro are the masked aldehydes, as well as their “parent” aldehydes. A peptide or oligopeptide aldehyde may exert exceptionally potent inhibition, or even apparent inactivation of cysteine proteases, resulting from binding to a cysteine protease, followed by covalent adduction of the active-site thiolate of the conserved cysteine to form a hemithioacetal, which may or may slowly revert to aldehyde and pristine protease (FIG. 21). Many examples of peptide aldehyde inhibitors of cysteine proteases exist, including recently characterized inhibitors of cathepsin L and SARS-CoV-2 3CLpro.

Here we report characterized peptide-aldehyde inhibitors of the cysteine proteases cathepsin L, cathepsin B, cruzain, and 3CLpro. Some of these compounds as shown in Tables 1 and 2 exert exceptionally potent inhibition/inactivation of cathepsin L, cruzain, and SARS-CoV-2 3CLpro. Some of these peptide-aldehydes exert anti-trypanosomal activity in axenic cell cultures of Trypanosoma brucei brucei and in a cellular model of Chagas disease (amastigote forms of Trypanosoma cruzi; Table 1 and FIG. 11.

Peptide-aldehyde inhibitors of cysteine proteases, while exceptionally potent, are generally too reactive with cellular nucleophiles to furnish drug candidates as they will be lost to oxidative metabolism or inactivation by conjugation to cellular thiols, such as glutathione, or abundant proteins bearing salient cysteine or lysine residues. Our strategy to implement the use of peptide-aldehyde compounds as potential drug candidates, is to block (or “mask”) the aldehyde by its formation with an alcohol or bisulfite, to form an intermolecular (bisulfite or hemiacetal), or, more useful, intramolecular hemiacetals with an alcohol, amine, thiol, or other nucleophile that is contained in the same compound as the aldehyde substituent (Table 2 and FIG. 7).

We describe herein the concept of masked aldehydes. By this definition, a “masked aldehyde” comprises an intramolecular hemiacetal (a lactol) in which a hydroxy, carboxy, thiolate, or amino groups is proximal to the parent aldehyde group, and forms with it respectively, a lactol, an a-keto-lactol, a hemithioacetal, or an aminal.

For the masked aldehydes, all peptide analogues contain an aldehyde group on the C-terminus (Compounds 28-36 in Table 2). A compound of the structure Cbz-Phe-Phe-CHO (LL-205 (compound 28) and LL-127 (compound 29) (see Table 2)) are (unmasked) aldehydes that inhibit cruzain, respectively, at inhibition constants of 0.44 and 22 nM vs. cruzain, and for aldehyde 28, an inhibition constant of 0.31 nM vs. human cathepsin L.

Compound 28 (LL-205) is the structure of Formula 1 in Table 2 in which A is an aldehyde (a), R₁ is a benzyl group (1a, R₆=H), R₂ is Bz, and R₃ is a benzyloxycarbonyl group (Cbz). LL-205 is an inhibitor of cruzain in which its inhibition constants for cruzain and human cathepsin L are, respectively, Kᵢ*=0.44±0.02 nM and Kᵢ*=0.31±0.01 nM and the compound has anti-trypanosomal properties with values EC₅₀=8 and 3 micromolar, and inhibits CoV-2 infection of Vero E6 cells with a value of EC₅₀=0.5 micromolar. Compound 28 is not an inhibitor of 3CL protease, but has an EC₅₀=0.5 micromolar in Vero E6 cells infected with SARS-CoV-2.

Compound 29 (LL-127) is the structure of Formula 1 in Table 2 in which A is an aldehyde (a), R₁ is a a substituted benzyl group (1a, R₆=OMe), R₂ is Bz, and R₃ is a benzyloxycarbonyl group (Cbz). LL-127 is an inhibitor of cruzain in which its inhibition constant is Kᵢ*=22±2 nM.

Compound 30 (LL-166) is the structure of Formula 1 in Table 2 in which A is an aldehyde (a), R₁ is a substituted benzyl group (1b, R₆=OH), R₂ is Bz, and R₃ is a benzyloxycarbonyl group (Cbz). This compound is a masked aldehyde, is an inhibitor of cruzain, human cathepsin L, and human cathepsin B, with respective inhibition constants of Kᵢ*=49±2 nM, Kᵢ*=28±0.9 nM, and Kᵢ*=4500±100 nM, and the compound has anti-trypanosomal properties with values EC₅₀=5.4 and 17 micromolar, and inhibits CoV-2 infection of Vero E6 cells with a value of EC₅₀=5 micromolar; 30 is not an inhibitor of 3CL protease, but has an EC₅₀=5 micromolar in Vero E6 cells infected with SARS-CoV-2.

Compound 31 (LL-232) is the structure of Formula 1 in Table 2 in which A is an aldehyde (a), R₁ is a substituted benzyl group (1b, R₆=OMe), R₂ is Bz, and R₃ is a benzyloxycarbonyl group (Cbz). This compound is a masked aldehyde, is an inhibitor of cruzain, human cathepsin L, and human cathepsin B, with respective inhibition constants of Kᵢ*=350±30 nM, Kᵢ*=38±2 nM, and Kᵢ*=5500±400 nM, and the compound has anti-trypanosomal properties with values EC₅₀=3-11 micromolar, and inhibits CoV-2 infection of Vero E6 cells with a value of EC₅₀=2.5 micromolar; 31 is not an inhibitor of 3CL protease.

Compound 32 (LL-254) is the structure of Formula 1 in Table 2 in which A is an aldehyde (a), R₁ is a 2-hydroxybenzyl group (1b) in which R₆ is a methyl, R₂ is Bz, and R₃ is a benzyloxycarbonyl group (Cbz); Compound 31 inhibits cruzain, human cathepsin L, cathepsin B, and 3CL pro, respectively, at Kᵢ*=103±5 nM, Kᵢ*=58±2 nM, and Kᵢ*=6100±900 nM; and >10,000 nM. 32 has anti-trypanosomal properties with values of EC₅₀=0.5 and 7 micromolar, and 30 is not an inhibitor of 3CL protease.

Compound 33 (LL-291) is the structure of Formula 1 in Table 2 in which A is an aldehyde (a), R₁ is a 2-hydroxybenzyl group (1b) in which R₆ is a chlorine, R₂ is Bz, and R₃ is a benzyloxycarbonyl group (Cbz); Compound 33 inhibits cruzain, human cathepsin L, cathepsin B, and 3CL pro, respectively, at Kᵢ*=74±10 nM, Kᵢ*=27±0.7 nM, Kᵢ*=1400±100 nM; and >10,000 nM, respectively. Compound 33 has anti-trypanosomal properties with values EC₅₀=6-10 micromolar.

Compound 34 (LL-294) is the structure of Formula 1 in Table 2 in which A is an aldehyde (a), R₁ is a 2-hydroxybenzyl group (1b) in which R₆ is a fluorine, R₂ is Bz, and R₃ is a benzyloxycarbonyl group (Cbz); Compound 34 inhibits cruzain, human cathepsin L, cathepsin B, and 3CL pro, respectively, at Kᵢ*=48±2 nM, Kᵢ*=23±1 nM, Kᵢ*=2300±200 nM; and >10,000 nM, respectively. Compound 34 has anti-trypanosomal properties with values EC₅₀=3-11 micromolar, and inhibits CoV-2 infection of Vero E6 cells with a value of EC₅₀=4 micromolar.

Compound 35 (LL-322) is the structure of Formula 1 in Table 2 in which A is an aldehyde (a), R₁ is a 2-hydroxybenzyl group (1b) in which R₆ is a methylcarboxylate, R₂ is Bz, and R₃ is a benzyloxycarbonyl group (Cbz); Compound 35 inhibits cruzain, human cathepsin L, cathepsin B, and 3CL pro, respectively, at Kᵢ*=18±0.5 nM, Kᵢ*=10.8±0.8 nM, Kᵢ*=670±30 nM; and >10,000 nM, respectively. Compound 35 has anti-trypanosomal properties with values EC₅₀=3.5-6.7 micromolar, and inhibits CoV-2 infection of Vero E6 cells with a value of EC₅₀=2.5 micromolar.

Compound 36 (BC-552) is the structure of Formula 1 in Table 2 in which A is an aldehyde (a), R₁ is a 2-hydroxybenzyl group (1b) in which R₆ is a hydrogen, R₂ is Bz, and R₃ is a 4-methyl-piperazinyl group (N-MePip); Compound 36 inhibits cruzain, human cathepsin L, cathepsin B, and 3CL pro, respectively, at Kᵢ*=47±2 nM, Kᵢ*=20±0.9 nM, Kᵢ*=1270±70 nM; and >10,000 nM, respectively. Compound 36 has anti-trypanosomal properties with values EC₅₀=0.5-29 micromolar, and inhibits CoV-2 infection of Vero E6 cells with a value of EC₅₀=7.5 micromolar.

Masked aldehydes (MAs) are similar dipeptide analogues which contain a 2-hydroxy group on the C-terminal sidechain (an ortho-tyrosine), for which LL-166 (Compound 30) is an analog of LL-205; Kᵢ=49 nM). Substitution of carbon-4 of LL-166 with electron-donating (R₆ is methoxy group in LL-232, compound 31, a methyl in LL-254, compound 32, and a methyl-carboxy group in LL322, compound 35, provided respective values for cruzain of Kᵢ=350, 103, and 18 nM), and electron-withdrawing groups (R₆ is are chloro and fluoro groups at carbon-4 in LL-291, compound 33 and LL-294, compound 34, respectively, provided for cruzain respective values of Kᵢ=74 and 48 nM, suggested that electron-withdrawing groups are preferred over electron-donating groups for the inhibition of cruzain. BC552A (Compound 36) is a masked aldehyde in a peptide scaffold similar to that of K11777, inhibited cruzain with a value of Kᵢ=47 nM, equivalent to LL-166, suggesting that substitution of the P₃ group is unimportant for potency.

Four masked aldehydes, LL-166 (30), LL-254 (32), LL-294 (34), and BC-552A (36), demonstrated anti-T. cruzi activity in infected murine cardiomyoblasts, with respective values of EC₅₀=3.0±0.9, 5.7±0.6, 3.5±0.6, and 29±0.3 μM. In Table 2, compounds 30-36 exhibited EC₅₀ values of 4-17 μM in axenic cultures of pro-cyclic forms of T. b. brucei, and MAs 31-34 and 36 are more potent in bloodstream forms of T. b. brucei (EC₅₀=0.5-6.0 μM), with BC-552 being the most potent, possibly due to its superior aqueous solubility.

It is noteworthy that some of these MAs, notably BC-552 (compound 36 in Table 2) is significantly more potent vs. procyclic and bloodstream forms of T. b. brucei than the parent aldehyde LL-205 (compound 28), which is a better inhibitor of cruzain than the MAs by two orders of magnitude.

We have synthesized prodrug forms of Compound 36 (BC-552) which are either (compounds 37-39) acylated forms of BC-552, or mixed acetals (compounds 40 and 41). We have shown that cellular esterases catalyze de-acylation of compounds (37-40) in cellulo and in vivo, as we have observed in vitro. Active MA inhibitors are released partially or fully from their O-acylated forms by treatment with esterase in reaction mixtures under 100 minutes of incubation.

Prodrug compounds 37-39 in axenic cultures of bloodstream forms T. b. brucei, and compound 37 exhibited anti-trypanosomal EC₅₀ values of 3.7 μM, which is equivalent to activity of the parent peptide aldehyde LL-205, demonstrating that value of O-acylated forms of compound 30 (BC552). Compound 37-39 have anti-CoV-2 activities of 0.3-0.6 micromolar in A549/ACE2 cells infected with SARS CoV-2, which is superior to their parent masked aldehyde, BC-552.

The mixed acetals compounds 40 and 41 (LL-490 and LL-491) were also anti-trypanosomal inhibitors of the bloodstream forms of T. b. brucei, with larger values of EC₅₀=11-14 μM. These results suggested that oxidative degradation of acetals, such as from a cytochrome P450 enzymes, occurs within the protozoan, to release active inhibitor BC-552.

In contrast, compounds 30-36 are poor inhibitors of human cathepsin B, ranging from Kᵢ=670-6100 nM, which are on average 43-times less potent than vs. cruzain. The potent inhibition of MA inhibitors vs. human cathepsin L is noteworthy, considering that cathepsin L is implicated in cell penetrance and uptake of SARS CoV-1⁶⁰

Neither the dipeptide aldehyde (compound 30, LL-205), nor any of the masked aldehydes (compounds 30-36), which contain a substituted benzyl sidechain at the P₁ position, are inhibitors of the cysteine protease 3CL-pro from the SARS-CoV-2 virus (Table 2)).

However, masked aldehydes compounds 30, 31, 34, and 36, along with the parent aldehyde compound 28, are effective anti-CoV-2 agents at EC₅₀ values of 0.5 μM (compound 28) and EC₅₀=2.5-7.5 μM for compounds 30, 31, 34, and 36.

We recently characterized the target of anti-SARS-CoV-2 activity of the irreversible inactivator K777 in Vero E6 as cathepsin L.⁶⁰ Accordingly, the anti-SARS-CoV-2 activities of compounds 28, 30, 31, 34, and 36 at micromolar concentrations are likely due to inhibition of the cathepsin L of Vero E6 or A549/ACE2 cells (Table 2).

Given the above data, we synthesized the P₁-2-pyridone aldehyde compound 42 (BC-666) as a potential inhibitor of cruzain, human cathepsin L, and CoV-2 3Cl-pro, due to the fact that P₁ benzyl sidechains are well tolerated for cruzain and cathepsin L, while the stringent substrate specificity requires either a glutamine or an oxo-pyrrolidine-alanyl group at the P₁-sidechain of substrates/inhibitors of SARS CoV-2.

Compound 42 (BC-666) is the structure of Formula 1 in Table 2 in which A is an aldehyde (a), R₁ is a 2-pyridone (1e), R₂ is Bz, and R₃ is a 4-methyl-piperazinyl group (N-MePip); Compound 42 inhibits cruzain, human cathepsin L, and 3CL pro, respectively, at Kᵢ*=2.1±0.1 nM, Kᵢ*=2.3±0.1 nM, and Kᵢ*=860±90 nM, respectively. Compound 42 inhibits CoV-2 infection of Vero E6 cells with a value of EC₅₀>10 micromolar.

Compound 43 (LL-478) is the structure of Formula 1 in Table 2 in which A is an aldehyde (a), R₁ is the 2-pyridone (1e), R₂ is a methylene-cyclohexyl group (Cha), and R₃ is Cbz-valinyl; Compound 43 inhibits human cathepsin L and 3CL pro, respectively, at Kᵢ*=59±10 nM, and Kᵢ*=9±2 nM. Compound 43 inhibits CoV-2 infection of A549/ACE2 cells with a value of EC₅₀=5 micromolar.

Compound 44 (LL-482) is the structure of Formula 1 in Table 2 in which A is an aldehyde (a), R₁ is the 2-pyridone (1e), R₂ is a methylene-cyclohexyl group (Cha), and R₃ is N-acetyl-valinyl; Compound 44 inhibits human cathepsin L and 3CL pro, respectively, at Kᵢ*=140±39 nM, and Kᵢ*=60±20 nM. Compound 43 inhibits CoV-2 infection of Vero E6 cells with a value of EC₅₀>20 micromolar.

Compound 45 (BC-671) is the structure of Formula 1 in Table 2 in which A is an aldehyde (a), R₁ is the 2-pyridone (1e), R₂ is a leucinyl group, and R₃ is a 2-carbonylindole; Compound 45 inhibits human cathepsin L and 3CL pro, respectively, at Kᵢ*=39±3 nM, and Kᵢ*=190±30 nM. Compound 45 inhibits CoV-2 infection of Vero E6 cells with a value of EC₅₀>20 micromolar.

Compound 46 (BC-674) is the structure of Formula 1 in Table 2 in which A is an aldehyde (a), R₁ is the 2-pyridone (1e), R₂ is a methylene-cyclohexyl group (Cha) and R₃ is a 2-carbonylindole; Compound 45 inhibits 3CL pro at Kᵢ*=60±10 nM, and inhibits CoV-2 infection of Vero E6 cells with a value of EC₅₀>20 micromolar.

Compound 47 (LL-482) is the structure of Formula 1 in Table 2 in which A is an aldehyde (a), R₁ is the 2-pyridone (1e), R₂ is an 4-nitrobenzyl group; R₃ is a 2-carbonylindole; Compound 47 inhibits 3CL pro at Kᵢ*=260±40 nM, and inhibits CoV-2 infection of Vero E6 cells with a value of EC₅₀>20 micromolar.

Compound 48 (BC-753A) is the structure of Formula 1 in Table 2 in which A is an aldehyde (a), R₁ is the 2-pyridone (1e), R₂ is a L-proline; R₃ is N-acetyl-L-valine; Compound 48 inhibits 3CL pro at Kᵢ*=1160±80 nM, and inhibits CoV-2 infection of Vero E6 cells with a value of EC₅₀>20 micromolar.

Compound 49 (BC-742B) is the structure of Formula 1 in Table 2 in which A is a nitrile (a), R₁ is the 2-pyridone (1e), R₂ is a L-proline; R₃ is N-acetyl-L-valine; Compound 49 inhibits 3CL pro at Kᵢ*=143±3 nM.

While compound 42 (BC-666) is a poor inhibitor of SARS-CoV-2 3CLpro (Kᵢ=860 nM; (Table 2), the P₁-2-pyridone comprises a sidechain of unprecedented structure that is accommodated by the highly selective Si sub-site of CoV-2 3CLpro. It exhibited no activity as an anti-CoV-2 agent at concentrations of ≤10 μM. Other compounds (43-48) which also contain the P₁-2-pyridone sidechain are good inhibitors of 3CLpro, indicating that the choice of the P₃ and P₂ is important for binding to 3CLpro.

Compound 42 is an exceptionally potent inhibitor of human cathepsin L and cruzain (Kᵢ=2 nM for both enzymes).

Compounds 43 (LL-478) and 44 (LL-482) both contain the P₁-2-pyridone group and an aldehyde warhead, with a P₂ cyclohexyl-alanyl (Cha) sidechain, a P₃ valinyl sidechain, and, respectively, an N-benzyloxycarbonyl (Cbz) or an N-acetyl group at the N-termina. LL-478 is a potent inhibitor of 3CLpro (Kᵢ=9 nM), a good inhibitor of human cathepsin L (Kᵢ=50 nM), and is an active anti-CoV-2 agent (EC₅₀=5 μM) in infected A549/ACE2 cells. LL-482 was a weaker inhibitor of both 3CLpro (Kᵢ=60 nM) and cathepsin L (Kᵢ=140 nM).

Compound 45 (BC-671) is a dipeptide that contains the P₁-2-pyridone group and an aldehyde warhead, with a P₂ cyclohexyl-alanyl (Cha) sidechain, and a carbonyl-2-indoyl group as the N-terminus. It inhibits 3CLpro and cathepsin L with Kᵢ=39 and 190 nM, respectively.

Compound 46 (BC-674) is identical to Compound 45 except that the P₂ cyclohexyl-alanyl (Cha) sidechain is replaced with a leucinyl group, which produces a more effective inhibitor of 3CLpro (Kᵢ=39 nM).

Compound 47 (LL-492) is identical to (BC-674) except that the P₂ cyclohexyl-alanyl (Cha) sidechain is replaced with a 4-nitro-benzyl group, which produces a less effective inhibitor of 3CLpro (Kᵢ=260 nM).

Compound 48 (LL-753A) is identical to (BC-674) except that the P₃ carbonyl-2-indoyl group is replaced with an N-acetyl-L-valine and the P₂ cyclohexyl-alanyl (Cha) sidechain is replaced with an L-proline group, which produces an inhibitor of 3CLpro (Kᵢ=1160 nM).

Compound 49 (LL-742A) is identical to (LL-753A) except that the aldehyde of A is replaced with a nitrile group, which produces an inhibitor of 3CLpro (Kᵢ=143 nM).

Data for existing aldehyde and masked aldehyde inhibitors of cruzain, human cathepsins L and B, SARS-CoV-2 are shown in Table 2, along with available anti-trypanosomal or anti-coronaviral activities.

Synthesis of Masked Aldehydes.

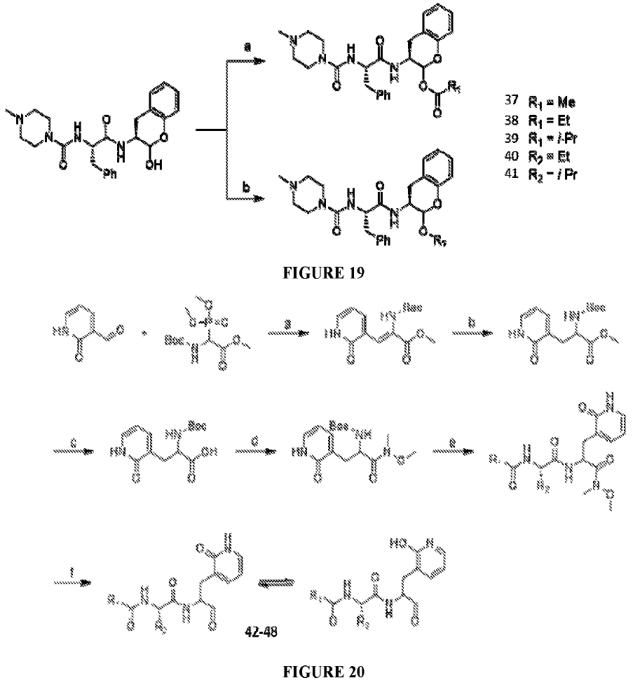

In FIG. 19 is delineated the synthesis of common SMAIs and their analogs starting from the HWE coupling reaction between salicylic aldehydes bearing different 5-substituents and (±)-Boc-α-phosphonoglycine trimethyl ester under basic conditions (step a). The double bond of product is reduced by Pd/C-catalyzed hydrogenation (step b), followed by removal of the Boc protecting group to give the key intermediate (step c), a substituted ortho-tyrosine methyl ester. This intermediate was coupled to different peptidomimetic scaffolds using T3P, yielding the methyl ester of the “full” inhibitor (step d). At this stage, the phenol group was protected as it was relatively acidic, and might affect subsequent reactions. The tert-butyldimethylsilyl (TBS) group was selected as the protecting group (step e) due to its stability and mild deprotection condition. Methoxy group was used for making compound 5 but not for protection as its removal was inefficient and demanded harsh conditions (step f). The TBS protection proceeded smoothly with the use of imidazole as base and catalyst. The protected product was treated with sodium borohydride to reduce the methyl ester to a primary alcohol (step g), which was further oxidized using Dess-Martin periodinane to form the aldehyde (step h).

Finally, removal of the TBS group by tetra-n-butylammonium fluoride (TBAF) rapidly generated the cyclic lactol compound (step i). While the scaffold Cbz-Phe-OH was commercially available, NMePip-Phe-OH was synthesized in lab. The amide coupling product between phenylalanine benzyl ester with 4-methylpiperazine-1-carbonyl chloride (step j) underwent Pd/C-catalyzed hydrogenolysis to produce the NMePip-Phe-OH. This acid was also used to prepare the “locked” cyclic compound 3. The route to its P₁ moiety started with the epoxidation of 1,4-dihydronaphthalene using mCPBA (step k). The epoxide ring was opened by nucleophilic attack of sodium azide (step 1) which was converted to free amine via catalytic hydrogenation.

Pro-drugs of Masked Aldehydes. Derivatization of the hydroxyl group of compound 36 included O-acylation and O-alkylation (FIG. 20). The O-acylation was performed by reacting 36 with the corresponding acetic/propionic/isobutyric anhydride using catalytic DMAP (step a). The O-alkylation was carried out by reacting 36 with corresponding ethanol/isopropanol using boron trifluoride etherate (BF₃OEt₂) as the Lewis acid catalyst (step b). On account of the anomeric carbon, SMAIs and their derivatives have two diastereomers which exist in equilibrium and cannot be isolated by flash column chromatography or preparative HPLC.

The preparation of 2-pyridone-based inhibitors for 3CL^{pro} began with 2-oxo-1,2-dihydropyridine-3-carbaldehyde instead of salicylaldehyde (FIG. 21). Because the 2-pyridone was fixed as P₁ moiety, its Weinreb amide was prepared on a relatively large scale prior to coupling to various scaffolds, allowing for an economical synthesis. To this end, after the HWE reaction (step a) and olefin hydrogenation (step b), the methyl ester was hydrolyzed under basic conditions (step c), and following reactions (step d-f) were similar to FIG. 19. Notably, this route does not require protection of the 2-hydroxyl group probably because 2-pyridone is the predominant form in reaction. In fact, we attempted to protect it with a TBS or benzyl group, but neither was successful.

### Example 3: Self-Masked Aldehyde Inhibitors: A Novel Strategy for Inhibiting Cysteine Proteases

Cysteine proteases comprise an important class of drug targets, especially for infectious diseases such as Chagas disease (cruzain) and COVID-19 (3CL protease, cathepsin L). Peptide aldehydes have proven to be potent inhibitors for all of these proteases. However, the intrinsic, high electrophilicity of the aldehyde group is associated with safety concerns and metabolic instability, limiting the use of aldehyde inhibitors as drugs. We have developed a novel class of self-masked aldehyde inhibitors (SMAIs) for cruzain, the major cysteine protease of the causative agent of Chagas disease-Trypanosoma cruzi. These SMAIs exerted potent, reversible inhibition of cruzain (Kᵢ*=18-350 nM) while apparently protecting the free aldehyde in cell-based assays. We synthesized prodrugs of the SMAIs that could potentially improve their pharmacokinetic properties. We also elucidated the kinetic and chemical mechanism of SMAIs, and applied this strategy to the design of anti-SARS-CoV-2 inhibitors.

### Introduction

Cysteine proteases are prospective drug targets for a variety of diseases caused by viruses (e.g., picornaviruses and coronaviruses),^{1,2} bacteria (e.g., Salmonella and Listeria),³ and particularly, many species of parasitic protozoa.⁴ One of these, Trypanosoma cruzi (T. cruzi), is the causative agent of Chagas disease, which affects an estimated 6-7 million people worldwide.⁵ During the life cycle of T. cruzi, its major cysteine protease, known as cruzain, plays a multifaceted role involving nutrient processing, morphological transformation, invasion into host cells, and evasion of the immune response.⁶ Consequently, cruzain has been widely studied, and also serves as a paradigm for inhibitor design for other cysteine proteases.

Substrate-analogue peptidomimetic compounds containing a suitable electrophilic warhead comprise the most common classes of inhibitors and inactivators of cruzain, as well as other cysteine proteases.⁷ A notable example is K777 (or K11777), a dipeptide analog containing a vinyl sulfone warhead, which undergoes irreversible thia-Michael addition to the active-site Cys₂₅ of cruzain,⁶ and other cysteine proteases including cathepsins K, L, and B (FIG. 21A).⁸ However, irreversible binding of small-molecules to protein targets is associated with idiosyncratic immunotoxicity,⁹ especially for drugs which require lengthy regimens. Accordingly, an emerging trend is the development of reversible covalent inhibitors which provide potent inhibition of the desired target, but will not permanently inactivate off-target proteins.¹⁰ Among the reversible covalent warheads explored for cruzain to date, an aldehyde group often, if not always, affords outstanding inhibitory potency compared to α-keto amides, nitriles, oximes, heterocycles, etc.¹¹⁻¹⁴ On the other hand, the inherent electrophilicity of aldehydes contributes to their rapid metabolism, immunotoxicities, and untoward drug-drug interactions,¹⁵ which have all but disqualified their use in drug design. Theoretically, these issues can be mitigated by confining the exposure of the aldehyde solely to the germane protein target while eschewing off-target proteins; however, such approaches have been challenging to implement.⁹

An alternative approach to incorporate aldehydes into drug design is to mask the aldehyde using another functional group. The anti-malarial aldehyde inhibitor of falcipain PG3b was “masked” by an 1,2,4-trioxolane structure (FIG. 21B), resulting in the prodrug PG4b from which the active aldehyde is liberated upon reaction with Fe(II) inside the vacuoles of the plasmodial parasite.¹⁶ Unfortunately, this clever masked aldehyde resulted in toxicity to the host cells, apparently from the action of free radical byproduct(s). Its utility is also limited by the variable availability of Fe(II) in target cells. The calpain inhibitor MN3 is another type of masked aldehyde in which a homoserine at the P₁ position¹⁷ spontaneously forms a cyclic hemiacetal (FIG. 21C). Compared to the potent free aldehyde SJA6017 (with Leu at P₁, IC₅₀=22 nM), protection of the aldehyde in MN3 greatly improved transcorneal permeability despite lower potency against calpain (IC₅₀=880 nM).^{18, 19} Herein we describe an unprecedented class of self-masked aldehyde inhibitors (SMAIs) for cruzain, their kinetic and chemical mechanisms of reversible covalent inhibition, and their effects on trypanosomal infection in cell cultures. In addition, we developed prodrugs of these SMAIs to improve their metabolic stability in order to optimize their pharmacokinetic properties. We adapted this concept to develop a novel inhibitor for the main cysteine protease (3CL protease) of SARS-CoV-2.

Results

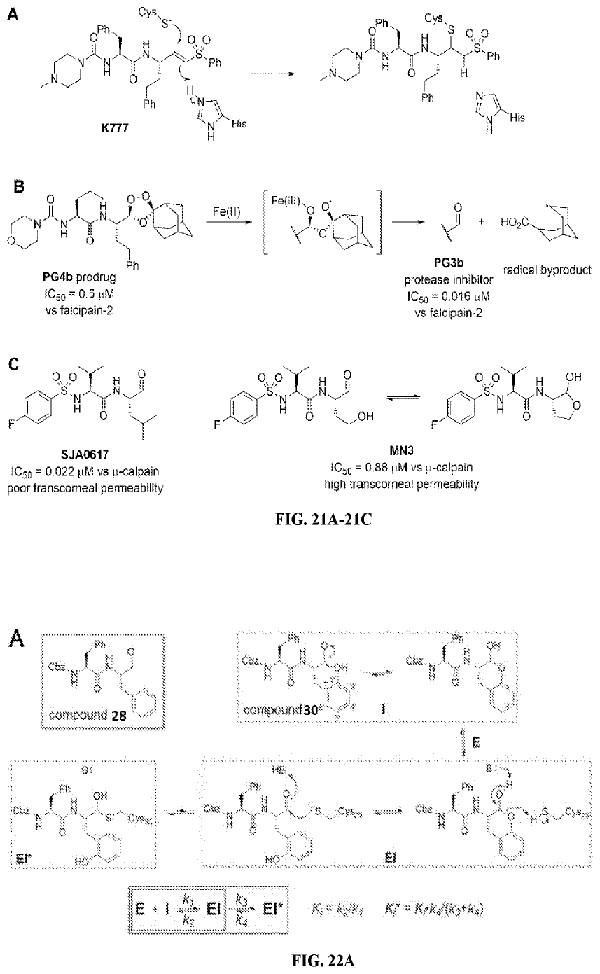

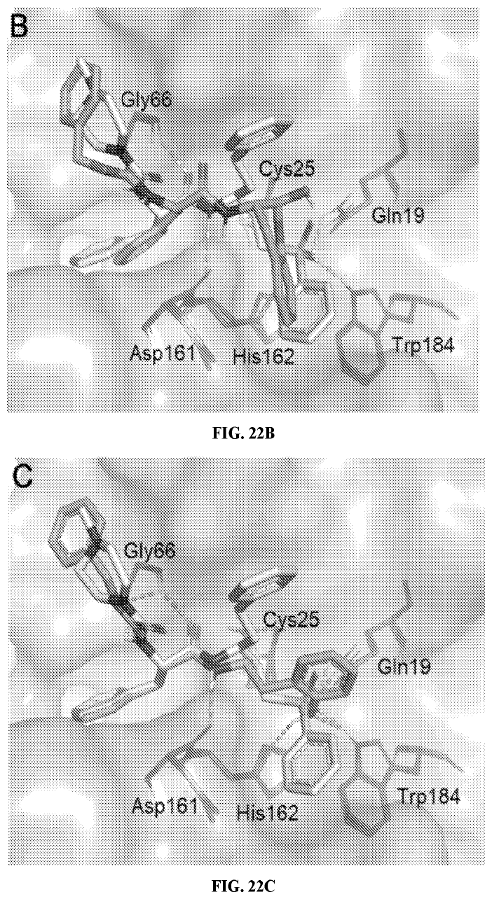

Design of SMAIs. The dipeptide aldehyde 28 (Cbz-Phe-Phe-H, FIG. 22A) was first prepared for use as an intermediate in the synthesis of other protease inhibitors, and proved to be an extraordinarily potent inhibitor of cruzain, as discussed below. In order to modify its P₁ structure to afford a self-masked aldehyde, we added a hydroxyl group to the 2′ position of the phenyl ring, converting the P₁ group to an ortho-tyrosine (30, Cbz-Phe-o-Tyr-H). The oxygen of the phenol group is in close proximity to the aldehydic carbon permitting a nucleophilic addition reaction to occur, producing a cyclic hemiacetal (δ-lactol) similar to the γ-lactol found in calpain inhibitor MN3.¹⁹ We anticipated that a SMAI would remain “locked” in its δ-lactol form before binding to cruzain, after which enzyme catalysis would elaborate the free aldehyde, followed by formation of a hemithioacetal adduct with cruzain (FIG. 22A). If true, then the potential advantages of SMAI include: (a) the intramolecular nature of the δ-lactol likely provides sustained protection of the aldehyde outside of an enzyme active site; (b) compared to the 1,2,3-trioxolane masked aldehyde,¹⁶ the cleavage of the hemiacetal does not produce reactive byproducts; and (c) the introduced hydroxyl group on the o-tyrosine sidechain is small enough so as to impose minimal perturbation of the original binding mode of the parent aldehyde 28.

The first question arising from our hypothesis is: could the lactol form of 30 bind to cruzain and remain closed? To preliminarily address this question, we employed molecular modeling using docking methods conforming to the binding of compound 30 in both its noncovalent, lactol form (FIG. 22B), or as its free aldehyde form which was docked as a hemithioacetal adduct with the catalytic Cys₂₅ (FIG. 22C). The covalent adduct of 30 is predicted to bind in a manner similar to that of the covalent inactivator K777 as seen in the cruzain-K777 co-crystal structure.²⁰ As expected, the phenoxy substituent of the covalently-bound inhibitor 30 is well tolerated in the cruzain active site, and may form hydrogen bonds with Gln₁₉, His₁₆₂, or Trp₁₈₄, similar to the sulfone oxygens of K777. Although the Cbz-Phe group in FIG. 22B adopts a similar orientation as the opened form (FIG. 22C), the lactol displays a puckered conformation in the active site. The bound lactol has a poorer binding free energy (ΔG_{predicted}=−5.08 kcal/mol) than that of the hemithioacetal (ΔG_{predicted}=−7.24 kcal/mol), apparently owing to the bicyclic lactol moiety. One may infer that the recognition of the Cbz-Phe scaffold of 30 by cruzain assists in the binding of the lactol group, and orients it for enzyme-catalyzed ring-opening to yield the high-affinity aldehyde.

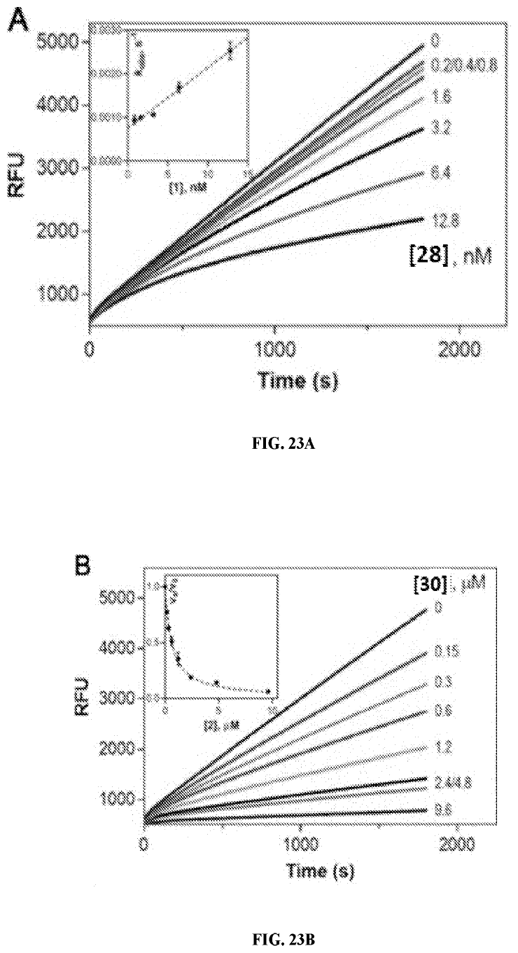

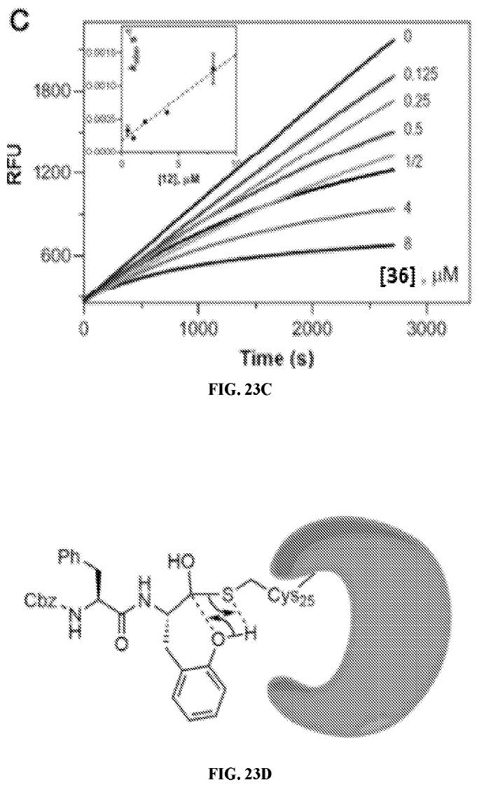

Kinetic Analysis. A series of SMAIs and related compounds 28-36, and 50-52 were prepared and evaluated as inhibitors of cruzain (Table 7). Time-course data for inhibition of cruzain by many of these compounds conformed to the kinetic scheme shown in FIG. 22A, that is, initiation of reaction by adding enzyme to substrate and inhibitor led to curvilinear time courses (FIG. 23A, C), in which reaction rates demonstrably decreased as the EI complex (characterized by Kᵢ) progressed to the tighter EI* complex (characterized by Kᵢ*). The observation of time-dependence may or may not indicate that covalent bond formation has occurred, but it does reflect that Kᵢ*<Kᵢ, arising from either the formation of a hemithioacetal between enzyme and inhibitor, or a slow isomerization step of EI to EI* not involving covalent bond formation.

TABLE 7
Inhibition data of SMAIs and related compounds

| Compound | Structure | | Cruzain Inhibition |
|---|---|---|---|
| Number | P₃-P₂ | P₁ | Kᵢ* (nM)^{a} |
| 28 | Chz-Phe- | 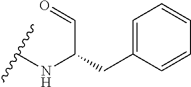 [SMILES: *N[C@H](C=O)Cc1ccccc1] | 0.44 ± 0.02 |
| 30 | Cbz-Phe- | 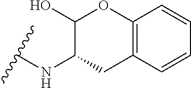 | 49 ± 2 |
| 50^{b} | NMePip-Phe- | 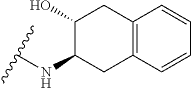 [SMILES: *N[C@@H]1Cc2ccccc2C[C@H]1O] | >100,000 |
| 51 | Cbz-Phe- | 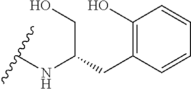 [SMILES: *N[C@H](CO)Cc1ccccc1O] | >10,000 |
| 29 | Cbz-Phe- | 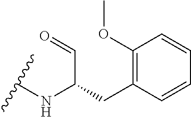 [SMILES: *N[C@H](C=O)Cc1ccccc1OC] | 22 ± 2 |
| 31 | Cbz-Phe- | 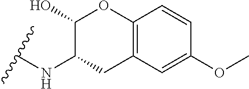 [SMILES: *N[C@H]1Cc2cc(OC)ccc2O[C@H]1O] | 350 ± 30 |
| 32 | Cbz-Phe- | 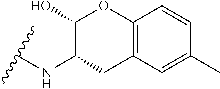 [SMILES: *N[C@H]1Cc2cc(C)ccc2O[C@H]1O] | 103 ± 5 |
| 33 | Chz-Phe- | 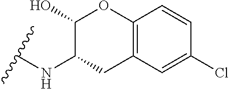 [SMILES: *N[C@H]1Cc2cc(Cl)ccc2O[C@H]1O] | 74 ± 10 |
| 34 | Chr-Phe- | 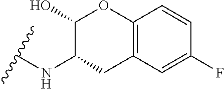 [SMILES: *N[C@H]1Cc2cc(F)ccc2O[C@H]1O] | 48 ± 2 |
| 35 | Cbz-Phe- | 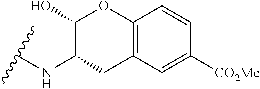 [SMILES: *N[C@H]1Cc2cc(C(=O)OC)ccc2O[C@H]1O] | 18 ± 0.5 |
| 52 | NMePip-Phe- | 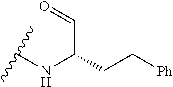 [SMILES: *N[C@H](C=O)CCc1ccccc1] | 0.5 ± 0.2 |
| 36 | NMePip-Phe- | 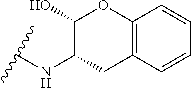 [SMILES: *N[C@H]1Cc2ccccc2O[C@H]1O] | 47 ± 2 |
| K777 | NMePip-Phe- | 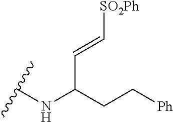 | 0.2^{c} |

ªTo the assay buffer (50 mM MES, 50 mM TAPSO, 1 mM CHAPS, 1 mM Na₂EDTA, 5 mM DTT, 10% DMSO (v/v), and pH 7.5) containing variable concentrations of inhibitors (0.02-20 μM) and 10 μM of substrate Cbz-Phe-Arg-AMC (Kₘ = 0.9 μM) was added cruzain (final concentration of 0.2 nM), and changes in fluorescence were monitored for 20-60 min. Values of Kᵢ* (n ≥ 2) were obtained as described. ^{b}50 contains two diastereomeric anti products, and only one is shown here; ^{c}Reported as an apparent IC₅₀ in ref 48.

We first evaluated cruzain inhibition by time-course data containing fixed concentrations of substrate and variable concentrations of inhibitors, for which reactions were initiated by the addition of enzyme. Inhibition of cruzain by the free aldehyde 28 was characterized by downward-concave curvilinear time courses, often referred to as “burst kinetics” (FIG. 23A), indicative of time-dependent inhibition for which equilibrium between cruzain and 28 was slowly established over 30 minutes. Each curve was fitted to eq. 1 and the resulting values of k_{obs} were replotted vs. inhibitor concentration [1] (FIG. 23A, inset). The replot was best fitted to eq. 3, implying a lack of saturation of the EI complex by 28, for which Kᵢ>>Kᵢ*. The unimolecular rate constant for conversion of EI* back to EI was extremely slow [k₄=(7.6±0.6)×10⁻⁴ s⁻¹]. The low value of k₄ is likely the reason for the slow onset of inhibition and potency of aldehyde 28.

Unlike aldehyde 28, inhibition of cruzain by 30 exhibited almost linear time courses with only slight curvature observed at early stages (FIG. 23B), as was also observed for SMAIs 31-35 (all containing the Cbz-Phe-scaffold, FIG. S1A in Supporting Information). The nominal burst phase was only observed within the first 1-2 min, after which an apparent steady-state reaction was established, indicating a significantly faster rate (greater k₄) for conversion of EI* back to EI than for compound 28. This difference suggests that, compared to a relatively stable hemithioacetal intermediate cruzain-28 species, the reverse reaction of this intermediate for a SMAI is likely facilitated by the attack of its phenoxy group on the hemithioacetal (FIG. 23D). To characterize these inhibitors, we obtained steady-state rates of vₛ and v₀ at assay times ≥20 min. For instance, upon plotting vₛ/v₀ vs. [30] (FIG. 23B, inset) and fitting using eq. 4, we obtained a value of the overall inhibition constant (Kᵢ*) of 49±2 nM. For the free aldehyde 52 and the SMAI 36, both of which incorporated the N-methylpiperazinyl-Phe scaffold (NMePip-Phe) as found in K777, the ratio of their activities [Kᵢ*(36)/Ki*(52)=94] as cruzain inhibitors was almost identical to that of their Cbz-Phe-containing counterparts 28 and 30 [Kᵢ*(30)/Kᵢ*(28)=110]. Similar to aldehyde 28, slow onset of inhibition was observed for 52 (Figure S1B). Surprisingly, unlike SMAIs containing Cbz-Phe-scaffold, 36 also exhibited time-dependent inhibition (FIG. 23C).

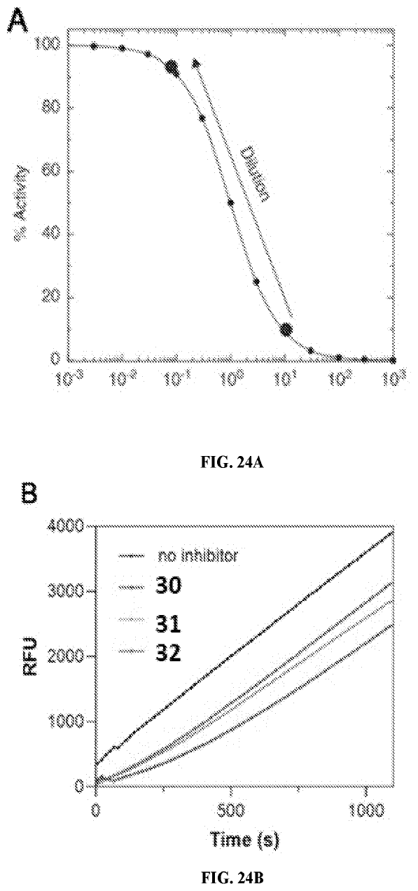

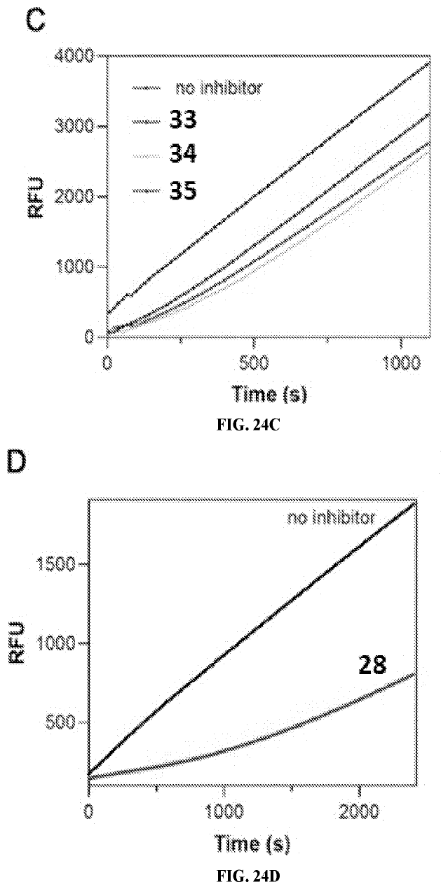

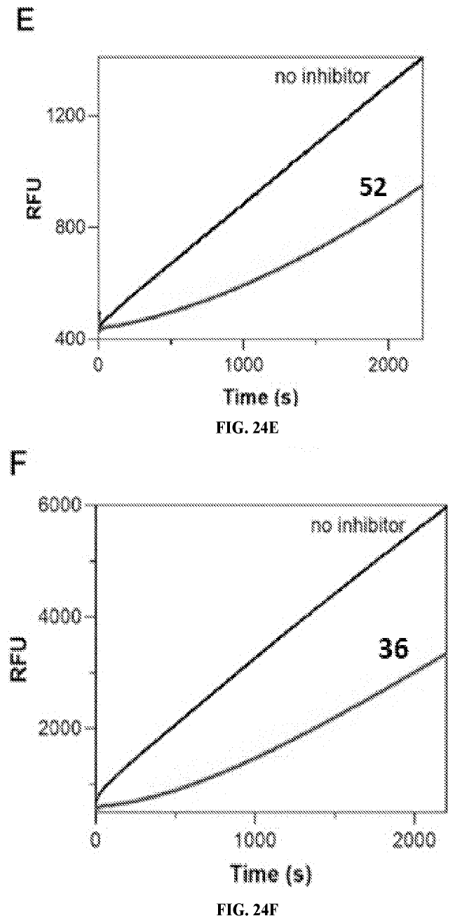

To explore the reversibility of cruzain inhibition by these compounds, we first pre-incubated each inhibitor with cruzain, followed by a 100-fold rapid dilution accompanying the addition of substrate (FIG. 24A). For inhibitors 30 and 31-35 (FIG. 24B, C), 91% recovery of cruzain activity was observed over the course of minutes, wherein the residual concentration of inhibitor was 0.1×Kᵢ*^{app}, capable of affording 9% inhibition, thereby demonstrating that binding of these inhibitors was fully reversible. In comparison, the activity of cruzain pre-incubated with aldehydes 28, 52, or SMAI 36 was restored at much slower rates (FIG. 24D-24F). After fitting these “lag” time courses to eq. 1, the calculated residence times (τ=1/k_{obs}) for compounds 30 and 31-35 ranged from 2.5 to 5.5 min, whereas 28, 52, and 36 exhibited significantly longer residence times of 52, 45, and 26 min, respectively (Table Si). While longer residence times were expected for aldehydes 28 and 52, compound 36 was the only SMAI to effect significant time-dependent inhibition as evidenced by the observation of both burst and lag kinetic time courses, and the rate of conversion of the EI* complex to EI was comparable to that of aldehyde 52. Considering that compound 36 was equipotent to 30, one could infer that the peptidomimetic scaffold of 36 might also affect the adduct formation so that k₃/k₄ remained nearly unchanged for both the SMAI and the free aldehyde. Therefore, a covalent docking study was conducted for 36, which predicted a binding free energy (ΔG_{predicted}=−7.15 kcal/mol) that was close to compound 30 (ΔG_{predicted}=−7.24 kcal/mol) as formerly mentioned, which was in accordance with their similar Kᵢ* values. Interestingly, an intramolecular hydrogen bond between P₁ phenol group and P₃ carbonyl group has dragged the two moieties away from their binding subsites (FIG. S2). The consequence of this conformation is that the phenol group in 36 is less likely to take part in the conversion of EI* back to EI (FIG. 23D), leading to slower dissociation from cruzain and its unique lag kinetics compared to 30.

Structure-Activity Relationships of SMAIs. Inhibition constants for all compounds prepared for this study are shown in Table 7. The equipotency of SMAIs 30 and 36 at −50 nM suggests that their different P₃ sidechains have no impact on their inhibition. We prepared compound 50, an analog of SMAI 36 in which its ether oxygen was replaced with a methylene group, and the resulting compound containing the stable tetrahydro-naphthol did not inhibit cruzain at ≤100 μM, despite its similarity to the chroman-2-ol group of the SMAIs. Substitution of the aldehyde of compound 30 with a primary alcohol also resulted in the poor inhibitor 51, which corroborates the importance of the formation of carbon-sulfur bond in the cruzain-SMAI complex. In the free aldehyde 29, the phenoxy group was methylated, affording an inhibitor that is 50-fold less active than aldehyde 28, implying that a bulkier 2′ substituent sterically hinders the formation of the hemithioacetal.

Another potential feature of SMAIs is the ability to tune the reactivity of the phenol group by installing substituents on the benzene ring. We introduced several substituents at the 5′ position of the ortho-tyrosine, including electron-donating groups (i.e., —OMe, -Me), weak electron-withdrawing groups (i.e., —F, —Cl), and moderate-to-strong electron withdrawing group (i.e., —CO₂Me). The electron-donating substituents on 31 (Kᵢ*=350 nM) and 32 (Kᵢ*=100 nM) appear to undermine their potency, as these inhibitors bind, respectively, 7- and 20-fold more weakly that un-substituted SMAI 30. The stronger electron-withdrawing capability of the ester substituent (35; Kᵢ*=18 nM) provided a 3-fold increase in potency vs. 30, suggesting that the 5′-methyl ester facilitates the opening of the lactol ring. In addition to the electronic effect, a steric effect is likely operative. The methoxy group of compound 31 is bulkier than the methyl group of 32, which may contribute to its over 3-fold lower activity. Chlorine (0.79 Å, 33) has a slightly larger radius than fluorine (0.42 Å, 34), and fluorine is similar in size to a hydrogen (0.53 Å, 30) (note: the C—F is longer than the C—H bond so that, at that increased length, the fluorine is effectively the same radius as hydrogen),²¹ consistent with the differences between their Kᵢ* values. It is interesting that a steric effect apparently dictates the differences in potency between compounds 33 and 34, probably because fluorine and chlorine are only weakly electron-withdrawing. The methyl ester of 35, while comprising the bulkiest substituent among this series of compounds, provided the SMAI of the highest potency in this study, apparently because the electron-withdrawing effect afforded by this large substituent overcomes any steric effect. These results suggested that appropriate substitution of the P₁ phenyl ring in SMAIs could optimize their potencies, and further, may affect the stability of enzyme-bound lactol.

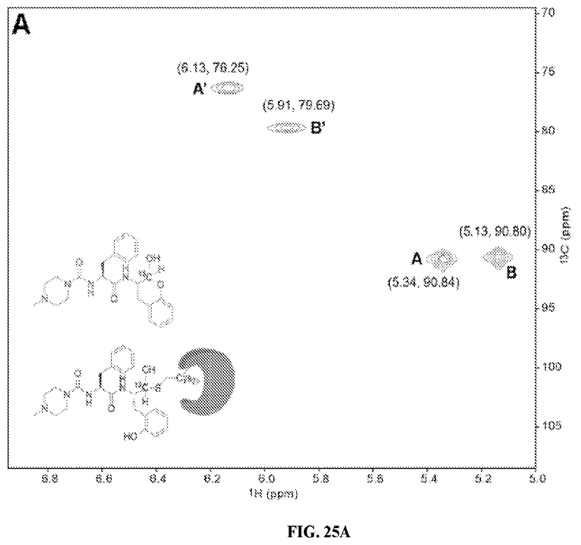

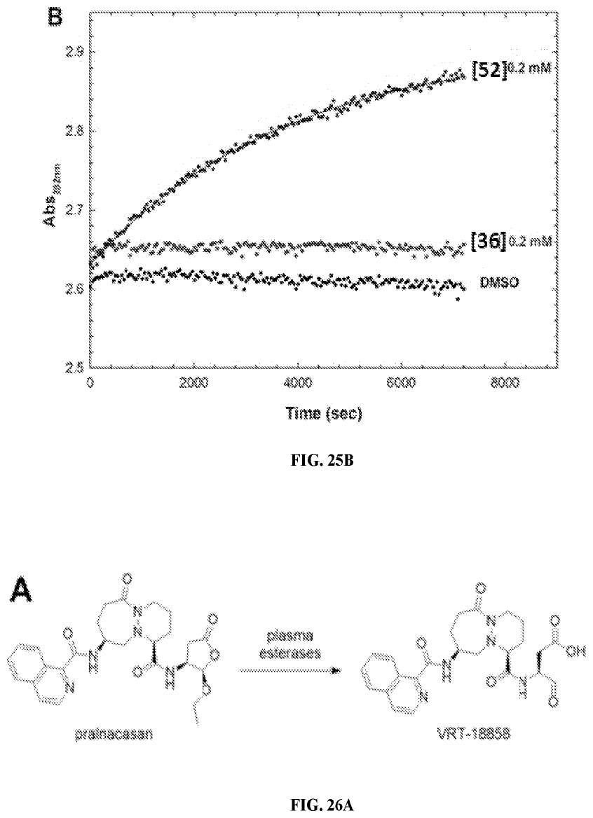

Chemical Mechanism of SMAI Binding to Cruzain. Next, we prepared compound 36 in which the aldehydic carbon was ¹³C-labeled, to be used in a study of ¹H-¹³C heteronuclear single quantum correlation (HSQC) NMR to elucidate the chemical specie(s) of 36 that are bound to cruzain. First, 0.4 mM of ¹³C-labeled 36 in phosphate buffer (pH 7.5) was analyzed by HSQC NMR. No discernable peak was present at δ(¹³C) >180 ppm, ruling out the existence of minute concentrations of free aldehyde in this aqueous solution (see Supporting Information for full-view spectrum).²² The two salient peaks, A and B, occurring near δ(¹³C) 90.8 ppm, were consistent with the signal of a carbon occurring in a hemiacetal (FIG. 25A, red). These signals could not be assigned as an aldehyde hydrate in view of the fact that analysis by LC-MS displayed a molecular ion peak corresponding to the hemiacetal but not the hydrate (FIG. S3). We propose that peaks A and B are associated with two δ-lactol anomers spontaneously generated during lactol formation. The ratio of anomer A to anomer B is 1:1.32 based on peak volumes (Table S2), yet the exact stereochemistry cannot be assigned at this point.

To the inhibitor sample was added an approximately stoichiometric amount of cruzain, and a new spectrum was acquired after 1 h (FIG. 25A, blue). The apparent hemiacetal peak B was eliminated while a trace of peak A remained, which indicates a slight preference of cruzain for anomer B over A, in consideration of the ratio of integrated volumes (A:B=1:1.32). The fact that the cross peaks shifted considerably upon addition of cruzain is strongly indicative of hemithioacetal formation. This is because the new signals observed at δ(¹³C) 76.2 and 79.7 ppm are very similar to those observed for the formation of a hemithioacetal adduct between N-acetyl-L-phenylalanine-[2-¹³C]glycinal and papain (δ(¹³C) 75.1 ppm).²³ The broadening of peaks A′ and B′ also suggested a protein-bound ligand, due to reduced tumbling and the increased relaxation time of the cruzain-bound ¹³C-labeled 36. In addition, mass spectrometric experiments of cruzain pre-incubated with 36 as well as three SMAIs with Cbz-Phe-scaffold (30, 32, and 34) clearly revealed the formation of corresponding enzyme-inhibitor adducts as told by the mass changes relative to apo-cruzain (FIG. S4), serving as the orthogonal evidence of covalent bond formation.

As phenylhydrazine readily forms phenylhydrazone adducts with aldehydes, we treated 0.2 mM of aldehyde 52 and SMAI 36 with 1 mM phenylhydrazine in the same buffer (lacking DTT) used in the NMR study (FIG. 25B) to determine the fraction of free aldehyde found in 36. While aldehyde 52 was rapidly, and apparently, completely converted to phenylhydrazone, 36 remained intact after 2 hours. This finding showed that even an excess amount of phenylhydrazine cannot drive the equilibrium of 36 towards the formation of open-form aldehyde, which not only corroborated the chemical stability of lactol, but also indicated that the ring-opening is likely an enzyme-catalyzed process. Therefore, we conclude that SMAI 30 predominantly maintains its lactol form in aqueous solution in the absence of cruzain, while its binding to cruzain apparently promotes opening of its ring followed by the formation of a covalent bond with Cys25.

SMAIs Display Anti-Trypanosomal Activity. Subspecies of pathogenic Trypanosoma brucei have essential cysteine proteases (TbCatL, brucipain, or rhodesain) which are close homologs of cruzain and perform similar biological functions.²⁴ We evaluated our compounds in axenic cultures of the bloodstream forms (BSFs) of Trypanosoma brucei brucei (T. b. brucei) which are found in infected mammals (Table 8, Figure S5). The most active inhibitors for BSFs, 32 and 36, exhibited respective EC₅₀ values of 0.5 and 0.6 μM, which are more potent than the anti-trypanosomal drug, diminazene (Berenil®, EC₅₀=0.99 μM in T. b. brucei BSFs).²⁵ Notably, the free aldehyde 28, despite its 100-fold higher potency vₛ. cruzain, was equally or less potent in inhibiting T. b. brucei BSFs compared to the SMAIs. Considering that aldehyde 28 is nearly four orders of magnitude less effective vs. T. b. brucei than vs. purified cruzain (EC₅₀/Kᵢ*=7,500), while the average value of EC₅₀/Kᵢ* for all SMAIs is 74, one may speculate that the SMAIs are more accessible to its cellular target(s) than free aldehyde 28 in cell culture media. In this sense, masking of the active aldehyde within the SMAIs affords significant protection of its electrophilic group.

TABLE 8
Anti-Trypanosomal Activities of (Self-Masked) Aldehyde
Cruzain Inhibitors and Their Pro-Drugs^{a}

| | Cruzain | T. b. brucei | T. cruzi-Infected |
|---|---|---|---|
| Compound | Inhibition Kᵢ* | BSFs EC₅₀ | Cardiomyoblasts |
| Number | (nM) | (μM)^{a} | EC₅₀ (μM)^{a} |
| 28 | 0.44 ± 0.02 | 3.3 ± 2.1 | 0.5 ± 0.4 |
| 30 | 49 ± 2 | 6.8 ± 1.1 | 3.0 ± 0.9 |
| 31 | 350 ± 30 | 2.6 ± 1.1 | ND^{d} |
| 32 | 103 ± 5 | 0.5 ± 0.2 | 5.7 ± 0.6 |
| 33 | 74 ± 10 | 5.8 ± 3.4 | ND |
| 34 | 48 ± 2 | 2.7 ± 0.2 | 3.5 ± 0.6 |
| 25 | 18 ± 0.5 | 4.0 ± 0.2 | 3.5 ± 0.3 |
| 36 | 47 ± 2 | 0.6 ± 0.1 | ~30 |
| 37^{b} | ~1,000 | 3.7 ± 0.2 | 13 ± 2 |
| 38^{b} | >5,000 | 3.1 ± 0.2 | ND |
| 39^{b} | >5,000 | 4.0 ± 0.2 | 14 ± 6 |
| 40^{b} | >5,000 | 12 ± 0.1 | ND |
| 41^{b} | >5,000 | 14 ± 0.9 | ND |
| K777 | 0.2^{c} | 0.09 ± 0.06 | 0.038 ± 0.009 |

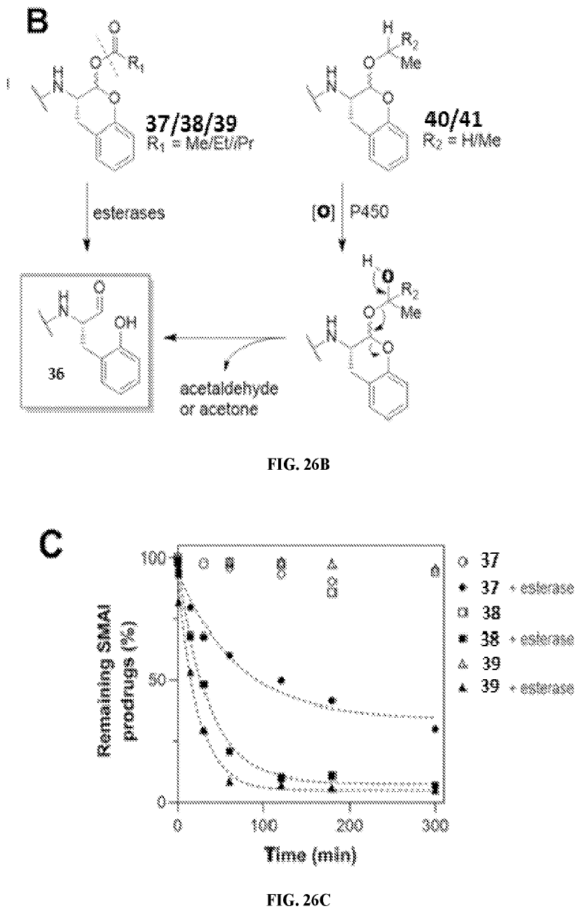

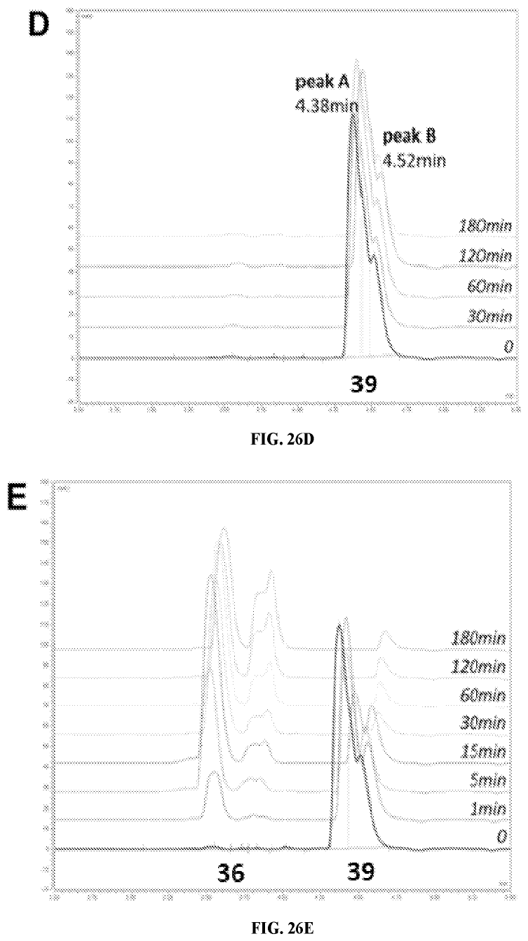

^{a}Trypanocidal activities of compounds in axenic cultures of T. b. brucei BSFs and in cardiomyoblasts infected with T. cruzi were all measured as EC₅₀ values (half maximal effective concentration), along with SEMs from at least two replicates. Typical test concentrations of inhibitors were 0.5-20 μM for T. b. brucei BSFs assays and 0.02-10 μM for T. cruzi infected cardiomyoblasts assays; ^{b}Structures of 37-41 are shown in FIG. 26B; ^{c}Reported as an apparent IC₅₀ in reference 48; ^{d}ND, not determined.

Additionally, aldehyde 28, SMAIs 30, 32, 34, 35, 36, and pro-drug SMAIs 37 and 39 (the latter two compounds as discussed in the next section) were analyzed in a murine cardiomyoblast model of T. cruzi infection, in which the disease-relevant amastigote forms of the parasite were evaluated (Table 8,). The parent aldehyde inhibitor 28 was the most potent inhibitor in this assay (EC₅₀=0.5 μM), and was 6-fold more active in this cellular model of T. cruzi rather than in the axenic cultures of T. b. brucei. SMAIs 30, 32, 34, and 35 killed T. cruzi at EC₅₀ values ranging from 3.0-5.7 μM, while, surprisingly, 36 was much weaker with an EC₅₀˜ 30 μM, despite the fact that it exerted a value of EC₅₀=0.6 μM in T. b. brucei. The anti-chagasic drug, benznidazole, was active in this assay at which EC₅₀=4±2 μM, which is similar in potency to the SMAIs. Apart from 36, these SMAIs were essentially equipotent in axenic cultures of T. b. brucei BSFs and T. cruzi-infected cardiomyoblasts, suggesting they target a cysteine protease homologue of cruzain in T. b. brucei. Additionally, they exerted no apparent toxicity against the host cardiomyoblasts at up to 10 μM, whereas both aldehyde 28 and K777 exhibited cytotoxicity at 10 mM (FIG. S6). Although free aldehyde 28 was the most potent inhibitor in this assay (EC₅₀=0.25±0.06 μM), it lost most of its activity considering its sub-nanomolar activity against cruzain. Accordingly, the self-masking of the aldehyde group in SMAIs provides the apparent delivery of these otherwise reactive compounds to trypanosomes harbored within mammalian cardiomyoblasts, with no apparent untoward effects on the host cells. These results encouraged progressing the SMAIs to pre-clinical analysis, as well as the development of prodrug forms of SMAIs.

Prodrugs of SMAIs. The pharmacokinetic evaluation of compound 36 in mice by intravenous administration indicated a short half-life arising from apparent first-pass metabolism (data not shown). Its analogue, K777 has superior pharmacokinetic properties to 36, and it was shown that the sites of oxidative metabolism on K777 were largely confined to the N-methylpiperazine ring, common to both inhibitors, and the homophenylalanyl sidechain.²⁶ Therefore, metabolism of the cyclic hemiacetal of 36 may be responsible for its rapid clearance in mice.

The cyclic α-keto-acetal prodrug for caspase-1, pralnacasan (VX-740), can be regarded as an SMAI, as it is rapidly converted to the active aldehyde inhibitor (VRT-18858) by plasma esterases via formation of a hemiacetal intermediate (FIG. 26A).²⁷ In kind we designed two types of derivatization of the hemiacetal hydroxyl group, that is, via O-acylation and O-alkylation (FIG. 26B). The O-acylated compounds should be hydrolyzed to compound 36 by the action of cellular esterases, which are ubiquitous in the ER lumen of mammalian tissues.²⁸ For the O-alkylated compounds, the enzymatic oxidation is likely to occur by action of liver cytochrome P450 enzymes, thereby elaborating a new hemiacetal that subsequently collapses to form compound 36.

To explore this prodrug approach, we prepared O-acylated compounds 37-39 and O-alkylated compounds 40 and 41 (Structures shown in FIG. 26B). These compounds exhibited negligible inhibition against cruzain, with the exception of 37, which weakly inhibited cruzain in a time-dependent fashion (Kᵢ*=1 μM, Figure S1C). Compounds 37-39 were treated with porcine esterase in buffer (pH 7.5), and the hydrolysis of these prodrugs was monitored by LC-MS (FIG. 26C). In control samples without esterase, these compounds largely remained intact. Compound 37 was an exception and degraded about 10% in buffer over 3 h, which likely explains the observed inhibition of cruzain by 37 due to the formation of 36. Upon addition of the esterase, compounds 37-39 were all converted to compound 36 at variable rates of reaction with respective half-lives of 48, 24, and 16 min (by fitting curves in FIG. 26C to eq. 6), and displayed a trend of increased hydrolysis with the increasing steric bulk of the acyl groups. A reasonable interpretation is that the main mammalian esterase, carboxylesterase-1 (CES1), has a preference for larger acyl groups.²⁸ Accordingly, modifying the acyl group of O-acylated compounds to obtain a suitable half-life can potentially overcome the apparent first-pass metabolism of 36. As observed for compound 39 (FIG. 26D, 26E), there are two peaks in the chromatograph that presumably correspond to the two anomers, but the conversion of one peak (A, t_{R}=4.38 min) to 36 is faster than the other (B, t_{R}=4.52 min). Peak A is largely eliminated after 30 min, while peak B, though it constitutes a smaller proportion of untreated compound, does not decompose completely even after 3 h, suggesting that the anomer in peak A is the more specific substrate for esterase. These results provide proof of concept that use of O-acylated prodrugs of SMAIs will provide a means to deliver these inhibitors in vivo.

Compound 36 was also modified to yield two mixed acetals 40 and 41 (FIG. 26B) to yield prodrugs that did not inhibit cruzain. Because cytochrome P450 forms 3A4, 2D6 and 2C9 together account for over 60% of drug-metabolizing P450 isoforms,²⁹ we used these enzymes to conduct in vitro assays to determine if 40 and 41 could be transformed to compound 36 as expected. Both compounds were incubated with different CYPs in the presence of a NADPH-regenerating system for up to 4 h. Unfortunately, no transformation to 36 was observed for either compound by any of the CYPs. However, the apparent stability vs. these purified P450s does not mean that (an)other microsomal oxidase(s) cannot release the active SMAIs from these prodrug forms.

These compounds were also tested in axenic cultures of T. b. brucei BSFs (Table 8). The pro-drug forms of compound 36, compounds 37 and 39, were considerably more potent than 36 in the infected cardiomyoblast assays: (Table 8, EC₅₀=13±2 μM and 14±6 μM, respectively). These encouraging results demonstrated that the O-acylated prodrug forms could well be converted to 36, presumably by a parasitic esterase. Interestingly, compounds 40 and 41 were able to kill the parasites in spite of lower activity (Kᵢ*>5 μM). Since treatment with P450s did not lead to transformation of these prodrugs, the data implied the existence of either a drug-metabolizing enzyme or another potential activating enzyme in T. b. brucei.

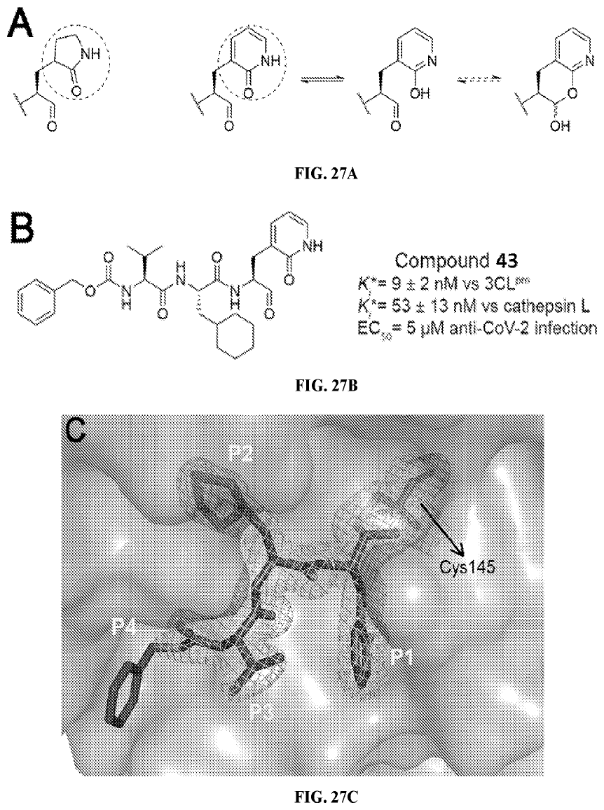

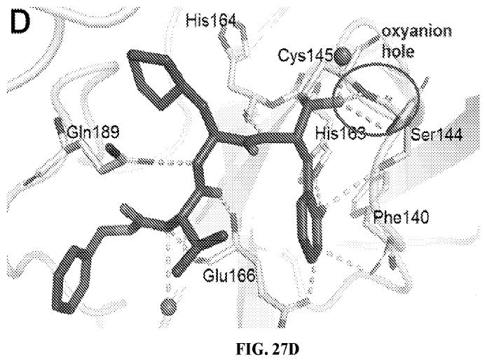

Application of SMAIs to SARS-CoV-2 3CL^{pro}. The SMAI strategy is not limited to cruzain, and may also find application to other cysteine proteases. To this end, we attempted to develop SMAIs for SARS-CoV-2 main protease (3CL^{pro}), a drug target for COVID-19.³⁰ Numerous inhibitors of the homologous cysteine proteases of SARS-CoV, MERS-CoV, and some picornaviruses, have shown different levels of inhibition against SARS-CoV-2. Peptidomimetic aldehyde inhibitors of 3CLpro contain a nearly-invariant 2-oxo-pyrrolidin-2-yl group (γ-lactam) as the P₁ side chain. We propose that 2-pyridone can act as a surrogate for the γ-lactam (FIG. 27A). Apart from their similarity in size and heteroatom substitution, the tautomerization of 2-pyridone towards 2-hydroxypyridine has been well characterized.³¹ Although the 2-pyridone tautomer is preferred in aqueous solution, the presence of the C-terminal aldehyde could well form a SMAI with the 2-hydroxypyridine.

A 2-pyridone compound 43 was synthesized (FIG. 27B), and the proton NMR of 43 in organic solvent confirmed the apparent absence of aldehyde, suggesting it forms a δ-lactol, though it is unclear what the exact species is in aqueous buffers. Compound 43 was such a potent inhibitor of 3CL^{pro} that its Kᵢ* (9 nM) was a result of apparent titration of the enzyme (40 nM used in the assay). It also demonstrated anti-CoV-2 activity (EC₅₀=5 μM) in SARS-CoV-2-infected A549/ACE2 cells (Table S3). Interestingly, 43 also inhibited human cathepsin L, which was recently shown to be essential to the penetrance of SARS-CoV-2 into mammalian cells, in that it catalyzes essential cleavage of the coronaviral Spike protein.^{8, 32} As a result, compound 43 has the potential to be a dual-acting inhibitor for two enzymes which are critical to the infection of human cells by SARS-CoV-2.

We also obtained a high-resolution (1.70 Å) crystal structure of SARS-CoV-2 3CL^{pro} in complex with 43. The well-defined electron density (FIG. 27C) confirmed the formation of a hemithioacetal of 43 with active-site Cys₁₄₅, and the resulting hydroxyl group of the hemithioacetal is stabilized by an oxyanion hole provided by Gly₁₄₃ and Cys₁₄₅ (FIG. 27D). The P₁ 2-pyridone establishes essential binding interactions with 3CL^{pro} analogous to that of the γ-lactam,³¹ in that the carbonyl oxygen of the 2-pyridone accepts two hydrogen bonds from His₁₆₃ and Ser₁₄₄, while the amide nitrogen acts as a hydrogen bond donor to Glu₁₆₆ and Phe₁₄₀. These interactions clearly demonstrate that the γ-lactam can be bioisosterically replaced by the 2-pyridone moiety which is a potential SMAI substructure. As with cruzain, 3CL^{pro} is apparently capable of catalyzing ring-opening of the putative masked aldehyde of 43.

Chemistry. The synthesis of SMAIs and their analogs involved the preparation of various P₁ building blocks and subsequent coupling with P₃-P₂ scaffolds (Scheme 1). A variety of 5-substituted salicylaldehydes were coupled with N-Boc-2-phosphonoglycine trimethyl ester in Horner-Wadsworth-Emmons (HWE) reactions. The formed double bond in S1a was reduced to afford the Boc-protected methyl ester of substituted ortho-tyrosine (S1b) as a mixture of enantiomers, which was then deprotected and reacted with the carboxylic acids of corresponding P₃-P₂ fragments, Cbz-Phe-OH or NMePip-Phe-OH. Cbz-Phe-OH was commercially available, while NMePip-Phe-OH (S1j) was simply prepared in two steps including amide coupling and hydrogenolytic cleavage of the benzyl group. The resulting S1d as well as subsequent intermediates were diastereomers that could be mostly separated on silica gel column. Since the phenol group is relatively reactive that may interfere with future reactions, a methyl group was originally used for its protection and eventually resulted in compound 5. In our effort to remove the methyl group, we experienced unwanted side reactions due to harsh conditions such as the use of BBr₃. Instead, a TBS group was employed as the protecting group. Next, the methyl ester of S1e was successively reduced to alcohol using NaBH₄ (S1f and 51), and oxidized to aldehyde using Dess-Martin periodinane (S1g). Finally, efficient removal of TBS by TBAF exposed the phenol group toward the reaction with aldehyde, leading to in situ formation of the lactol ring (S1h). It should be noted that each SMAI was a mixture of anomers that could be observed by HPLC or NMR (see Supporting Information). Since the spontaneous interconversion existed in between the anomers, it was neither necessary nor possible to chromatographically isolate them.

The P₁ building block of 50 was synthesized using a different strategy. 1,4-Dihydronaphthalene was subject to epoxidation (S1k), followed by nucleophilic attack by NaN₃ to produce S1l. The azide was then converted to amino group which was coupled with NMePip-Phe-OH to yield compound 50. Notably, 50 contains two diastereomeric anti products as the P₁ NH and OH are not on the same side of the ring. The preparation of free aldehyde 52 was similar to other SMAIs, except for that the latter required one more step for TBS removal.

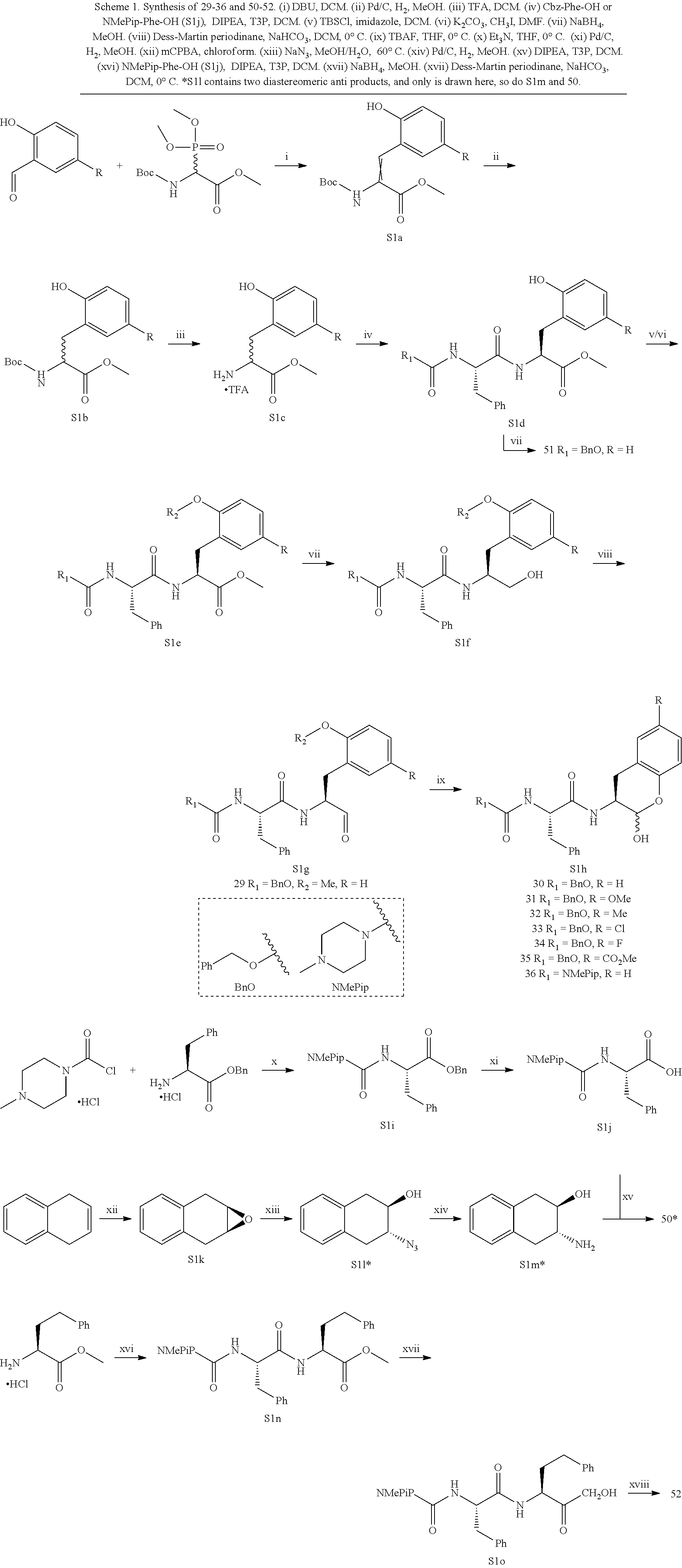

The derivatizations of the hydroxy group of 36 were summarized in Scheme 2. Acylation was achieved by reacting 36 with corresponding acid anhydrides under basic conditions (S2a); alkylation was performed by treating 36 with boron trifluoride etherate in presence of corresponding alcohols (S2b).

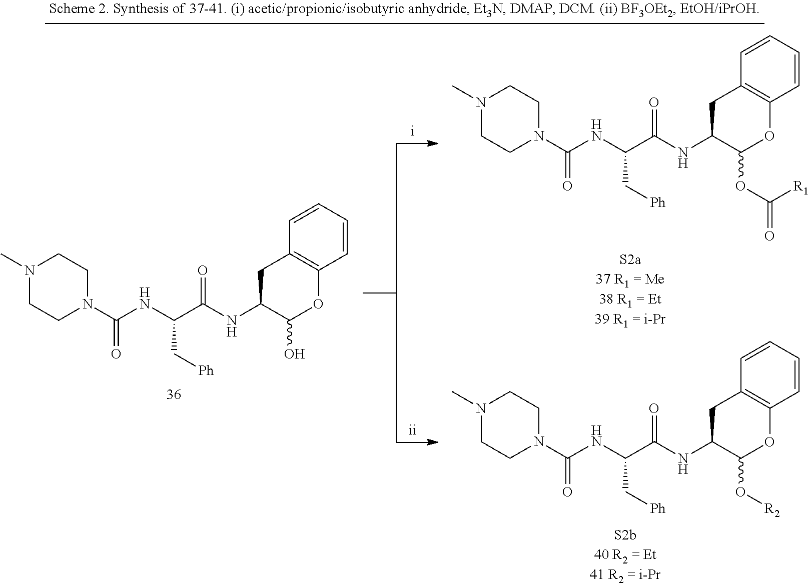

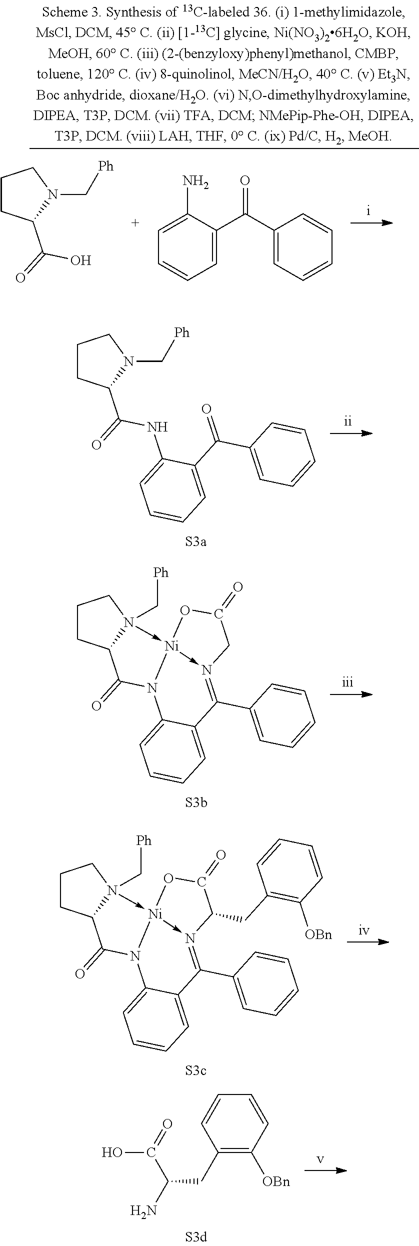

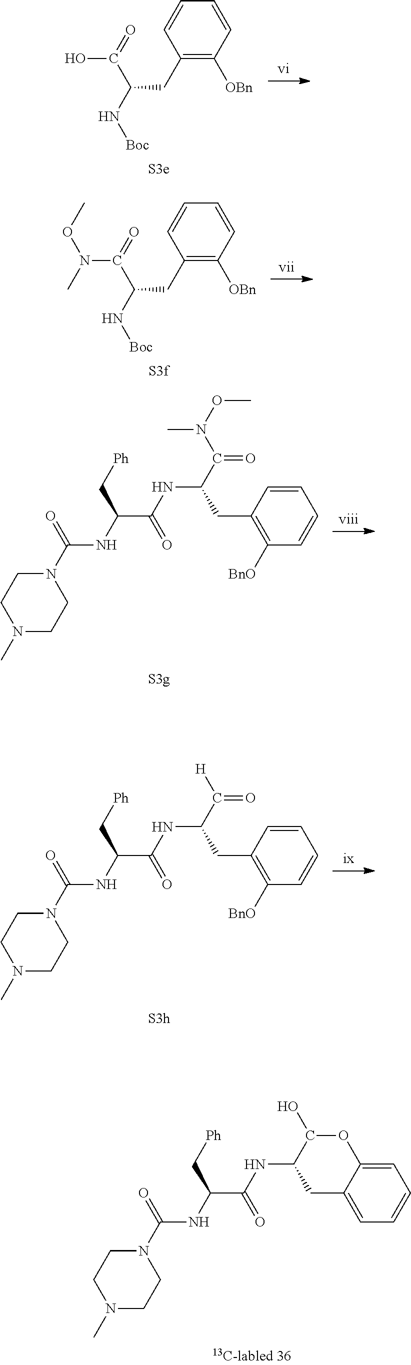

Because ¹³C-lableled N-Boc-2-phosphonoglycine trimethyl ester was not purchasable, we adopted a Ni(II) complex-based synthetic route for ¹³C-labeled 36 (Scheme 3).³³ A chiral auxiliary S3a (2-(N-benzylprolyl)aminobenzophenone, BPB) was synthesized and then treated with [1-¹³C] glycine and nickel(II) nitrate hexahydrate under strongly alkaline condition to form the Ni(II) complex (S3b), which could increase the acidity of α-proton in the glycine. Next, a Mitsunobu-Tsunoda reaction using cyanomethylenetributylphosphorane (CMBP) was done to introduce the side chain onto glycine with high stereoselectivity (S3c).³³ We employed 8-quinolinol for the decomplexation to release the ¹³C-labeled benzylated o-Tyr (S3d). After protecting the amine with Boc (S3e), the carboxylic acid was converted to Weinreb amide (S3f). The latter was deprotected and coupled to NMePip-Phe-OH (S3g), followed by reduction to aldehyde with equimolar amount of lithium aluminum hydride (S3h) and removal of benzyl protecting group, resulting in ¹³C-labeled 36.

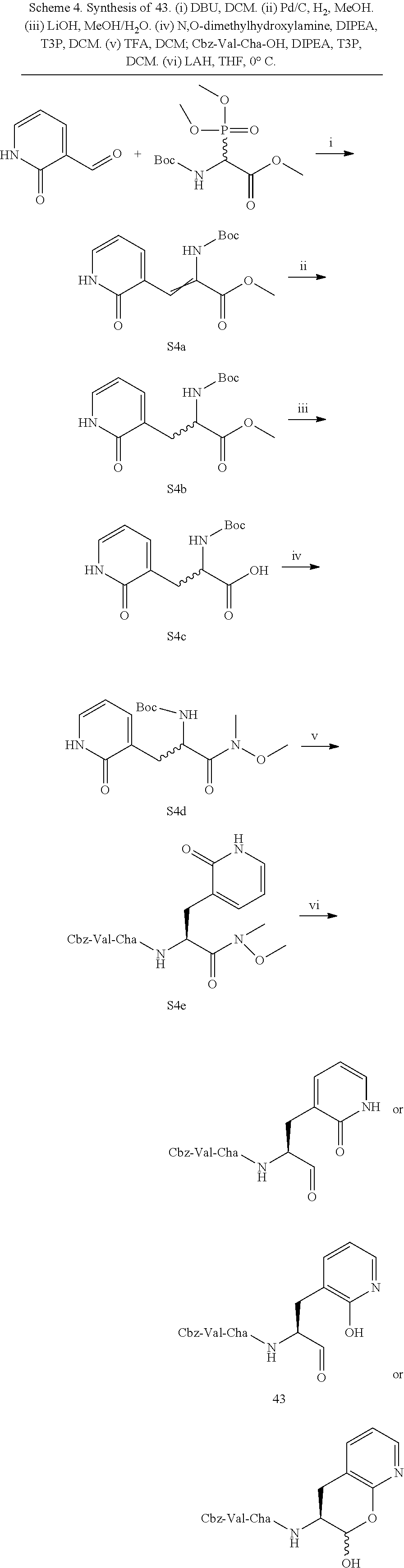

Similar to Scheme 1, the 2-pyridone inhibitor 43 was also synthesized from 2-oxo-1,2-dihydro-3-pyridinecarbaldehyde (Scheme 4). After hydrogenation of S4a, the methyl ester of S4b was hydrolyzed (S4c) and converted to Weinreb amide S4d. The incorporation of Cbz-Val-Cha-scaffold resulted in S4e as diastereomers that could be separated on silica gel column. It turned out that no protection was needed for the 2-pyridone/2-hydroxypyridine, so that the LAH reduction of the Weinreb amide afforded the final product 43. Although there is no apparent aldehydic peak in NMR spectra when using CDCl₃ as solvent, the two inherent equilibria (lactol/aldehyde, pyridone/hydroxypyridine) has led to greater complexity. The exact species of 43 are dependent on multiple factors including solvents, temperature, pH, and so on, as exemplified by its HPLC-MS data (see Supporting Information): there are several crowded peaks in HPLC, yet all of which correspond to the mass of 43 (m/z 552.29).

While the aldehyde group has frequently been employed in chemical probes in drug discovery, rarely has this reactive functionality been advanced in earnest to drug discovery campaigns. However, the masking of aldehydes in drugs and clinical agents has provided a few examples of the successful “masking” of aldehydes for drug discovery. Here, we sequestered the potent free aldehyde inhibitor 28 into an intramolecular masked aldehyde by the introduction of a 2-hydroxyl group onto the P₁ benzyl group, which resulted in a surprisingly stable d-lactol derivative that effected potent yet reversible inhibition of cruzain. The apparent stability of d-lactol in examples of these SMAIs suggests that the observed potent inhibition of cruzain by these SMAIs most likely arises from cruzain-catalyzed ring-opening of the d-lactol, followed by formation of a hemithioacetal. Where evaluated, these self-masked aldehyde inhibitors inhibited axenic and infected cell cultures of trypanosomes with potency equaling or exceeding that of the parent aldehydes. Furthermore, the O-acylation of SMAIs yielded prodrugs that might possess improved metabolic stability and pharmacokinetic properties. Finally, we developed and characterized a novel SMAI of SARS-CoV-2 3CL^{pro}, suggesting a broad applicability of SMAI strategy to other cysteine proteases.

General Information of Chemistry. Unless otherwise noted, all starting materials, reagents and solvents were obtained commercially and used without further purification or distillation. Reactions were conducted under an inert atmosphere (Ar or N₂), and reaction progress was monitored by TLC or HPLC-MS. TLC experiments were performed with silica gel plates on aluminum foil (Sigma-Aldrich, cat. no. 60778). HPLC analysis was conducted on an UltiMate 3000 HPLC system coupled to MS analysis by an ISQ™ EM single quadrupole mass spectrometer. The typical settings for HPLC-MS were as follows. Column used: Phenomenex Luna 5 μm C18(2) 100 Å, 4.6 mm×50 mm; mobile phase A: water containing 0.1% formic acid (v/v); mobile phase B: MeCN containing 0.1% formic acid (v/v); method: gradient elution at 10%-100% B over 6 mins, then isocratic elution at 100% B for 2 mins; flow rate: 1 mL/min; MS parameters: HESI, vaporizer temperature 350° C., source CID voltage 20 V, gas flow: sheath=40-50 psi, aux=sweep=2.0 psi. Most compounds were purified by flash column chromatography (FCC) on silica gel (200-300 mesh) with different solvent systems. Some compounds were purified by semi-preparative HPLC (Prep-HPLC) on the same UltiMate 3000 HPLC system which was connected to a fraction collector. The typical settings for Prep-HPLC were as follows: Column used: Phenomenex Luna 5 μm C18(2) 100 Å, 21.2 mm×250 mm; mobile phases A and B were the same as those for analytical HPLC; method: gradient elution at 10%-100% B over 25 mins, then isocratic elution at 100% B for 5 mins; flow rate: 21.2 mL/min. ¹H and ¹³C NMR spectra of compounds, except for ¹³C-labeled compound 36, were recorded on a Bruker AVANCE III 400 MHz using tetramethylsilane (TMS, 0.00 ppm) or residual solvent (CDCl₃, 7.26 ppm; CD₃OD, 3.31 ppm; (CD₃)₂SO, 2.50 ppm) as the internal standard. All final compounds used for testing in assays had purities that were determined to be >95% as evaluated by their NMR spectra and/or their LC-MS.

Synthetic Procedures. Detailed procedures and characterization of compounds for all reactions in Schemes 1-4 were as follows. Some of them were general procedures which were described by representative compounds.

Methyl 2-((tert-butoxycarbonyl)amino)-3-(2-hydroxy-5-methylphenyl)acrylate (S1a, R=Me). To a solution of (±)-Boc-α-phosphonoglycine trimethyl ester (4.22 g, 14.2 mmol, 1.2 eq) in DCM (20 mL) was added DBU (2.12 mL, 14.2 mmol, 1.2 eq) dropwise at −10° C., and the resulting mixture was stirred for 20 min. Then 4-methylsalicylaldehyde (1.61 g, 11.8 mmol, 1.0 eq) in DCM (10 mL) was slowly added to the mixture over 10 min. The reaction was stirred at room temperature overnight. The resulting mixture was concentrated under reduced pressure, diluted with EtOAc (100 mL), and washed successively with saturated aqueous NH₄Cl (20 mL), saturated aqueous NaHCO₃ (20 mL) and brine (20 mL). The organic layer was dried over Na₂SO₄, filtered, and concentrated under reduced pressure. The obtained crude product was purified by FCC (30-50% EtOAc in hexane, v/v) to give S1a (R=Me, 2.91g, 80%). ¹H NMR (400 MHz, CD₃OD) δ 1.45 (s, 9H, Boc), 2.25 (s, 3H, PhCH₃), 3.83 (s, 3H, COOCH₃), 6.77 (d, J=8.3 Hz, 1H, olefin), 7.02 (dd, J=2.2, 8.4 Hz, 1H, Ph), 7.41 (d, J=2.2 Hz, 1H, Ph), 7.53 (s, 1H, Ph). ¹³C NMR (100 MHz, CD₃OD) δ 19.22, 27.18, 51.36, 79.97, 115.23, 120.46, 124.58, 128.22, 129.63, 131.02, 153.42, 154.76, 166.73. LC-MS: t_{R}=4.93 min; C₁₁H₁₄NO₃⁺ [M+H−Boc]⁺, m/z calcd 208.10, found 208.08.

Methyl 2-((tert-butoxycarbonyl)amino)-3-(2-hydroxy-5-methylphenyl)propanoate (S1b, R=Me). S1a (R=Me, 1.15 g, 3.74 mmol, 1.0 eq) was placed in a two-necked round bottom and charged with N₂ gas. 10% palladium on carbon powder (Pd/C, 110 mg, cat.) was quickly added to the flask, followed by addition of MeOH (20 mL). The flask was degassed and backfilled with H₂ for three cycles. The reaction mixture was stirred at room temperature overnight with a balloon of H₂ for replenishment. The balloon was removed, and the mixture was filtered under reduced pressure. Notice that the operation should be rapid, and the Pd/C powder must be kept wet to avoid catching fire, and was appropriately disposed of in a water-filled, cap-closed container. The filtrate was concentrated under reduced pressure and purified by FCC (30% EtOAc in hexane, then 6% MeOH in DCM) to give S1b (R=Me, 1.1 g, 95%). ¹H NMR (400 MHz, CDCl₃) δ 1.39 (s, 9H, Boc), 2.36 (s, 3H, PhCH₃), 2.79-3.02 (m, 1H, βCH₂), 3.07 (m, 1H, βCH₂), 3.75 (s, 3H, COOCH₃), 4.43 (d, J=6.2 Hz, 1H, αCH), 5.54 (d, J=7.4 Hz, 1H, NH), 6.65-6.73 (m, 2H, Ph), 6.73-6.79 (m, 1H, Ph). ¹³C NMR (100 MHz, CDCl₃) δ 20.57, 28.24, 32.84, 51.98, 54.97, 79.50, 113.30, 116.22, 119.28, 127.84, 147.51, 153.95, 155.33, 172.70. LC-MS: t_{R}=4.98 min; C₁₆H₂₄NO₅⁺ [M+H]⁺, m/z calcd 310.16, found 310.14.

Methyl 2-amino-3-(2-hydroxy-5-methylphenyl)propanoate TFA salt (S1c, R=Me). To a suspension of S1b (R=Me, 1.1 g, 3.56 mmol, 1.0 eq) in DCM (6 mL) was added TFA (3 mL) at 0° C., and the resulting mixture was stirred for 30 min. The mixture was then co-concentrated with toluene three times to remove most of the residual TFA. The obtained crude product S1c (R=Me) was used without further purification.

Methyl (S)-2-((S)-2-(((benzyloxy)carbonyl)amino)-3-phenylpropanamido)-3-(2-hydroxy-5-methylphenyl)propanoate (S1d, R Me, R₁ BnO). A mixed suspension of S1c (R=Me, ˜1.15 g, 3.56 mmol, 1.0 eq), Cbz-Phe-OH (1.07g, 3.56 mmol, 1.0 eq) and DIPEA (2.17 mL, 12.46 mmol, 3.5 eq) in DCM (20 mL) was cooled to 0° C. followed by dropwise addition of T3P (>50% in MeCN, 3.53 mL, 5.34 mmol, 1.5 eq). The resulting mixture was stirred at room temperature for 2 h. The reaction was concentrated under reduced pressure, diluted with EtOAc (75 mL), and washed successively with 5% citric acid (15 mL), saturated aqueous NaHCO₃ (15 mL) and brine (15 mL). The organic layer was dried over Na₂SO₄, filtered, and concentrated under reduced pressure. The obtained crude product was purified by FCC (40% EtOAc in hexane) to give S1d (R=Me, R₁=BnO, 1.11 g, 63% for two steps). ¹H NMR (400 MHz, CDCl₃) δ 2.30 (s, 3H, PhCH₃), 2.83-3.16 (m, 4H, 2×βCH₂), 3.69 (s, 3H, COOCH₃), 4.50 (s, 1H, αCH), 4.75 (s, 1H, αCH), 4.98-5.14 (m, 2H, Cbz CH₂), 5.70 (s, 1H, OH), 6.68 (t, J=8.8 Hz, 1H, NH), 6.91-7.10 (m, 3H, Ph), 7.22 (dd, J=20.7, 26.3 Hz, 7H, Ph), 7.33 (s, 3H, Ph), 8.08 (s, 1H, NH). ¹³C NMR (100 MHz, CDCl₃) δ 20.66, 33.10, 38.37, 52.47, 53.19, 56.10, 67.24, 117.11, 124.49, 127.01, 127.88, 128.22, 128.32, 128.42, 128.53, 128.61, 129.25, 130.87, 135.98, 136.14, 153.57, 156.27, 171.72, 172.08. LC-MS: t_{R}=5.39 min; C₂₈H₃₁N₂O₆⁺ [M+H]⁺, m/z calcd 491.22, found 491.19.

Methyl (S)-2-((S)-2-(((benzyloxy)carbonyl)amino)-3-phenylpropanamido)-3-(2-((tert-butyldimethylsilyl)oxy)-5-methylphenyl)propanoate (S1e, R=Me, R₁=BnO, R₂=TBS). A mixed solution of S1d (R=Me, R₁=BnO, 990 mg, 2.02 mmol, 1.0 eq), TBSCl (609 mg, 4.04 mmol, 2.0 eq), and imidazole (412 mg, 6.06 mmol, 3.0 eq) was stirred at room temperature overnight. The reaction was quenched by addition of 0.5M HCl (10 mL), and was stirred for another 15 min. The mixture was concentrated under reduced pressure, and was partitioned between EtOAc (50 mL) and water (10 mL). The organic layer was washed with saturated aqueous NaHCO₃ (10 mL) and brine (10 mL), and was dried over Na₂SO₄, filtered, and concentrated under reduced pressure. The obtained crude product was purified by FCC (20% EtOAc in hexane) to give S1e (R=Me, R₁=BnO, R₂=TBS, 897 mg, 73%). ¹H NMR (400 MHz, CDCl₃) δ 0.16 (s, 6H, TBS Si(CH₃)₂), 0.93 (s, 9H, TBS C(CH₃)₃), 2.37 (s, 3H, PhCH₃), 2.59-2.87 (m, 2H, βCH₂), 2.89-3.05 (m, 2H, βCH₂), 3.54 (d, J=4.3 Hz, 3H, COOCH₃), 4.36 (d, J=22.6 Hz, 1H, αCH), 4.64 (q, J=8.3 Hz, 1H, αCH), 4.95 (p, J=12.1 Hz, 2H, Cbz CH₂), 5.32-5.55 (m, 1H, NH), 6.56-6.65 (m, 1H, Ph), 6.83-7.02 (m, 3H, Ph), 7.05-7.25 (m, 9H, Ph). ¹³C NMR (100 MHz, CDCl₃) δ −3.50, 18.27, 20.53, 25.81, 32.94, 38.57, 52.20, 52.57, 56.08, 66.96, 119.79, 125.91, 126.90, 127.89, 127.95, 128.02, 128.04, 128.07, 128.14, 128.44, 128.47, 128.57, 128.71, 129.28, 129.35, 130.82, 136.25, 136.47, 152.62, 155.90, 170.76, 171.62, 172.00. LC-MS: t_{R}=7.23 min; C₃₄H₄₅N₂O₆Si⁺ [M+H]⁺, m/z calcd 605.30, found 605.24.

Methyl (S)-2-((S)-2-(((benzyloxy)carbonyl)amino)-3-phenylpropanamido)-3-(2-methoxyphenyl)propanoate (S1e, R=H, R₁=BnO, R₂=Me). To a solution of S1d (R=H, R¹=BnO, 172 mg, 0.36 mmol, 1.0 eq) in DMF (2 mL) was successively added K₂CO₃ (100 mg, 0.72 mmol, 2.0 eq) and iodomethane (67 μL, 1.08 mmol, 3.0 eq) at 0° C. The resulting mixture was stirred at room temperature for 20 h during which the system should be kept securely sealed to avoid evaporation of iodomethane. The mixture was diluted with EtOAc (50 mL), and washed with water extensively (5×10 mL). The organic layer was dried over Na2SO4, filtered, and concentrated under reduced pressure. The obtained crude product was purified by FCC (30% EtOAc in hexane) to give S1e (R=H, R₁=BnO, R₂=Me, 167 mg, 95%). ¹H NMR (400 MHz, CDCl₃) δ 3.02 (qd, J=5.7, 13.6, 16.9 Hz, 4H, 2×βCH₂), 3.61 (s, 3H, COOCH₃), 3.93 (s, 3H, PhOCH₃), 4.48 (s, 1H, αCH), 4.72 (s, 1H, αCH), 4.88-5.12 (m, 2H, Cbz CH₂), 5.69 (d, J=8.2 Hz, 1H, NH), 6.64-6.88 (m, 2H, Ph), 6.92-7.06 (m, 3H, Ph), 7.08-7.18 (m, 4H, Ph), 7.24 (d, J=18.6 Hz, 5H, Ph), 7.77 (d, J=43.8 Hz, 1H, NH). ¹³C NMR (100 MHz, CDCl₃) δ 32.65, 32.96, 38.40, 52.33, 52.40, 53.70, 56.04, 67.11, 115.84, 120.35, 122.73, 126.88, 126.90, 127.88, 128.16, 128.52, 128.72, 129.30, 131.30, 136.14, 154.71, 156.21, 156.23, 171.98, 172.34. LC-MS: tR=5.71 min; C₂₈H₃₁N₂O₆⁺ [M+H]⁺, m/z calcd 491.22, found 491.33.

Benzyl ((S)-1-(((S)-1-(2-((tert-butyldimethylsilyl)oxy)-5-methylphenyl)-3-hydroxypropan-2-yl)amino)-1-oxo-3-phenylpropan-2-yl)carbamate (S1f, R=Me, R₁=BnO, R₂=TBS). To a solution of S1e (R=Me, R₁=BnO, R₂=TBS, 897 mg, 1.48 mmol, 1.0 eq) in MeOH (10 mL) was added NaBH₄ (1.2 g, 31.7 mmol, >20 eq) in multiple portions every 30 min. The reaction was stirred at room temperature for another 2 h, and then quenched by addition of saturated aqueous NH₄Cl (10 mL). The mixture was concentrated under reduced pressure, and diluted with EtOAc (50 mL). The organic layer was washed with saturated aqueous NaHCO₃ (10 mL) and brine (10 mL), and was dried over Na₂SO₄, filtered, and concentrated under reduced pressure. The obtained crude product was purified by FCC (35% EtOAc in hexane) to give S1f (R=Me, R₁=BnO, R₂=TBS, 450 mg, 53%). ¹H NMR (400 MHz, CDCl₃) δ 0.26 (s, 6H, TBS Si(CH₃)₂), 1.03 (s, 9H, TBS C(CH₃)₃), 2.40 (s, 3H, PhCH₃), 2.74 (dt, J=7.0, 13.1 Hz, 2H, βCH₂), 2.97 (dd, J=6.8, 40.5 Hz, 2H, βCH₂), 3.25-3.60 (m, 2H, CH₂OH), 4.02-4.15 (m, 1H, αCH), 4.39 (s, 1H, αCH), 4.97-5.15 (m, 2H, Cbz CH₂), 5.69 (dd, J=7.8, 66.8 Hz, 1H, NH), 6.54 (dd, J=6.8, 121.3 Hz, 1H, Ph), 6.72-6.81 (m, 1H, Ph), 7.04-7.14 (m, 2H, Ph), 7.15-7.30 (m, 6H, Ph & NH), 7.34 (q, J=6.2, 7.1 Hz, 4H, Ph). ¹³C NMR (100 MHz, CDCl₃) δ −4.05, 18.27, 21.02, 25.87, 31.17, 39.00, 52.34, 56.69, 63.66, 67.03, 119.96, 126.28, 127.00, 127.56, 127.93, 128.03, 128.16, 128.47, 128.65, 129.29, 130.27, 131.00, 136.24, 136.53, 152.38, 155.94, 170.98, 171.35. LC-MS: t_{R}=6.90 min; C₃₃H₄₅N₂O₅Si⁺ [M+H]⁺, m/z calcd 577.31, found 577.37.

Benzyl ((S)-1-(((S)-1-(2-((tert-butyldimethylsilyl)oxy)-5-methylphenyl)-3-oxopropan-2-yl)amino)-1-oxo-3-phenylpropan-2-yl)carbamate (S1g, R=Me, R₁=BnO, R₂=TBS). To a solution of S1f (R=Me, R₁=BnO, R₂=TBS, 151 mg, 0.26 mmol, 1.0 eq) in DCM (8 mL) at 0° C. was added Dess-Martin periodinane (133 mg, 0.31 mmol, 1.2 eq) and NaHCO₃ powder (55 mg, 0.65 mmol, 2.5 eq). The resulting mixture was stirred at 0° C. for 1 h, and then quenched by addition of saturated aqueous Na₂S₂O₃ (2 mL). The reaction was concentrated under reduced pressure, diluted with EtOAc (50 mL), and washed with brine (3×10 mL). The organic layer was dried over Na₂SO₄, filtered, and concentrated under reduced pressure. The obtained crude product was purified by FCC (25% EtOAc in hexane) to give S1g (R=Me, R₁=BnO, R₂=TBS, 68 mg, 45%). ¹H NMR (400 MHz, CDCl₃) δ 0.24 (d, J=4.6 Hz, 6H, TBS Si(CH₃)₂), 1.01 (s, 9H, TBS C(CH₃)₃), 2.24 (s, 3H, PhCH₃), 2.77-3.20 (m, 4H, 2× βCH₂), 4.51 (q, J=6.7 Hz, 1H, αCH), 5.02-5.15 (m, 2H, Cbz CH₂), 5.35 (s, 1H, αCH), 6.52 (d, J=5.5 Hz, 1H, NH), 6.71 (d, J=8.2 Hz, 1H, Ph), 6.85 (d, J=2.2 Hz, 1H, Ph), 6.94 (dd, J=2.3, 8.2 Hz, 1H, Ph), 7.16 (d, J=7.1 Hz, 2H, Ph), 7.21-7.28 (m, 3H, Ph), 7.35 (dt, J=4.7, 6.9 Hz, 5H, Ph), 9.41 (s, 1H, CHO). ¹³C NMR (100 MHz, CDCl₃) δ −4.14, 18.28, 20.43, 25.90, 29.92, 38.80, 56.11, 59.57, 67.01, 118.77, 125.68, 127.09, 128.01, 128.16, 128.50, 128.67, 128.91, 129.26, 130.94, 131.91, 136.13, 151.28, 170.96, 198.75. LC-MS: t_{R}=6.81 min; C₃₃H₄₃N₂O₅Si⁺ [M+H]⁺, m/z calcd 575.29, found 575.36.

Benzyl ((2S)-1-(((3S)-2-hydroxy-6-methylchroman-3-yl)amino)-1-oxo-3-phenylpropan-2-yl)carbamate (S1h, 7, R=Me, R₁=BnO, R₂=TBS). To a solution of S1g (R=Me, R₁=BnO, R₂=TBS, 68 mg, 0.12 mmol, 1.0 eq) in THE (3 mL) was slowly added 1.0M TBAF in THF (131 μL, 0.13 mmol, 1.1 eq) at 0° C. The resulting mixture was stirred at 0° C. for 1 h, and concentrated under reduced pressure. The residue was diluted with EtOAc (50 mL) and washed with saturated aqueous NH₄Cl (10 mL) and brine (10 mL). The organic layer was dried over Na₂SO₄, filtered, and concentrated under reduced pressure. The obtained crude product was purified by FCC (35% EtOAc in hexane) to give S1h (R=Me, R₁=BnO, R₂=TBS, i.e., compound 7, 20 mg, 36%). ¹H NMR (400 MHz, CDCl₃) δ 2.10-2.18 (m, 3H, PhCH₃), 2.37-3.10 (m, 4H, 2× βCH₂), 4.10-4.45 (m, 2H, αCH & NH), 4.71-5.10 (m, 3H, Cbz CH₂ & NH), 5.53 (dd, J=7.2, 51.9 Hz, 1H, αCH), 5.97-6.41 (m, 1H, lactol C(O)OH), 6.59 (td, J=10.3, 31.2, 32.4 Hz, 2H, Ph), 6.78 (dd, J=8.2, 18.7 Hz, 1H, Ph), 6.98-7.28 (m, 10H, Ph). ¹³C NMR (100 MHz, CDCl₃) δ 20.46, 25.96, 39.10, 46.02, 56.24, 67.20, 91.04, 91.93, 116.68, 118.34, 119.01, 119.23, 128.04, 128.12, 128.20, 128.52, 128.67, 128.77, 129.19, 129.32, 129.64, 130.50, 130.63, 136.02, 136.26, 148.52, 156.15, 171.23. LC-MS: t_{R}=5.32 min; C₂₇H₂₉N₂O₅⁺ [M+H]⁺, m/z calcd 461.21, found 461.27.

Benzyl (4-methylpiperazine-1-carbonyl)-L-phenylalaninate (S1i). To a solution of 4-methylpiperazine-1-carbonyl chloride hydrochloride (1.365 g, 6.86 mmol, 1.1 eq) in THE (20 mL) at −10° C. was added Et₃N (2.08 mL, 14.96 mmol, 2.4 eq) dropwise. The resulting mixture was stirred for 15 min, and then was added a solution of benzyl L-phenylalaninate hydrochloride (1.82 g, 6.23 mmol, 1.0 eq) in THE (20 mL) dropwise. The reaction was stirred at room temperature overnight, quenched by addition of water (10 mL), and concentrated under reduced pressure. The residue was diluted with EtOAc (100 mL) and washed successively with saturated aqueous NH₄Cl (20 mL), saturated aqueous NaHCO₃ (20 mL) and brine (20 mL). The organic layer was dried over Na₂SO₄, filtered, and concentrated under reduced pressure. The obtained crude product was purified by FCC (5-10% MeOH in DCM) to give S1i (1.55 g, 60%). ¹H NMR (400 MHz, CDCl₃) δ 2.31 (s, 3H, NCH₃), 2.37 (t, J=5.1 Hz, 4H, 2× piperazinyl CH₂), 3.14 (d, J=4.2 Hz, 2H, βCH₂), 3.37 (q, J=4.9 Hz, 4H, 2× piperazinyl CH₂), 4.87 (d, J=5.1 Hz, 2H, Cbz CH₂), 5.05-5.28 (m, 2H, αCH & NH), 7.02 (dd, J=2.9, 6.5 Hz, 2H, Ph), 7.20-7.27 (m, 3H, Ph), 7.30-7.42 (m, 5H, Ph). ¹³C NMR (100 MHz, CDCl₃) δ 38.31, 43.69, 46.09, 54.32, 54.59, 67.11, 126.93, 128.44, 128.53, 128.57, 129.38, 135.26, 136.11, 156.46, 172.45. LC-MS: t_{R}=3.42 min; C₂₂H₂₈N₃O₃⁺ [M+H]⁺, m/z calcd 382.21, found 382.2.

(4-methylpiperazine-1-carbonyl)-L-phenylalanine (S1j). Prepared from S1i in a similar procedure as described for Sib. The obtained crude product S1j was used without further purification.

(1aR,7aS)-1a,2,7,7a-tetrahydronaphtho[2,3-b]oxirene (S1k). To a solution of 1,4-dihydronaphthalene (2.09 mg, 16.1 mmol, 1.0 eq) in chloroform (40 mL) was slowly added 70% mCPBA (4.74 g, 19.3 mmol, 1.2 eq) at 0° C. The resulting mixture was stirred at room temperature overnight, and quenched by addition of 2M KOH (60 mL). The reaction was concentrated under reduced pressure, and diluted with EtOAc (100 mL). The organic layer was washed with brine (3×20 mL), and was dried over Na₂SO₄, filtered, and concentrated under reduced pressure. The obtained crude product was purified by FCC (10% EtOAc in hexane) to give S1k (2.03 g, 86%)¹H NMR (400 MHz, CDCl₃) δ 3.05 (d, J=17.7 Hz, 2H, CH₂), 3.18 (d, J=16.8 Hz, 2H, CH₂), 3.29-3.36 (m, 2H, 2× epoxide CH), 6.95 (dd, J=3.5, 5.6 Hz, 2H, Ph), 7.06 (dd, J=3.4, 5.7 Hz, 2H, Ph). ¹³C NMR (100 MHz, CDCl₃) δ 29.84, 51.71, 126.57, 129.33, 131.74.

(2R,3R)-3-azido-1,2,3,4-tetrahydronaphthalen-2-ol and (2S,3S)-3-azido-1,2,3,4-tetrahydronaphthalen-2-ol (S1l). To a solution of S1k (2.02 g, 13.8 mmol, 1.0 eq) in a mixture of MeOH (30 mL) and H₂O (10 mL) was added NaN₃ (1.8 g, 27.7 mmol, 2.0 eq) and NH₄Cl (1.11 g, 20.8 mmol, 1.5 eq). The resulting mixture was heated to 60° C. and stirred overnight. The reaction was cooled to room temperature, concentrated under reduced pressure, and diluted with EtOAc (100 mL). The organic layer was washed with brine (3×20 mL), and was dried over Na₂SO₄, filtered, and concentrated under reduced pressure. The obtained crude product S1l (2.22 g, 85%) was used without further purification. ¹H NMR (400 MHz, CDCl₃) δ 2.77-2.86 (m, 2H, CH₂), 3.17 (ddd, J=2.9, 5.8, 16.5 Hz, 2H, CH₂), 3.66 (td, 1H, CH—N₃), 3.87 (td, J=5.8, 9.4 Hz, 1H, CH—OH), 7.04-7.09 (m, 2H, Ph), 7.09-7.16 (m, 2H, Ph). ¹³C NMR (100 MHz, CDCl₃) δ 33.64, 36.57, 63.62, 70.47, 126.50, 126.68, 128.59, 128.99, 132.62, 133.41.

(2R,3R)-3-amino-1,2,3,4-tetrahydronaphthalen-2-ol and (2S,3S)-3-amino-1,2,3,4-tetrahydronaphthalen-2-ol (S1m). Prepared from S1l in a similar procedure as described for S1b. The obtained crude product Sim was used without further purification.

N—((S)-1-(((2S,3S)-3-hydroxy-1,2,3,4-tetrahydronaphthalen-2-yl)amino)-1-oxo-3-phenylpropan-2-yl)-4-methylpiperazine-1-carboxamide and N—((S)-1-(((2R,3R)-3-hydroxy-1,2,3,4-tetrahydronaphthalen-2-yl)amino)-1-oxo-3-phenylpropan-2-yl)-4-methylpiperazine-1-carboxamide (50). Prepared from S1j and Sim in a similar procedure as described for S1d. The obtained crude product was purified by FCC (10% MeOH in DCM) to give compound 3. ¹H NMR (400 MHz, DMSO-d₆) δ 2.17 (s, 3H), 2.20 (q, J=3.3 Hz, 3H) 2.61 (ddd, J=6.7, 16.9, 27.1 Hz, 2H), 2.83 (dd, J=10.2, 13.6 Hz, 1H), 2.95 (ddd, J=4.7, 8.9, 12.8 Hz, 2H), 3.04-3.33 (m, 5H), 3.79 (td, J=4.8, 6.8 Hz, 1H), 3.93 (p, J=7.0 Hz, 1H), 4.33 (ddd, J=4.6, 8.3, 10.1 Hz, 1H), 6.48 (d, J=8.3 Hz, 1H), 7.05-7.10 (m, 3H), 7.13-7.21 (m, 1H), 7.22-7.27 (m, 3H), 7.77 (d, J=7.8 Hz, 1H), 8.15 (s, 1H). ¹³C NMR (100 MHz, DMSO-d₆) δ 32.84, 35.71, 38.13, 43.63, 45.94, 50.11, 54.62, 56.33, 67.15, 126.12, 126.24, 126.48, 128.35, 128.99, 129.38, 129.73, 134.39, 134.78, 139.14, 157.37, 163.56, 172.89.

4-Methyl-N—((S)-1-oxo-1-(((S)-1-oxo-4-phenylbutan-2-yl)amino)-3-phenylpropan-2-yl)piperazine-1-carboxamide (52). The preparation of S1n, S1o and 52 was similar to the procedures for S1d and S1f and S1g, respectively. ¹H NMR (400 MHz, CDCl₃) δ 1.82 (dt, J=7.6, 14.6 Hz, 1H, piperazinyl CH₂), 2.09-2.17 (m, 1H, piperazinyl CH₂), 2.28 (s, 3H, NCH₃), 2.34 (dt, J=3.8, 7.1 Hz, 4H, piperazinyl CH₂ & homoPhe βCH₂), 2.55 (dt, J=7.9, 27.5 Hz, 2H, homoPhe γCH₂), 3.11 (p, J=5.9, 6.5 Hz, 2H, Phe βCH₂), 3.29-3.39 (m, 4H, 2× piperazinyl CH₂), 4.33 (td, J=5.3, 7.5 Hz, 1H, homoPhe αCH), 4.64 (q, J=7.3 Hz, 1H, Phe αCH), 5.02 (d, J=7.3 Hz, 1H, NH), 6.75 (d, J=7.1 Hz, 1H, NH), 7.08-7.13 (m, 2H, Ph), 7.18-7.25 (m, 5H, Ph), 7.27-7.31 (m, 3H, Ph), 9.43 (s, 1H, CHO). C₂₅H₃₃N₄O₃⁺ [M+H]⁺, m/z calcd 437.25, found 481.21.

(3S)-3-((S)-2-(4-methylpiperazine-1-carboxamido)-3-phenylpropanamido)chroman-²-yl acetate (S2a, 37, R₁=Me). To a solution of compound 36 (50 mg, 0.114 mmol, 1.0 eq) in DCM (3 mL) was added acetic anhydride (32 μL, 0.342 mmol, 3.0 eq), Et₃N (48 μL, 0.342 mmol, 3.0 eq) and DMAP (2.8 mg, 0.023 mmol, 0.2 eq). The resulting mixture was stirred at room temperature overnight, and concentrated under reduced pressure. The residue was diluted with EtOAc (50 mL), and washed successively with saturated aqueous NH₄Cl (10 mL), saturated aqueous NaHCO₃ (10 mL) and brine (10 mL). The organic layer was dried over Na₂SO₄, filtered, and concentrated under reduced pressure. The obtained crude product was purified by FCC (10% MeOH in DCM) to give S2a (R₁=Me, i.e., compound 37, 31 mg, 57%). ¹H NMR (400 MHz, CDCl₃) δ 2.01 (d, J=23.4 Hz, 3H, CH₃CO), 2.29 (d, J=3.5 Hz, 3H, NCH₃), 2.30-2.38 (m, 4H, 2× piperazinyl CH₂), 2.69-2.97 (m, 2H, lactol βCH₂), 2.98-3.15 (m, 2H, Phe βCH₂), 3.25-3.41 (m, 4H, 2× piperazinyl CH₂), 4.49 (dt, J=6.9, 13.8 Hz, 1H, αCH), 5.18 (dd, J=7.4, 40.0 Hz, 1H, Phe αCH), 6.12 (dd, J=2.5, 17.4 Hz, 1H, lactol CH), 6.31-6.54 (m, 1H, NH), 6.82-6.91 (m, 1H, Ph), 6.91-7.07 (m, 2H, Ph), 7.07-7.19 (m, 2H, Ph), 7.19 (s, 3H, Ph & NH), 7.26-7.34 (m, 2H, Ph). ¹³C NMR (100 MHz, CDCl₃) δ 20.99, 26.61, 29.67, 38.69, 43.71, 45.99, 54.48, 56.23, 88.88, 89.33, 117.07, 119.40, 121.89, 127.09, 128.06, 128.70, 128.74, 129.15, 129.20, 136.94, 150.22, 157.02, 169.38, 171.96. LC-MS: t_{R}=3.29 min; C₂₆H₃₃N₄O₅⁺ [M+H]⁺, m/z calcd 481.24, found 481.3.

N-((2S)-1-(((3S)-2-ethoxychroman-3-yl)amino)-1-oxo-3-phenylpropan-2-yl)-4-methylpiperazine-1-carboxamide (S2b, 40, R₂ Et). To a solution of compound 36 (40 mg, 0.091 mmol, 1.0 eq) in EtOH (2 mL) was added BF₃OEt₂ (300 μL, 2.43 mmol, >20 eq) dropwise at 0° C. The resulting mixture was stirred at room temperature overnight, and quenched by addition of saturated aqueous NH₄Cl (2 mL). The reaction was concentrated under reduced pressure and then partitioned between DCM (20 mL) and water (20 mL). The water layer was further washed with DCM (2×20 mL). The combined organic layers were dried over Na₂SO₄, filtered, and concentrated under reduced pressure. The obtained crude product was purified by FCC (8% MeOH in DCM) to give S2b (R₂=Et, i.e., compound 40, 18.3 mg, 43%). ¹H NMR (400 MHz, CDCl₃) δ 1.00 (t, 3H, OCH₂CH₃), 2.16-2.36 (m, 7H, NCH₃ & 2× piperazinyl CH₂), 2.69 (dd, J=9.2, 14.2 Hz, 1H, βCH₂), 2.83-2.95 (m, 1H, βCH₂), 3.10 (dt, J=7.2, 13.4 Hz, 1H, Phe βCH₂), 3.21 (dd, J=4.0, 6.1 Hz, 1H, Phe βCH₂), 3.31 (dt, J=6.0, 11.1 Hz, 3H, OCH₂CH₃ & piperazinyl CH₂), 3.58-3.76 (m, 1H, piperazinyl CH₂), 4.13-4.27 (m, 1H, piperazinyl CH₂), 4.41 (q, J=7.3 Hz, 1H, piperazinyl CH₂), 4.68 (dd, J=2.3, 49.7 Hz, 1H, αCH), 4.90-5.15 (m, 1H, Phe αCH), 5.78 (dd, J=8.9, 24.8 Hz, 1H, lactol CH), 6.73 (d, J=8.1 Hz, 1H, Ph), 6.80 (q, J=6.8, 7.4 Hz, 1H, Ph), 6.90 (d, J=7.2 Hz, 1H, Ph), 7.03 (t, J=7.2 Hz, 1H, Ph), 7.07-7.30 (m, 5H, Ph). ¹³C NMR (100 MHz, CDCl₃) δ 14.94, 14.98, 27.08, 39.59, 43.72, 45.10, 46.06, 54.58, 56.30, 64.01, 96.02, 96.34, 116.79, 120.48, 121.16, 126.99, 127.67, 128.65, 128.75, 129.20, 129.33, 136.84, 137.17, 150.58, 150.70, 156.56, 156.68, 171.26, 171.66. LC-MS: t_{R}=3.53 min; C₂₆H₃₅N₄O₄⁺ [M+H]⁺, m/z calcd 467.27, found 467.3.

(S)—N-(2-benzoylphenyl)-1-benzylpyrrolidine-2-carboxamide (S3a). To a mixed solution of benzyl-L-proline (1.03 g, 5.0 mmol, 1.0 eq) and 1-methylimidazole (877 μL, 11.0 mmol, 2.2 eq) in DCM (10 mL) was added MsCl (387 μL, 5.0 mmol, 1.0 eq) dropwise at 0° C. The resulting mixture was stirred at room temperature for 10 min, and then was added a solution of 2-aminobenzophenone (888 mg, 4.5 mmol, 0.9 eq) in DCM (10 mL). The reaction was heated to 45° C., and stirred overnight. The reaction was quenched by addition of NH₄Cl (15 mL), and concentrated under reduced pressure. The residue was diluted with EtOAc (75 mL), washed with saturated aqueous NaHCO₃ (15 mL) and brine (15 mL). The organic layer was dried over Na₂SO₄, filtered, and concentrated under reduced pressure. The obtained crude product was purified by FCC (15-25% EtOAc in hexane) to give S3a (1.344 g, 70%). ¹H NMR (400 MHz, CDCl₃) δ 1.72-1.85 (m, 2H, Pro γCH₂), 1.91-2.01 (m, 1H, Pro βCH₂), 2.17-2.31 (m, 1H, Pro βCH₂), 2.40 (td, J=6.8, 9.5 Hz, 1H, Pro δCH₂), 3.20 (ddd, J=2.4, 6.4, 9.1 Hz, 1H, Pro δCH₂), 3.31 (dd, J=4.8, 10.1 Hz, 1H, Pro αCH), 3.58 (d, J=12.9 Hz, 1H, Bn CH₂), 3.91 (d, J=12.9 Hz, 1H, Bn CH₂), 7.07 (td, J=1.1, 7.6 Hz, 1H, Ph), 7.10-7.17 (m, 3H, Ph), 7.33-7.40 (m, 2H, Ph), 7.49 (qd, J=6.5, 7.9 Hz, 4H, Ph), 7.56-7.61 (m, 1H, Ph), 7.73-7.83 (m, 2H, Ph), 8.57 (dd, J=1.0, 8.4 Hz, 1H, Ph), 11.50 (s, 1H, NH). ¹³C NMR (100 MHz, CDCl₃) δ 24.18, 31.03, 53.89, 59.87, 68.32, 121.53, 122.19, 125.36, 127.06, 128.16, 128.31, 129.13, 130.10, 132.45, 132.54, 133.35, 138.15, 138.59, 139.22, 174.60, 197.99. LC-MS: tR=3.50 min; C₂₅H₂₅N₂O₂⁺ [M+H]⁺, m/z calcd 385.19, found 384.85.

### (S,E)-2-(((2-(1-benzylpyrrolidine-2-carboxamido)phenyl)(phenyl)methylene)amino)acetate-1-¹³C in complex with Ni(II) (S3b)

To a mixed suspension of S3a (860 mg, 2.24 mmol, 1.0 eq), [1-¹³C] glycine (425 mg, 5.59 mmol, 2.5 eq), and Ni(NO₃)_{2·}6H₂O (1.30 g, 4.47 mmol, 2.0 eq) in MeOH (7 mL) at 45° C. was added a solution of ground KOH (752 mg, 13.4 mmol, 6.0 eq) in MeOH (3 mL). The resulting mixture was stirred at 60° C. for 1 h (note that prolonged heating might lead to racemization). The reaction was neutralized by addition of acetic acid (800 μL), and diluted with water to a volume of 50 mL. The reaction was concentrated under reduced pressure and extracted with DCM (75 mL). The organic layer was dried over Na₂SO₄, filtered, and concentrated under reduced pressure. The obtained crude product was purified by FCC (20-30% acetone in DCM) to give S3b (715 mg, 64%). ¹H NMR (400 MHz, CDCl₃) δ 2.07 (dddd, J=2.4, 5.6, 8.5, 15.1 Hz, 1H, Pro γCH₂), 2.12-2.21 (m, 1H, Pro γCH₂), 2.42 (dddd, J=8.3, 9.5, 10.7, 13.4 Hz, 1H, Pro βCH₂), 2.57 (dddd, J=2.6, 6.3, 9.2, 14.4 Hz, 1H, Pro βCH₂), 3.25-3.40 (m, 1H, Pro δCH₂), 3.46 (dd, J=5.4, 10.7 Hz, 1H, Pro δCH₂), 3.61-3.74 (m, 3H, N-Bn CH₂ & Pro αCH), 3.77 (dd, J=5.5, 20.1 Hz, 1H, Gly CH-₂), 4.48 (d, J=12.7 Hz, 1H, Gly CH₂), 6.69 (ddd, J=1.2, 6.9, 8.1 Hz, 1H, Ph), 6.80 (dd, J=1.7, 8.3 Hz, 1H, Ph), 6.92-7.04 (m, 1H, Ph), 7.05-7.13 (m, 1H, Ph), 7.20 (ddd, J=1.7, 6.9, 8.7 Hz, 1H, Ph), 7.28-7.33 (m, 1H, Ph), 7.42 (t, J=7.6 Hz, 2H, Ph), 7.52 (ddd, J=3.4, 7.7, 18.9 Hz, 3H, Ph), 8.01-8.12 (m, 2H, Ph), 8.31 (dd, J=1.1, 8.6 Hz, 1H, Ph). ¹³C NMR (100 MHz, CDCl₃) δ 23.69, 30.76, 57.56, 61.57, 63.16, 69.94, 124.26, 125.16, 126.27, 128.91, 129.11, 129.34, 129.59, 131.73, 132.22, 132.77, 133.17, 133.37, 134.67, 142.62, 143.81, 177.21, 179.27. LC-MS: t_{R}=3.71 min; C₂₆¹³CH₂₆N₃NiO₃⁺ [M+H]⁺, m/z calcd 499.14, found 498.83.

(S)-3-(2-(benzyloxy)phenyl)-2-(((E)-(2-((S)-1-benzylpyrrolidine-2-carboxamido)phenyl)(phenyl)methylene)amino)propanoate-1-¹³C in complex with Ni(II) (S3c). A mixed slurry of S3b (715 mg, 1.43 mmol, 1.0 eq), (2-(benzyloxy)phenyl)methanol (613 mg, 2.86 mmol, 2.0 eq), and CMBP (750 μL, 2.86 mmol, 2.0 eq) in toluene (5 mL) was heated to 120° C. and stirred overnight. The reaction was cooled to room temperature and concentrated under reduced pressure. The obtained crude product was purified by FCC (10% acetone in DCM) to give S3c (417 mg, 40%). ¹H NMR (400 MHz, CDCl₃) δ 1.48-1.59 (m, 1H, Pro γCH₂), 1.99 (ddd, J=6.5, 8.4, 11.1 Hz, 1H, Pro δCH₂), 2.04-2.14 (m, 1H, Pro γCH₂), 2.31 (tdd, J=2.9, 6.7, 9.7 Hz, 2H, Pro βCH₂), 2.76 (ddd, J=1.6, 4.4, 13.6 Hz, 1H, o-Tyr βCH₂), 2.90-3.05 (m, 1H, Pro δCH₂), 3.22 (ddd, J=5.0, 7.2, 13.6 Hz, 1H, o-Tyr βCH₂), 3.33 (dd, J=8.0, 9.3 Hz, 1H, Pro αH), 3.44 (d, J=12.6 Hz, 1H, N-Bn CH₂), 4.18 (q, J=4.4 Hz, 1H, o-Tyr αCH), 4.23 (d, J=12.6 Hz, 1H, N-Bn CH₂), 4.55 (d, J=10.8 Hz, 1H, OBn CH₂), 4.90 (d, J=10.8 Hz, 1H, OBn CH₂), 6.14 (dt, J=1.5, 7.7 Hz, 1H, Ph), 6.41 (dd, J=1.7, 8.2 Hz, 1H, Ph), 6.63 (ddd, J=1.2, 6.9, 8.2 Hz, 1H, Ph), 6.83-6.94 (m, 2H, Ph), 6.96-7.22 (m, 9H, Ph), 7.28 (d, J=7.6 Hz, 2H, Ph), 7.33-7.46 (m, 4H, Ph), 7.95-8.08 (m, 2H, Ph), 8.35 (dd, J=1.1, 8.7 Hz, 1H, Ph). ¹³C NMR (100 MHz, CDCl₃) δ 22.35, 25.60, 28.10, 59.19, 63.38, 68.12, 69.68, 71.86, 112.85, 122.38, 123.33, 123.36, 124.10, 127.39, 127.74, 127.80, 128.06, 128.35, 128.51, 128.73, 128.88, 129.20, 129.27, 129.61, 130.06, 130.30, 130.32, 134.10, 134.82, 137.07, 145.11, 155.82, 158.83, 170.83, 175.71. LC-MS: t_{R}=4.70 min; C₄₀¹³CH₃₈N₃NiO₄⁺ [M+H]⁺, m/z calcd 695.22, found 694.96.

(S)-2-amino-3-(2-(benzyloxy)phenyl)propanoic-1-¹³C acid (S3d). A mixed solution of S3c (522 mg, 0.75 mmol, 1.0 eq) and 8-quinolinol (272 mg, 1.88 mmol, 2.5 eq) in MeCN (10 mL) and H₂O (1 mL) was heated to 40° C. and stirred overnight. The reaction was filtered, and the filtrate was concentrated. The residue was diluted with DCM (30 mL), and washed with water (3×30 mL). The organic layer containing the retrieved BPB was concentrated and saved for future use. The combined water layers were concentrated to a small volume that was subsequently purified by Prep-HPLC to give S3d (87 mg, 43%). ¹H NMR (400 MHz, CDCl₃) δ 2.96 (ddd, J=2.5, 9.4, 14.3 Hz, 1H, βCH₂), 3.49 (ddd, J=2.9, 4.5, 14.3 Hz, 1H, βCH₂), 3.93 (dt, J=4.7, 9.4 Hz, 1H, αCH), 5.20 (s, 2H, OBn CH₂), 6.92 (td, J=1.1, 7.4 Hz, 1H, Ph), 7.05 (d, J=8.1 Hz, 1H, Ph), 7.24 (dd, J=6.7, 8.2 Hz, 2H, Ph), 7.28-7.34 (m, 1H, Ph), 7.38 (dd, J=6.6, 8.3 Hz, 2H, Ph), 7.45-7.53 (m, 2H, Ph). ¹³C NMR (100 MHz, CDCl₃) δ 33.52, 55.91, 71.50, 114.97, 120.74, 124.67, 127.65, 127.97, 128.07, 128.32, 130.64, 139.00, 157.44, 173.03. LC-MS: t_{R}=2.70 min; C₁₅¹³CH₁₈NO₃⁺ [M+H]⁺, m/z calcd 273.13, found 272.75.

(S)-3-(2-(benzyloxy)phenyl)-2-((tert-butoxycarbonyl)amino)propanoic-1-¹³C acid (S3e). To a solution of S3d (87 mg, 0.32 mmol, 1.0 eq) in dioxane (5 mL) and H₂O (5 mL) was added Et₃N (134 μL, 0.96 mmol, 3.0 eq) and Boc anhydride (84 mg, 0.38 mmol, 1.2 eq) at 0° C. The resulting mixture was stirred at room temperature for 2.5 h, and then concentrated under reduced pressure. The residue was acidified by addition of 0.1M HCl to pH 2-3 and then diluted with EtOAc (50 mL). The organic layer was washed with brine (3×10 mL), dried over Na₂SO₄, filtered, and concentrated under reduced pressure. The obtained crude product S3e (113 mg, 94%) was used without further purification. ¹H NMR (400 MHz, CDCl₃) δ 1.28 (s, 9H, Boc), 2.74-3.06 (m, 1H, βCH₂), 3.15 (d, J=14.5 Hz, 1H, βCH2), 4.33-4.55 (m, 1H, αCH), 5.00 (d, J=6.7 Hz, 2H, OBn CH₂), 6.82 (d, J=8.0 Hz, 2H, Ph), 7.04-7.15 (m, 2H, Ph), 7.21 (t, J=7.3 Hz, 1H, Ph), 7.28 (d, J=14.8 Hz, 2H, Ph), 7.36 (d, J=7.4 Hz, 2H, Ph), 9.86 (s, 1H, COOH). ¹³C NMR (100 MHz, CDCl₃) δ 28.30, 29.71, 32.44, 54.81, 70.26, 111.95, 121.12, 127.22, 127.93, 128.46, 128.64, 131.40, 176.93. LC-MS: t_{R}=4.28 min; C₂₀¹³CH₂₅NNaO₅⁺ [M+Na]⁺, m/z calcd 395.17, found 394.83; C₁₅¹³CH₁₈NO₃⁺ [M+H−Boc]⁺, m/z calcd 273.13, found 272.82.

tert-Butyl (S)-(3-(2-(benzyloxy)phenyl)-1-(methoxy(methyl)amino)-1-oxopropan-2-yl-1-¹³C)carbamate (S3f). Prepared from S3e (113 mg, 0.30 mmol, 1.0 eq) and N,O-dimethylhydroxylamine hydrochloride (44.4 mg, 0.45 mmol, 1.5 eq) in a similar procedure as described for S1d. The obtained crude product was purified by FCC (25% EtOAc in hexane) to give S3f (136 mg, 100%). ¹H NMR (400 MHz, CDCl₃) δ 1.25 (s, 9H, Boc), 2.79-2.96 (m, 2H, o-Tyr βCH₂), 2.99 (d, J=5.6 Hz, 3H, OCH₃), 3.44 (s, 3H, NCH₃), 4.90 (s, 1H, αCH), 4.99 (d, J=4.1 Hz, 2H, OBn CH₂), 5.21 (d, J=8.9 Hz, 1H, NH), 6.79 (t, J=7.7 Hz, 2H, Ph), 7.00-7.14 (m, 2H, Ph), 7.18-7.32 (m, 3H, Ph), 7.38 (d, J=7.5 Hz, 2H, Ph). ¹³C NMR (100 MHz, CDCl₃) δ 28.30, 29.66, 32.03, 45.85, 61.26, 70.23, 79.09, 111.68, 120.70, 125.50, 127.52, 127.87, 128.15, 128.54, 131.42, 137.08, 155.20, 157.10, 172.86. LC-MS: t_{R}=4.60 min; C₂₂¹³CH₃₀N₂NaO₅⁺ [M+Na]⁺, m/z calcd 438.21, found 437.81; C₁₇¹³CH₂₂N₂O₅⁺[M+H−Boc]⁺, m/z calcd 316.17, found 315.73.

N—((S)-1-(((S)-3-(2-(benzyloxy)phenyl)-1-(methoxy(methyl)amino)-1-oxopropan-2-yl-1-¹³C)amino)-1-oxo-3-phenylpropan-2-yl)-4-methylpiperazine-1-carboxamide (S3g). The Boc group of S3f (136 mg, 0.33 mmol, 1.0 eq) was first deprotected in a similar procedure as described for Sic. Subsequently, the intermediate product was coupled to NMePip-Phe-OH (114 mg, 0.39 mmol, 1.2 eq) in a similar procedure as described for S1d. The obtained crude product was purified by FCC (8% MeOH in DCM) to give S3g (107 mg, 55%).¹H NMR (400 MHz, CDCl₃) δ 2.27 (s, 3H, piperazinyl NCH₃), 2.30 (dt, J=3.7, 6.5 Hz, 4H, 2× piperazinyl CH₂), 2.96 (dd, J=2.9, 6.3 Hz, 3H, OCH₃), 3.07 (s, 4H, 2× βCH₂), 3.28 (ddd, J=7.7, 13.5, 17.9 Hz, 4H, 2× piperazinyl CH₂), 3.48 (s, 3H, NCH₃), 4.49 (q, J=6.5 Hz, 1H, αCH), 4.99 (d, J=7.0 Hz, 1H, αCH), 5.05 (s, 2H, OBn CH₂), 5.19 (s, 1H, NH), 6.62 (s, 1H, NH), 6.79 (t, J=7.3 Hz, 1H, Ph), 6.90 (dd, J=7.3, 19.1 Hz, 2H, Ph), 7.12-7.25 (m, 6H, Ph), 7.30 (t, J=7.3 Hz, 1H, Ph), 7.37 (t, J=7.4 Hz, 2H, Ph), 7.46 (d, J=7.2 Hz, 2H, Ph). ¹³C NMR (100 MHz, CDCl₃) δ 32.03, 32.92, 38.74, 43.62, 46.03, 49.23, 54.53, 55.10, 61.22, 70.22, 111.82, 120.68, 125.09, 126.70, 127.57, 127.97, 128.30, 128.36, 128.59, 129.68, 131.43, 136.95, 137.02, 156.53, 156.99, 171.19, 171.76. LC-MS: t_{R}=3.72 min; C₃₂¹³CH₄₂N₅O₅⁺ [M+H]⁺, m/z calcd 589.32, found 589.02.

N—((S)-1-(((S)-1-(2-(benzyloxy)phenyl)-3-oxopropan-2-yl-3-¹³C)amino)-1-oxo-3-phenylpropan-2-yl)-4-methylpiperazine-1-carboxamide (S3h). To a solution of S3g (107 mg, 0.182 mmol, 1.0 eq) in THE (8 mL) at −10° C. was added 2.0 M LAH in THE (218 μL, 0.218 mmol, 1.2 eq) dropwise. The resulting mixture was stirred at 0° C. for 1 h, and then quenched by tiny pieces of ice. The reaction was concentrated under reduced pressure, and diluted with EtOAc (50 mL). The organic layer was washed successively with a saturated solution of Rochelle salt (15 mL), NaHCO₃ (15 mL) and brine (15 mL), dried over Na₂SO₄, filtered, and concentrated under reduced pressure. The obtained crude product was purified by FCC (10% MeOH in DCM) to give S3h (81 mg, 84%). ¹H NMR (400 MHz, CDCl₃) δ 2.28 (s, 3H, piperazinyl NCH₃), 2.31 (t, J=4.2 Hz, 4H, 2× piperazinyl CH₂), 2.95-3.13 (m, 4H, 2× βCH₂), 3.29 (dt, J=5.2, 9.7 Hz, 4H, 2× piperazinyl CH₂), 4.52 (m, 2H, αCH & NH), 4.95 (d, J=7.3 Hz, 1H, αCH), 5.04 (dd, J=3.6, 10.5 Hz, 2H, OBn CH₂), 6.82 (s, 3H, Ph & NH), 7.10-7.24 (m, 6H, Ph), 7.29-7.50 (m, 6H, Ph), 9.36 (s, 1H, CHO). ¹³C NMR (100 MHz, CDCl₃) δ 29.66, 38.59, 43.67, 46.04, 54.52, 55.31, 59.49, 70.25, 97.62, 111.96, 121.04, 126.93, 127.55, 128.19, 128.55, 128.60, 128.73, 129.33, 129.43, 131.61, 136.52, 136.76, 156.39, 156.60, 171.89, 198.37. LC-MS: t_{R}=3.76 min; C₃₀¹³CH₃₆N₄O₄⁺ [M+H]⁺, m/z calcd 530.28, found 530.03.

N-((2S)-1-(((3S)-2-hydroxychroman-3-yl-2-¹³C)amino)-1-oxo-3-phenylpropan-2-yl)-4-methylpiperazine-1-carboxamide (³C-labeled 12). Prepared from S3h (81 mg, 0.152 mmol, 1.0 eq) in a similar procedure as described for Sib. The obtained crude product was purified by Prep-HPLC to give ¹³C-labeled compound 36 (45 mg, 67%). ¹H NMR (400 MHz, CDCl₃) δ 1.28 (s, 1H, lactol OH), 2.45 (s, 3H, NCH₃), 2.65 (p, J=6.5 Hz, 4H, 2× piperazinyl CH₂), 3.03 (dt, J=7.6, 21.2 Hz, 2H, o-Tyr βCH₂), 3.40-3.60 (m, 4H, 2× piperazinyl CH₂), 4.21-4.40 (m, 1H, Phe βCH₂), 4.64 (dq, J=7.7, 46.1 Hz, 1H, Phe βCH₂), 5.38 (dd, J=30.9, 170.9 Hz, 1H, o-Tyr αCH), 6.16 (d, J=8.0 Hz, 1H, Phe αCH), 6.50 (dd, J=8.0, 141.5 Hz, 1H, lactol CH), 6.86-7.08 (m, 3H, Ph), 7.13 (dd, J=3.0, 6.9 Hz, 1H, Ph), 7.20 (dt, J=2.3, 6.5 Hz, 2H, Ph), 7.25 (d, J=6.7 Hz, 2H, Ph), 7.71 (s, 2H, Ph & NH), 8.27 (s, 1H, NH). ¹³C NMR (100 MHz, CDCl₃, scanned for only a few times) δ 91.69, 92.25. LC-MS: t_{R}=2.77 min; C₂₃¹³CH₃₁N₄O₄⁺ [M+H]⁺, m/z calcd 440.24, found 439.94.

In Scheme 4, the preparation of S4a and S4b was similar to the procedures for S1a and Sib, respectively; the preparation of S4d, S4e, and S4f was similar to the procedures for S3f, S3g and S3h, respectively. Therefore, only the preparation of S4c was described in detail as below.

2-((tert-butoxycarbonyl)amino)-3-(2-oxo-1,2-dihydropyridin-3-yl)propanoic acid (S4c). A mixed solution of S4b (1.18 g, 4.0 mmol, 1.0 eq) and LiOH (192 mg, 8.0 mmol, 2.0 eq) in MeOH (10 mL) and H₂O (5 mL) was stirred at room temperature for 2 h. The reaction was acidified by addition of 0.1M HCl to pH 2-3, and then concentrated under reduced pressure. The residue was extracted with DCM (3×50 mL). The organic layers were combined, dried over Na₂SO₄, filtered, and concentrated under reduced pressure. The obtained crude product S4c was used without further purification. ¹H NMR (400 MHz, CDCl₃) δ 1.39 (s, 9H, Boc), 2.86 (dd, J=8.9, 14.0 Hz, 1H, βCH₂), 3.11 (dd, J=4.3, 13.9 Hz, 1H, βCH₂), 4.42 (dd, J=4.3, 8.8 Hz, 1H, αCH), 6.32 (t, J=6.7 Hz, 1H, pyridine), 7.30 (dd, J=2.0, 6.6 Hz, 1H, pyridine), 7.39-7.54 (m, 1H, pyridine).

Enzyme Assays. The preparation of cruzain and 3CL^{pro} were described in previous publications.^{8, 34} The evaluation of inhibitors was modified from our lab protocol and described briefly as follows.³⁵ All enzyme assays were conducted in 96-well, clear bottom, black plates at 25° C., and fluorescence emanating from product formation was measured using either a SpectraMax M5 (Molecular Devices) or a Synergy HTX (Biotek) microplate reader, with excitation at λₑₓ=360 nm and emission at λₑₘ=460 nm. For evaluation of inhibitors that exhibited time-dependent time courses (“burst kinetics”), variable concentrations of inhibitor and a fixed concentration of substrate [typically, for cruzain: 10 μM Cbz-Phe-Arg-AMC (Kₘ=0.9 μM), for 3CL^{pro}: 40 μM ACC-SAVLQSGFRK(DNP)-NH₂ (Kₘ=26 μM)] were first added in assay buffer [for cruzain: 50 mM MES (pH 7.5), 50 mM TAPSO, 1 mM CHAPS, 1 mM Na₂EDTA, 5 mM DTT, and 10% DMSO (v/v); for 3CLpro: 20 mM Tris HCl (pH 7.5), 150 mM NaCl, 0.1 mM EDTA-Na₂, 2 mM DTT, and 10% DMSO (v/v)]. Reactions were started with addition of enzyme (typically, to a final concentration of 0.2 nM for cruzain or 40 nM for 3CL^{pro}), and the time courses were recorded for 0.5-1 h. In rapid dilution assay, 100×concentration of enzyme and inhibitor were pre-incubated for 1-2 h, and then diluted by 100-fold into assay buffer containing standard concentration of substrate, followed by data collection.

In vitro Cell Assays. Bloodstream form T. b. brucei strain SM427 was grown in HM1-9 medium that included fetal bovine serum (10%) and antibiotics (100 U/mL penicillin-streptomycin, Gibco™). Inhibitors dissolved in 100% (v/v) DMSO stock solutions were added at final concentrations of 0.5-20 μM (final concentrations of DMSO were <1% (v/v)). T. b. brucei were seeded at ˜1×10⁶ cells at 37° C., and diluted daily to maintain a log phase of growth for up to 96 h. Treated cells were typically grown for 4 days. After each dilution, the cell cultures were supplemented with inhibitor or DMSO (control) to keep concentrations constant. Cell density was determined and graphed as previously reported.³⁶ Trypanosomes were quantified using a Z2 Coulter Counter, and values of EC₅₀ were determined after 3-4 days of treatment by the interpolation method of Huber and Koella.³⁷

T. cruzi infection was evaluated in C2C12 murine cardiomyoblast cells. 384-microwell plates containing C2C12 murine cardiomyoblast cells were infected with 10³ and 10⁴ cells of T. cruzi (Ca-I/72), to which was added 3-fold serially-dilutions of inhibitor with maximal concentrations of 10 μM, followed by incubation at 37° C. for 2 days. The wells were fixed with 2% PFA in PBS, and stained by 5 μg/ml of DAPI. After >30 min of incubation in a dark environment, the plates were read on an ImageXpress MicroXL microscope (Molecular Devices). The images were analyzed to quantify parasites as well host cells.

Evaluation of the anti-CoV-2 activity of compound 43 using human A549 cells transduced with ACE2 viral receptor (A549/ACE2) was as described in our recent publication.⁸

Evaluation of SMAI Prodrugs. Esterase from porcine liver was obtained as an ammonium sulfate suspension (Sigma-Aldrich, E2884, 10 U/μL). Compounds 37-39 in 100% (v/v) DMSO solutions were diluted by 10-fold into cruzain assay buffer to make 1 mL-samples, each containing 2 mM compound. For the experimental sample, 4 μL of esterase suspension was added to the reaction mixtures (40 U/mL), which was immediately placed on a low-speed orbital shaker in a 37° C. incubator. In the meantime, a negative control (lacking esterase) and a blank control (buffer only) were included. Aliquots (140 μL) of the reaction mixtures were removed at 0, 15, 30, 60, 120, 180, and 300 min, and reactions were quenched by the addition of MeCN (260 μL). The resulting solution was vortexed, followed by centrifugation to precipitate the protein, and the resulting compounds in the supernatant were analyzed by HPLC-MS with detection at 270 nm.

Commercially-available CYPs 3A4, 2D6 and 2C9 (CypExpress™; Sigma-Aldrich, MTOXCE3A4, MTOXCE3D6, and MTOXCE2C9) containing glucose-6-phosphate dehydrogenase (G6PDH) and Mg²⁺ were used to evaluate the stability of acetal prodrugs to these oxidases. The following components were combined in 800-μL reaction mixtures: 0.5 mM 40 or 41, 5 mM sodium glucose 6-phosphate, 2 mM NADP⁺, cruzain assay buffer (pH 7.5), and 2.5% DMSO (v/v). For the experimental samples, 64 mg of CypExpress™ P450 as a lyophilized powder was added to the reaction vial (80 mg/mL), which was gently shaken to dissolve the solid, and the mixtures were stirred at 30° C. in an incubator. Sample vials were capped loosely with aluminum foil to allow aeration as well as to reduce evaporation. Additionally, a negative control lacking CypExpress™ P450, and a blank control containing only buffer were included. Aliquots (65 μL) were removed from mixtures at 0, 30, 60, 120, 180, and 240 min, and reactions were quenched with the addition of MeCN (65 μL). The resulting solution was vortexed, followed by centrifugation to precipitate the protein, and the resulting compounds in the supernatant were analyzed by HPLC-MS with detection at 270 nm.

2D NMR Study. Wilmad precision NMR tubes (541-PP-8, thin wall, 800 MHz, 5 mm×8 inch) were used for this experiment. The total volume of sample in each tube was 660 μL, and the final concentrations of the components in the NMR buffer were as follows: 18 mM phosphate (K₂HPO₄/KH₂PO₄), 45 mM NaCl, 4.5 mM DTT, pH 7.5, 10% DMSO-d₆ (v/v). A 10 mM stock of ¹³C-labeled 36 in DMSO-d₆ was diluted into the NMR buffer to specified concentrations. A control sample contained 0.6 mM of ¹³C-labeled 36, and an experimental sample contained 0.4 mM of ¹³C-labeled 36 and 0.44 mM of cruzain freshly purified by size exclusion chromatography, directly after thawing from a −80° C. stock to minimize auto-proteolysis. Both samples were analyzed on a Bruker AVANCE III HD 800 MHz (18.8 Tesla) equipped with a 5-mm triple resonance cryoprobe (TCI). ¹H-¹³C HSQC NMR spectra were acquired at 25° C. with 72 scans for the enzyme-free control sample and 128 scans for experimental sample containing cruzain. Spectral widths were obtained at 9615 Hz and 28179 Hz in the ¹³C and ¹H dimensions, respectively. Spectra were processed using NMRpipe,³⁸ and cross-peak intensities were analyzed using Sparky³⁹ and MestreNova⁴⁰.

Detection of Aldehyde Content Using Phenylhydrazine. In a 96-well clear microplate, compounds 52 and 36 in DMSO solutions, or pure DMSO as a control, were diluted into the above NMR buffer without addition of DTT to make 250 μL-samples, each containing 0.2 mM of compounds and 10% DMSO (v/v). Upon addition of 1M phenylhydrazine (0.25 μL), the formation of phenylhydrazone in these wells were continuously monitored for 2 hours by measuring UV absorbance at the maximal wavelength of 282 nm.

X-Ray Crystallography. The procedure of crystallization for 3CL^{pro}-43 complex is based on the previous publication.⁴¹ The crystals were obtained at 25° C. from 0.2 M ammonium phosphate dibasic, 17% w/v PEG3350, pH8.0, with a protein concentration of 14 mg/ml. Soaking was performed to produce 3CL^{pro}-43 complex crystals. The crystals were washed three times with reservoir solution plus 0.5 mM inhibitor and 2% DMSO (Inhibitor was dissolved to 25 mM in 100% DMSO). The mixture was incubated at 25° C. for 48 h. The cryo-protectant solution contained mother liquor plus 30% glycerol, 0.5 mM inhibitor and 2% DMSO. Cryo-protected crystals were fished for data collection.

The 3CL^{pro}-43 data were collected at 1.70 Å on a Rigaku R-AXIS IV++ image plate detector. Reflections were indexed, integrated, scaled, and merged to space group I1 2 1 using iMosflm.⁴² The structure was solved by molecular replacement using the Phaser module of the Phenix package using a high-resolution structure of the apo SARS-CoV-2 3CL^{pro} (PDB accession code: 6Y2E) as the search model.^{43, 44} The atomic coordinates and geometric restraints for the inhibitor were generated in CCP4 suite.⁴² The inhibitor was built into the Fₒ-F_{c} electron density omit maps using Coot.⁴⁵ Structural refinement was performed with the Real-space Refinement module of the Phenix package. Crystallographic data were summarized in Table S4. All structural figures were generated with PyMOL (https://www.pymol.org).

Molecular Modeling. Docking studies of compound 30 binding to cruzain were carried out using the Schrödinger Suite program package. The receptor file (PDB accession code: 20Z2) was processed by the Protein Preparation Wizard function, and the compound was energetically minimized by the LigPrep function. Non-covalent docking was executed and scored by the Glide module. For covalent docking, a cubic box of 18 Å³ was set to encompass any simulated poses with K777 as a reference ligand at its centroid. The SMARTS pattern for 30 was manually defined as [C; H1]=[O], and Cys₂₅ was checked as the reactive residue. The binding affinity was scored, and the best three poses were visually inspected.

Kinetic Data Analysis. All time-course data were fitted to eq. 1, wherein P is the fluorescence generated by formation of AMC or liberated ACC, C is the background constant, vᵢ and vₛ are respectively initial-state and steady-state reaction rates, t is time, and k_{obs} is the observed rate of conversion of the initial inhibited rate to the final inhibited rate.⁴⁶

$$P = {v}_{s} t + ( \frac{{v}_{i} - {v}_{s}}{{k}_{obs}} ) ( 1 - {e}^{- {k}_{obs} t} ) + C \qquad ( 1 )$$

Values of k_{obs} vs. [I] were then re-plotted and fitted to eq. 2, wherein k₃ and k₄ represent the respective rates of formation and dissolution of the EI* complex as depicted in FIG. 22A.

$${k}_{obs} = {k}_{4} + \frac{{k}_{3} [ I ]}{{K}_{i} ( 1 + \frac{[ S ]}{{K}_{m}} ) + [ I ]} \qquad ( 2 )$$

As described in the main text, the linearity of k_{obs} vs. [I] of compounds 28 and 36 arises when Kᵢ>>Kᵢ*. Under these conditions, the concentration of inhibitor required to observe time-dependent inhibition would be much less than the value of Kᵢ for the EI complex. In this case, eq. 2 reduces to eq. 3, which is a linear function with a slope=k₄/Kᵢ*^{app} and an y-intercept=k₄. For the rapid dilution assay, [I] was fixed at 0.1×Kᵢ*^{app} and after dilution, k₄ could be estimated as k_{obs}/1.1 according to eq. 3.

$${k}_{obs} = {k}_{4} [ 1 + \frac{[ I ]}{{K}_{i}^{*} ( 1 + \frac{[ S ]}{{K}_{m}} )} ] \qquad ( 3 )$$

Overall inhibition constants (Kᵢ* values) were obtained by fitting vₛ/v₀ to eq. 4, wherein vₛ and v₀ are steady-state reaction rates in the presence and absence of inhibitor respectively, [S] and [I] represent the fixed concentration of substrate and variable inhibitor, Kₘ is the Michaelis constant of the substrate.

$$\frac{{v}_{s}}{{v}_{0}} = \frac{1}{1 + [ I ] / [ {K}_{i}^{*} ( 1 + \frac{[ S ]}{{K}_{m}} ) ]} \qquad ( 4 )$$

As a tight-binding inhibitor, the Kᵢ* values of 1 for cruzain and 43 for 3CL^{pro} were obtained by fitting vᵢ/v₀ data to eq. 5 in which v, and v₀ are the initial rates obtained with or without inhibitor, respectively, [E]ₜ, [I]ₜ, [S], Kᵢ*, and Kₙ, are respectively, concentrations of enzyme, inhibitor, and substrate, and Kᵢ* and Kₘ are the inhibition and Michaelis constants, respectively.⁴⁷

$$\frac{{v}_{i}}{{v}_{0}} = \text{?} \qquad ( 5 )$$

$$\text{?} \text{indicates text missing or illegible when filed}$$

Rates of de-acylation reactions of SMAI prodrugs 37-39 were obtained upon evaluation of the first-order loss of reagent as fitted to eq. 6, in which A₀ and C are the initial and final fraction of unhydrolyzed compound, respectively, k is the pseudo-first order rate constant, from which the half-life was t_{1/2}=ln 2/k.

A=(A₀−C)e^{-kt}+C (6)

### REFERENCES

1. Bedard, K. M.; Semler, B. L. Regulation of picornavirus gene expression. Microbes Infect. 2004, 6, 702-13.
2. Morse, J. S.; Lalonde, T.; Xu, S.; Liu, W. R. Learning from the past: Possible urgent prevention and treatment options for severe acute respiratory infections caused by 2019-nCoV. ChemBioChem 2020, 21, 730-738.
3. Steele-Mortimer, O. Exploitation of the ubiquitin system by invading bacteria. Traffic 2011, 12, 162-9.
4. Sajid, M.; McKerrow, J. H. Cysteine proteases of parasitic organisms. Mol. Biochem. Parasitol. 2002, 120, 1-21.
5. Rassi, A., Jr.; Rassi, A.; Marcondes de Rezende, J. American trypanosomiasis (Chagas disease). Infect. Dis. Clin. North Am. 2012, 26, 275-91.
6. Branquinha, M. H.; Oliveira, S. S.; Sangenito, L. S.; Sodre, C. L.; Kneipp, L. F.; d'Avila-Levy, C. M.; Santos, A. L. Cruzipain: An update on its potential as chemotherapy target against the human pathogen Trypanosoma cruzi. Curr. Med Chem. 2015, 22, 2225-35.
7. Hernandez, A. A.; Roush, W. R. Recent advances in the synthesis, design and selection of cysteine protease inhibitors. Curr. Opin. Chem. Biol. 2002, 6, 459-65.
8. Mellott, D.; Tseng, C. T.; Drelich, A.; Fajtova, P.; Chenna, B. C.; Kostomiris, D.; Hsu, J. C.; Zhu, J.; Taylor, Z.; Tat, V.; Katzfuss, A.; Li, L.; Giardini, M. A.; Skinner, D.; Hirata, K.; Beck, S.; Carlin, A. F.; Clark, A. E.; Berreta, L.; Maneval, D.; Frueh, F.; Hurst, B. L.; Wang, H.; Kocurek, K. I.; Raushel, F. M.; O'Donoghue, A.; Siqueira-Neto, J. L.; Meek, T. D.; McKerrow, J. H. A cysteine protease inhibitor blocks SARS-CoV-2 infection of human and monkey cells. bioRxiv 2020, 2020.10.23.347534.
9. Gampe, C.; Verma, V. A. Curse or cure? A perspective on the developability of aldehydes as active pharmaceutical ingredients. J. Med Chem. 2020, 63, 14357-14381.
10. Bandyopadhyay, A.; Gao, J. Targeting biomolecules with reversible covalent chemistry. Curr. Opin. Chem. Biol. 2016, 34, 110-116.
11. Choe, Y.; Brinen, L. S.; Price, M. S.; Engel, J. C.; Lange, M.; Grisostomi, C.; Weston, S. G.; Pallai, P. V.; Cheng, H.; Hardy, L. W.; Hartsough, D. S.; McMakin, M.; Tilton, R. F.; Baldino, C. M.; Craik, C. S. Development of alpha-keto-based inhibitors of cruzain, a cysteine protease implicated in chagas disease. Bioorg. Med Chem. 2005, 13, 2141-56.
12. Scheidt, K. A.; Roush, W. R.; McKerrow, J. H.; Selzer, P. M.; Hansell, E.; Rosenthal, P. J. Structure-based design, synthesis and evaluation of conformationally constrained cysteine protease inhibitors. Bioorg. Med Chem. 1998, 6, 2477-94.
13. Silva, D. G.; Ribeiro, J. F. R.; De Vita, D.; Cianni, L.; Franco, C. H.; Freitas-Junior, L. H.; Moraes, C. B.; Rocha, J. R.; Burtoloso, A. C. B.; Kenny, P. W.; Leitao, A.; Montanari, C. A. A comparative study of warheads for design of cysteine protease inhibitors. Bioorg. Med Chem. Lett. 2017, 27, 5031-5035.
14. Bonatto, V.; Batista, P. H. J.; Cianni, L.; De Vita, D.; Silva, D. G.; Cedron, R.; Tezuka, D. Y.; de Albuquerque, S.; Moraes, C. B.; Franco, C. H.; Lameira, J.; Leitao, A.; Montanari, C. A. On the intrinsic reactivity of highly potent trypanocidal cruzain inhibitors. RSC Med Chem. 2020, 11, 1275-1284.
15. O'Brien, P. J.; Siraki, A. G.; Shangari, N. Aldehyde sources, metabolism, molecular toxicity mechanisms, and possible effects on human health. Crit. Rev. Toxicol. 2005, 35, 609-62.
16. Gibbons, P.; Verissimo, E.; Araujo, N. C.; Barton, V.; Nixon, G. L.; Amewu, R. K.; Chadwick, J.; Stocks, P. A.; Biagini, G. A.; Srivastava, A.; Rosenthal, P. J.; Gut, J.; Guedes, R. C.; Moreira, R.; Sharma, R.; Berry, N.; Cristiano, M. L.; Shone, A. E.; Ward, S. A.; O'Neill, P. M. Endoperoxide carbonyl falcipain 2/3 inhibitor hybrids: Toward combination chemotherapy of malaria through a single chemical entity. J. Med Chem. 2010, 53, 8202-6.
17. Schechter, I.; Berger, A. On the size of the active site in proteases. I. Papain. Biochem. Biophys. Res. Commun. 1967, 27, 157-62.
18. Cuerrier, D.; Moldoveanu, T.; Inoue, J.; Davies, P. L.; Campbell, R. L. Calpain inhibition by alpha-ketoamide and cyclic hemiacetal inhibitors revealed by X-ray crystallography. Biochemistry 2006, 45, 7446-52.
19. Nakamura, M.; Yamaguchi, M.; Sakai, O.; Inoue, J. Exploration of cornea permeable calpain inhibitors as anticataract agents. Bioorg. Med Chem. 2003, 11, 1371-1379.
20. Kerr, I. D.; Lee, J. H.; Farady, C. J.; Marion, R.; Rickert, M.; Sajid, M.; Pandey, K. C.; Caffrey, C. R.; Legac, J.; Hansell, E.; McKerrow, J. H.; Craik, C. S.; Rosenthal, P. J.; Brinen, L. S. Vinyl sulfones as antiparasitic agents and a structural basis for drug design. J. Biol. Chem. 2009, 284, 25697-703.
21. Clementi, E.; Raimondi, D. L.; Reinhardt, W. P. Atomic screening constants from SCF functions. II. Atoms with 37 to 86 electrons. J. Chem. Phys 1967, 47, 1300-1307.
22. Clayden, J.; Greeves, N.; Warren, S. G. Organic chemistry. Second ed.; Oxford University Press: Oxford, 2012.
23. Mackenzie, N. E.; Grant, S. K.; Scott, A. I.; Malthouse, J. P. ¹³C NMR study of the stereospecificity of the thiohemiacetals formed on inhibition of papain by specific enantiomeric aldehydes. Biochemistry 1986, 25, 2293-8.
24. Abdulla, M. H.; O'Brien, T.; Mackey, Z. B.; Sajid, M.; Grab, D. J.; McKerrow, J. H. RNA interference of Trypanosoma brucei cathepsin B and L affects disease progression in a mouse model. PLoS Negl. Trop. Dis. 2008, 2, e298.
25. Enanga, B.; Ariyanayagam, M. R.; Stewart, M. L.; Barrett, M. P. Activity of megazol, a trypanocidal nitroimidazole, is associated with DNA damage. Antimicrob. Agents Chemother. 2003, 47, 3368-70.
26. Jacobsen, W.; Christians, U.; Benet, L. Z. In vitro evaluation of the disposition of a novel cysteine protease inhibitor. Drug Metab. Dispos. 2000, 28, 1343-51.
27. Charrier, J. D.; Durrant, S. J.; Studley, J.; Lawes, L.; Weber, P. Synthesis and evaluation of novel prodrugs of caspase inhibitors. Bioorg. Med. Chem. Lett. 2012, 22, 485-8.
28. Wang, D.; Zou, L.; Jin, Q.; Hou, J.; Ge, G.; Yang, L. Human carboxylesterases: A comprehensive review. Acta Pharm. Sin. B 2018, 8, 699-712.
29. Zanger, U. M.; Schwab, M. Cytochrome P450 enzymes in drug metabolism: Regulation of gene expression, enzyme activities, and impact of genetic variation. Pharmacol. Ther. 2013, 138, 103-41.
30. Fu, L.; Ye, F.; Feng, Y.; Yu, F.; Wang, Q.; Wu, Y.; Zhao, C.; Sun, H.; Huang, B.; Niu, P.; Song, H.; Shi, Y.; Li, X.; Tan, W.; Qi, J.; Gao, G. F. Both boceprevir and GC376 efficaciously inhibit SARS-CoV-2 by targeting its main protease. Nat. Commun. 2020, 11, 4417.
31. Frank, J.; Katritzky, A. R. Tautomeric pyridines. Part xv. Pyridone-hydroxypyridine equilibria in solvents of differing polarity. J. Chem. Soc., Perkin Trans. 2 1976, 1428-1431.
32. Zhao, M.-M.; Yang, W.-L.; Yang, F.-Y.; Zhang, L.; Huang, W.; Hou, W.; Fan, C.; Jin, R.; Feng, Y.; Wang, Y.; Yang, J.-K. Cathepsin L plays a key role in SARS-CoV-2 infection in humans and humanized mice and is a promising target for new drug development. medRxiv 2020, 2020.10.25.20218990.
33. Noisier, A. F. M.; Harris, C. S.; Brimble, M. A. Novel preparation of chiral α-amino acids using the Mitsunobu-Tsunoda reaction. Chem. Commun. 2013, 49, 7744-7746.
34. Zhai, X.; Meek, T. D. Catalytic mechanism of cruzain from Trypanosoma cruzi as determined from solvent kinetic isotope effects of steady-state and pre-steady-state kinetics. Biochemistry 2018, 57, 3176-3190.
35. Chenna, B. C.; Li, L.; Mellott, D. M.; Zhai, X.; Siqueira-Neto, J. L.; Calvet Alvarez, C.; Bernatchez, J. A.; Desormeaux, E.; Alvarez Hernandez, E.; Gomez, J.; McKerrow, J. H.; Cruz-Reyes, J.; Meek, T. D. Peptidomimetic vinyl heterocyclic inhibitors of cruzain effect antitrypanosomal activity. J. Med. Chem. 2020, 63, 3298-3316.
36. Wang, Z.; Morris, J. C.; Drew, M. E.; Englund, P. T. Inhibition of Trypanosoma brucei gene expression by RNA interference using an integratable vector with opposing t7 promoters. J. Biol. Chem. 2000, 275, 40174-9.
37. Huber, W.; Koella, J. C. A comparison of three methods of estimating EC₅₀ in studies of drug resistance of malaria parasites. Acta Trop. 1993, 55, 257-261.
38. Delaglio, F.; Grzesiek, S.; Vuister, G. W.; Zhu, G.; Pfeifer, J.; Bax, A. NMRPipe: A multidimensional spectral processing system based on Unix pipes. J. Biomol. NMR 1995, 6, 277-293.
39. Lee, W.; Tonelli, M.; Markley, J. L. NMRFAM-Sparky: Enhanced software for biomolecular NMR spectroscopy. Bioinformatics 2014, 31, 1325-1327.
40. Willcott, M. R. Mestre Nova. J. Am. Chem. Soc. 2009, 131, 13180-13180.
41. Yang, K. S.; Ma, X. R.; Ma, Y.; Alugubelli, Y. R.; Scott, D. A.; Vatansever, E. C.; Drelich, A. K.; Sankaran, B.; Geng, Z. Z.; Blankenship, L. R.; Ward, H. E.; Sheng, Y. J.; Hsu, J. C.; Kratch, K. C.; Zhao, B.; Hayatshahi, H. S.; Liu, J.; Li, P.; Fierke, C. A.; Tseng, C. K.; Xu, S.; Liu, W. R. A quick route to multiple highly potent SARS-CoV-2 main protease inhibitors*. ChemMedChem 2020.
42. Winn, M. D.; Ballard, C. C.; Cowtan, K. D.; Dodson, E. J.; Emsley, P.; Evans, P. R.; Keegan, R. M.; Krissinel, E. B.; Leslie, A. G. W.; McCoy, A.; McNicholas, S. J.; Murshudov, G. N.; Pannu, N. S.; Potterton, E. A.; Powell, H. R.; Read, R. J.; Vagin, A.; Wilson, K. S. Overview of the CCP4 suite and current developments. Acta Crystallogr. D 2011, 67, 235-242.
43. Zhang, L.; Lin, D.; Sun, X.; Curth, U.; Drosten, C.; Sauerhering, L.; Becker, S.; Rox, K.; Hilgenfeld, R. Crystal structure of SARS-CoV-2 main protease provides a basis for design of improved α-ketoamide inhibitors. Science 2020, 368, 409-412.
44. Adams, P. D.; Afonine, P. V.; Bunkoczi, G.; Chen, V. B.; Davis, I W.; Echols, N.; Headd, J. J.; Hung, L.-W.; Kapral, G. J.; Grosse-Kunstleve, R. W.; McCoy, A. J.; Moriarty, N. W.; Oeffner, R.; Read, R. J.; Richardson, D. C.; Richardson, J. S.; Terwilliger, T. C.; Zwart, P. H. Phenix: A comprehensive Python-based system for macromolecular structure solution. Acta Crystallogr. D 2010, 66, 213-221.
45. Emsley, P.; Lohkamp, B.; Scott, W. G.; Cowtan, K. Features and development of Coot. Acta Crystallogr. D 2010, 66, 486-501.
46. Morrison, J. F.; Walsh, C. T. The behavior and significance of slow-binding enzyme inhibitors. In Adv. Enzymol. Relat. Areas mol. Biol., VCH: 1988; Vol. 61, pp 201-301.
47. Copeland, R. A. Tight binding inhibitors. In Enzymes: A practical introduction to structure, mechanism, and data analysis, VCH: New York, 2000; pp 305-317.
48. Chen, Y. T.; Brinen, L. S.; Kerr, I. D.; Hansell, E.; Doyle, P. S.; McKerrow, J. H.; Roush, W. R. In vitro and in vivo studies of the trypanocidal properties of WRR-483 against Trypanosoma cruzi. PLoS Negl. Trop. Dis. 2010, 4, e825

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound having Formula I: 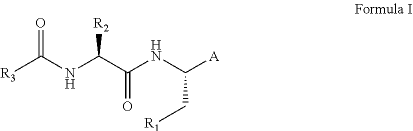
wherein
R₁ is a substituted or unsubstituted aryl, heteroaryl, or alkyl;
R₂ and R₃ are independently hydrogen, substituted or unsubstituted alkyl, aryl, cycloalkyl, or heteroaryl, or R₂ and the adjacent N atom, together with the atoms to which they are attached, combine to form a 3 to 6 membered heterocycle;
A is 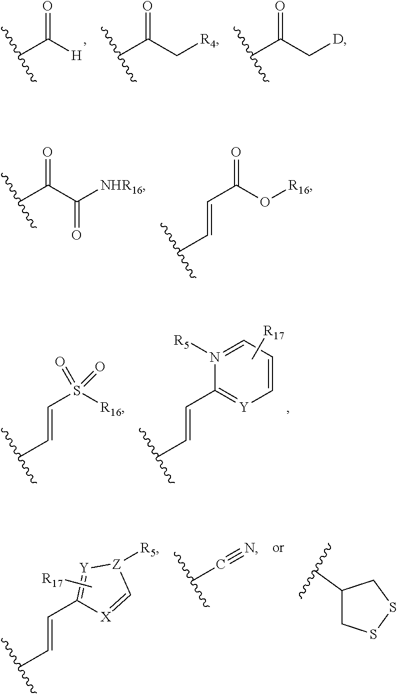
D is a halogen;
X and Y are independently NR₁₉ or CR₁₉;
Z is NR₁₉, S, O, or CR₁₉;
R₄ is substituted or unsubstituted alkyl, substituted or unsubstituted aryl, hydroxyl, thiol, or amino;
R₅ is absent, or substituted or unsubstituted alkyl;
R₁₆ is substituted or unsubstituted alkyl or aryl;
R₁₇ is a hydrogen or an electron-donating such as an alkyl, alkoxy, amino, or electron-withdrawing group such as a F, Cl, Br, trifluoromethyl, cyano, or nitro; and
R₁₉ is absent or hydrogen,
or pharmaceutically acceptable salts thereof.

2. The compound of claim 1, wherein R₁ is 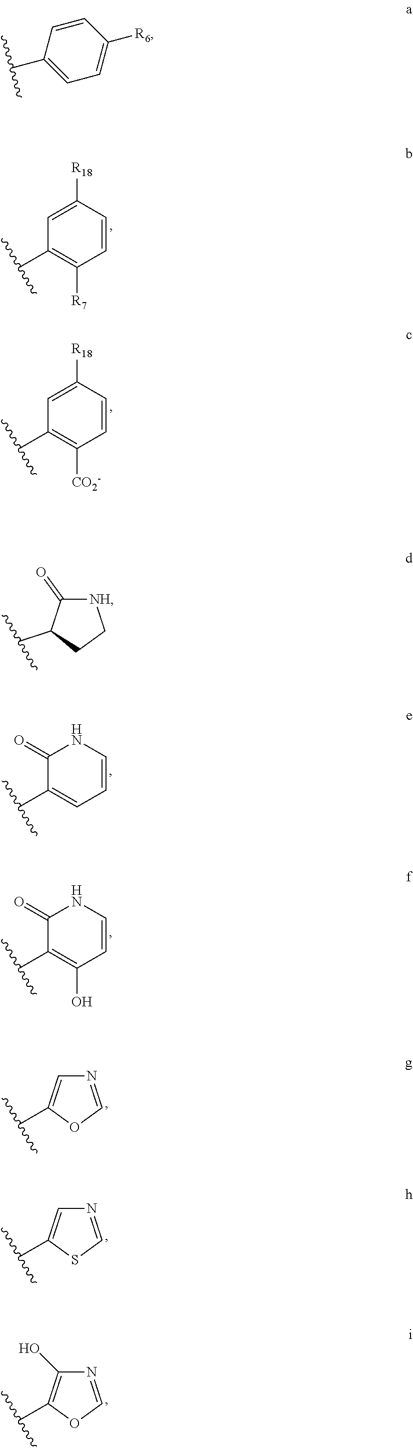 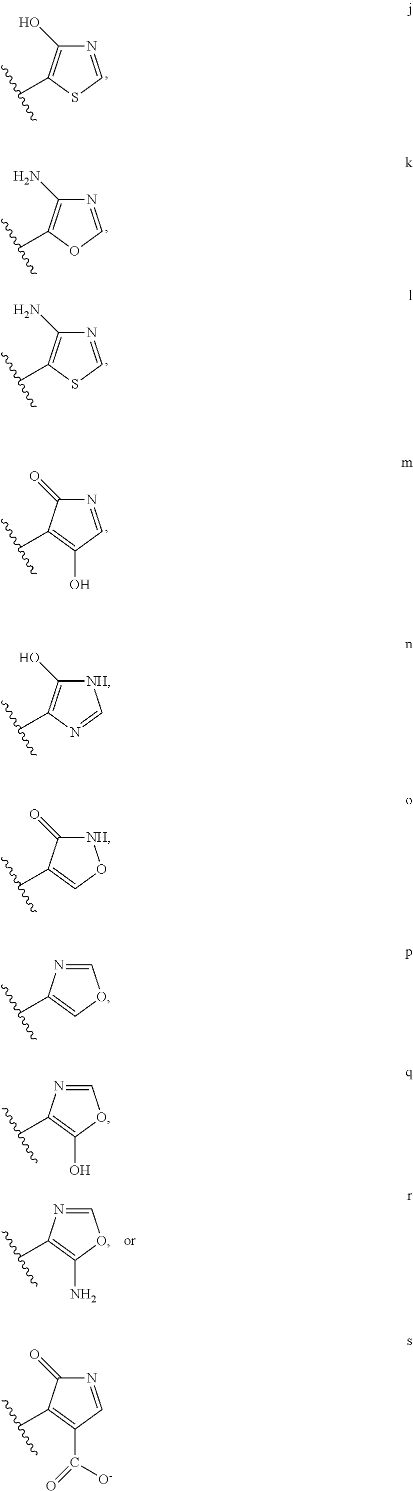
wherein
R₆, R₇, and R₁₈ are independently hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl.

3. The compound of claim 1, R₁₈ is hydrogen, substituted or unsubstituted alkyl, substituted or unsubstituted alkoxy, amino, halogen, nitro, cyano, —CF.

4. The compound of claim 1, R₇ is hydrogen, amino, hydroxy, or thiol.

5. The compound of claim 1, wherein R₁ is benzyl, 2-benzyl, 2-carboxybenzyl, (3R)-pyrrolidin-2-on-3-yl, pyridin-2(1H)-on-3-yl, 4-hydroxy-pyridin-2(1H)-on-3-yl, 1,3-oxazo-5-yl, 1,3-thiazo-5-yl, 4-hydroxy-1,3-oxazo-5-yl, 4-hydroxy-1,3-thiazo-5-yl, 4-amino-1,3-oxazo-5-yl, 4-amino-1,3-thiazo-5-yl, 4-hydroxy-2H-pyrrol-2-on-3-yl, 5-hydroxy-1H-imidazo-4-yl, isoxazole-3(2H)-on-4-yl, 1,3-oxazo-4-yl, 5-hydroxy-1,3-oxazo-4-yl, 5-hydroxy-1,3-oxazo-4-yl, or carboxy-2H-pyrrol-2-on-3-yl.

6. The compound of claim 1, wherein R₃ is (D-) or (L-) amino acid, acyl-amino acid, 4N-alkyl-piperazinyl, alkyloxycarbonyl, or aryloxycarbonyl.

7. The compound of claim 1, wherein R₃ is benzyloxy 4-N-methyl-piperazinyl, or benzyloxycarbonyl.

8. The compound of claim 1, wherein R₂ is benzyl, substituted benzyl, 4-pyridine, 3-amino-propyl, cyclohexyl, isopropyl, or 4-nitrobenzyl.

9. A compound having Formula Ia: 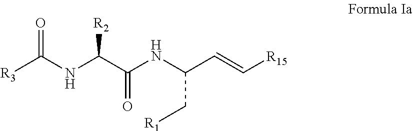
wherein
R₁ is a substituted or unsubstituted aryl, heteroaryl, or alkyl;
R₂ and R₃ are independently hydrogen, substituted or unsubstituted alkyl, aryl, cycloalkyl, or heteroaryl; or R₂ and the adjacent N atom, together with the atoms to which they are attached, combine to form a 3 to 6 membered heterocycle;
R₁₅ is selected from the group consisting of: 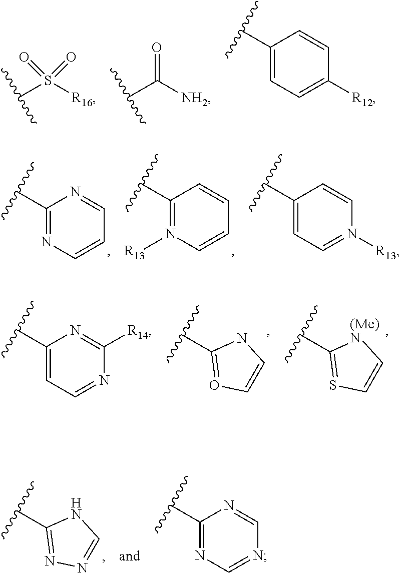
R₁₂ and R₁₄ are independently hydrogen, substituted or unsubstituted alkyl, amino, or nitro; and
R₁₆ is a substituted or unsubstituted alkyl or aryl;
R₁₃ is a substituted or unsubstituted alkyl.

10. A compound having Formula II: 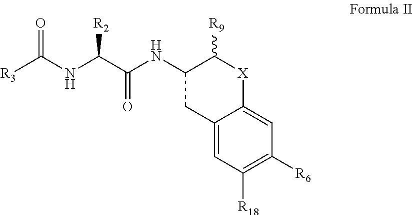
wherein
X is O, NH, and S,
R₂ and R₃ are independently hydrogen, substituted or unsubstituted alkyl, aryl, cycloalkyl, or heteroaryl, or R₂ and the adjacent N atom, together with the atoms to which they are attached, combine to form a 3 to 6 membered heterocycle;
R₁₈ and R₆ are independently hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl; and R₉ is hydroxy, or alkoxy;
or pharmaceutically acceptable salts thereof.

11. (canceled)

12. (canceled)

13. The compound of claim 10, wherein the compound is: 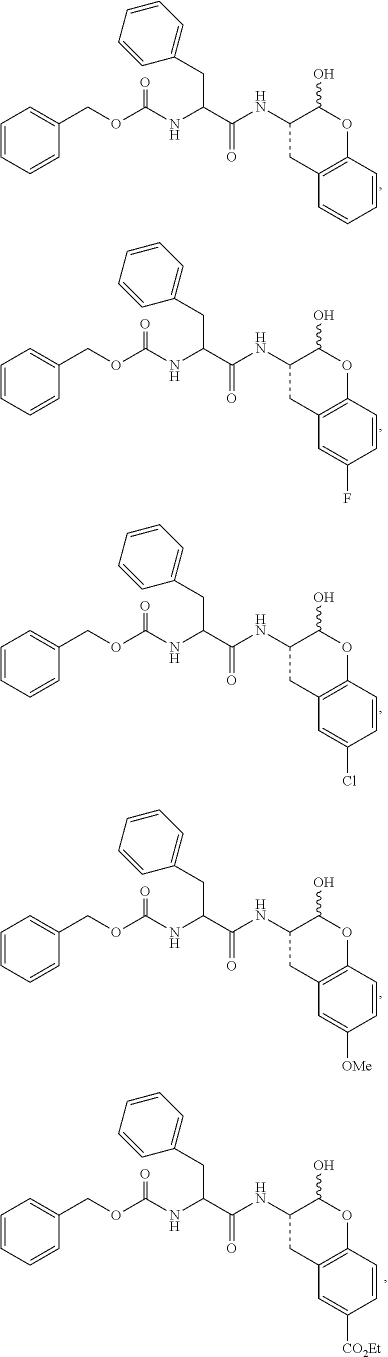 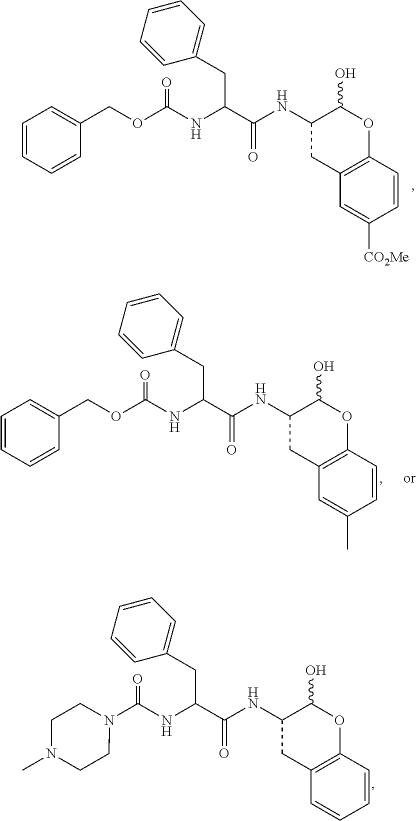
or pharmaceutically acceptable salts thereof.

14. A compound having Formula Ib: 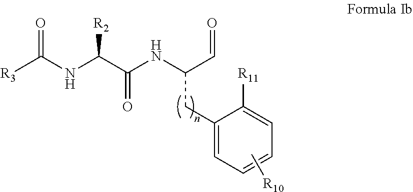
wherein
R₂ and R₃ are independently hydrogen, substituted or unsubstituted alkyl, aryl, cycloalkyl, or heteroaryl, or R₂ and the adjacent N atom, together with the atoms to which they are attached, combine to form a 3 to 6 membered heterocycle;
R₁₁ is hydroxy, thiol, or —COO—;
R₁₀ is hydrogen, hydroxy, thiol, substituted or unsubstituted alkyl, substituted or unsubstituted alkoxy, amino, halogen, nitro, cyano, —CF₃, —CO₂Rₐ, —COOH, or —CONH₂, Rₐ is a substituted or unsubstituted alkyl or aryl;
n is an integer from 1-3.

15. The compound of claim 14, wherein R₁₀ is hydrogen, methyl, or other alkyl groups, —CF₃, —NO₂, —CN, —F, —Cl, —Br, —OMe, NH₂, —COOH, —CO₂Rₐ, or —CONH₂; Rₐ is substituted or unsubstituted alkyl or aryl; and n is an integer from 1-3.

16. (canceled)

17. (canceled)

18. The compound of claim 1, wherein the compound is present in an effective amount to inhibit a cysteine protease.

19. (canceled)

20. (canceled)

21. (canceled)

22. A pharmaceutical composition comprising a compound of claim 1 and one or more pharmaceutically acceptable carriers.

23-26. (canceled)

27. A method of inhibiting coronaviral cysteine proteases 3CL-PR or PL-PR comprising administering a compound of claim 1 to a subject.

28. A method of treating a coronavirus infection comprising administering a compound of claim 1 to a subject.

29. (canceled)

30. (canceled)

31. (canceled)

32. A method of inhibiting cruzain, cruzipain, human cathepsin B, human cathepsin L, TbCatB, TbCatL, rhodesain, brucipain, falcipain-2, and falcipain-3, or coronaviral cysteine proteases 3CL-PR or PL-PR, or a combination thereof comprising administering a compound of claim 1 to a subject.

33. A method of treating a coronavirus infection, Chagas disease, African trypanosomiasis, or malaria comprising administering a compound of claim 1 to a subject.

34. A method of killing or inhibiting the growth of a protozoan, the method comprising: contacting the protozoan with the compound of claim 1, in an amount effective to kill or inhibit the growth of the protozoan.

35. (canceled)

36. (canceled)
